SEQUENCE LISTING (5 sequences)

SEQ ID NO: 1 - DNA, 60 bp, Homo sapiens
tggtggcggcggttggggctcggcgctcgctcgctcgctcgctgggcgggcggtgcgatg

SEQ ID NO: 2 - DNA, 42 bp, Artificial Sequence
  Description of Artificial Sequence Synthetic oligonucleotide
gcccgggcaaagcccgggcgtcgggcgacctttggtcgcccg

SEQ ID NO: 3 - protein, 6 aa, Artificial Sequence
  Description of Artificial Sequence Synthetic peptide
HHHHHH

SEQ ID NO: 4 - DNA, 159 bp, Adeno-associated virus
aggaacccctagatggagttggccactccctctctgcgcgctcgctcgctcactgaggccgggcccgaaacgggcccgtcgggcgacctttggtcgcccggcctcagtgagcgagcgagcgcgcagagagggagtggccaactccatctaggggttcct

SEQ ID NO: 5 - DNA, 469 bp, Homo sapiens
aggttacagagtggtgaaggcactctgcatttcttggttgagacagagaaaaaaagtggtcagaactgggtaaccctccccccaccatattatcacagtgatcccttttgtctttcttcaggctccagccccaccctacagcccctgctccctggattcactagagctaacttcagtaaagtacaaagaaaatggggccatatgactggccaaaaaaaaaatatctattcacgtggatgaccagatagtatgaatggattgaaaatttatcaggaaaaaaggatgagaggaaatgccaggagatgagggcagagagcaggccgttctgggggagggattctgtggggacagggtggcctactgggtgtgccccttttctcttctctgtctcccttagataagaccagcagttttgtcatcctctccctctcattccatggtcccgcagccccaggcccacactgaaagc

(19) United States
(12) Patent Application Publication
(10) Pub. No.: US 2025/0087304 A1
(43) Pub. Date: Mar. 13, 2025

(54) GENOMIC SAFE HARBORS

(71) Applicants: Synteny Therapeutics, Inc., Cambridge, MA (US); University of Massachusetts, Boston, MA (US)

(72) Inventors: Robert KOTIN, Cambridge, MA (US); Charlotte McGuinness, Cambridge, MA (US); Sebastian Aquirre, Cambridge, MA (US); Shannon Loncar, Southborough, MA (US); Robert Gifford, Glasgow (GB); Matthew A. Campbell, Palmer, AR (US); Marco Antonio Quezada Ramirez, Worcester, MA (US)

(21) Appl. No.: 18/562,737

(22) PCT Filed: May 19, 2022
(86) PCT No.: PCT/US22/30024

§ 371 Date: Nov. 20, 2023

Related U.S. Application Data
(60) Provisional application No. 63/190,996, filed on May 20, 2021.

(51) Int. Cl. G16B 25/10; A01K 67/0278; C12N 15/86

(52) U.S. Cl. CPC G16B 25/10; A01K 67/0278; C12N 15/86; A01K 2217/072; A01K 2227/105; C12N 2750/14143

(57) ABSTRACT

Disclosed are compositions comprising genomic safe harbor (GSH) loci and methods using same. Further disclosed are methods of identifying novel GSH loci.

### CROSS-REFERENCE TO RELATED APPLICATIONS

This application claims the benefit of priority to U.S. Provisional Application No. 63/190,996, filed May 20, 2021; the entire contents of which are incorporated herein in their entirety by this reference.

### BACKGROUND

The modification of the human genome by the stable insertion of functional transgenes and other genetic elements is of great value in biomedical research and medicine (e.g., for gene therapy). Genetically modified human cells are also valuable for the study of gene function, and for tracking and lineage analyses using reporter systems. All these applications depend on the reliable function of the introduced genes in their new environments. However, randomly inserted genes are subject to position effects and silencing, making their expression unreliable and unpredictable. Centromeres and sub-telomeric regions are particularly prone to transgene silencing.

Reciprocally, newly integrated genes may affect the surrounding endogenous genes and chromatin, potentially altering cell behavior or favoring cellular transformation. Thus, despite the successes of therapeutic gene transfer, there have been cases of malignant transformation associated with insertional activation of oncogenes following stem cell gene therapy, emphasizing the importance of where newly integrated DNA locates. In addition, the insertion of foreign DNA into the genome of progenitor cells may adversely affect terminal differentiation into specific cell types.

A genomic safe harbor (GSH) refers to a genetic locus that accommodates the insertion of exogenous DNA with either constitutive or conditional/inducible expression activity without significantly affecting the viability of somatic cells, progenitor cells, or germ line cells and ontogeny. The availability of the GSH loci is extremely useful to express reporter genes, suicide genes, selectable genes, or therapeutic genes.

Three intragenic sites have been proposed as GSHs (AAVS1, CCR5 and ROSA26 and albumin in murine cells) (see, e.g., U.S. Pat. Nos. 7,951,925; 8,771,985; 8,110,379; 7,951,925; U.S. Publication Nos. 20100218264; 20110265198; 20130137104; 20130122591; 20130177983; 20130177960; 20150056705 and 20150159172; all are incorporated by reference). However, these proposed GSHs are in relatively gene-rich regions and are near genes that have been implicated in cancer. Genes that are adjacent to AAVS1 may be spared by some promoters, but safety validation in multiple tissues remains to be carried out. Also, the dispensability of the disrupted gene, especially after biallelic disruption, as is often the case with endonuclease-mediated targeting, remains to be investigated further.

Accordingly, there is a great need for identification and validation of additional GSH loci, as well as various compositions and methods for the identified GSH loci.

### SUMMARY OF INVENTION

The present invention is based, at least in part, on the discovery that the novel GSH loci identified herein are particularly useful in stable insertion and predictable expression of various transgenes necessary for e.g., treating patients (e.g., via gene therapy) or preparing medicament (e.g., biologics or vaccines).

In certain aspects, provided herein are various methods of identifying novel GSH loci. Such methods include functional assays as well as in silico approaches. Further provided herein are various in vitro, ex vivo, and in vivo methods for validating the identified GSHs, which include: de novo targeted insertion of a marker gene into the GSH locus in a cell (e.g., human cell) to assess the insertion efficiency and the level of expression of the marker gene; targeted insertion of a marker gene into the GSH locus in a progenitor cell or stem cell to determine its impact on the differentiation of the progenitor cell or stem cell in vitro; targeted insertion of a marker gene into the locus in a progenitor cell or stem cell and engraft the cell into immune-depleted mice to determine the marker gene expression in all developmental lineages in vivo; targeted insertion of a marker gene into the GSH locus in a cell and determine the global cellular transcriptional profile (e.g., using RNAseq or microarray) to determine the impact of insertion at a GSH locus on the overall transcriptional profile of the cell; and/or generate a transgenic knock-in mouse where the genomic DNA of the mouse has a marker gene inserted in the locus.

In certain aspects, provided herein are various compositions comprising the GSH loci described herein. For example, provided herein are nucleic acid vectors comprising at least a portion of the GSH nucleic acid described herein. In preferred embodiments, the sequences with homology to GSH loci (5′ and 3′ homology arms) flank at least one non-GSH nucleic acid, such that the homology arms facilitate integration of the at least one non-GSH nucleic acid into the GSH locus. Such non-GSH nucleic acid may comprise a nucleic acid encoding a protein or a fragment thereof, e.g., a human protein or a fragment thereof; a therapeutic protein or a fragment thereof, an antigen-binding protein, or a peptide; a suicide gene, e.g., Herpes Simplex Virus-1 Thymidine Kinase (HSV-TK); a viral protein or a fragment thereof; a nuclease; a marker; and/or a drug resistance protein. Also provided herein are viral vectors comprising various nucleic acid vectors of the present disclosure. Further provided herein are cells comprising the nucleic acid vectors of the present disclosure, as well as cells comprising at least one non-GSH nucleic acid integrated into a GSH in the genome. In addition, pharmaceutical compositions comprising the nucleic acid vectors, viral vectors, and/or cells are provided, along with transgenic organisms comprising at least one non-GSH nucleic acid integrated into a GSH in the genome of a cell.

In certain aspects, provided here are methods of using and producing the compositions described herein. Such methods include a method of preventing or treating various diseases; a method of modulating the level and/or activity of a protein in a cell or in a subject (e.g., increasing a protein level by introducing an extra copy of the gene encoding said protein, or decreasing a protein level by introducing non-coding RNA and/or CRISPR gene editing that downregulates or eliminates the gene encoding said protein); a method of manufacturing biologics, such as antigen-binding proteins and/or therapeutic proteins (e.g., insulin); a method of manufacturing viral vectors, including those for gene therapy. Further provided herein are compositions and methods for integrating a viral surface protein at a GSH locus of the present disclosure, which allows in vivo immunization by exposing a viral antigen to a subject to induce immune response. Importantly, such viral antigen can be turned on and off intermittently by using an inducible promoter of the present disclosure that allow pulsatile expression of the viral antigen.

### BRIEF DESCRIPTION OF FIGURES

FIG. 1 shows current challenges for a safe gene therapy and the possible consequences of indiscriminate (random) DNA integration. There is mounting evidence that indiscriminate gene therapeutic integration can drive insertional mutagenesis, genotoxicity, or affect the gene of interest (e.g., encompassed herein by a non-GSH nucleic acid) expression, representing a major barrier to realizing the promise of gene therapy.

FIG. 2A and FIG. 2B show targeted integration into a GSH enables predictable transgene expression and reduces the risk of insertional mutagenesis in the host genome.

FIG. 2B shows that syntenic GSH bring predictability across relevant research models, facilitating non-clinical and clinical development. The use of safe, well characterized genomic loci for permanent transgenesis may well become a pre-requisite for safe and successful ex vivo and in vivo gene therapy treatments.

FIG. 3 shows a diagram of a representative method for identifying GSH loci.

FIG. 4A-FIG. 4C show characterization of a novel GSH locus. CFU (colony forming unit) assay to test differentiation potential of human CD34+ hematopoietic stem cell (HSC). FIG. 4A is a schematic diagram showing the assays performed herein. Gene directed integration into SYNTX-GSH1, a novel GSH locus identified herein, allowed successful HSC differentiation to committed erythroid progenitors. FIG. 4B shows high transgene expression (GFP) in committed erythroid progenitors. FIG. 4C shows a diagram illustrating HSC differentiation (erythropoiesis).

FIG. 5A-FIG. 5B show gene editing of a marker gene into GSH loci identified herein. FIG. 5A shows the efficiency of gene editing into the GSHs in CD34+ HSC identified herein. AAVS1, a previously known GSH locus was used as a positive control.

FIG. 5B shows that differentiation of primary CD34+ HSC into committed CD71+/CD235a+ erythroblasts was not affected after gene insertion into SYNTX-GSHs (SYNTX-GSH1 and SYNTX-GSH2).

FIG. 6A-FIG. 6B show the expression of the marker gene (GFP) integrated into different GSH loci. The GFP expression was determined 14 days after gene editing into the SYNTX-GSHs and AAVS1 (a positive control) in CD34+ HSC. (SYNTX-GSH1 and SYNTX-GSH2). Gene editing into SYNTX-GSH was more efficient than editing into AAVS1. The edited cells stably expressed GFP two weeks after gene editing and proceeded with differentiation from CD34+ HSC to erythroid progenitors. SYNTX-GSH1 and 2 edited cells expressed higher levels of transgene (GFP) than AAVS1 edited cells. (SYNTX-GSH1 and SYNTX-GSH2).

FIG. 7A-FIG. 7D show the impact of transgene knock-in into the SYNTX-GSH on global transcriptional profile of the cell. FIG. 7A shows the cell perturbation analysis experimental design by RNAseq. FIG. 7B shows the RNAseq analysis performed for SYNTX-GSH1 and SYNTX-GSH2 as compared with the wild-type cell and AAVS1. FIG. 7C shows the principal component analysis. FIG. 7D shows the integrated marker gene GFP expression in knock-in cell lines. Transgene integration into SYNTX-GSH had a lower impact on the cellular transcriptional profile than integration into AAVS1 site. SYNTX-GSH1 and SYNTX-GSH2 showed higher and more stable transgene expression than AAVS1 in human cells.

FIG. 8A-FIG. 8C assess the GSH performance by determining the stability of GFP expression over cell passages. FIG. 8A shows a schematic diagram of the experiment. FIG. 8B and FIG. 8C show the expression of the marker gene (GFP) inserted at the SYNTX-GSH loci. Transgene integration into four different SYNTX-GSH loci resulted in different editing efficiency and transgene expression. SYNTX-GSH1 and SYNTX-GSH2 showed higher and more stable transgene expression than AAVS1. SYNTX-GSH3 and SYNTX-GSH4 showed lower level of expression, and may be useful in insertion of a gene that requires lower level of expression (e.g., lethal gene). The GSH loci identified herein provide a palette of individual GSH with different characteristics to adapt to specific gene therapy programs.

FIG. 9A and FIG. 9B show a secondary structure of AAV ITR and a schematic diagram of a rolling hairpin replication model. FIG. 9A shows the structure of AAV ITR that forms an extensive secondary structure. The ITR can acquire two configurations (flip and flop). FIG. 9B shows a schematic diagram showing the rolling hairpin replication model by which a viral nucleic acid replicates.

FIG. 10 shows schematic diagrams representing a heterologous nucleic acid/a transgene construct containing a β-globin gene operably linked to a β-globin promoter flanked at the 5′ terminus by one or more HS sequences. Mammalian β-globin gene is regulated by a regulatory region called the locus control region (LCR) containing a series of 5 DNase I hypersensitive sites (HS1-HS5). The HSs is required for efficient expression of the β-globin gene. Each transgene construct is placed between two homology arms (a 5′ homology arm and a 3′ homology arm), which facilitates site-specific integration at a target cell genome by homologous recombination.

FIG. 11 shows schematic diagrams representing a heterologous nucleic acid/a transgene construct containing various promoters. Each promoter (e.g., CAG promoter, AHSP promoter, MND promoter, W-A promoter, PKLR promoter) is operably linked to a transgene of interest, and the entire construct is placed between two homology arms (a 5′ homology arm and a 3′ homology arm), which facilitates site-specific integration at a GSH locus of a target cell genome by homologous recombination.

FIG. 12 shows partial DNA sequence of the erythroid-specific promoter of PKLR. A 469-bp region comprising the upstream regulatory domain. Conserved elements between the human and rat PK-R promoter are depicted by dotted lines. The cytosine of the PK-R transcriptional start site is underlined. GATA-1, CAC/Sp1 motifs, and the regulatory element PKR-REI in the upstream 270-bp region are shown in boxes (orientation indicated by arrows).

FIG. 13A and FIG. 13B show exemplary miRNAs that can be targeted by the recombinant virions described herein. The erythroparvoviral recombinant virions may comprise the miRNA sequences. Alternatively, the recombinant virions may comprise a nucleic acid sequence that inactivates the miRNAs.

FIG. 14 shows pulsatile transgene expression systems. The schematic diagrams show both negative and positive regulation of expression. Example I (upper panel) shows that an ASO (an antisense oligonucleotides ASO or AON) can negatively regulate gene expression post-transcriptionally. Without ASO, a primary transcript (left) is spliced into a translatable mRNA (top line). The addition of an ASO (red line) complementary to the splice acceptor at the 3′ end of the intron/5′ end of Exon 2 interferes with splicing. Thus, in the presence of ASO, the intron remains in the transcript. The unprocessed RNA is either untranslatable or produces a non-functional protein upon translation. Example II (lower panel) illustrates that an ASO can positively affect gene expression post-transcriptionally. A primary transcript (left) contains 4 exons: exon 1, exon 3, and exon 4 encode the therapeutic protein, and exon 2 contains either a nonsense mutation(s) or an out-of-frame-mutation (OOF). Such exon 2 can be engineered into any transgene. Without the ASO, the transcript is processed into a mature mRNA comprising 4 exons (bottom line), i.e., exon 2 with a nonsense mutation(s) or an OOF mutation remains. Thus, the resulting mRNA translates into a truncated or non-functional protein. By contrast, the addition of ASO interferes with splicing, and the mature mRNA consists of exon 1, exon 3, and exon 4, i.e., exon 2 with a nonsense mutation(s) or an OOF mutation is spliced out. Thus, at the default state (no ASO), the therapeutic protein is not produced. Only upon the addition of ASO, the therapeutic protein is produced, thereby resulting in positive regulation.

FIG. 15 shows ATACseq Coverage and Peaks. The EVE insertion site is shown as a vertical black line at the center of plots. For each donor, ATACseq coverage is shown as a smoothed grey line with called peaks as vertical bars color-coded by donor. The distance from the EVE insertion to nearest peak across donors is 1,144 base pairs indicating accessible chromatin.

### DETAILED DESCRIPTION OF THE INVENTION

In certain aspects, provided herein are novel methods of identifying and validating GSH loci, newly identified GSH loci, compositions comprising the sequences of said GSH loci, and methods of using the GSH loci and compositions comprising same for treating patients (e.g., via gene therapy or cell therapy), preparing medicament (e.g., biologics or vaccines), and other applications described herein.

### Definitions

The articles “a” and “an” are used herein to refer to one or to more than one (i.e. to at least one) of the grammatical object of the article. By way of example, “an element” means one element or more than one element.

The term “administering” is intended to include routes of administration which allow a therapy to perform its intended function. Examples of routes of administration include injection (intramuscular, subcutaneous, intravenous, parenterally, intraperitoneally, intrathecal, intratumoral, intranasal, intracranial, intravitreal, subretinal, etc.) routes. The routes of administration also include inhalation as well as direct injection to the bone marrow. The injection can be a bolus injection or can be a continuous infusion. Depending on the route of administration, the agent can be coated with or disposed in a selected material to improve absorption or to protect it from natural conditions which may detrimentally affect its ability to perform its intended function.

The term “cetacea” refers to the taxonomic (infra) order of aquatic marine mammals comprising among others, baleen whales, toothed whales, dolphins and porpoises, and related forms and that have a torpedo-shaped nearly hairless body, paddle-shaped forelimbs but no hind limbs, one or two nares opening externally at the top of the head, and a horizontally flattened tail used for locomotion.

The term “chiroptera” refers to the taxonomic order of mammals capable of true flight, and comprise bats.

As used herein, “a donor sequence” refers to a polynucleotide that is to be inserted into, or used as a repair template for, a host cell genome. The donor sequence can comprise the modification which is desired to be made during gene editing. The sequence to be incorporated can be introduced into the target nucleic acid molecule via homology directed repair at the target sequence, thereby causing an alteration of the target sequence from the original target sequence to the sequence comprised by the donor sequence. Accordingly, the sequence comprised by the donor sequence can be, relative to the target sequence, an insertion, a deletion, an indel, a point mutation, a repair of a mutation, etc. The donor sequence can be, e.g., a single-stranded DNA molecule; a double-stranded DNA molecule; a DNA/RNA hybrid molecule; and a DNA/modRNA (modified RNA) hybrid molecule. In embodiments, the donor sequence is foreign to the homology arms. The editing can be RNA as well as DNA editing. The donor sequence can be endogenous to or exogenous to the host cell genome, depending upon the nature of the desired gene editing.

The term “endogenous viral element” or “EVE” is a DNA sequence derived from a virus, and present within the germline of a non-viral organism. EVEs may be entire viral genomes (proviruses), or fragments of viral genomes. They arise when a viral DNA sequence becomes integrated into the genome of a germ cell that goes on to produce a viable organism. The newly established EVE can be inherited from one generation to the next as an allele in the host species, and may even reach fixation.

The term “homologous recombination” is art-recognized, and when used in relation to a nucleic acid insertion in a target genome, it is intended to include homology-dependent repair.

The term “homology” or “homologous” as used herein is defined as the percentage of nucleotide residues in the homology arm that are identical to the nucleotide residues in the corresponding sequence on the target chromosome, after aligning the sequences and introducing gaps, if necessary, to achieve the maximum percent sequence identity. Identity as between regions of nucleic acid sequences can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I): 387 (1984)), BLASTP, BLASTN, FASTA Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). In some embodiments, a nucleic acid sequence (e.g., DNA sequence), for example of a homology arm of a repair template, is considered “homologous” when the sequence is at least or about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the corresponding native or unedited nucleic acid sequence (e.g., genomic sequence) of the host cell.

As used herein, a “homology arm” refers to a polynucleotide that is suitable to target a donor sequence to a genome through homologous recombination. Typically, two homology arms flank the donor sequence, wherein each homology arm comprises genomic sequences upstream and down-stream of the loci of integration.

The term “lagomorpha” refers to the taxonomic order of gnawing herbivorous mammals having two pairs of incisors in the upper jaw one behind the other, usually soft fur, and short or rudimentary tail, made up of two families (Leporidae and Ochotonidae genera that comprise the Leporidae family) comprising the rabbits, hares, and pikas.

The term “Macropodidae” refers to the taxonomic family of diprotodont marsupial mammals comprising the kangaroos, wallabies, and rat kangaroos that are all saltatory animals with long hind limbs and weakly developed forelimbs and are typically inoffensive terrestrial herbivores.

The term “monotremata” refers to the taxonomic order of egg-laying mammals comprising the platypuses and echidnas.

The term “provirus” refers to the genome of a virus when it is integrated or inserted into a host cell's DNA. Provirus refers to the duplex DNA form of the retroviral genome linked to a cellular chromosome. The provirus is produced by reverse transcription of the RNA genome and subsequent integration into the chromosomal DNA of the host cell.

The term “primates” refers to the taxonomic order of mammals that are characterized especially by advanced development of binocular vision resulting in stereoscopic depth perception, specialization of the hands and feet for grasping, and enlargement of the cerebral hemispheres and include humans, apes, monkeys, and related forms (such as lemurs and tarsiers).

As used herein, “Rep” refers to any non-structural replicase, a Rep protein, or a combination of Rep proteins that is/are capable of providing the necessary function(s) to allow for replication of the viral genome.

The term “Rodentia” refers to the taxonomic order of relatively small gnawing mammals (such as a mouse, squirrel, or beaver) that have in both jaws a single pair of incisors with a chisel-shaped edge. It includes all rodents.

The term “subject” or “patient” refers to any healthy or diseased animal, mammal or human, or any animal, mammal or human. In some embodiments, the subject is afflicted with a hematologic disease. In various embodiments of the methods of the present invention, the subject has not undergone treatment. In other embodiments, the subject has undergone treatment.

The term “syntenic” refers to similar organization or ordering of a series of genes in different species.

A “therapeutically effective amount” of a substance or cells or virions is an amount capable of producing a medically desirable result (e.g., clinical improvement) in a treated patient with an acceptable benefit: risk ratio, preferably in a human or non-human mammal.

The term “taxonomic order” refers to orderly classification of plants and animals according to their presumed natural relationships. Species relatedness, based on analysis of genomic sequence data provides a quantitative alternative approach to the natural relationships deduced from physical relationships.

The term “treating” includes prophylactic and/or therapeutic treatments. The term “prophylactic or therapeutic” treatment is art-recognized and includes administration to the subject one or more of the compositions described herein. If it is administered prior to clinical manifestation of the unwanted condition (e.g., disease or other unwanted state of the subject), then the treatment is prophylactic (i.e., it protects the subject against developing the unwanted condition): whereas, if it is administered after manifestation of the unwanted condition, the treatment is therapeutic (i.e., it is intended to diminish, ameliorate, or stabilize the existing unwanted condition or side effects thereof).

### Genomic Safe Harbors (GSHs)

The term “Genomic Safe Harbor,” also interchangeably referred to herein as “GSH” or “safe harbor gene” or “safe harbor locus,” refers to a location within a genome, including a region of genomic DNA or a specific site, that can be used for integrating an exogenous nucleic acid wherein the integration does not cause any significant deleterious effect on the growth of the host cell by the addition of the exogenous nucleic acid alone. That is, a GSH refers to a gene or locus in the genome that a nucleic acid sequence can be inserted such that the sequence can integrate and function in a predictable manner (e.g., express a protein of interest) without significant negative consequences to endogenous gene activity, or the promotion of cancer. For example, a GSH is a site in the host cell genome that is able to accommodate the integration of new genetic material in a manner that ensures that the newly inserted genetic elements (i) function predictably (e.g., predictable expression) and (ii) do not cause significant alterations of the host genome thereby averting a risk to the host cell or organism, and (iii) preferably the inserted nucleic acid is not perturbed by any read-through expression from neighboring genes, and (iv), does not activate nearby genes. GSHs can be a specific site, or can be a region of the genomic DNA. A GSH can be a chromosomal site where transgenes can be stably and reliably expressed in all tissues of interest without adversely affecting endogenous gene structure or expression. In some embodiments, a GSH is a locus or gene where an insertion of an exogenous nucleic acid does not alter significantly the cell's ability to differentiate properly (e.g., differentiation of a stem cell). In some embodiments, a GSH is also a locus or gene where an inserted nucleic acid sequence can be expressed efficiently and at higher levels than a non-safe harbor site.

Accordingly, GSHs comprise intragenic, intergenic, or extragenic regions of the human and model species genomes that are able to accommodate the predictable expression of newly integrated DNA without significant adverse effects on the host cell or organism. GSHs may comprise intronic or exonic gene sequences as well as intergenic or extragenic sequences. While not being limited to theory, a useful safe harbor must permit sufficient transgene expression to yield desired levels of the transgene-encoded protein or non-coding RNA. A GSH also should not predispose cells to malignant transformation, nor interfere with progenitor cell differentiation, nor significantly alter normal cellular functions. What distinguishes a GSH from a fortuitous good integration event is the predictability of outcome, which is based on prior knowledge and validation of the GSH.

In some embodiments, GSH allows safe and targeted gene delivery that has limited off-target activity and minimal risk of genotoxicity, or causing insertional oncogenesis upon integration of foreign DNA, while being accessible to highly specific nucleases with minimal off-target activity.

### Identifying Genomic Safe Harbors

Provided herein are exemplary methods of identifying GSH loci. In some embodiments, any one of the exemplary methods is used to identify GSH loci. In some embodiments, a combination of at least two exemplary methods are used to identify GSH loci. In some embodiments, a combination of at least three exemplary methods are used to identify GSH loci. Any one or combination of multiple exemplary methods may optionally further comprise at least one assay (in vitro, ex vivo, or in vivo) to validate the identified GSH loci.

### Method 1: Functional Identification of GSH Loci Via Random Integration of a Marker

In certain aspects, provided herein is a method of identifying a genomic safe harbor (GSH) locus, comprising: (a) inducing a random insertion of at least one marker gene into a genome in a cell; (b) determining the stability and/or level of the marker gene expression; and (c) identifying a genomic locus, wherein the inserted marker gene shows the stable and/or high level of the expression, as a GSH. In preferred embodiments, the method further comprises (a) identifying a genomic locus, wherein the inserted marker gene does not affect cell viability; and/or (b) identifying a genomic locus, wherein the inserted marker does not affect the cell's ability to differentiate. Accordingly, in some embodiments, an insertion of a marker gene in the GSH locus does not affect the pluripotency, totipotency, or mulipotency of a cell (e.g., a stem cell or a progenitor cell).

In some embodiments, the cell used in the method is selected from a cell line, a primary cell, a stem cell, or a progenitor cell. In some embodiments, the cell is a stem cell. In some such embodiments, the stem cell is selected from an embryonic stem cell, a tissue-specific stem cell, a mesenchymal stem cell, and an induced pluripotent stem cell (iPSC).

In some embodiments, the cell used in the method is selected from a hematopoietic stem cell, a hematopoietic CD34+ cell, and epidermal stem cell, an epithelial stem cell, neural stem cell, a lung progenitor cell, and a liver progenitor cell.

In some embodiments, the cell used in the method is a mammalian cell. In some such embodiments, the mammalian cell is a mouse cell, a dog cell, a pig cell, a non-human primate (NHP) cell, or a human cell.

In certain embodiments, the random insertion of at least one marker gene into a genome in a cell is induced by: (a) transfecting the cell with a nucleic acid molecule comprising the marker gene, optionally wherein the nucleic acid is a plasmid; or (b) transducing the cell with an integrating virus comprising the marker gene. In some embodiments, the random insertion is induced by transducing the cell with an integrating virus comprising the marker gene; and the integrating virus is a retrovirus. In some embodiments, the retrovirus is a gamma retrovirus.

In certain embodiments, the method uses the at least one marker gene comprising a screenable marker and/or a selectable marker. In some embodiments, the screenable marker gene encodes a green fluorescent protein (GFP), beta-galactosidase, luciferase, and/or beta-glucuronidase. In some embodiments, the selectable marker gene is an antibiotic resistance gene. In some such embodiments, the antibiotic resistance gene encodes blasticidin S-deaminase or amino 3′-glycosyl phosphotransferase (neomycin resistance gene).

In certain embodiments, the method uses a marker gene that is not operably linked to a promoter. Here, the use of a promoter-less marker allows identification of the GSH loci that permits expression of an exogenous nucleic acid using the neighboring promoter and regulatory elements. In some embodiments, the neighboring promoter is a tissue-specific promoter.

In certain embodiments, the marker gene is operably linked to a promoter. In some embodiments, the promoter is a tissue-specific promoter.

In some embodiments, the identified GSH is intragenic (e.g., exonic or intronic) or intergenic. In preferred embodiments, the identified GSH is intronic or intergenic.

### Method 2: Identifying GSH Loci Using an Endogenous Virus Elements (EVE)

In certain aspects, provided herein is a method of identifying a GSH locus using evolutionary biology to identify, e.g., any provirus remnants (e.g., parvovirus remnants), referred to as endogenous virus elements (EVEs), in the genome of a metazoan species. The results described herein demonstrate that EVEs can be acquired into the germline of a progenitor species prior to the radiation of the species, such that all evolved or descendent species retain the EVE allele. Whereas closely related species that evolved or radiated prior to the “endogenization” event retain empty loci. As an illustrative example only, the locus occupied by intergenic EVE in the Macropodidae (kangaroos and related species) is identifiable in other marsupials, including Didelphis virgiana (North American opossum). These unoccupied loci are identifiable in other taxonomic families and although the EVE open reading frames are disrupted, the virus sequence represents foreign DNA inserted into the genome of the totipotent germ cell, thus identifying candidate genomic safe-harbor loci. The rationale for identifying an EVE as a GSH locus is that an insertion at the EVE locus did not affect viability, function, growth, differentiation, and speciation of an organism, thereby providing an inert site that allows insertion of an exogenous nucleic acid.

In some embodiments, the EVE is intragenic or intergenic. In some embodiments, the EVE is intragenic. In some embodiments, the EVE is intronic or exonic. In some embodiments, the EVE is intronic. For instance, in some embodiments, the GSH locus is an exonic locus that has tolerated an insertion of EVE(s) in the evolutionary lineage. In preferred embodiments, the GSH is an intronic or intergenic locus. For such a locus, there is a lower chance of disrupting the function and structure of nearby genes or regulatory sequences via an insertion of an exogenous nucleic acid that is actively transcribed.

In certain aspects, provided herein is a method of identifying a GSH locus, the method comprising: (a) determining the presence and location of an endogenous virus element (EVE) in the genome of a metazoan species; (b) determining intergenic or intronic boundaries proximal to the EVE; and (c) identifying an intergenic or intronic locus comprising the EVE as a GSH locus.

In some embodiments, the presence and location of an EVE are determined by searching in silico for sequences homologous to a virus element. In some embodiments, the EVE in the metazoan species comprises a sequence that is at least, about, or no more than 10%, 15%, 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the sequence of a virus element.

In some embodiments, the intergenic or intronic boundaries proximal to the EVE are determined by aligning the sequences flanking the EVE and its orthologous sequences of one or more species whose intergenic or intronic boundaries are known. In some embodiments, the intergenic or intronic boundaries proximal to the EVE comprise a sequence that is at least, about, or no more than 10%, 15%, 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the sequence of an orthologous sequence in one or more species whose intergenic or intronic boundaries are known.

In some embodiments, the method identifies a GSH locus is in a mammalian genome, optionally wherein the mammalian genome is a mouse genome, a dog genome, a pig genome, a NHP genome, or a human genome.

In some embodiments, the EVE comprises a provirus, which is the virus genome integrated into the DNA of a non-virus host cell. In some embodiments, the EVE comprises a portion or fragment of a viral genome. In some embodiments, the EVE comprises a provirus from a retrovirus. In some embodiments, the EVE is not from a retrovirus. In some embodiments, the EVE comprises a provirus or fragment of a viral genome from a non-retrovirus.

In some embodiments, the EVE comprises a viral nucleic acid, viral DNA, or a DNA copy of viral RNA. In some embodiments, the EVE comprises viral nucleic acid. In some embodiments, EVE or viral nucleic acid in EVE encodes a structural or a non-structural viral protein, or a fragment thereof.

In some embodiments, the EVE comprises viral nucleic acid from a retrovirus. In some embodiments, the EVE comprises viral nucleic acid from a non-retrovirus, parvovirus, and/or circovirus. In some embodiments, the parvovirus is selected from B19, minute virus of mice (mvm), RA-1, AAV, bufavirus, hokovirus, bocavirus, and any one of the parvoviruses described herein (e.g., a parvovirus listed in Tables 1A-1D). In some embodiments, the parvovirus is AAV. In some embodiments, the viral nucleic acid is from a circovirus. In some embodiments, the circovirus is porcine circovirus (PCV) (e.g., PCV-1, PCV-2). In some embodiments, the viral nucleic acid in the EVE comprises a non-retroviral nucleic acid. In some embodiments, the non-retroviral nucleic acid encodes a non-structural or a structural viral protein (e.g., rep (replication) protein, or cap (capsid) protein, respectively).

In some embodiments, the EVE or the viral nucleic acid encodes a structural or a non-structural viral protein. In some embodiments, the EVE or the viral nucleic acid encodes the Rep and assembly activating non-structural (NS) proteins (e.g., those required for viral replication, capsid assembly, etc.), and/or the structural(S) viral proteins (capsid proteins, e.g., VP). Such proteins include, but are not limited to, Rep (replication) proteins, including but not limited to Rep78, Rep68, Rep52, and Rep40; and Cap (capsid) proteins, including but not limited to VP1, VP2 and VP3, e.g., from AAV. Structural proteins also include but are not limited to structural proteins A, B, and C, for example, from AAV. In some embodiments, the EVE is a nucleic acid encoding all, or part of a non-structural (NS) protein or a structural(S) protein disclosed in Supplemental Table S2 in Francois et al. “Discovery of parvovirus-related sequences in an unexpected broad range of animals.” Nature Scientific reports 6 (2016).

In some embodiments, the method to identify a GSH in a mammalian genome comprises an initial sequencing and/or in silico analysis of the sequence of genomic DNA inferred from an progenitor species by multiple species within a taxonomic rank to identify endogenous virus element (EVE) or provirus nucleic acid insertions in the genomic DNA.

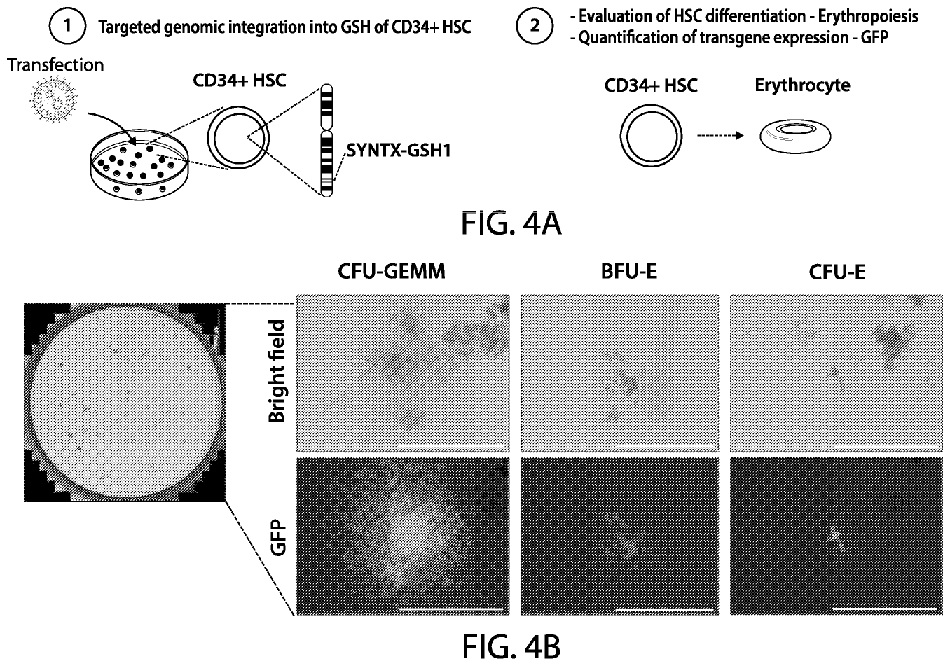

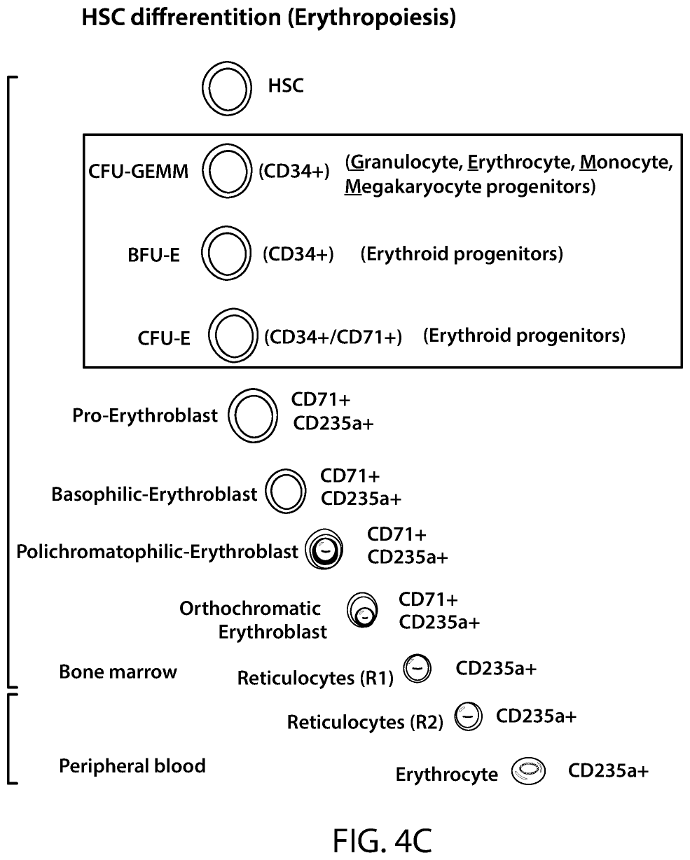

In some embodiments, the genome sequence of a metazoan species is analyzed for the presence of the EVE. The metazoan species can be from any phylogenetic taxa including, but not limited to, Cetacea, Chiropetera, Lagomorpha, and Macropodiadae. Accordingly, in some embodiments, the metazoan species is selected from Cetacea, Chiropetera, Lagomorpha, and Macropodiadae. Other metazoan species can also be assessed, for example, rodentia, primates, monotremata. Other species can be used, for example, as listed in FIG. 4A, 4B of Lui et al, J Virology 2011:9863-9876 which is incorporated herein in its entirety by reference.

In some embodiments, the EVE comprises nucleic acid from a parvovirus, a virus of the family Parvoviridae. The Parvoviridae family contains two subfamilies; Parvovirinae, which infect vertebrate hosts and Densovirinae, which infect invertebrate hosts. Each subfamily has been subdivided into several genera.

In some embodiments, the EVE comprises a nucleic acid from a Densovirinae, from any one of the following genera: Ambidensovirus, Brevidensovirus, Hepandensovirus, Iteradensovirus, and Penstyldensovirus.

In some embodiments, the EVE comprises a nucleic acid from a Parvovirinae, from any one of the following genera: Amdoparvovirus, Aveparvovirus, Bocaparvovirus, Copiparvovirus, Dependoparvovirus, Erythroparvovirus, Protoparvovirus, and Tetraparvovirus. In some embodiments, the EVE comprises a nucleic acid from Erythroparvovirus or Dependoparvovirus.

In some embodiments, the EVE is from the subfamily of Densovirinae include the following genera:
a. Genus Ambidensovirus. Type species: Lepidopteran Ambidensovirus 1. Genus includes 11 recognized species.
b. Genus Brevidensovirus. Type species: Dipteran Brevidensovirus 1. Genus includes 2 recognized species.
c. Genus Hepandensovirus. Type species: Decapod Densovirus 1. Genus includes a single recognized species.
d. Genus Iteradensovirus. Type species: Lepidopteran Iteradensovirus 1. Genus includes 5 recognized species.
e. Genus Penstyldensovirus. Type species: Decapod Penstyldensovirus 1. Genus includes a single recognized species.
f. Unassigned Genus. Type species: Orthopteran Densovirus 1. Genus includes a single recognized species.

In some embodiments, the EVE is from the subfamily of Parvovirinae include the following genera:
a. Genus Amdoparvovirus. Type species: Carnivore Amdoparvovirus 1. Genus includes 4 recognized species, infecting minks and foxes.
b. Genus Aveparvovirus. Type species: Galliform Aveparvovirus 1. Genus includes a single species, infecting turkeys and chickens.
c. Genus Bocaparvovirus. Type species: Ungulate Bocaparvovirus 1. Genus includes 21 recognized species, infecting mammals from multiple orders, including primates.
d. Genus Copiparvovirus. Type species: Ungulate Copiparvovirus 1. Genus includes 2 recognized species, infecting pigs and cows.
e. Genus Dependoparvovirus. Type species: Adeno-associated Dependoparvovirus A. Genus includes 7 recognized species, infecting mammals, birds or reptiles.
f. Genus Erythroparvovirus. Type species: Primate Erythroparvovirus 1. Genus includes 6 recognized species, infecting mammals, specifically primates, chipmunk or cows.
g. Genus Protoparvovirus. Type species: Rodent protoparvovirus 1. Genus includes 11 recognized species, infecting mammals from multiple orders, including primates.
h. Genus Tetraparvovirus. Type species: Primate tetraparvovirus 1. Genus includes 6 recognized species, infecting primates, bats, pigs, cows and sheep.

TABLE 1A
Exemplary viruses of Erythroparvovirus in Parvovirinae Subfamily

| | | Accession | |
|---|---|---|---|
| Species | Virus Name | No. | Ref Seq No. |
| Primate erythroparvovirus 1 | Human erythroparvovirus | AY386330 | NC_000883 |
| | B19 | | |
| Primate erythroparvovirus 2 | Simian parvovirus | U26342 | NC_038540 |
| Primate erythroparvovirus 3 | Rhesus macaque parvovirus | AF221122 | NC_038541 |
| Primate erythroparvovirus 4 | Pig-tailed macaque parvovirus | AF221123 | NC_038542 |
| Rodent erythroparvovirus 1 | Chipmunk parvovirus | GQ200736 | NC_038543 |
| Ungulate erythroparvovirus 1 | Bovine parvovirus 3 | AF406967 | N/A |

TABLE 1B
Exemplary viruses in Parvovirinae Subfamily

| Parvovirinae | | Accession |
|---|---|---|
| Genus | Virus species or variant | number |
| Amdoparvovirus | Aleutian mink disease virus | JN040434 |
| | Grey fox amdovirus | JN202450 |
| Aveparvovirus | Aveparvovirus Turkey parvovirus | JN202450 |
| Bocaparvovirus | California sea lion bocavirus 1 | JN202450 |
| | Canine bocavirus 1 | JN648103 |
| | Canine minute virus | FJ214110 |
| | Feline bocavirus | JQ692585 |
| | Human bocavirus 1 | JQ692585 |
| | Human bocavirus 4 | FJ973561 |
| | Porcine bocavirus 1 | HM053693 |
| | Porcine bocavirus 3 | JF429834 |
| | Porcine bocavirus 5 | HQ223038 |
| Copiparvovirus | Bovine parvovirus 2 | AF406966 |
| | Porcine parvovirus 4 | GQ387499 |
| Dependoparvovirus | Adeno-associated virus 1 | GQ387499 |
| | Adeno-associated virus 2 | NC_001401 |
| | Adeno-associated virus 3 | NC001729 |
| | Adeno-associated virus 3B | NC_001863 |
| | Adeno-associated virus 4 | NC_001829 |
| | Adeno-associated virus 5 | AF085716 |
| | Adeno-associated virus 6 | NC_001862 |
| | Adeno-associated virus 7 | AF513851 |
| | Adeno-associated virus 8 | AF513852 |
| | Avian-AAV ATCC VR-865 | NC_004828 |
| | Avian-AAV ATCC DA-1 | NC_006263 |
| | Bat adeno-associated virus | GU226971 |
| | Califonia sea lion adeno-associated | JN420372 |
| | virus 1 | |
| | Bovine AAV | NC_003889 |
| | Goose parvovirus | U25749 |
| Erythroparvovirus | Erythroparvovirus Human | M13178 |
| | parvovirus B19 | |
| Protoparvovirus | Bufavirus 1 | JX027296 |
| | Canine parvovirus | M19296 |
| | Mouse parvovirus 1 | U12469 |
| | Mouse parvovirus 3 | DQ196318 |
| | Porcine parvovirus PT4 | U44978 |
| | Rat parvovirus NTU1 | AF036710 |
| Tetraparvovirus | Bovine hokovirus | EU200669 |
| | Eidolon helvum parvovirus 1 | JQ037753 |
| | Human parvovirus 4 | AY622943 |
| | Porcine hokovirus | EU200677 |

TABLE 1C
Exemplary viruses of Protoparvovirus in Parvovirinae Subfamily

| | |
|---|---|
| Species of Protoparvovirus | Carnivore protoparvovirus |
| | Carnivore protoparvovirus 1 |
| | Chiropteran protoparvovirus 1 |
| | Eulipotyphla protoparvovirus 1 |
| | Primate protoparvovirus 1 |
| | Primate protoparvovirus 2 |
| | Primate protoparvovirus 3 |
| | Primate protoparvovirus 4 |
| | Rodent protoparvovirus 1 |
| | Rodent protoparvovirus 2 |
| | Rodent protoparvovirus 3 |
| | Ungulate protoparvovirus 1 |
| | Ungulate protoparvovirus 2 |

| Exemplary Viruses | Accession No. | Ref Seq No. |
|---|---|---|
| Sea otter parvovirus | KU561552 | NC_030837 |
| Canine parvovirus | M19296 | NC_001539 |
| Megabat bufavirus 1 | LC085675 | NC_029797 |
| Mpulungu (shrew) bufavirus | AB937988 | NC_026815 |
| Bufavirus 1a (human) | JX027296 | NC_038544 |
| Wuharv (rhesus) parvovirus 1 | JX627576 | NC_039049 |
| Cutavirus (human); | KT868811 | NC_039050 |
| Human Cutavirus 1 | | |
| Tusavirus; | KJ495710 | |
| Human tusavirus | | |
| Minute virus of mice | J02275 | NC_001510 |
| Rat parvovirus 1 | AF036710 | NC_038545 |
| Rat bufavirus SY-2015 | KT716186 | NC_028650 |
| Porcine parvovirus; | L23427 | NC_001718 |
| Porcine parvovirus 5 | | |
| Porcine bufavirus; | KT965075 | NC_043446 |
| Protoparvovirus (porcine) | | |
| Porcine parvovirus 2 | | NC_025965 |
| Porcine parvovirus 6 | | NC_023860 |
| Feline panelukepenia virus | FJ231389; | |
| | KP769859 | |
| Human bufavirus 1 | JQ918261 | |
| Human bufavirus 2 | JX027297 | |
| Human bufavirus 3 | AB847989 | |

TABLE 1D
Exemplary viruses of Tetraparvovirus in Parvovirinae Subfamily

| | |
|---|---|
| Species of Tetraparvovirus | Chiropteran tetraparvovirus 1 |
| | Primate tetraparvovirus 1 |
| | Ungulate tetraparvovirus 1 |
| | Ungulate tetraparvovirus 2 |
| | Ungulate tetraparvovirus 3 |
| | Ungulate tetraparvovirus 4 |

| Exemplary Viruses | Accession No. | Ref Seq No. |
|---|---|---|
| Eidolon helvum parvovirus | JQ037753 | NC_016744 |
| Human parvovirus 4 | AY622943 | NC_007018 |
| (Genotype 1) | | |
| Human parvovirus 4 | DQ873389.1 | |
| (Taxonomy ID: 289365) | | |
| Human parvovirus 4 | DQ873391.1 | |
| (Genotype 2) | | |
| (Taxonomy ID: 1511920) | | |
| Human parvovirus 4 | EU874248.1 | |
| (Genotype 3) | | |
| Bovine hokovirus 1 | EU200669 | NC_038898 |
| Bovine hokovirus 1 | EU200670.1 | |
| Bovine hokovirus 2 | KU172423.1 | |
| Porcine hokovirus; | EU200677 | NC_038546 |
| Porcine parvovirus 3 | | |
| Porcine parvovirus 2; | GU938300 | NC_038883 |
| Parvovirus YX-2010/CHN | | |
| Porcine Cnvirus | GU938301.1 | |
| (Taxonomy ID: 754189) | | |
| Ovine hokovirus | JF504699 | NC_038547 |
| Chimpanzee Parvovirus 4 | MH215556.1 | |
| (Taxonomy ID: 1511922) | | |
| Yak Parvovirus | | NC_028136 |
| Opossum tetraparvovirus | MG745671 | |
| Rodent tetraparvovirus | MG745670.1 | |
| Tetraparvovirus sp. | | NC_031670.1 |

The Parvovirinae subfamily is associated with mainly warm-blooded animal hosts. Of these, the RA-1 virus of the parvovirus genus, the B19 virus of the Erythrovirus genus, and the adeno-associated viruses (AAV) 1-9 of the Dependovirus genus are human viruses. In some embodiments, the EVE comprises a nucleic acid from a virus that can infect humans, which are recognized in 5 genera: Bocaparvovirus (human bocavirus 1-4, HboV1-4), Dependoparvovirus (adeno-associated virus; at least 12 serotypes have been identified), Erythroparvovirus (parvovirus B19, B19), Protoparvovirus (Bufavirus 1-2, BuV1-2) and Tetraparvovirus (human parvovirus 4 G1-3, PARV4 G1-3).

In some embodiments, the EVE is from a parvovirus, and in some embodiments the EVE comprises nucleic acid from an AAV (adeno-associated virus). Adeno-associated virus (AAV), a member of the Parvovirus family, is a small nonenveloped, icosahedral virus with single-stranded linear DNA genomes of 4.7 kilobases (kb) to 6 kb. AAV is assigned to the genus, Dependoparvovirus, because the virus was discovered as a contaminant in purified adenovirus stocks, was originally designated as adenovirus associated (or satellite) virus. AAV's life cycle includes a latent phase at which AAV genomes, after infection, may integrate into host cell chromosomal DNA frequently at a defined locus, such as, e.g., AAVS1, and a lytic phase in which, in which cells are co-infected with either adenovirus or herpes simplex virus and AAV, or superinfecting latent infected cells, the integrated genomes are subsequently rescued, replicated, and packaged into infectious viruses. Based on serological surveillance analyses, exposure to AAV is highly prevalent in humans and other primates and several serotypes have been isolated from various tissue samples. Serotypes 2, 3, 6, and 13 were discovered in cultured human cells, and AAV5 was isolated from a clinical specimen, whereas AAV serotypes 1, 4, and 7-12 were isolated from nonhuman primate (NHP) tissue samples or cells. As of 2013, there have been 13 AAV serotypes described. Weitzman, et al. (2011). “Adeno-Associated Virus Biology.” In Snyder, R. O.; Moullier, P. Adeno-associated virus methods and protocols. Totowa, NJ: Humana Press. ISBN 978-1-61779-370-7; Mori S, et al., (2004). “Two novel adeno-associated viruses from cynomolgus monkey: pseudotyping characterization of capsid protein.” Virology 330 (2): 375-83).

In some embodiments, the EVE comprises a nucleic acid or a portion of a nucleic acid from any of the parvoviruses listed in Tables 1A-1D; or a nucleic acid comprising a sequence with at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a nucleic acid or a portion of a nucleic acid from any of the parvoviruses listed in Tables 1A-1D

In some embodiments, the EVE comprises a nucleic acid or a portion of a nucleic acid from any serotype of AAV; or a nucleic acid comprising a sequence with at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a nucleic acid or a portion of a nucleic acid from any serotype of AAV. In some embodiments, the AAV is selected from the serotypes AAV1, AAV2, AAV3, AAV4, AAV5, AAV6, AAV7, AAV8, AAV9, AAV 10, AAV11, AAV12, or AAV13.

In some embodiments, the EVE comprises a nucleic acid sequence from any of the group selected from: B19, minute virus of mice (MVM), RA-1, AAV, bufavirus, hokovirus, bocavirus, or any of the viruses listed in Tables 1A-1D, or variants thereof, that is, virus with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% nucleic acid or amino acid sequence identity.

### Method 3: A Method of Identifying a GSH Locus in an Orthologous Organism

In certain aspects, provided herein is a method of identifying a GSH locus in an orthologous organism, the method comprising: (a) identifying a GSH locus in Species A according to any one of the methods described herein (e.g., using a functional method (Method 1), or a method utilizing an EVE (Method 2)); (b) determining the location of (i) at least one cis-acting element proximal to the GSH locus in Species A and (ii) the corresponding cis-acting element(s) in Species B; and (c) identifying a locus in Species B as a GSH locus, wherein the distance between the locus and the at least one cis-acting element in Species B is substantially proportional to the distance between the GSH locus and the corresponding cis-acting element(s) in Species A.

As described herein, the at least one cis-acting element proximal to a GSH locus in Species A and/or Species B may be known, or alternatively, the location of such elements may be determined by sequence analysis (e.g., by aligning the sequences flanking a GSH locus and their orthologous sequences in one or more organisms, wherein the at least one cis-acting element proximal to the GSH locus is known). In some embodiments, the at least one cis-acting element in Species A or Species B comprises a sequence that is at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the known cis-acting element in at least one orthologous organism. In some embodiments, the at least one cis-acting element proximal to the GSH locus in Species A is at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the at least one cis-acting element proximal to the GSH locus in Species B.

Alternatively, an ordinary skilled artisan would understand how to determine at least one cis-acting element proximal to the GSH locus by experimentation (e.g., determining the RNA sequence by RNA seq or by cloning a cDNA; and comparing it to the genomic sequence to map the splicing donor sites, splicing acceptor sites, polyadenylation sites, etc.).

Many cis-acting elements are known in the art. In some embodiments, the at least one cis-acting element is selected from a splicing donor site, a splicing acceptor site, a polypyrimidine tract, a polyadenylation signal, an enhancer, a promoter, a terminator, a splicing regulatory element, an intronic splicing enhancer, and an intronic splicing silencer.

In certain embodiments, the at least one cis-acting element comprises two or more cis-acting elements.

In some embodiments, the at least one cis-acting element comprises two cis-acting elements; and the first cis-acting element is located upstream (i.e., 5′ to) of the GSH locus, and the second cis-acting element is located downstream (i.e., 3′ to) of the GSH locus.

In some embodiments, the distance between the at least one cis-acting element and the GSH locus relative to the distance between two cis-acting elements in Species B is substantially proportional to the distance between the corresponding cis-acting element and the GSH locus relative to the distance between two cis-acting elements in Species A.

In some embodiments, the distance between the at least one cis-acting element to the GSH locus in Species B is at least, about, or no more than 1%, 5%, 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, or 1000% of the distance between the at least one cis-acting element to the GSH locus in Species A.

In some embodiments, the distance between the at least one cis-acting element to the GSH locus in Species B is at least 20% but no more than 500% of the distance between the at least one cis-acting element to the GSH locus in Species A.

In some embodiments, the distance between the at least one cis-acting element to the GSH locus in Species B is at least 80% but no more than 250% of the distance between the at least one cis-acting element to the GSH locus in Species A.

In some embodiments, the distance between the at least one cis-acting element to the GSH locus in Species B is at least 90% but no more than 110% of the distance between the at least one cis-acting element to the GSH locus in Species A.

In some embodiments, the method identifies a GSH locus in a mammalian genome. In some embodiments, the mammalian genome is a mouse genome, a dog genome, a pig genome, a NHP genome, or a human genome.

As indicated above, any one method of identifying a GSH locus may further comprise the steps and/or considerations in any other method, i.e., any number of methods described herein may be combined in any sequence. For example, the functional identification of a GSH locus by Method 1 may further comprise the steps and/or consideration of Method 2 (e.g., identifying EVEs). The Method 1 may further comprise the steps and/or consideration of Method 3 (e.g., identifying a GSH locus in an orthologous organism). Similarly, the Method 2 may further comprise the steps and/or consideration of Method 3. Alternatively, The Method 1 may further comprise the steps and/or consideration of Method 2 and Method 3.

### Optional Criteria for Selecting a GSH Locus or a Nucleic Acid Region of the GSH

In some embodiments, a GSH identified according to the methods described herein herein is an extragenic site or intergenic site that is remote from a known gene or a genomic regulatory sequence, or an intragenic site (within a gene) whose disruption is deemed to be tolerable.

In some embodiments, the GSH may comprise genes, including intragenic DNA comprising intronic or exonic gene sequences.

In some embodiments, in addition to validating the identified GSH using functional in vitro and in vivo analysis as disclosed herein, a candidate GSH can be optionally assessed using bioinformatics, e.g., determining if the candidate GSH meets certain criteria, for example, but not limited to assessing for any one or more of the following: proximity to cancer genes or proto-oncogenes, location in a gene or location near the 5′ end of a gene, location in selected housekeeping genes, location in extragenic regions, proximity to mRNA, proximity to ultra-conserved regions and proximity to long noncoding RNAs and other such genomic regions. By way of Example, the previously identified GSH AAVS1 (adeno-associated virus integration site 1), was identified as the adeno-associated virus common integration site on chromosome 19 and is located in chromosome 19 (position 19q13.42) and was primarily identified as a repeatedly recovered site of integration of wild-type AAV in the genome of cultured human cell lines that have been infected with AAV in vitro. Integration in the AAVS1 locus interrupts the gene phosphatase 1 regulatory subunit 12C (PPP1R12C; also known as MBS85), which encodes a protein with a function that is not clearly delineated. The organismal consequences of disrupting one or both alleles of PPP1R12C are currently unknown. No gross abnormalities or differentiation deficits were observed in human and mouse pluripotent stem cells harboring transgenes targeted in AAVS1. Previous assessment of the AAVS1 site typically used Rep-mediated targeting which preserved the functionality of the targeted allele and maintained the expression of PPP1R12C at levels that are comparable to those in non-targeted cells. AAVS1 was also assessed using ZFN-mediated recombination into iPSCs or CD34+ cells.

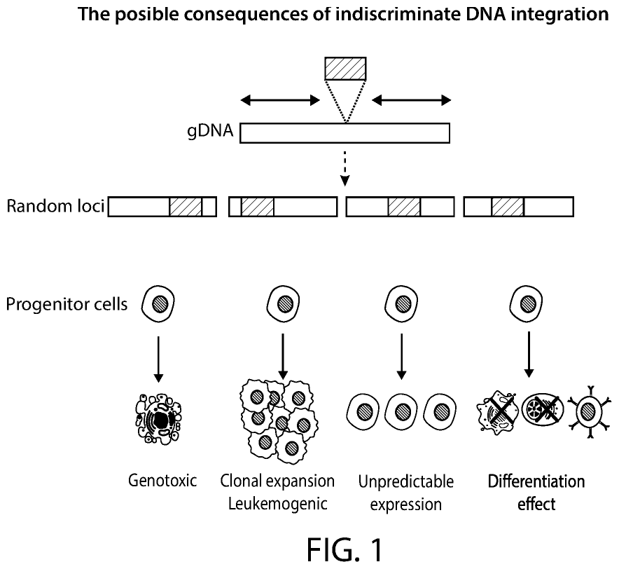

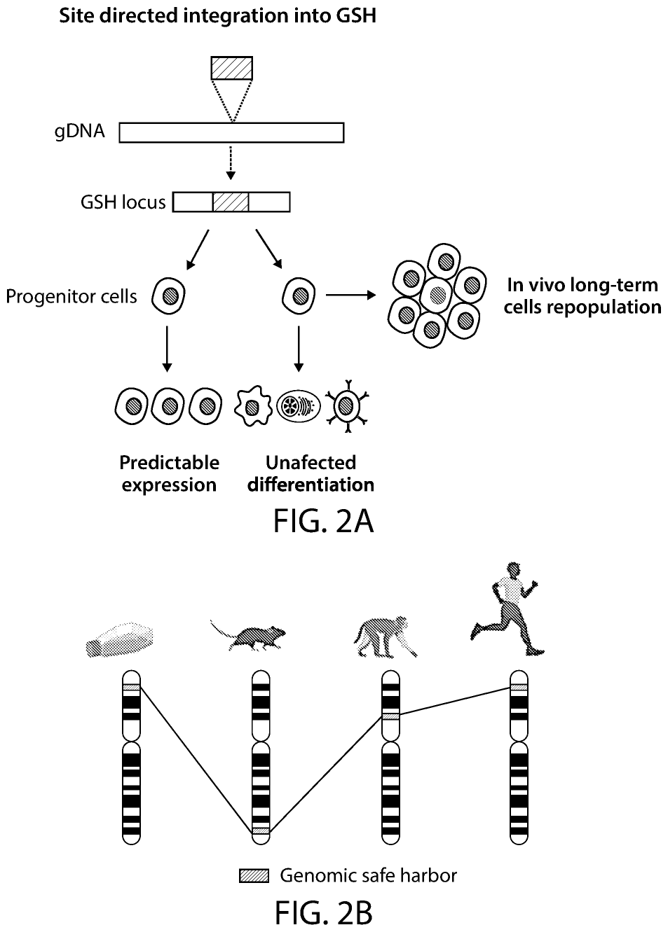

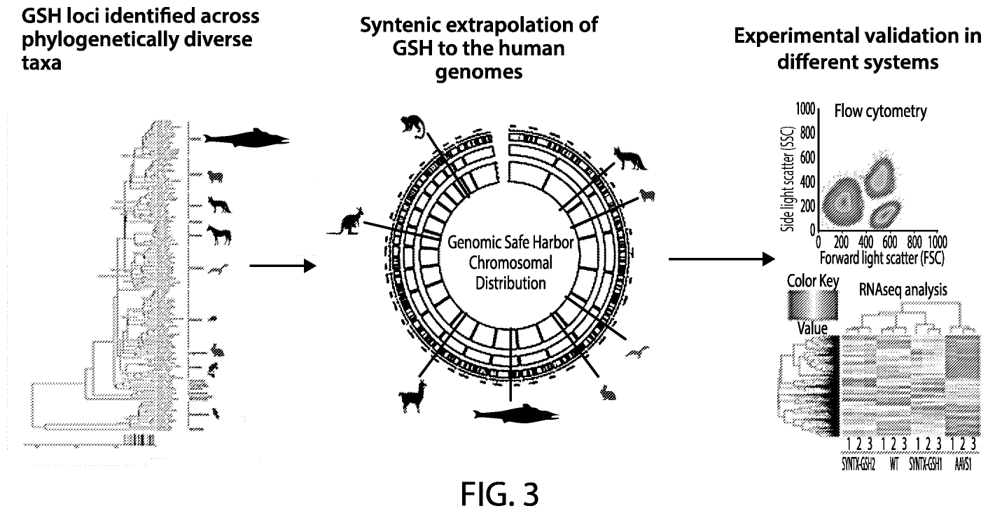

As originally characterized, the AAVS1 locus is >4 kb and is identified as chromosome 19 nucleotides 55,113,873-55,117,983 (human genome assembly GRCh38/hg38) and overlaps with exon 1 of the PPP1R12C gene that encodes protein phosphatase 1 regulatory subunit 12C. This >4 kb region is extremely G+C nucleotide content rich and is a gene-rich region of particularly gene-rich chromosome 19 (see FIG. 1A of Sadelain et al, Nature Revs Cancer, 2012; 12; 51-58), and some integrated promoters can indeed activate or cis-activate neighboring genes, the consequence of which in different tissues is presently unknown.

AAVS1 GSH was identified by characterizing the AAV provirus structure in latently infected human cell lines with recombinant bacteriophage genomic libraries generated from latently infected clonal cell lines (Detroit 6 clone 7374 IIID5) (Kotin and Bems 1989), Kotin et al isolated non-viral, cellular DNA flanking the provirus and used a subset of “left” and “right” flanking DNA fragments as probes to screen panels of independently derived latently infected clonal cell lines. In approximately 70% of the clonal isolates, AAV DNA was detected with the cell-specific probe (Kotin et al. 1991; Kotin et al. 1990). Sequence analysis of the pre-integration site identified near homology to a portion of the AAV inverted terminal repeat (Kotin, Linden, and Bems 1992). Although lacking the characteristic interrupted palindrome, the AAVS1 locus retained the p5 Rep proteins binding and nicking, also referred to as the terminal resolution sites (Chiorini et al. 1994; Chiorini et al. 1995; Im and Muzyczka 1989, 1990, 1992). Interestingly, the human orthologue functioned as a p5 Rep in vitro origin of DNA synthesis, thus supporting the early conjecture that AAVS1 integration is a Rep-dependent process (Kotin et al., 1990; Kotin et al., 1992; Urcelay et al. 1995; Weitzman et al. 1994). The Rep binding elements in cis were shown to be required for AAV integration and providing additional support for Rep protein involvement in the targeted, non-homolgous recombination process (Urabe, et al., Linden, Bems). These elements define the minimum origin of Rep-mediated DNA synthesis as the arrangement of Rep binding and nicking sites that allow RNA-primer independent strand-displacement DNA (leading strand) synthesis.

The wild-type adeno-associated virus may cause either a productive or latent infection, where the wild-type virus genome integrates frequently in the AAVS1 locus on human chromosome 19 in cultured cells (Kotin and Bems 1989; Kotin et al. 1990). This unique aspect of AAV has been exploited as one of the first so-called “safe-harbors” for iPSC genetic modification. AAVS1, as originally defined (Kotin et al., 1991) is situated on chromosome 19 between nucleotides 55,113,873-55,117,983 (human genome assembly GRCh38/hg38) and overlaps with exon 1 of the PPP1R12C gene that encodes protein phosphatase 1 regulatory subunit 12C. Interesting, PPP1R12C exon 1, 5′untranslated region contains a functional AAV origin of DNA synthesis indicated within the following sequences (Urcelay et al. 1995): The GCTC Rep-binding motifs and terminal resolution site (GGTTGG) are indicated with bold font: 55,117,600-TGGTGGCGGCGGTTGGGGCTCGGCGCTCGCTCGCTCGCTCGCTGGGCGGGC GGTGCGAIG-55,117,540.

Surprisingly, the human chromosome 19 AAVS1 safe-harbor is within an exonic region of PPP1R12C, the gene encoding protein phosphatase regulatory 1 regulatory subunit 12C. The selection of the exonic integration site is non-obvious, and perhaps counter-intuitive, since insertion and expression of foreign DNA will likely disrupt the expression of the endogenous genes. Apparently, insertion of the AAV genome into this locus does not adversely affect cell viability or iPSC differentiation (DeKelver et al. 2010; Wang et al. 2012; Zou et al. 201 1). Integration occurs by non-homologous recombination that requires the presence of AAV Rep proteins in trans and the minimum origin of AAV DNA synthesis in cis on both recombination substrates which then permits Rep-protein mediated juxtapositioning of the AAV and genomic DNAs (Weitzman et al. 1994).

The Rep-dependent minimum origin of DNA synthesis consists of the p5 Rep protein binding elements (RBE) and properly positioned terminal resolution site (trs) as exemplified by the AAV2 trs AGT|TGG and the AAV5 trs AGTG|TGG (the vertical line indicates the nicking position). In addition, the involvement of cell protein complexes has been inferred, but not yet identified or characterized.

These virus replication elements must function very efficiently or the virus would become extinct due to lack of replicative fitness, whereas, the small, non-coding, ca. 35 bp element in AAVS1 may have no function in the host. However, the AAVS1 locus has been established as a somatic cell safe harbor and disruption of the locus in totipotent or germline cells may interfere with ontogeny.

The AAVS1 locus is within the 5′ UTR of the highly conserved PPP1R12C gene. The Rep-dependent minimal origin of DNA synthesis is conserved in the 5′ UTR of the human, chimpanzee, and gorilla PPP1R12C gene. However, in rodent species (mouse and rat), substitutions occur with increased frequency within the preferred terminal resolution site compared to adjacent non-coding DNA. The incidental rather than selected or acquired genotype may affect the efficiency of the other species the specific sequences in the 5′ UTR.

In some embodiments, a candidate GSH identified according to embodiments herein is identified to meet the criteria of a GSH if it is safe and targeted gene delivery can be achieved that has limited off-target activity and minimal risk of genotoxicity, or causing insertional oncogenesis upon integration of foreign DNA, while being accessible to highly specific nucleases with minimal off-target activity.

While the GSH is validated based on in vitro and in vivo assays as described herein, in some embodiments, additional selection can be used based on determining whether the GSH falls into a particular criterion. For example, in some embodiments, a GSH locus identified herein is located in an exon, intron or untranslated region of a dispensable gene. Analysis shows that integration sites of provirus in tumors commonly are near the starting point of transcription, either upstream or just within the transcription unit, often within a 5′ intron. Proviruses at these locations have a tendency to dysregulate expression by increasing the rate of transcription either via virus promoter or via virus enhancer insertions. Accordingly, in some embodiments, a GSH locus identified herein is selected based on not being proximal to a cancer gene. In some embodiments, a GSH does not have an integration site located near the starting point of transcription of a cancer gene, e.g. upstream or in the 5′ intron of a cancer gene or proto-oncogene. Such cancer genes are well known to one of ordinary skill in the art, and are disclosed in Table 1 in Sadelain et al., Nature Revs Cancer, 2012; 12; 51-58, which is incorporated herein in its entirety. Exemplary databases of genes implicated in cancer are well known, e.g., Atlas gene set, CAN gene sets, CIS (RTCGD) gene set, and those described in Table 2 below.

TABLE 2
Exemplary databases of genes implicated in cancer

| | Number | | | |
|---|---|---|---|---|
| Gene set* | of genes | Species | Description | Refs |
| Atlas | 999 | human | This gene set is from the Atlas of genetics and cytogenetics in | 41 |
| | | | oncology and hematology. | |
| | | | It lists both hybrid genes found in at least one cancer case and | |
| | | | gene amplifications or | |
| | | | homozygous deletions found in a significant subset of cases in a | |
| | | | given cancer type | |
| Miscellaneous | 187 | Multiple | This gene set is from Retroviruses (Cold Spring Harbor | 35 |
| | | | Laboratory Press), an early version | |
| | | | of the CIS database, a list from T. Hunter, The Salk Institute, La | |
| | | | Jolla, California, USA, and | |
| | | | miscellaneous additions from the scientific literature | |
| CAN genes | 192 | | This gene set includes 192 common genes that were mutated at | 42 |
| | | | significant frequency in all tumors of human breast and | |
| | | | colorectal cancers | |
| CIS | 593 | Mouse | This gene set is from the Mouse Variation Resource and lists | 36 |
| (RTCGD) | | | retroviral insertional mutagenesis in mouse hematopoietic tumors | |
| Human | 38 | Human | This gene set is a list of lymphoid-specific oncogenes that was | |
| lymphoma | | | compiled by M. Cavazzana-Calvo and colleagues, Hôpital | |
| | | | Necker, Paris, France | |
| Sanger | 452 | Human | This gene set is from the Cancer Gene Census, a compilation | 43 |
| | | | from the scientific literature of “mutated genes that are causally | |
| | | | implicated in oncogenesis.” | |
| Waldman | 455 | Human | This gene set is from the Waldman gene database and lists cancer | |
| | | | genes sorted by chromosomal locus and includes links to OMIM | |
| AllOnco | 2,070 | Mouse | This database is a master set of the seven sets described above in | |
| | | and | which all genes are converted to their human homologues | |
| | | human | | |

*Gene lists and links to original sources are available at The Bushman lab cancer gene list website (see World Wide Web at bushmanlab.org/links/genelists).
CAN, cancer; CIS, common insertion site; References in the last column represent the reference number in Sadelain et al., Nature Revs Cancer (2012) 12: 51-58.

In some embodiments, a GSH loci identified herein has one or more properties selected from: (i) outside a gene transcription unit; (ii) located between 5-50 kilobases (kb) away from the 5′ end of any gene; (iii) located between 5-300 kb away from cancer-related genes; (iv) located 5-300 kb away from any identified microRNA; and (v) outside ultra-conserved regions and long noncoding RNAs. In some embodiments, a GSH locus identified herein has any or more of the following properties: (i) outside a gene transcription unit; (ii) located >50 kilobases (kb) from the 5′ end of any gene; (iii) located >300 kb from cancer-related genes; (iv) located >300 kb from any identified microRNA; and (v) outside ultra-conserved regions and long noncoding RNAs. In studies of lentiviral vector integrations in transduced induced pluripotent stem cells, analysis of over 5,000 integration sites revealed that ˜17% of integrations occurred in safe harbors. The vectors that integrated into these safe harbors were able to express therapeutic levels of β-globin from their transgene without perturbing endogenous gene expression.

### Homology and Sequence Alignment

Homology, as used herein, refers to the percentage of nucleotide sequence identity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

For nucleic acids, the term “substantial homology” indicates that two nucleic acids, or designated sequences thereof, when optimally aligned and compared, are identical, with appropriate nucleotide insertions or deletions, in at least about 60% of the nucleotides, usually at least about at least or about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% and more preferably at least about 97%, 98%, 99% or more of the nucleotides. Alternatively, substantial homology exists when the segments will hybridize under selective hybridization conditions, to the complement of the strand.

The percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available on the world wide web at the GCG company website), using a NWSgapdna. CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11 17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48): 444 453 (1970)) algorithm which has been incorporated into the GAP program in the GCG software package (available on the world wide web at the GCG company website), using either a Blosum 62 matrix or a PAM250 matrix, and a gap weight of 16, 14, 12, 10, 8, 6, or 4 and a length weight of 1, 2, 3, 4, 5, or 6.

The nucleic acid and protein sequences of the present invention can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403 10. BLAST nucleotide searches can be performed with the NBLAST program, score=100, wordlength=12 to obtain nucleotide sequences homologous to the nucleic acid molecules of the present invention. BLAST protein searches can be performed with the XBLAST program, score=50, wordlength=3 to obtain amino acid sequences homologous to the protein molecules of the present invention. To obtain gapped alignments for comparison purposes, Gapped BLAST can be utilized as described in Altschul et al., (1997) Nucleic Acids Res. 25 (17): 3389 3402. When utilizing BLAST and Gapped BLAST programs, the default parameters of the respective programs (e.g., XBLAST and NBLAST) can be used (available on the world wide web at the NCBI website).

### Validation of a GSH Using In Vitro and In Vivo Assays

While not being limited to theory, a useful GSH region must permit sufficient transgene expression to yield desired levels of the vector-encoded protein or non-coding RNA, and should not predispose cells to malignant transformation nor significantly negatively alter cellular functions.

Methods and compositions for validating the candidate GSH regions disclosed herein include, but are not limited to: bioinformatics, in vitro gene expression assays, in vitro and in vivo expression arrays to query nearby genes, in vitro-directed differentiation or in vivo reconstitution assays in xenogeneic transplant models, transgenesis in syntenic regions and analyses of patient databases from individuals. Accordingly, any one or combination of the methods for identifying GSH loci described herein may further comprise performing at least one in vitro, ex vivo, and/or in vivo.

In some embodiments, the validation of the GSH is determined to check that there is no germline integration of the introduced gene, reducing risks that there is germline transmission of the gene therapy vector.

Following identification of a target loci or candidate GSH, a series of in vitro and in vivo assays can be used to establish safety and in particular, the absence of oncogenic potential. In vitro oncogenicity assays can be based on the experience in previous gene therapy T-cell product characterizations.

In some embodiments, the GSH can be validated by a number of assays. In some embodiments, functional assays are selected from any one or more of: (a) insertion of a marker gene into the loci in human cells and measure marker gene expression in vitro; (b) insertion of marker gene into orthologous loci in progenitor cells or stem cells and engraft the cells into immunodepleted mice and/or assess marker gene expression in all developmental lineages; (c) differentiate hematopoietic CD34+ cells into terminally differentiated cell types, wherein the hematopoietic CD34+ cells have a marker gene inserted into the candidate GSH loci; or (d) generate transgenic knock-in mouse wherein the genomic DNA of the mouse has a marker gene inserted in the candidate GSH locus, wherein the marker gene is operatively linked to a tissue specific or inducible promoter.

In some embodiments, the at least one in vitro, ex vivo, and/or in vivo assay is selected from: (a) de novo targeted insertion of a marker gene into the locus in a cell (e.g., human cell) and determine (i) cell viability, (ii) the insertion efficiency and/or (iii) marker gene expression;
(b) targeted insertion of a marker gene into the locus in a progenitor cell or stem cell and differentiate in vitro and determine (i) marker gene expression in all developmental lineages, and/or (ii) whether the insertion of the marker gene affects differentiation of the said progenitor cell or stem cell;
(c) targeted insertion of a marker gene into the locus in a progenitor cell or stem cell and engraft the cell into immune-depleted mice and assess marker gene expression in all developmental lineages in vivo;
d) targeted insertion of a marker gene into the locus in a cell and determine the global cellular transcriptional profile (e.g., using RNAseq or microarray); and
e) generate a transgenic knock-in mouse wherein the genomic DNA of the mouse has a marker gene inserted in the locus, optionally wherein the marker gene is operatively linked to a tissue specific or inducible promoter.

In some embodiments, the stem cell used in the validation assay is selected from an embryonic stem cell, a tissue-specific stem cell, a mesenchymal stem cell, and an induced pluripotent stem cell (iPSC). In some embodiments, the cell, the progenitor cell or the stem cell is selected from a hematopoietic stem cell, a hematopoietic CD34+ cell, and epidermal stem cell, an epithelial stem cell, neural stem cell, a lung progenitor cell, muscle satellite cell, intestinal K cell, and a liver progenitor cell.

### Exemplary In Vitro Assays to Validate the GSH

In some embodiments, a functional assay to validate the GSH involves insertion of a marker gene into the loci of a human cell and determination of expression of the marker in vitro. In some embodiments, the marker gene is introduced by homologous recombination. In some embodiments, the marker gene is operatively linked to a promoter, for example, a constitutive promoter or an inducible promoter. The determination and quantification of gene expression of the marker gene can be performed by any method commonly known to a person of ordinary skill in the art, e.g., gene expression using e.g., RT-PCR, Affymetrix gene array, transcriptome analysis; and/or protein expression analysis (e.g., western blot) and the like. In some embodiments, the effect of the integrated marker transgene on neighboring gene expression is determined in cultured cells in vitro.

In some embodiments, the marker gene is introduced into is a mammalian cell, e.g., a human cell or a mouse cell or a rat cell. In some embodiments, the cell is a cell line, e.g., a fibroblast cell line, HEK293 cells and the like. In some embodiments, the cell used in the assay are pluripotent cells, e.g., iPSCs or clonable cell types, such as T lymphocytes. In some embodiments, the gene expression of the insertion of a marker gene into a variety of different cell populations, including primary cells is assessed. In some embodiments, a iPSC that has an introduced marker gene is differentiated into multiple lineages to check consistent and reliable gene expression of the marker gene in different lineages.

In some embodiments, a marker gene is inserted into a candidate GSH loci in the genome of hematopoietic cells, such as, for example, CD34+ cells, and differentiated into different terminally differentiated cell types.

In some embodiments, a cell population that has a marker gene introduced into the candidate GSH can be assessed for possible tissue malfunction and/or transformation. For example, a CD34+ cells or iPSCs are assessed for aberrant differentiation away from normal lineage differentiation, and/or increased proliferation which would indicate a risk of cancer.

In some embodiments, the gene expression levels of proximal genes are determined. For instance, in some embodiments, if the integrated marker gene results in aberrant gene expression of surrounding or neighboring gene expression, or other dysregulation, such as a downregulation or upregulation of gene expression of the neighboring genes, the candidate loci is not selected as a suitable GSH. In some embodiments, if no change is detected in the expression level of a neighboring gene, the candidate loci is nominated, or selected, as a GSH. In some embodiments, the gene expression of flanking, proximal or neighboring genes is determined, where a proximal or neighboring gene can be within about 350 kb, or about 300 kb, or about 250 kb or about 200 kb or about 100 kb, or between 10-100 kb, or between about 1-10 kb or less than 1 kb distance (upstream or downstream) from the site of insertion of the marker gene (i.e., genes or RNA sequences flanking either in the 5′ or 3′ of the insertion locus).

In some embodiments, the epigenetic features and profile of the targeted a candidate GSH locus is assessed before and after introduction of the marker gene to determine whether the introduction of the marker gene affects the epigenetic signature (e.g., histone modifications, DNA modifications, association of euchromatin or heterochromatin proteins, etc.) of the GSH, and/or surrounding or neighboring genes within about 350 kb upstream and downstream of the site of integration.

In some embodiments, insertion of a marker gene into a candidate GSH locus is assessed to see if the locus can accommodate different integrated transcription units. In some embodiments, the gene expression of a marker gene operatively linked to a range of different genetic elements, including promoters, enhancers, and chromatin determinants, including locus control regions, matrix attachments regions and insulator elements is assessed, as well as, in some embodiments, the gene expression of neighboring genes within about 350 kb, or about 300 kb, or about 250 kb or about 200 kb or about 100 kb, or between 10-100 kb, or between about 1-10 kb or less than 1 kb distance (upstream or downstream) from the site of insertion of the marker gene.

In some embodiments, a marker gene that is not operably linked to a promoter is inserted into a GSH locus to assess the effect of any promoter and/or other regulatory elements of the neighboring genes.

In some embodiments, as demonstrated herein, insertion of a marker gene into a candidate GSH locus is assessed to see if it changes the global transcription pattern. Such analysis can be accomplished by e.g., next-generation sequencing (NGS) of DNA or RNA, Affymetrix gene array, etc.

In some embodiments, where a GSH locus is associated with a specific gene, knock-down of the gene can be assessed to validate that the gene is either not necessary or is dispensable. As an exemplary example, as disclosed herein, SYNTX-GSH2 is surrounded by several different coding genes and RNA genes. Accordingly, in some embodiments, the effect on the cell function and gene expression of neighboring cells on RNAi knockdown of SYNTX-GSH2 could be assessed, and where knock-down of the candidate gene in the GSH locus does not have significant effects, the gene can be validated as a GSH. Also, in vitro assays using RNAi to knock down the GSH gene are important to determine the dispensability of the gene, especially resulting from biallelic disruption, as is often the case with endonuclease-mediated targeting.

In some embodiments, because cancer chemotherapy cytotoxic agents have genotoxic and carcinogenic potential, standard in vitro studies for preclinical evaluations of these types of drugs can also be used to assess GSH locus disruption. For example, the ability of a primary T cell to grow without cytokines and cell signaling is a feature of carcinogenic transformation.

For example, in some embodiments, one can introduce the marker gene into the candidate GSH locus of T-cells, e.g., SB-728-T cells and culture without cytokine support for several weeks and demonstrate that normal cell death occurs.

In other embodiments, the classic biological cell transformation assay is anchorage-independent growth of fibroblasts and is a stringent test of carcinogenesis. Accordingly, in some embodiments, a marker gene can be inserted into a target GSH locus in fibroblasts and assessed for anchorage-independent growth. Other in vitro assays or tests for evaluating oncogenicity can be used, e.g., mouse micronucleus test, anchorage independent growth, and mouse lymphoma TK gene mutation assay.

In some embodiments, the marker gene is selected from any of fluorescent reporter genes, e.g., GFP, RFP and the like, as well as bioluminescence reporter genes. Exemplary marker genes are described herein.

In some embodiments, the marker gene, or reporter gene sequences include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. In some embodiments, where the marker gene is green fluorescent protein or luciferase, the vector carrying the signal may be measured colorimetrically based on visible light absorbance or light production in a luminometer, respectively. Such reporters can, for example, be useful in verifying the tissue-specific targeting capabilities and tissue specific promoter regulatory activity of a nucleic acid.

In some embodiments, bioinformatics can be used to validate the GSH, for example, reviewing sequences of databases of patient-derived autologous iPSC, as described in Papapetrou et al., 2011, Na. Biotechnology, 29:73-78, which is incorporated herein in its entirety.

Additionally, once a GSH and target integration site in GSH is identified, bioinformatics and or web-based tools can be used to identify potential off-target sites. For example, bioinformatics tools such as Predicted Report of Genome-wide Nuclease Off-Target Sites (PROGNOS, World Wide Web at baolab.bme.gatech.edu/Research/BioinformaticTools/prognos.html) and CRISPOR (World Wide Web at crispor.tefor.net/) for designing CRISPR/Cas9 target and predicting off-target sites. CRISPOR and PROGNOS can provide a report of potential genome-wide nuclease target sites for ZFNs and TALENs. Once a particular target site is identified, the programs can provide a list ranking potential off-target sites.

### In Vivo Assays to Validate the GSH

In some embodiments, in vivo assays to functionally validate the GSH can be performed. In some embodiments, in vivo evaluation of GSHs can be performed in transgenic mice bearing a transgene that are integrated into syntenic regions.

In some embodiments, an in vivo functional assay to validate the GSH involves insertion of a marker gene into the loci of a iPSC and transplantation to immunodeficient mice. In some embodiments, the insertion of a marker gene into a iPSC and the modified iPSC implanted into immunodeficient mice and assessed over a period of time. Such an in vivo assay allows any genotoxic event to be assessed, including atypical or aberrant differentiation (e.g., changes in hematopoietic transformation and/or clonal skewing of hematopoiesis), as well as the outgrowth of tumorigenic cells to be assessed from a rare event.

Such in vivo methods in immunodeficient mice with hematopoietic cells are well known to one of ordinary skill in the art, and are disclosed in Zhou, et al. “Mouse transplant models for evaluating the oncogenic risk of a self-inactivating XSCID lentiviral vector.” PloS one 8.4 (2013): e62333, which is incorporated herein in its entirety by reference, where the malignancy incidence from the introduced modified hematopoetic cells or iPSC can be assessed as compared to control or cells where no marker gene is introduced at the target loci in the GSH. In some embodiments, hematopoietic malignancy can be assessed. In some embodiments, lineage distribution of peripheral blood cells in the recipient immunodeficient mice is assessed to determine myeloid skewing and a signal of insertional transformation or adverse effects due to the marker gene inserted at the GSH loci.

In some embodiments, because the recipient mouse strains are immunodeficient, if tumors do arise in such mice, one can characterize these tumors and evaluate whether they are of human origin. If tumors are of human origin, then it will be necessary to further evaluate their clonality with respect to the insertion of the marker gene at the GSH loci or any dysregulation gene expression (upregulation or downregulation) of on- or off-target sites, such as flanking RNA sequences or genes. However, clonality observed in a marker-gene introduced cell does not necessarily equal causality and may instead be an innocent label that merely reflects the tumor's clonal origin.

In some embodiments, in vivo assays can be used that rely on the fact that human T cells can be maintained in immunodeficient NOG mice. Such an assay requires the marker gene to be introduced into the target GSH loci and modified human T cells allowed to live and expand for months in the NOG model, and compared to non-modified T cells. In some embodiments, a model with human T-cell xeno-GVHD can be used, where 2 months is allowed for a maximal time for proliferation of cells before animals died of GVHD, and defining a dose and donors that gave reliable GVHD in the NOG mice. After 2 months, the animals are euthanized and tissues evaluated by histology for neoplasms, immunostaining to detect human cells, and gene expression analysis (e.g., Affymetrix array or RT-PCR of flanking genes surrounding the GSH insertion loci) for detection of modified gene expression of on-target and off-target sites.

In some embodiments, another in vivo assay to functionally validate the candidate loci as GSH is generating knock-in transgenic animals or transgenic mice.

Testing for Successful Gene Editing of a Marker Gene into a GSH of an iPSC or T-Lymphocyte or Other Host Cell

Assays well known in the art can be used to test the efficiency of insertion of the marker gene in both in vitro and in vivo models. Expression of the marker gene can be assessed by one skilled in the art by measuring mRNA and protein levels of the desired transgene (e.g., reverse transcription PCR, western blot analysis, and enzyme-linked immunosorbent assay (ELISA)). In some embodiments, the expression of the marker or reporter protein that can be used to assess the expression of the desired transgene, for example by examining the expression of the reporter protein by fluorescence microscopy or a luminescence plate reader. For in vivo applications, protein function assays can be used to test the functionality of a given gene and/or gene product to determine if gene editing has successfully occurred. It is contemplated herein that the effects of gene editing in a cell or subject can last for at least, about, or no more than 1 month, 2 months, 3 months, 4 months, 5 months, 6 months, 10 months, 12 months, 18 months, 2 years, 5 years, 10 years, 20 years, or can be permanent.

### Marker/Reporter Genes

Marker/reporter genes may be screenable or selectable.

Exemplary marker genes include but not limited to any of fluorescent reporter genes, e.g., GFP, RFP and the like, as well as bioluminescence reporter genes. Exemplary marker genes include, but are not limited to, glutathione-S-transferase (GST), horseradish peroxidase (HRP), chloramphenicol acetyltransferase (CAT) beta-galactosidase, beta-glucuronidase, luciferase, green fluorescent proteins (e.g., GFP, GFP-2, tagGFP, turboGFP, sfGFP, EGFP, Emerald, Azami Green, Monomeric Azami Green, CopGFP, AceGFP, ZsGreen1), HcRed, DsRed, cyan fluo-rescent protein (CFP), yellow fluorescent proteins (e.g., YFP, EYFP, Citrine, Venus YPet, PhiYFP, ZsYellow1), cyan fluorescent proteins (e.g., ECFP, Cerulean, CyPet AmCyanl, Midoriishi-Cyan) red fluorescent proteins (e.g., mKate, mKate2, mPlum, DsRed monomer, mCherry, mRFP1, DsRed-Express, DsRed2, HcRed-Tandem, HcRed 1, AsRed2, eqFP61 1, mRaspberry, mStrawberry, Jred), orange fluorescent proteins (e.g., mOrange, mKO, Kusabira-Orange, monomeric Kusabira-Orange, mTangerine, tdTomato) and autofluorescent proteins including blue fluorescent protein (BFP).

Marker genes may also include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for β-galactosidase activity. In some embodiments, where the marker gene is green fluorescent protein or luciferase, the vector carrying the signal may be measured colorimetrically based on visible light absorbance or light production in a luminometer, respectively. Such reporters can, for example, be useful in verifying the tissue-specific targeting capabilities and tissue specific promoter regulatory activity of a nucleic acid.

Marker genes include, but are not limited to, sequences encoding proteins that mediate antibiotic resistance (e.g., ampicillin resistance, neomycin resistance, G418 resistance, puromycin resistance) (e.g., blasticidin S-deaminase, amino 3′-glycosyl phosphotransferase), sequences encoding colored or fluorescent or luminescent proteins (e.g., green fluorescent protein, enhanced green fluorescent protein, red fluorescent protein, luciferase), and proteins which mediate cellular metabolism resulting in enhanced cell growth rates and/or gene amplification (e.g., dihydrofolate reductase).

### Vectors Comprising at Least a Portion of GSH

In certain aspects, provided herein are vector compositions (e.g., a nucleic acid vector, viral vector) comprising at least a portion or region of the GSH identified using the methods disclosed herein. The portion or region of the GSH can be modified, e.g., where a point mutation can disrupt or knock-out the gene function of the GSH gene identified herein. In other embodiments, the portion or region of the GSH in the vector can be modified to comprise a guide RNA (gRNA) inserted, e.g., a guide RNA for a nuclease as disclosed herein. In some embodiments, the GSH vector can comprise a target site for a guide RNA (gRNA) as disclosed herein, or alternatively, a restriction cloning site for introduction of a nucleic acid of interest as disclosed herein. In other embodiments, a recombinase recognition site such as loxP may be introduced to facilitate directed recombination using a Cre recombinase expressed from rAAV or other gene transfer vector. The loxP site inserted into the GSH may also be used by breeding with tg mice that express Cre in a tissue specific manner.

As an exemplary example, the vector compositions can be a plasmid, cosmid, or artificial chromosome (e.g., BAC), minicircle nucleic acid, or recombinant viral vector (e.g., rAd, AAV, rHSV, BEV or variants thereof). In some embodiments, the vector can comprise recombinase recognition sites (RRS), for example, LoxP sites, attP, AttB sites and the like.

In certain embodiments, a nucleic acid in the vectors comprises at least a portion of the GSH nucleic acid identified as a genomic safe harbor (GSH) in the methods described herein. For example, in some embodiments, the nucleic acid is present in a vector, e.g., a plasmid, cosmid or artificial chromosome, such as, for example, a BAC. In some embodiments, the nucleic acid composition comprises at least a target site of integration in a GSH, and 5′ and 3′ portions of the GSH nucleic acid flanking the target site of integration.

In some embodiments, the vector composition comprises a GSH nucleic acid sequence that is between 30-1000 nucleotides, between 1-3 kb, between 3-5 kb, between 5-10 kb, or between 10-50 kb, between 50-100 kb, or between 100-300 kb, or between 100-350 kb, or any integer between 10 base pairs and 350 kb in length.

In some embodiments, the vector composition comprises a nucleic acid sequence comprising a first nucleic acid sequence comprising a 5′ region of the GSH, and/or a second nucleic sequence comprising a 3′ region of the GSH. In some embodiments, the 5′ region is within close proximity and upstream of a target site of integration and the 3′ region of the GSH is in close proximity and downstream of a target site of integration.

Any vector systems may be used including, but not limited to, plasmid vectors, retroviral vectors, lentiviral vectors, adenovirus vectors, poxvirus vectors: herpesvirus (HSV) vectors and adeno-associated virus vectors, vaccinia virus vectors, bacteriophage vectors etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more of the sequences needed for treatment. Thus, when one or more nucleic acids of interests are introduced into the cell, if the nucleic acid of interest is a gene editing nucleic acid of interest, additional nucleases and/or donor sequences may be carried on the same vector or on different vectors. When multiple vectors are used, each vector may comprise one or more nucleic acid of interest as described herein.

### Nucleic Vectors Comprising at Least a Portion of GSH

In certain aspects, provided herein are nucleic acid vectors comprising at least a portion of the GSH nucleic acid identified in any one of the methods described herein. In some embodiments, the GSH nucleic acid comprises an untranslated sequence or an intron. In some embodiments, the GSH comprises a sequence that is at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the sequence of GSH or a fragment thereof listed in Table 3. In some embodiments, the GSH comprises a sequence that is at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the sequence of the genomic DNA or a fragment thereof of SYNTX-GSH1, SYNTX-GSH2, SYNTX-GSH3, or SYNTX-GSH4.

In some embodiments, the nucleic acid vectors of the present disclosure comprises at least one non-GSH nucleic acid (see below for further description).

In some embodiments, the nucleic acid vectors of the present disclosure further comprises: (a) a transcription regulatory element (e.g., an enhancer, a transcription termination sequence, an untranslated region (5′ or 3′ UTR), a proximal promoter element, a locus control region (e.g., a β-globin LCR or a DNase hypersensitive site (HS) of β-globin LCR), a polyadenylation signal sequence), and/or (b) a translation regulatory element (e.g., Kozak sequence, woodchuck hepatitis virus post-transcriptional regulatory element).

In some embodiments, a nucleic acid vector is selected from a plasmid, minicircle, comsid, artificial chromosome (e.g., BAC), linear covalently closed (LCC) DNA vector (e.g., minicircles, minivectors and miniknots), a linear covalently closed (LCC) vector (e.g., MIDGE, MiLV, ministering, miniplasmids), a mini-intronic plasmid, a pDNA expression vector, or variants thereof.

In some embodiments, nucleic acid vectors can transform prokaryotic or eukaryotic cells and be replication and/or expression. Vectors can be prokaryotic vectors, e.g., plasmids, or shuttle vectors, insect vectors, or eukaryotic vectors. Expression vectors can also be for administration to a plant cell, animal cell, preferably a mammalian cell or a human cell, fungal cell, bacterial cell, or protozoal cell using standard techniques described for example in Sambrook et al, supra and United States Patent Publications 20030232410; 20050208489; 20050026157; 20050064474; and 20060188987, and International Publication WO 2007/014275.

Nucleic acid vectors of the present disclosure include, for example, DNA plasmids, naked nucleic acid, naked phage DNA, minicircle DNA, and linear plasmids (e.g., disclosed in US2009/0263900), and nucleic acid complexed with a delivery vehicle such as a liposome or poloxamer. Circular DNA expression vectors or minicircle vectors are disclosed in WO2002/083889, WO2014/170,238, WO2004/099420, WO20 102/026099, U.S. Pat. Nos. 6,143,530, 5,622,866, 7,622,252, 8,460,924, 6,277,608, U.S. application 2003/0032092, 2004/0214329, which are incorporated herein in their entirety by reference.

Nucleic acid vectors suitable in the methods and compositions as disclosed herein include linear covalently closed DNA vectors (e.g., described in Nafissi and Slavcev “Construction and characterization of an in-vivo linear covalently closed DNA vector production system.” Microbial cell factories 11.1 (2012): 154), as well as linear covalently closed (UCC) mini-plasmids (e.g., described by Slavcev, Sum, and Nafissi “Optimized production of a safe and efficient gene therapeutic vaccine versus HIV via a linear covalently closed DNA minivector.” BMC Infectious Diseases 14. S2 (2014): P74), DNA ministrings (e.g., described in U.S. Pat. No. 9,290,778; Nafiseh, et al. “DNA ministrings: highly safe and effective gene delivery vectors.” Molecular Therapy—Nucleic Acids 3.6 (2014): e165; Wong, Shirley, et al. “Production of double-stranded DNA ministrings.” Journal of visualized experiments: JoVE 108 (2016)), or ceDNA vectors (e.g., Ui U, et al, (2013) Production and Characterization of Novel Recombinant Adeno-Associated Virus Replicative-Form Genomes: A Eukaryotic Source of DNA for Gene Transfer. PLoS ONE 8 (8): e69879).

Nucleic acid vectors also include, for example, minimized vectors, plasmids (including antibiotic free plamids), miniplasmids, minicircle, minivectors, such as those described in Hardee, Cinnamon L., et al. “Advances in non-viral DNA vectors for gene therapy.” Genes 8.2 (2017): 65. Examples of circular covalently closed vectors (CCC vectors) include minicircles, minivectors and miniknots. Examples of linear covalently closed (LCC) vectors include MIDGE, MiLV, ministring. Mini-intronic plasmids can also be used. These are described in Table 2 in Hardee, Cinnamon L., et al. “Advances in non-viral DNA vectors for gene therapy.” Genes 8.2 (2017): 65.

Nucleic acid vectors further include, for example, plasmids DNA vectors (pDNA expression vectors), as discussed in review article Gill, et al, “Progress and prospects: the design and production of plasmid vectors.” Gene therapy 16.2 (2009): 165-171, and Yin, Hao, et al. “Non-viral vectors for gene-based therapy.” Nature Reviews Genetics 15.8 (2014): 541-555.

### Nucleci Acid Vectors for Integration to a GSH Locus of a Target Genome

In certain aspects, provided herein are nucleic acid vectors described herein (e.g., nucleic acid vectors comprising at least a portion of GSH) that are used for integration into a GSH locus of a target genome of interest. In some embodiments, the nucleic acid vectors (e.g., nucleic acid vectors comprising at least a portion of GSH) further comprise additional sequences or modifications (e.g., certain orientation of the sequences homologous to the GSH sequence) for integration into a GSH locus of a target genome. Integration to the target genome may be driven by cellular processes, such as homologous recombination or non-homologous end-joining (NHEJ). The integration may also be initiated and/or facilitated by an exogenously introduced nuclease.

In preferred embodiments, the nucleic acid vectors comprise at least one non-GSH nucleic acid. In some embodiments, the non-GSH nucleic acid is destined for integration to a GSH locus of a target genome.

In some embodiments, the at least one non-GSH nucleic acid (either forward or reverse orientation) is flanked by a GSH 5′ homology arm and/or a GSH 3′ homology arm, wherein the homology arm comprises a nucleic acid sequence that is at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to the target GSH nucleic acid.

In some embodiments, the GSH homology arm is between 10-5000 base pairs, between 50-3000 base pairs, between 100-1500 base pairs, or any integer between 10-10,000 base pairs in length. In some embodiments, the GSH homology arm is between 100-1500 base pairs in length. In some embodiments, the GSH homology arm is at least 30 base pairs in length. In preferred embodiments, the GSH homology arm is sufficient in length to mediate homology-dependent integration into the GSH locus in the genome of a cell.

In some embodiments, the at least one non-GSH nucleic acid flanked by the GSH homology arm(s) is in an orientation for integration in the GSH in a forward orientation. In some embodiments, the at least one non-GSH nucleic acid is in an orientation for integration in the GSH in a reverse orientation.

In some embodiments, the nucleic acid comprises a restriction cloning site. In some embodiments, the restriction cloning site is flanked by the GSH-5′ homology arm and/or a 3′GSH homology as to facilitate cloning of at least one non-GSH nucleic acid destined for integration into a GSH locus of a target genome.

Accordingly, in some embodiments, a nucleic acid vector composition comprises: (a) a GSH 5′ homology arm, (b) a nucleic acid sequence comprising a restriction cloning site, and (c) a GSH 3′ homology arm, where the 5′ homology arm and the 3′ homology arm bind to a target site located in a GSH locus identified according to the methods as disclosed herein, and wherein the 5′ and 3′ homology arms allow insertion (of the nucleic acid located between the homology arms) by homologous recombination into a loci located within the genomic safe. In some embodiments, such nucleic acid vector further comprises at least one non-GSH nucleic acid destined for integration into a GSH locus of a target genome.

The 5′ and 3′ homology arms may be any sequence that is homologous with the GSH target sequence in the genome of the host cell. In some embodiments, the 5′ and 3′ homology arms may be homologous to portions of the GSH described herein. Furthermore, the 5′ and 3′ homology arms may be non-coding or coding nucleotide sequences.

In some embodiments, the 5′ and/or 3′ homology arms can be homologous to a sequence immediately upstream and/or downstream of the integration or DNA cleavage site on the chromosome. Alternatively, the 5′ and/or 3′ homology arms can be homologous to a sequence that is distant from the integration or DNA cleavage site, such as at least, about, or no more than 1, 2, 5, 10, 15, 20, 25, 30, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, or more base pairs away from the integration or DNA cleavage site, or partially or completely overlapping with the DNA cleavage site (e.g., can be a DNA break induced by an exogenously-introduced nuclease). In some embodiments, the 3′ homology arm of the nucleotide sequence is proximal to an ITR of a viral vector.

In some embodiments, the nucleic acid is integrated into the target genome by homologous recombination followed by a DNA break formation induced by an exogenously-introduced nuclease. In some embodiments, the nuclease is TALEN, ZFN, a meganuclease, a megaTAL, or a CRISPR endonuclease (e.g., a Cas9 endonuclease or a variant thereof). In some embodiments, the CRISPR endonuclease is in a complex with a guide RNA.

Accordingly, in some embodiments, a nucleic acid vector of the present disclosure further comprises a nucleic acid encoding a nuclease (e.g., Cas9 or a variant thereof, ZFN, TALEN) and/or a guide RNA, wherein the nuclease or the nuclease/gRNA complex makes a DNA break at the GSH, which is repaired using the donor nucleic acid, thereby integrating at least one non-GSH nucleic acid at GSH. In other embodiments, the nucleic acid encoding a nuclease and/or a guide RNA is provided in one or more independent nucleic acid vectors.

For integration of the nucleic acid located between the 5′ and 3′ homology arms, the 5′ and/or 3′ homology arms should be long enough for targeting to the GSH and allow (e.g., guide) integration into the genome by homologous recombination. To increase the likelihood of integration at a precise location and enhance the probability of homologous recombination, the 5′ and/or 3′ homology arms may include a sufficient number of nucleotides. In some embodiments, the 5′ and/or 3′ homology arms may include at least 10 base pairs but no more than 5,000 base pairs, at least 50 base pairs but no more than 5,000 base pairs, at least 100 base pairs but no more than 5,000 base pairs, at least 200 base pairs but no more than 5,000 base pairs, at least 250 base pairs but no more than 5,000 base pairs, or at least 300 base pairs but no more than 5,000 base pairs. In some embodiments, the 5′ and/or 3′ homology arms include about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 base pairs. Detailed information regarding the length of homology arms and recombination frequency is art-known, see e.g., Zhang et al. “Efficient precise knock in with a double cut HDR donor after CRISPR/Cas9-mediated double-stranded DNA cleavage.” Genome biology 18.1 (2017): 35, which is incorporated herein in its entirety by reference.

A nucleic acid vector of the present disclosure may be introduced into a target cell for integration into its genome by any method known in the art, e.g., chemical methods, electroporation, fusion with a cell comprising a nucleic acid vector, transduction, etc. In some embodiments, a nucleic acid vector of the present disclosure is integrated into the genome of a target cell upon transduction.

### Non-GSH Nucleic Acids

A vector (e.g., a nucleic acid vector, viral vector) of the present disclosure may comprise at least one non-GSH nucleic acid. The non-GSH nucleic acid may refer to any nucleic acid that does not comprise the sequence of GSH identified herein, e.g., a nucleic acid having sequences that are heterologous to GSH, e.g., nucleic acid sequences not natively present in the GSH locus, e.g., a transgene. The non-GSH nucleic acid may comprise sequence necessary for replication and/or maintaining the vector, e.g., replication origin, selection marker (e.g., antibiotic resistance gene, e.g., a marker that helps selecting or screening for successful integration), etc. In preferred embodiments, the non-GSH nucleic acid comprises a nucleic acid sequence destined for integration into a target genome. In preferred embodiments, such non-GSH nucleic acid may comprise sequences that serve therapeutic or research purposes, e.g., those down-regulating deleterious endogenous gene, those up-regulating deficient gene, etc.

In certain embodiments, the at least one non-GSH nucleic acid is not operably linked to a promoter. In some embodiments, the non-GSH nucleic acid may comprise sequences that are not intended for expression. In other embodiments, the non-GSH nucleic acid may comprise sequences that are intended for expression, and the expression may be driven by an endogenous promoter near the site of integration. Use of a neighboring promoter has been used for expression of a therapeutic gene (e.g., see LogicBio Therapeutic's integration of a gene of interest into an albumin locus, wherein the gene expression is facilitated by the albumin promoter).

In certain embodiments, the at least one non-GSH nucleic acid is operably linked to a promoter. In some embodiments, the at least one non-GSH nucleic acid is operably linked to a promoter, and the promoter is selected from: (a) a promoter heterologous to the nucleic acid to which it is operably linked; (b) a promoter that facilitates the tissue-specific expression of the nucleic acid; (c) a promoter that facilitates the constitutive expression of the nucleic acid; (d) an inducible promoter; (e) an immediate early promoter of an animal DNA virus; (f) an immediate early promoter of an insect virus; and (g) an insect cell promoter.

As described herein, in some embodiments, the inducible promoter is modulated by an agent selected from a small molecule, a metabolite, an oligonucleotide, a riboswitch, a peptide, a peptidomimetic, a hormone, a hormone analog, and light. In some embodiments, the agent is selected from tetracycline, cumate, tamoxifen, estrogen, and an antisense oligonucleotide (ASO), rapamycin, FKCsA, blue light, abscisic acid (ABA), and riboswitch.

In some embodiments, the promoter facilitates tissue-specific expression in a hematopoietic stem cell, a hematopoietic CD34+ cell, and epidermal stem cell, an epithelial stem cell, neural stem cell, a lung progenitor cell, a muscle satellite cell, an intestinal K cell, a neuronal cell, an airway epithelial cell, or a liver progenitor cell.

In some embodiments, the promoter is selected from the CMV promoter, β-globin promoter, CAG promoter, AHSP promoter, MND promoter, Wiskott-Aldrich promoter, PKLR promoter, polyhedron (polh) promoter, and immediately early 1 gene (IE-1) promoter.

In some embodiments, the at least one non-GSH nucleic acid increases or restores the expression of an endogenous gene of a target cell.

In other embodiments, the at least one non-GSH nucleic acid decreases or eliminates the expression of an endogenous gene of a target cell.

In some embodiments, the at least one non-GSH nucleic acid further comprises additional regulatory elements. In some embodiments, the at least one non-GSH nucleic acid comprises: (a) a transcription regulatory element (e.g., an enhancer, a transcription termination sequence, an untranslated region (5′ or 3′ UTR), a proximal promoter element, a locus control region (e.g., a β-globin LCR or a DNase hypersensitive site (HS) of β-globin LCR), a polyadenylation signal sequence), and/or (b) a translation regulatory element (e.g., Kozak sequence, woodchuck hepatitis virus post-transcriptional regulatory element).

In some embodiments, the at least one non-GSH nucleic acid may encode a coding RNA or non-coding RNA as described below.

Further provided herein are methods of inserting at least one non-GSH nucleic acid into a GSH locus of a cell, the method comprising introducing any one of the nucleic acid vectors described herein, any one of the viral vectors described herein, or any one of the pharmaceutical compositions described herein, into the cell, whereby homologous recombination of the GSH 5′ homology arm and the GSH 3′ homology arm flanking the non-GSH nucleic acid with the GSH locus in the genome integrates the non-GSH nucleic acid into the GSH locus. In some embodiments, the non-GSH nucleic acid is integrated into the GSH in a forward orientation. In other embodiments, the non-GSH nucleic acid is integrated into the GSH in a reverse orientation.

### Non-Coding RNA & Coding RNA

In certain aspect, provided herein is at least one non-GSH nucleic acid, wherein the non-GSH nucleic acid comprises a sequence that encodes a coding RNA.

In some embodiments, the sequence encoding a coding RNA is codon-optimized for expression in a target cell. In some embodiments, the at least one non-GSH nucleic acid encoding a coding RNA further comprises a sequence encoding a signal peptide, which allows production of membrane-localized or secreted polypeptides.

In some embodiments, the at least one non-GSH nucleic acid comprises a sequence encoding: (a) a protein or a fragment thereof, preferably a human protein or a fragment thereof;
(b) a therapeutic protein or a fragment thereof, an antigen-binding protein, or a peptide; (c) a suicide gene, optionally Herpes Simplex Virus-1 Thymidine Kinase (HSV-TK); (d) a viral protein or a fragment thereof; (e) a nuclease, optionally a Transcription Activator-Like Effector Nuclease (TALEN), a zinc-finger nuclease (ZFN), a meganuclease, a megaTAL, or a CRISPR endonuclease, (e.g., a Cas9 endonuclease or a variant thereof); (f) a marker, e.g., luciferase or GFP; and/or (g) a drug resistance protein, e.g., antibiotic resistance gene, e.g., neomycin resistance.

In some embodiments, the at least one non-GSH nucleic acid comprises a sequence encoding a viral protein or a fragment thereof. In some embodiments, the viral protein or a fragment thereof comprises a structural protein (e.g., VP1, VP2, VP3) or a non-structural protein (e.g., Rep protein). Such non-GSH nucleic acid may be useful in engineering a cell to produce a recombinant viral protein (e.g., for a vaccine production), and/or engineering a cell to produce a recombinant viral particle (e.g., AAV, etc.). In some embodiments, the viral protein or a fragment thereof comprises: (a) a parvovirus protein or a fragment thereof, optionally VP1, VP2, VP3, NS1, or Rep; (b) a retrovirus protein or a fragment thereof, optionally an envelope protein, gag, pol, or VSV-G; (c) an adenovirus protein or a fragment thereof, optionally E1A, E1B, E2A, E2B, E3, E4, or a structural protein (e.g., A, B, C); and/or (d) a herpes simplex virus protein or a fragment thereof, optionally ICP27, ICP4, or pac.

In some embodiments, the at least one non-GSH nucleic acid encoding a viral protein encodes a surface protein, or a fragment thereof, of a virus. In some embodiments, (a) the surface protein or a fragment thereof is an immunogenic surface protein that elicits immune response in a host, (b) the surface protein or a fragment thereof further comprises a signal peptide, (c) the gene encoding the surface protein or a fragment thereof is operably linked to an inducible promoter, and/or (d) the nucleic acid encoding the surface protein or fragment thereof further comprises a suicide gene. In some embodiments, the surface protein is of a coronavirus (e.g., MERS, SARS), influenza virus, respiratory syncytial virus, hepatitis A, hepatitis B, hepatitis C, hepatitis D, hepatitis E, human papillomavirus, dengue virus serotype 1, dengue virus serotype 2, dengue virus serotype 3, dengue virus serotype 4, zika, virus, West Nile virus, yellow fever virus, Chikungunya virus, Mayaro virus, Ebola virus, Marburg virus, or Nipa virus. In some embodiments, the surface protein is the spike protein of SARS-COV-2.

In some embodiments, the at least one non-GSH nucleic acid comprising a sequence encoding a protein, or a fragment thereof. In some embodiments, the at least one non-GSH nucleic acid comprising a sequence encoding a protein, or a fragment thereof, is selected from a hemoglobin gene (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), GBA1, preproinsulin, insulin, GIP, GLP-1, CEP290, ATPB1, ATPB11, ABCB4, CPS1, ATP7B, KRT5, KRT14, PLEC1, Col7A1, ITGB4, ITGA6, LAMA3, LAMB3, LAMC2, KIND1, INS, F8 or a fragment thereof (e.g., fragment encoding B-domain deleted polypeptide (e.g., VIII SQ, p-VIII)), IRGM, NOD2, ATG2B, ATG9, ATG5, ATG7, ATG16L1, BECN1, EI24/PIG8, TECPR2, WDR45/WIP14, CHMP2B, CHMP4B, Dynein, EPG5, HspB8, LAMP2, LC3b UVRAG, VCP/p97, ZFYVE26, PARK2/Parkin, PARK6/PINK1, SQSTM1/p62, SMURF, AMPK, ULK1, RPE65, CHM, RPGR, PDE6B, CNGA3, GUCY2D, RS1, ABCA4, MYO7A, HFE, hepcidin, a gene encoding a soluble form (e.g., of the TNFα receptor, IL-6 receptor, IL-12 receptor, or IL-1β receptor), and cystic fibrosis transmembrane conductance regulator (CFTR).

In some embodiments, the at least one non-GSH nucleic acid comprises a sequence encoding an antigen-binding protein. In some embodiments, the antigen-binding protein is an antibody or an antigen-binding fragment thereof, optionally wherein the antibody or an antigen-binding fragment thereof is selected from an antibody, Fv, F(ab)2, Fab′, dsFv, scFv, sc (Fv)2, half antibody-scFv, tandem scFv, Fab/scFv-Fc, tandem Fab′, single-chain diabody, tandem diabody (TandAb), Fab/scFv-Fc, scFv-Fc, heterodimeric IgG (CrossMab), DART, and diabody.

In some embodiments, the antigen-binding protein specifically binds TNFα, CD20, a cytokine (e.g., IL-1, IL-6, BLyS, APRIL, IFN-gamma, etc.), Her2, RANKL, IL-6R, GM-CSF, CCR5, or a pathogen (e.g., bacterial toxin, viral capsid protein, etc.).

In some embodiments, the antigen-binding protein is selected from adalimumab, etanercept, infliximab, certolizumab, golimumab, anakinra, rituximab, abatacept, tocilizumab, natalizumab, canakinumab, atacicept, belimumab, ocrelizumab, ofatumumab, fontolizumab, trastuzumab, denosumab, sarilumab, lenzilumab, gimsilumab, siltuximab, leronlimab, and an antigen-binding fragment thereof.

Accordingly, in some embodiments, the at least one non-GSH nucleic acid encodes a receptor, toxin, a hormone, an enzyme, a marker protein encoded by a marker gene (see above), or a cell surface protein or a therapeutic protein, peptide or antibody or fragment thereof. In some embodiments, a nucleic acid of interest for use in the vector compositions as disclosed herein encodes any polypeptide of which expression in the cell is desired, including, but not limited to antigen-binding proteins (e.g., antibodies), antigens, enzymes, receptors (cell surface or nuclear), hormones, lymphokines, cytokines, marker polypeptides, growth factors, and functional fragments of any of the above. The coding sequences may be, for example, cDNAs.

A coding RNA may further comprise the sequence encoding a tag, e.g., epitope tags, such that tags are fused to a protein of interest to facilitated detection and/or purification. Exemplary tages include, for example, one or more copies of FLAG, His, myc, Tap, HA or any detectable amino acid sequence.

A person of ordinary skill in the art understands that proteins intended for secretion comprises a signal peptide, and the nucleic acid encoding such protein comprises the nucleic acid sequence encoding the signal peptide.

In certain embodiments, the at least one non-GSH nucleic acid for use in the vector compositions as disclosed herein comprises a nucleic acid sequence that encodes a marker gene (described herein), allowing selection of cells that have undergone targeted integration, and a linked sequence encoding an additional functionality.

In some embodiments, at least one non-GSH nucleic acid comprises a nucleic acid for use in methods of preventing or treating one or more genetic deficiencies or dysfunctions in a mammal, such as for example, a polypeptide deficiency or polypeptide excess in a mammal, and particularly for preventing, treating or reducing the severity or extent of deficiency in a human manifesting one or more of the disorders linked to a deficiency in such polypeptides in cells and tissues. The method involves administration of the nucleic acid (e.g., a nucleic acid as described by the disclosure) that encodes one or more therapeutic peptides, polypeptides, siRNAs, microRNAs, antisense nucleotides, etc. in a nucleic acid vector, viral vector, or cells comprising said nucleic acid vector or viral vector as described herein, preferably in a pharmaceutically acceptable composition, to the subject in an amount and for a period of time sufficient to prevent or treat the deficiency or disorder in the subject suffering from such a disorder.

Thus, in some embodiments, the at least one non-GSH nucleic acid for use in the vector compositions as disclosed herein can encode one or more peptides, polypeptides, or proteins, which are useful for the treatment or prevention of a disease in a mammalian subject.

Exemplary non-GSH nucleic acids for use in the compositions and methods as disclosed herein include but not limited to: BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10 (187A), viral IL-10, IL-11, IL-12, IL-13, IL-14, IL-15, IL-16, IL-17, IL-18, VEGF, FGF, SDF-1, connexin 40, connexin 43, SCN4a, HIFia, SERCa2a, ADCY1, and ADCY6.

In some embodiments, the nucleic acid may comprise a coding sequence or a fragment thereof selected from the group consisting of a mammalian β globin gene (e.g., HBA1, HBA2, HBB, HBG1, HBG2, HBD, HBE1, and/or HBZ), alpha-hemoglobin stabilizing protein (AHSP), a B-cell lymphoma/leukemia 11A (BCL11A) gene, a Kruppel-like factor 1 (KLF1) gene, a CCR5 gene, a CXCR4 gene, a PPP1R12C (AAVS1) gene, an hypoxanthine phosphoribosyltransferase (HPRT) gene, an albumin gene, a Factor VIII gene, a Factor IX gene, a Leucine-rich repeat kinase 2 (LRRK2) gene, a Huntingtin (HTT) gene, a rhodopsin (RHO) gene, a Cystic Fibrosis Transmembrane Conductance Regulator (CFTR) gene, F8 or a fragment thereof (e.g., fragment encoding B-domain deleted polypeptide (e.g., VIII SQ, p-VIII)), a surfactant protein B gene (SFTPB), a T-cell receptor alpha (TRAC) gene, a T-cell receptor beta (TRBC) gene, a programmed cell death 1 (PD1) gene, a Cytotoxic T-Lymphocyte Antigen 4 (CTLA-4) gene, an human leukocyte antigen (HLA) A gene, an HLA B gene, an HLA C gene, an HLA-DPA gene, an HLA-DQ gene, an HLA-DRA gene, a LMP7 gene, a Transporter associated with Antigen Processing (TAP) 1 gene, a TAP2 gene, a tapasin gene (TAPBP), a class II major histocompatibility complex transactivator (CUT A) gene, a dystrophin gene (DMD), a glucocorticoid receptor gene (GR), an IL2RG gene, an RFX5 gene, a FAD2 gene, a FAD3 gene, a ZP15 gene, a KASII gene, a MDH gene, and/or an EPSPS gene.

In some embodiments, a non-GSH nucleic acid can be used to restore the expression of genes that are reduced in expression, silenced, or otherwise dysfunctional in a subject (e.g., a tumor suppressor that has been silenced in a subject having cancer). Similarly, in some embodiments, a non-GSH nucleic acid can also be used to knockdown the expression of genes that are aberrantly expressed in a subject (e.g., an oncogene that is expressed in a subject having cancer).

In some embodiments, the dysfunctional gene is a tumor suppressor that has been silenced in a subject having cancer. In some embodiments, the dysfunctional gene is an oncogene that is aberrantly expressed in a subject having a cancer. Exemplary genes associated with cancer (oncogenes and tumor suppressors) include but not limited to: AARS, ABCB 1, ABCC4, ABI2, ABL1, ABL2, ACK1, ACP2, ACY1, ADSL, AKI, AKR1C2, AKT1, ALB, ANPEP, ANXAS, ANXA7, AP2M1, APC, ARHGAPS, ARHGEFS, ARID4A, ASNS, ATF4, ATM, ATPSB, ATPSO, AXL, BARD1, BAX, BCL2, BHLHB2, BLMH, BRAF, BRCA1, BRCA2, BTK, CANX, CAP1, CAPN1, CAPNS1, CAV1, CBFB, CBLB, CCL2, CCND1, CCND2, CCND3, CCNE1, CCTS, CCYR61, CD24, CD44, CD59, CDC20, CDC25, CDC25A, CDC25B, CDC2LS, CDK10, CDK4, CDK5, CDK9, CDKL1, CDKN1A, CDKN1B, CDKN1C, CDKN2A, CDKN2B, CDKN2D, CEBPG, CENPC1, CGRRF1, CHAFIA, CIB1, CKMT1, CLK1, CLK2, CLK3, CLNS1A, CLTC, COLIA1, COL6A3, COX6C, COX7A2, CRAT, CRHR1, CSF1R, CSK, CSNKIG2, CTNNA1, CTNNB1, CTPS, CTSC, CTSD, CUL1, CYR61, DCC, DCN, DDX10, DEK, DHCR7, DHRS2, DHX8, DLG3, DVL1, DVL3, E2F1, E2F3, E2F5, EGFR, EGR1, EIF5, EPHA2, ERBB2, ERBB3, ERBB4, ERCC3, ETV1, ETV3, ETV6, F2R, FASTK, FBN1, FBN2, FES, FGFR1, FGR, FKBP8, FN1, FOS, FOSL1, FOSL2, FOXG1A, FOXO1A, FRAP1, FRZB, FTL, FZD2, FZDS, FZD9, G22P1, GAS6, GCNSL2, GDF1S, GNA13, GNAS, GNB2, GNB2L1, GPR39, GRB2, GSK3A, GSPT1, GTF21, HDAC1, HDGF, HMMR, HPRT1, HRB, HSPA4, HSPAS, HSPA8, HSPB1, HSPH1, HYAL1, HYOU1, ICAM1, ID1, ID2, IDUA, IER3, IFITM1, IGF1R, IGF2R, IGFBP3, IGFBP4, IGFBPS, IL1B, ILK, ING1, IRF3, ITGA3, ITGA6, ITGB4, JAK1, JARID1A, JUN, JUNB, JUND, K-ALPHA-1, KIT, KITLG, KLK10, KPNA2, KRAS2, KRT18, KRT2A, KRT9, LAMB1, LAMP2, LCK, LCN2, LEP, LITAF, LRPAP1, LTF, LYN, LZTR1, MADH1, MAP2K2, MAP3K8, MAPK12, MAPK13, MAPKAPK3, MAPRE1, MARS, MAS1, MCC, MCM2, MCM4, MDM2, MDM4, MET, MGST1, MICB, MLLT3, MME, MMP1, MMP14, MMP17, MMP2, MNDA, MSH2, MSH6, MT3, MYB, MYBL1, MYBL2, MYC, MYCL1, MYCN, MYD88, MYL9, MYLK, NEO1, NF1, NF2, NFKB I, NFKB2, NFSF7, NID, NINJ1, NMBR, NME1, NME2, NME3, NOTCH 1, NOTCH2, NOTCH4, NPM1, NQO1, NRID1, NR2F1, NR2F6, NRAS, NRG1, NSEP1, OSM, PA2G4, PABPC1, PCNA, PCTK1, PCTK2, PCTK3, PDGFA, PDGFB, PDGFRA, PDPK1, PEA15, PFDN4, PFDN5, PGAM1, PHB, PIK3CA, PIK3CB, PIK3CG, PIM1, PKM2, PKMYT1, PLK2, PPARD, PPARG, PPIH, PPP1CA, PPP2RSA, PRDX2, PRDX4, PRKAR1A, PRKCBP1, PRNP, PRSS15, PSMA1, PTCH, PTEN, PTGS1, PTMA, PTN, PTPRN, RABSA, RAC1, RADSO, RAF1, RALBP1, RAP1A, RARA, RARB, RASGRF1, RB1, RBBP4, RBL2, REA, REL, RELA, RELB, RET, RFC2, RGS19, RHOA, RHOB, RHOC, RHOD, RIPK1, RPN2, RPS6 KB 1, RRM1, SARS, SELENBP1, SEMA3C, SEMA4D, SEPP1, SERPINH1, SFN, SFPQ, SFRS7, SHB, SHH, SIAH2, SIVA, SIVA TP53, SKI, SKIL, SLC16A1, SLCIA4, SLC20A1, SMO, SMPD1, SNAI2, SND1, SNRPB2, SOCS1, SOCS3, SOD1, SORT1, SPINT2, SPRY2, SRC, SRPX, STAT1, STAT2, STAT3, STAT5B, STC1, TAF1, TBL3, TBRG4, TCF1, TCF7L2, TFAP2C, TFDP1, TFDP2, TGFA, TGFB1, TGFBR1, TGFBR2, TGFBR3, THBS1, TIE, TIMP1, TIMP3, TJP1, TK1, TLE1, TNF, TNFRSF10A, TNFRSF10B, TNFRSF1A, TNFRSF1B, TNFRSF6, TNFSF7, TNK1, TOB1, TP53, TP53BP2, TP5313, TP73, TPBG, TPTI, TRADD, TRAMI, TRRAP, TSG101, TUFM, TXNRD1, TYR03, UBC, UBE2L6, UCHL1, USP7, VDAC1, VEGF, VHL, VIL2, WEEI, WNT1, WNT2, WNT2B, WNT3, WNTSA, WT1, XRCC 1, YES 1. YWHAB, YWHAZ, ZAP70, and ZNF9.

In some embodiments, the dysfunctional gene is HBB. In some embodiments, the HBB comprises at least one nonsense, frameshift, or splicing mutation that reduces or eliminates the β-globin production. In some embodiments. HBB comprises at least one mutation in the promoter region or polyadenylation signal of HBB. In some embodiments, the HBB mutation is at least one of c. 17A>T, c.−1360G, c.92+1G>A, c.92+6T>C, c.93−21G>A, c.1180T, c.316−106OG, c.25_26delAA, c.27_28insG, c.92+5G>C, c. 1180T, c. 135delC, c.315+1G>A, c.−78A>G, c.52A>T, c.59A>G, c.92+5G>C, c. 124_127delTTCT, c.316−1970T, c.−78A>G, c.52A>T, c. 124_127delTTCT, c.316−197C>T, C.−1380T, c.−79A>G, c.92+5G>C, c.75T>A, c.316−2A>G, and c.316−2A>C.

In certain embodiments, the sickle cell disease is improved by gene therapy (e.g., stem cell gene therapy) that introduces an HBB variant that comprises one or more mutations comprising anti-sickling activity. In some embodiments, the HBB variant may be a double mutant (βAS2; T87Q and E22A). In other embodiments, the HBB variant may be a triple-mutant β-globin variant (βAS3; T87Q, E22A, and G16D). A modification at β16, glycine to aspartic acid, serves a competitive advantage over sickle globin (βS, HbS) for binding to α chain. A modification at β22, glutamic acid to alanine, partially enhances axial interaction with α20 histidine. These modifications result in anti-sickling properties greater than those of the single T87Q-modified variant and comparable to fetal globin. In a SCD murine model, transplantation of bone marrow stem cells transduced with SIN lentivirus carrying βAS3 reversed the red blood cell physiology and SCD clinical symptoms. Accordingly, this variant is being tested in a clinical trial (Identifier no: NCT02247843), Cytotherapy (2018) 20 (7): 899-910.

In some embodiments, the dysfunctional gene is CFTR. In some embodiments, CFTR comprises a mutation selected from ΔF508, R553X, R74W, R668C, S977F, L997F, K1060T, A1067T, R1070Q, R1066H, T3381, R334W, G85E, A46D, 1336K, H1054D, MIV, E92K, V520F, H1085R, R560T, L927P, R560S, N1303K, M1101K, L1077P, R1066M, R1066C, L1065P, Y569D, A561E, A559T, S492F, L467P, R347P, S341P, 1507del, G1061R, G542X, W1282X, and 2184InsA.

A skilled artisan will realize that the nucleic acids of interest can encode proteins or polypeptides, and that mutations that results in conservative amino acid substitutions may be made in a transgene to provide functionally equivalent variants, or homologs of a protein or polypeptide. In some aspects the disclosure embraces sequence alterations that result in conservative amino acid substitution of a transgene. In some embodiments, a non-GSH nucleic acid encodes a gene having a dominant negative mutation. For example, a nucleic acid of interest as defined herein encodes a mutant protein that interacts with the same elements as a wild-type protein, and thereby blocks some aspect of the function of the wild-type protein.

In some embodiments, the at least one non-GSH nucleic acid can further comprise a suicide gene, operatively linked to an inducible promoter and/or tissue specific promoter. Thus, such a vector can be used to kill cells upon a signal, or induce cells to undergo apoptosis or programmed cell death upon a specific and discrete signal. Such a vector comprising a suicide gene can be used as an escape hatch should the gene targeting or gene editing system not function as expected. Alternatively, a suicide gene can be used to kill cancer cells or sensitize cancer cells to e.g., chemotherapy. Exemplary suicide gene is well known in the art, and include thymidine kinase (TK, Viral), cytosine deaminase (CD, bacterial and yeast), carboxypeptidase G2 (CPG2, bacterial) and nitroreductase (NTR, bacterial). In some embodiments, the suicide gene is Herpes Simplex Virus-1 Thymidine Kinase (HSV-TK).

Further described herein are methods of targeted insertion of any sequence of interest into a cell. In some embodiments, a nucleic acid of interest is a nucleic acid that encodes a gene or groups of genes whose expression is known to be associated with a particular differentiation lineage of a stem cell. Sequences comprising genes involved in cell fate or other markers of stem cell differentiation can also be inserted. For example a promoterless construct containing such a gene can be inserted into a specified region (locus) such that the endogenous promoter at that locus drives expression of the gene product.

Similarly, in certain embodiments, genomic modifications (e.g., transgene integration) at a GSH locus identified herein allow integration of a nucleic acid of interest that may either utilize the promoter found at that safe harbor locus, or allow the expressional regulation of the transgene by an exogenous promoter or control element, as described herein, that is fused to the nucleic acid of interest prior to insertion.

In certain embodiments, the at least one non-GSH nucleic acid comprises a sequence encoding a non-coding RNA. In some embodiments, the non-coding RNA comprises antisense polynucleotides, lncRNA, piRNA, miRNA, shRNA, siRNA, antisense RNA, snoRNA, snRNA, scaRNA, and/or guide RNA. In some embodiments, the non-coding RNA targets a gene selected from DMT-1, ferroportin, TNFα receptor, IL-6 receptor, IL-12 receptor, IL-1β receptor, a gene encoding a mutated protein (e.g., a mutated HFE, CFTR).

The small nucleic acid may modulate the expression of a gene product associated with cancer (e.g., oncogenes) may be used to prevent or treat the cancer. In some embodiments, a non-GSH nucleic acid encodes a gene product associated with cancer (or a functional RNA that inhibits the expression of a gene associated with cancer) for use, e.g., for treatment, for research purposes, e.g., to study the cancer or to identify therapeutics that prevent or treat the cancer.

An ordinarily skilled artisan also appreciates that the non-GSH nucleic acid can comprise one or more mutations that result in conservative amino acid substitutions which may provide functionally equivalent variants, or homologs of a protein or polypeptide. Additionally contemplated in this disclosure is a nucleic acid of interest integrated in a GSH locus described herein, having a dominant negative mutation. For example, a nucleic acid of interest can encode a mutant protein that interacts with the same elements as a wild-type protein, and thereby blocks some aspects of the function of the wild-type protein.

In some embodiments, the at least one non-GSH nucleic acid comprises a non-coding RNA that mediates RNA interference. For example, the non-coding RNA comprises a short interfering RNA. Short interfering RNA (siRNA) is an agent which functions to inhibit expression of a target nucleic acid, e.g., by RNAi. An siRNA may be chemically synthesized, may be produced by in vitro transcription, or may be produced within a host cell. In some embodiments, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In other embodiments, an siRNA is a small hairpin (also called stem loop) RNA (shRNA). In some embodiments, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA Apr; 9 (4): 493-501 incorporated by reference herein).

In some embodiments, the non-coding RNA comprises piRNA. Piwi-interacting RNA (piRNA) is the largest class of small non-coding RNA molecules. piRNAs form RNA-protein complexes through interactions with piwi proteins. These piRNA complexes have been linked to both epigenetic and post-transcriptional gene silencing of retrotransposons and other genetic elements in germ line cells, particularly those in spermatogenesis. They are distinct from microRNA (miRNA) in size (26-31 nt rather than 21-24 nt), lack of sequence conservation, and increased complexity. However, like other small RNAs, piRNAs are thought to be involved in gene silencing, specifically the silencing of transposons. The majority of piRNAs are antisense to transposon sequences, suggesting that transposons are the piRNA target. In mammals it appears that the activity of piRNAs in transposon silencing is most important during the development of the embryo, and in both C. elegans and humans, piRNAs are necessary for spermatogenesis. piRNA has a role in RNA silencing via the formation of an RNA-induced silencing complex (RISC).

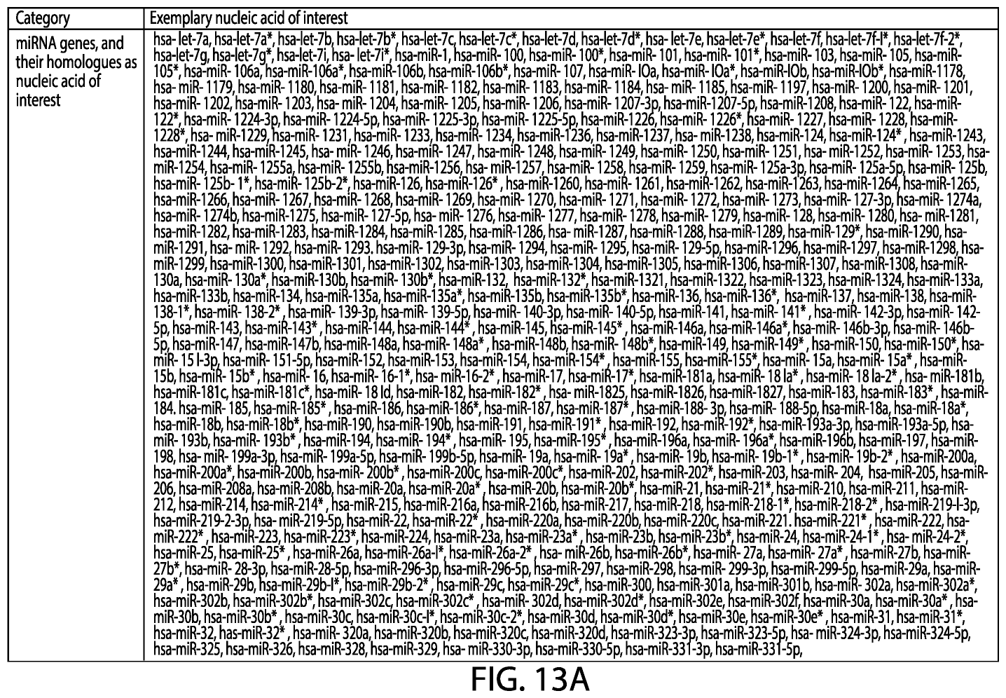

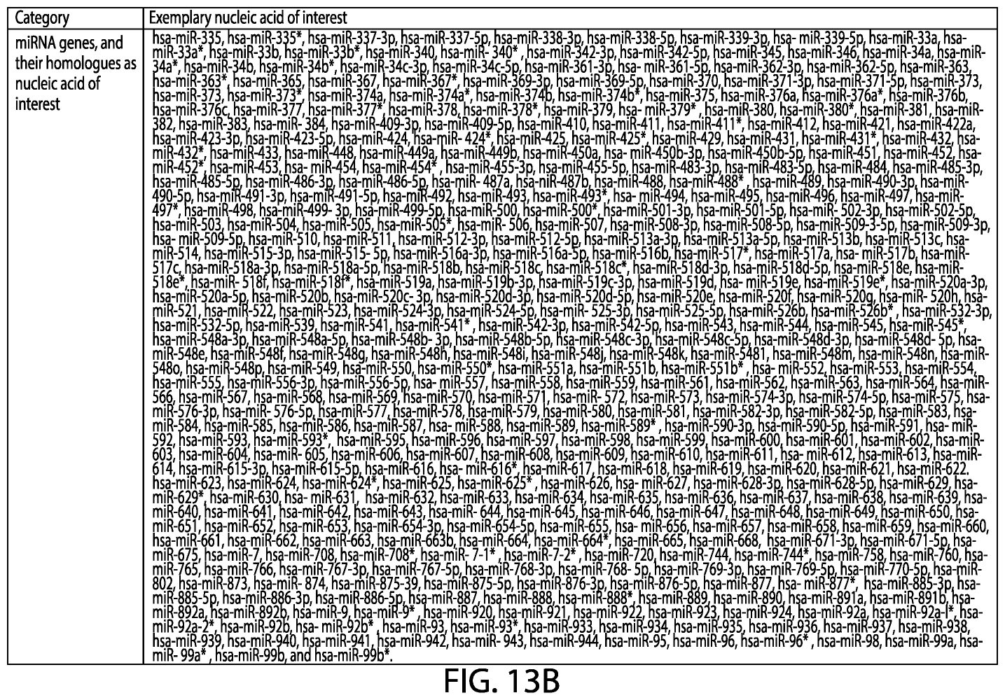

In some embodiments, the non-coding RNA comprises a miRNA. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA. FIG. 13A and FIG. 13B disclose a non-limiting list of miRNA genes, and their homologues, or as targets for small interfering nucleic acids encoded by the nucleic acid described herein (e.g., miRNA sponges, antisense oligonucleotides, TuD RNAs).

A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (de-repress the polypeptide). In some embodiments, de-repression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, an small interfering nucleic acid that is substantially complementary to a miRNA is one that is capable of hybridizing with a miRNA, and blocking the miRNA's activity. In some embodiments, a small interfering nucleic acid that is substantially complementary to a miRNA is a small interfering nucleic acid that is complementary with the miRNA at all but 1, 2, 3, 4, 5, 6, 7, 8, 9, 10, 11, 12, 13, 14, 15, 16, 17, or 18 bases. In some embodiments, an small interfering nucleic acid sequence that is substantially complementary to a miRNA, is an small interfering nucleic acid sequence that is complementary with the miRNA at, at least, one base.

### Gene-Editing Systems

In some embodiments, the methods and compositions described herein are used to integrate a nucleic acid into a GSH of the present disclosure within the target genome. In some embodiments, the integration is initiated and/or facilitated by an exogenously introduced nuclease, and the DNA break induced by the nuclease is repaired using the homology arms as a guide for homologous recombination, thereby inserting the nucleic acid flanked by the said homology arms into the target genome.

In some embodiments, the gene-editing system is introduced into a GSH to knock-down expression of an endogenous gene by introducing certain modifications in the gene or regulatory elements. In some embodiments, the gene-editing system may be introduced into a GSH to knock-out or delete all or a portion of an endogenous gene to remove a deleterious copy of the gene. In some embodiments, such negative modulation of gene expression is regulated, for example, the gene-editing system may be under an inducible promoter or a tissue-specific promoter, which allows selective gene down regulation, e.g., with temporal control (e.g., a gene can be deleted at a certain stage in differentiation), and/or tissue-specific knock-down or knock-out of a gene.

For example, a double-strand break (DSB) can be created by a site-specific nuclease such as a zinc-finger nuclease (ZFN) or TAL effector domain nuclease (TALEN). See, for example, Urnov et al. (2010) Nature 435(7042): 646-51; U.S. Pat. Nos. 8,586,526; 6,534,261; 6,599,692; 6,503,717; 6,689,558; 7,067,317; 7,262,054, the disclosures of which are incorporated by reference.

Another nuclease system involves the use of a so-called acquired immunity system found in bacteria and archaea known as the CRISPR/Cas system. CRISPR/Cas systems are found in 40% of bacteria and 90% of archaea and differ in the complexities of their systems. See, e.g., U.S. Pat. No. 8,697,359. The CRISPR loci (clustered regularly interspaced short palindromic repeat) are regions within the organism's genome where short segments of foreign DNA are integrated between short repeat palindromic sequences. These loci are transcribed and the RNA transcripts (“pre-crRNA”) are processed into short CRISPR RNAs (crRNAs). There are three types of CRISPR/Cas systems which all incorporate these RNAs and proteins known as “Cas” proteins (CRISPR associated). Types I and III both have Cas endonucleases that process the pre-crRNAs, that, when fully processed into crRNAs, assemble a multi-Cas protein complex that is capable of cleaving nucleic acids that are complementary to the crRNA.

In type II systems, crRNAs are produced using a different mechanism where a transactivating RNA (tracrRNA) complementary to repeat sequences in the pre-crRNA, triggers processing by a double strand-specific RNase III in the presence of the Cas9 protein or a variant thereof. Cas9 is then able to cleave a target DNA that is complementary to the mature crRNA however cleavage by Cas9 is dependent both upon base-pairing between the crRNA and the target DNA, and on the presence of a short motif in the crRNA referred to as the PAM sequence (protospacer adjacent motif) (see Qi et al (2013) Cell 152: 1173). In addition, the tracrRNA must also be present as it base pairs with the crRNA at its 3′ end, and this association triggers Cas9 activity.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand. The variants of Cas9 are art-recognized, e.g., Cas9 nickase mutant that reduces off-target activity (see e.g., Ran et al. (2014) Cell 154(6): 1380-1389), nCas, Cas9-D10A.

The requirement of the crRNA-tracrRNA complex can be avoided by use of an engineered “single-guide RNA” (sgRNA) that comprises the hairpin normally formed by the annealing of the crRNA and the tracrRNA (see Jinek et al (2012) Science 337:816 and Cong et al (2013) Sciencexpress/10.1126/science.1231143). Thus, exogenously introduced CRISPR endonuclease (e.g., Cas9 or a variant thereof) and a guide RNA (e.g., sgRNA or gRNA) can induce a DNA break at a specific locus within the genome of a target cell. Non-limiting examples of single-guide RNA or guide RNA (sgRNA or gRNA) sequences suitable for targeting are shown in Table 1 in U.S. Application 2015/0056705, which is incorporated herein in its entirety by reference. In addition, a sgRNA or gRNA may comprise a sequence of GSH loci described herein.

In some embodiments, the gene editing nucleic acid sequence encodes a molecule selected from the group consisting of: a sequence specific nuclease, one or more guide RNA (gRNA), CRISPR/Cas, a ribonucleoprotein (RNP) or any combination thereof. In some embodiments, the sequence-specific nuclease comprises: a TAL-nuclease, a zinc-finger nuclease (ZFN), a meganuclease, a megaTAL, or an RNA guide endonuclease of a CRISPR/Cas system (e.g., Cas proteins e.g. CAS 1-9, Csy, Cse, Cpfl, Cmr, Csx, Csf, cpfl, nCAS, or others). These gene editing systems are known to those of skill in the art, See for example, TALENS described in International Patent Application No, PCT/US2013/038536, and U.S. Patent Publication No. 2017-0191078-A9 which are incorporated by reference in their entirety. CRISPR cas9 systems are known in the art and described in U.S. patent application Ser. No. 13/842,859 filed on March 2013, and U.S. Pat. Nos. 8,697,359, 8,771,945, 8,795,965, 8,865,406, 8,871,445. The GSH is also useful for deactivated nuclease systems, such as CRISPRi or CRISPRa dCas systems, nCas, or Cas13 systems.

Guide RNAS (gRNAS)

In general, a guide sequence is any polynucleotide sequence having sufficient complementarity with a target polynucleotide sequence to hybridize with the target sequence and direct sequence-specific targeting of an RNA-guided endonuclease complex to the selected genomic target sequence. In some embodiments, a guide RNA binds to a target sequence and e.g., a CRISPR associated protein that can form a ribonucleoprotein (RNP), for example, a CRISPR/Cas complex.

In some embodiments, the guide RNA (gRNA) sequence comprises a targeting sequence that directs the gRNA sequence to a desired site in the genome, is fused to a crRNA and/or tracrRNA sequence that permit association of the guide sequence with the RNA-guided endonuclease. In some embodiments, the degree of complementarity between a guide sequence and its corresponding target sequence, when optimally aligned using a suitable alignment algorithm, is at least, about, or no more than 20%, 25%, 30%, 35%, 40%, 45%, 50%, 55%, 60%, 75%, 80%, 85%, 90%, 95%, 97.5%, 99%, or more. Optimal alignment can be determined with the use of any suitable algorithm for aligning sequences, such as the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP, and Maq.

A guide sequence can be selected to target any target sequence. In some embodiments, the target sequence is a sequence within a genome of a cell or within a GSH as disclosed herein. In some embodiments, the guide RNA can be complementary to either strand of the targeted DNA sequence. It is appreciated by one of skill in the art that for the purposes of targeted cleavage by an RNA-guided endonuclease, target sequences that are unique in the genome are preferred over target sequences that occur more than once in the genome. Bioinformatics software can be used to predict and minimize off-target effects of a guide RNA (see e.g., Naito et al. “CRISPRdirect: software for designing CRISPR/Cas guide RNA with reduced off-target sites” Bioinformatics (2014), epub: Heigwer et al. “E-CRISP: fast CRISPR target site identification” Nat. Methods 11:122-123 (2014); Bae et al. “Cas-OFFinder: a fast and versatile algorithm that searches for potential off-target sites of Cas9 RNA-guided endonucleases” Bioinformatics 30 (10): 1473-1475 (2014); Aach et al. “CasFinder: Flexible algorithm for identifying specific Cas9 targets in genomes” BioRxiv (2014)).

In general, a “crRNA/tracrRNA fusion sequence,” as that term is used herein refers to a nucleic acid sequence that is fused to a unique targeting sequence and that functions to permit formation of a complex comprising the guide RNA and the RNA-guided endonuclease. Such sequences can be modeled after CRISPR RNA (crRNA) sequences in prokaryotes, which comprise (i) a variable sequence termed a “protospacer” that corresponds to the target sequence as described herein, and (ii) a CRISPR repeat. Similarly, the tracrRNA (“transactivating CRISPR RNA”) portion of the fusion can be designed to comprise a secondary structure similar to the tracrRNA sequences in prokaryotes (e.g., a hairpin), to permit formation of the endonuclease complex. In some embodiments, the single transcript further includes a transcription termination sequence, such as a polyT sequence, for example six T nucleotides. In some embodiments, a guide RNA can comprise two RNA molecules and is referred to herein as a “dual guide RNA” or “dgRNA.” In some embodiments, the dgRNA may comprise a first RNA molecule comprising a crRNA, and a second RNA molecule comprising a tracrRNA. The first and second RNA molecules may form a RNA duplex via the base pairing between the flagpole on the crRNA and the tracrRNA. When using a dgRNA, the flagpole need not have an upper limit with respect to length.

In other embodiments, a guide RNA can comprise a single RNA molecule and is referred to herein as a “single guide RNA” or “sgRNA.” In some embodiments, the sgRNA can comprise a crRNA covalently linked to a tracrRNA. In some embodiments, the crRNA and tracrRNA can be covalently linked via a linker. In some embodiments, the sgRNA can comprise a stem-loop structure via the base-pairing between the flagpole on the crRNA and the tracrRNA. In some embodiments, a single-guide RNA is at least, about, or no more than 50, 60, 70, 80, 90, 100, 110, 120 or more nucleotides in length (e.g., 75-120, 75-110, 75-100, 75-90, 75-80, 80-120, 80-110, 80-100, 80-90, 85-120, 85-110, 85-100, 85-90, 90-120, 90-110, 90-100, 100-120, 100-120 nucleotides in length). In some embodiments, a nucleic acid vector as described herein for integration of a nucleic acid of interest into a GSH loci, or composition thereof comprises a nucleic acid that encodes at least 1 gRNA. For example, the second polynucleotide sequence may encode between 1 gRNA and 50 gRNAs, or at least, about, or no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 gRNAs. Each of the polynucleotide sequences encoding the different gRNAs can be operably linked to a promoter. In some embodiments, the promoters that are operably linked to the different gRNAs may be the same promoter. The promoters that are operably linked to the different gRNAs may be different promoters. The promoter may be a constitutive promoter, an inducible promoter, a repressible promoter, or a regulatable promoter.

In some embodiments, a non-GSH nucleic acid comprises or is introduced into a target cell in conjunction with another vector comprising a nucleic acid that encodes a Cas nickase (nCas: e.g., Cas9 nickase or Cas9-D10A). It is contemplated herein that such an nCas enzyme is used in conjunction with a guide RNA that comprises homology to a GSH as described herein and can be used, for example, to release physically constrained sequences or to provide torsional release. Releasing physically constrained sequences can, for example, “unwind” the vector such that a homology directed repair (HDR) template homology arm(s) are exposed for interaction with the genomic sequence.

In some embodiments, zinc finger nuclease is used to induce a DNA break that facilitates integration of the desired nucleic acid. “Zinc finger nuclease” or “ZFN” as used interchangeably herein refers to a chimeric protein molecule comprising at least one zinc finger DNA binding domain effectively linked to at least one nuclease or part of a nuclease capable of cleaving DNA when fully assembled. “Zinc finger” as used herein refers to a protein structure that recognizes and binds to DNA sequences. The zinc finger domain is the most common DNA-binding motif in the human proteome. A single zinc finger contains approximately 30 amino acids and the domain typically functions by binding 3 consecutive base pairs of DNA via interactions of a single amino acid side chain per base pair.

In some embodiments, a nucleic acid for integration described herein is integrated into a target genome in a nuclease-free homology-dependent repair systems, e.g., as described in Porro et al., Promoterless gene targeting without nucleases rescues lethality of a Crigler-Najjar syndrome mouse model, EMBO Molecular Medicine, (2017). In some embodiments, the in vivo gene targeting approaches are suitable for the insertion of a donor sequence, without the use of nucleases. In some embodiments, the donor sequence may be promoterless.

In some embodiments, the nuclease located between the restriction sites can be a RNA-guided endonuclease. As used herein, the term “RNA-guided endonuclease” refers to an endonuclease that forms a complex with an RNA molecule that comprises a region complementary to a selected target DNA sequence, such that the RNA molecule binds to the selected sequence to direct endonuclease activity to a selected target DNA sequence in a GSH identified herein.

### CRISPR CAS Systems

As art-recognized and described above, a CRISPR-CAS9 system includes a combination of protein and ribonucleic acid (“RNA”) that can alter the genetic sequence of an organism (see, e.g., U.S. publication 2014/0170753). CRISPR-Cas9 provides a set of tools for Cas9-mediated genome editing via nonhomologous end joining (NHEJ) or homologous recombination in mammalian cells. One of ordinary skill in the art may select between a number of known CRISPR systems such as Type I, Type II, and Type III. In some embodiments, a nucleic acid described herein for integration of a nucleic acid of interest into a GSH loci can be designed to include the sequences encoding one or more components of these systems such as the guide RNA, tracrRNA, or Cas (e.g., Cas9 or a variant thereof). In certain embodiments, a single promoter drives expression of a guide sequence and tracrRNA, and a separate promoter drives Cas (e.g., Cas9 or a variant thereof) expression. One of skill in the art will appreciate that certain Cas nucleases require the presence of a protospacer adjacent motif (PAM) adjacent to a target nucleic acid sequence.

RNA-guided nucleases including Cas (e.g., Cas9 or a variant thereof) are suitable for initiating and/or facilitating the integration of a nucleic acid described herein. The guide RNAs can be directed to the same strand of DNA or the complementary strand.

In some embodiments, the methods and compositions described herein can comprise and/or be used to deliver CRISPRi (CRISPR interference) and/or CRISPRa (CRISPR activation) systems to a host cell. CRISPRi and CRISPRa systems comprise a deactivated RNA-guided endonuclease (e.g., Cas9 or a variant thereof) that cannot generate a double strand break (DSB). This permits the endonuclease, in combination with the guide RNAs, to bind specifically to a target sequence in the genome and provide RNA-directed reversible transcriptional control.

Accordingly, in some embodiments, the nucleic acid compositions and methods described herein for integration of a nucleic acid of interest into a GSH locus can comprise a deactivated endonuclease, e.g., RNA-guided endonuclease and/or Cas9 or a variant thereof, wherein the deactivated endonuclease lacks endonuclease activity, but retains the ability to bind DNA in a site-specific manner, e.g., in combination with one or more guide RNAs and/or sgRNAs. In some embodiments, the vector can further comprise one or more tracrRNAs, guide RNAs, or sgRNAs. In some embodiments, the de-activated endonuclease can further comprise a transcriptional activation domain.

In some embodiments, the nucleic acid compositions and methods described herein for integration of a nucleic acid of interest into a GSH locus can comprise a hybrid recombinase. For example, Hybrid recombinases based on activated catalytic domains derived from the resolvase/invertase family of serine recombinases fused to Cys2-His2 zinc-finger or TAL effector DNA-binding domains are a class of reagents capable improved targeting specificity in mammalian cells and achieve excellent rates of site-specific integration. Suitable hybrid recombinases include those described in Gaj et al. Enhancing the Specificity of Recombinase-Mediated Genome Engineering through Dimer Interface Redesign, Journal of the American Chemical Society, (2014).

The nucleases described herein can be altered, e.g., engineered to design sequence specific nuclease (see, e.g., U.S. Pat. No. 8,021,867). Nucleases can be designed using the methods described in e.g., Certo et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, nuclease with site specific cutting characteristics can be obtained using commercially available technologies e.g., Precision BioSciences' Directed Nuclease Editor™ genome editing technology.

### MegaTALs

In some embodiments, the nuclease described herein can be a megaTAL. MegaTALs are engineered fusion proteins which comprise a transcription activator-like (TAL) effector domain and a meganuclease domain. MegaTALs retain the ease of target specificity engineering of TALs while reducing off-target effects and overall enzyme size and increasing activity. MegaTAL construction and use is described in more detail in, e.g., Boissel et al. 2014 Nucleic Acids Research 42 (4): 2591-601 and Boissel 2015 Methods Mol Biol 1239:171-196. Protocols for megaTAL-mediated gene knockout and gene editing are known in the art, see, e.g., Sather et al. Science Translational Medicine 2015 7(307): ra156 and Boissel et al. 2014 Nucleic Acids Research 42 (4): 2591-601. MegaTALs can be used as an alternative endonuclease in any of the methods and compositions described herein.

### Regulatory Sequences

A nucleic acid vector disclosed herein may also comprise transcriptional or translational regulatory sequences, for example, promoters, enhancers, insulators, internal ribosome entry sites, sequences encoding 2A peptides and/or polyadenylation signals.

In some embodiments, the regulatory sequence includes a suitable promoter sequence, being able to direct transcription of a gene operably linked to the promoter sequence, such as a nucleic acid of interest as described herein. In embodiments, an enhancer sequence is provided upstream of the promoter to increase the efficacy of the promoter. In some embodiments, the regulatory sequence includes an enhancer and a promoter, wherein the second nucleotide sequence includes an intron sequence upstream of the nucleotide sequence encoding a nuclease, wherein the intron includes one or more nuclease cleavage site(s), and wherein the promoter is operably linked to the nucleotide sequence encoding the nuclease.

Suitable promoters, including those described herein, can be derived from viruses and can therefore be referred to as viral promoters, or they can be derived from any organism, including prokaryotic or eukaryotic organisms. In some embodiments, promoters are derived from insect cells or mammalian cells. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (Miyagishi et al., Nature Biotechnology 20, 497-500 (2002)), an enhanced U6 promoter (e.g., Xia et al.,
Nucleic Acids Res. 2003 Sep. 1; 31(17)), a human H1 promoter (H1), and the like. In some embodiments, these promoters are altered to include one or more nuclease cleavage sites.

A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter, as well as the promoters listed below. Such promoters and/or enhancers can be used for expression of any gene of interest, e.g., the gene editing molecules, donor sequence, therapeutic proteins etc.). For example, the nucleic acid may comprise a promoter that is operably linked to the DNA endonuclease or CRISPR/Cas9-based system. The promoter operably linked to the CRISPR/Cas9-based system or the site-specific nuclease coding sequence may be a promoter from simian virus 40 (SV40), a CAG promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. The promoter may also be a tissue specific promoter, such as a liver specific promoter, natural or synthetic. In some embodiments, delivery to the liver can be achieved using endogenous ApoE specific targeting of the composition comprising a vector to hepatocytes via the low density lipoprotein (LDL) receptor present on the surface of the hepatocyte. In some embodiments, use is made of in silico designed synthetic promoters having an assembly of regulatory elements. These synthetic promoters are not naturally occurring and are designed either for optimal expression in the target tissue, regulated expression, or for accommodation in a virus capsid.

In some embodiments, the promoter may be selected from: (a) a promoter heterologous to the nucleic acid, (b) a promoter that facilitates the tissue-specific expression of the nucleic acid, preferably wherein the promoter facilitates hematopoietic cell-specific expression or erythroid lineage-specific expression, (c) a promoter that facilitates the constitutive expression of the nucleic acid, and (d) a promoter that is inducibly expressed, optionally in response to a metabolite or small molecule or chemical entity. Examples of inducible promoters include those regulated by tetracycline, cumate, rapamycin, FKCsA, ABA, tamoxifen, blue light, and riboswitch. Additional details are provided in e.g., Kallunki et al. (2019) Cells 8:E796, which is incorporated by reference. In some embodiments, the promoter is selected from the CMV promoter, β-globin promoter, CAG promoter, AHSP promoter, MND promoter, Wiskott-Aldrich promoter, and PKLR promoter. See also the section on “Pulsatile Gene Expression and Tunable Gene Expression.”

A significant number of genes and their control elements (promoters and enhancers) are known which direct the developmental and lineage-specific expression of endogenous genes. Accordingly, the selection of control element(s) and/or gene products inserted into stem cells will depend on what lineage and what stage of development is of interest. In addition, as more detail is understood on the finer mechanistic distinctions of lineage-specific expression and stem cell differentiation, it can be incorporated into the experimental protocol to fully optimize the system for the efficient isolation of a broad range of desired stem cells.

Any lineage-specific or cell fate regulatory element (e.g. promoter) or cell marker gene can be used in the compositions and methods described herein. Lineage-specific and cell fate genes or markers are well-known to those skilled in the art and can readily be selected to evaluate a particular lineage of interest. Non limiting examples of include, but not limited to, regulatory elements obtained from genes such as Ang2, Flk1, VEGFR, MHC genes, aP2, GFAP, Otx2 (see, e.g., U.S. Pat. No. 5,639,618), Dlx (Porteus et al. (1991) Neuron 7:221-229), Nix (Price et al. (1991) Nature 351:748-751), Emx (Simeone et al. (1992) EMBO J. 11:2541-2550), Wnt (Roelink and Nuse (1991) Genes Dev. 5:381-388), En (McMahon et al.), Hox (Chisaka et al. (1991) Nature 350:473-479), acetylcholine receptor beta chain (ACHRP) (Otl et al. (1994) J. Cell. Biochem. Supplement 18A: 177). Other examples of lineage-specific genes from which regulatory elements can be obtained are available on the NCBI-GEO web site which is easily accessible via the Internet and well known to those skilled in the art.

### Sequences

As used herein, coding region refers to regions of a nucleotide sequence comprising codons which are translated into amino acid residues, whereas noncoding region refers to regions of a nucleotide sequence that are not translated into amino acids. Transcribed non-coding sequences may be upstream (5′-UTR), downstream (3′-UTR), or intronic. Non-transcribed non-coding sequences may have cis-acting. regulatory functions, e.g., enhancer and promoter, or act as “spacers,” non-transcribed DNA used to separate functional groups in the DNA, e.g., polylinkers or “stuffer” DNA used to increase the size of the vector genome.

“Complement to” or “complementary” refers to the broad concept of sequence complementarity between regions of two nucleic acid strands or between two regions of the same nucleic acid strand. It is known that an adenine residue of a first nucleic acid region is capable of forming specific hydrogen bonds (base pairing) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% of the nucleotide residues of the first portion are capable of base pairing with nucleotide residues in the second portion. In other embodiments, all nucleotide residues of the first portion are capable of base pairing with nucleotide residues in the second portion.

A nucleic acid is operably linked when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

### Genetic Code

Alanine (Ala, A) GCA, GCC, GCG, GCT
Arginine (Arg, R) AGA, ACG, CGA, CGC, CGG, CGT
Asparagine (Asn, N) AAC, AAT
Aspartic acid (Asp, D) GAC, GAT
Cysteine (Cys, C) TGC, TGT
Glutamic acid (Glu, E) GAA, GAG
Glutamine (Gln, Q) CAA, CAG
Glycine (Gly, G) GGA, GGC, GGG, GGT
Histidine (His, H) CAC, CAT
Isoleucine (Ile, I) ATA, ATC, ATT
Leucine (Leu, L) CTA, CTC, CTG, CTT, TTA, TTG
Lysine (Lys, K) AAA, AAG
Methionine (Met, M) ATG
Phenylalanine (Phe, F) TTC, TTT
Proline (Pro, P) CCA, CCC, CCG, CCT
Serine (Ser, S) AGC, AGT, TCA, TCC, TCG, TCT
Threonine (Thr, T) ACA, ACC, ACG, ACT
Tryptophan (Trp, W) TGG
Tyrosine (Tyr, Y) TAC, TAT
Valine (Val, V) GTA, GTC, GTG, GTT
Termination signal (end) TAA, TAG, TGA

An important and well-known feature of the genetic code is its degeneracy, whereby, for most of the amino acids used to make proteins, more than one coding nucleotide triplet may be employed (illustrated above). Therefore, a number of different nucleotide sequences may code for a given amino acid sequence. The universality of the genetic code provides that such nucleotide sequences are considered functionally equivalent since they result in the production of the same amino acid sequence in all organisms, although mitochondria and plastids and similar symbiotic organelles have a slightly different genetic code. Although not all codons are utilized with similar translation efficiency, rare codons may lower the protein production due to limiting tRNA pools. Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

It is also known in the art that a nucleic acid encoding a polypeptide can be codon-optimized for certain host cells, without altering the amino acid sequence. Codon-optimization describes gene engineering approaches that use synonymous codon changes to increase protein production. This is possible because most amino acids are encoded by more than one codon. Replacing rare codons with frequently used ones have shown to increase protein expression.

In view of the foregoing, the nucleotide sequence of a DNA or RNA encoding a nucleic acid (or any portion thereof) described herein (e.g., a therapeutic nucleic acid) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for nucleic acid and polypeptide molecules useful in the present invention are well-known in the art and readily available on publicly available databases, such as the National Center for Biotechnology Information (NCBI).

TABLE 3
Exemplary Sequences of GSH loci

| Name | EVE | Type | Coordinates | Length |
|---|---|---|---|---|
| SYNTX- | dependo.2- | Intronic | 6:39420983-39427106 | 6124 |
| GSH1 | vespertilionidae | | | |
| SYNTX- | dependo.1- | Intronic | 9:36851490-36855495 | 4006 |
| GSH2 | whippomorpha | | | |
| SYNTX- | dependo.13- | Intergenic | 21:32096069-32100078 | 4010 |
| GSH3 | cercopithecidae | | | |
| SYNTX- | dependo.22-laurasiatheria | Intergenic | X:98173246-98177811 | 4566 |
| GSH4 | | | | |
| SYNTX- | dependo.0-macropus | Intergenic | 16:62187061-62192580 | 5,520 |
| GSH5 | | | | |
| SYNTX- | dependo.3-lagomorpha | Intergenic | 7:23216502-23222554 | 6053 |
| GSH6 | | | | |
| SYNTX- | dependo.4-rhinocerotidae | Intergenic | 2:56543541-56549589 | 6049 |
| GSH7 | | | | |
| SYNTX- | dependo.5-rhinocerotidae | Intronic | 4:143207807-143214243 | 6437 |
| GSH8 | | | | |
| SYNTX- | dependo.6-elephas | Intergenic | 18:38853056-38868234 | 15179 |
| GSH9 | | | | |
| SYNTX- | dependo.7-bradypus | Intronic | 20:52077189-52086514 | 9326 |
| GSH10 | | | | |
| SYNTX- | dependo.8-dasypus | Intergenic | 3:19731117-19735755 | 4639 |
| GSH11 | | | | |
| SYNTX- | dependo.10-diprotodontia | Intergenic | 8:80042442-80051437 | 8996 |
| GSH12 | | | | |
| SYNTX- | dependo.14- | Intergenic | 12:52480086-52484121 | 4036 |
| GSH13 | cercopithecidae | | | |
| SYNTX- | dependo.15-colobus | Intergenic | 3:115524837-115534489 | 9653 |
| GSH14 | | | | |
| SYNTX- | dependo.16-canid | Intergenic | 2:200141438-200149283 | 7846 |
| GSH15 | | | | |
| SYNTX- | dependo.20-laurasiatheria | Intronic | 22:24233627-24245930 | 12304 |
| GSH16 | | | | |
| SYNTX- | dependo.23-camelidae | Intergenic | 11:104769682- | 4268 |
| GSH17 | | | 104773949 | |
| SYNTX- | dependo.26-strepsirrhini | Intergenic | 11:134267756- | 8025 |
| GSH18 | | | 134275780 | |
| SYNTX- | dependo.27-eulemur | Intergenic | 5:120914982-120919271 | 4290 |
| GSH19 | | | | |
| SYNTX- | dependo.28-daubentonia | Intergenic | 12:40655895-40661452 | 5558 |
| GSH20 | | | | |
| SYNTX- | dependo.33-pteropodidae | Intergenic | 5:50956143-50960144 | 4002 |
| GSH21 | | | | |
| SYNTX- | dependo.36- | Intergenic | 6:130740286-130771217 | 30932 |
| GSH22 | phyllostomidae | | | |
| SYNTX- | dependo.37-rhinolophus | Intronic | 7:118260621-118265513 | 4893 |
| GSH23 | | | | |
| SYNTX- | dependo.40- | Intergenic | 14:86449965-86593451 | 143487 |
| GSH24 | phyllostomidae | | | |
| SYNTX- | dependo.44-bathyergidae | Intergenic | 6:133178282-133185376 | 7095 |
| GSH25 | | | | |
| SYNTX- | dependo.45-bathyergidae | Intronic | 20:50798439-50802719 | 4281 |
| GSH26 | | | | |
| SYNTX- | dependo.46-gliridae | Intergenic | 16:61490881-61499511 | 8631 |
| GSH27 | | | | |
| SYNTX- | dependo.47-pedetes | Intronic | 9:80969957-80976255 | 6299 |
| GSH28 | | | | |
| SYNTX- | dependo.50-muridae | Intergenic | 8:83008299-83040100 | 31802 |
| GSH29 | | | | |
| SYNTX- | dependo.54-cavia | Intergenic | 22:36143028-36187276 | 44249 |
| GSH30 | | | | |
| SYNTX- | dependo.55-rodent | Intergenic | 1:210271206-210283564 | 12359 |
| GSH31 | | | | |
| SYNTX- | dependo.56-cavia | Intronic | 3:108911357-108915358 | 4002 |
| GSH32 | | | | |
| SYNTX- | dependo.76-muridae | Intergenic | 16:55704192-55762599 | 58408 |
| GSH33 | | | | |
| SYNTX- | dependo.86- | Intergenic | 6:122259200-122266526 | 7327 |
| GSH34 | phyllostomidae | | | |
| SYNTX- | dependo.87-megaderma | Intergenic | 2:157638867-157645082 | 6216 |
| GSH35 | | | | |
| SYNTX- | dependo.88-megaderma | Intergenic | 5:179441107-179446029 | 4923 |
| GSH36 | | | | |
| SYNTX- | dependo.175-procavia | Intergenic | 1:8312557-8316629 | 4073 |
| GSH37 | | | | |
| SYNTX- | dependo.180-rhinolophus | Intergenic | 3:96718524-96722525 | 4002 |
| GSH38 | | | | |
| SYNTX- | dependo.187-glis | Intergenic | 4:96323055-96343599 | 20545 |
| GSH39 | | | | |
| SYNTX- | dependo.190-sarcophilus | Intergenic | 3:20857114-20872793 | 15680 |
| GSH40 | | | | |
| SYNTX- | dependo.191-sarcophilus | Intronic | 3:169508698-169527289 | 18592 |
| GSH41 | | | | |
| SYNTX- | dependo.192-sarcophilus | Intergenic | 3:118728528-118746362 | 17835 |
| GSH42 | | | | |
| SYNTX- | dependo.197- | Intergenic | 2:103571837-103607012 | 35176 |
| GSH43 | ornithorhynchus | | | |
| SYNTX- | dependo.199- | Intergenic | 6:141806202-141847712 | 41511 |
| GSH44 | ornithorhynchus | | | |
| SYNTX- | dependo.365- | Intergenic | 10:90996790-91165307 | 168518 |
| GSH45 | passeriformes | | | |
| SYNTX- | dependo.366- | Intergenic | 4:86813141-87049470 | 236330 |
| GSH46 | passeriformes | | | |
| SYNTX- | dependo.367-otididae | Intergenic | 12:84549762-84818096 | 268335 |
| GSH47 | | | | |
| SYNTX- | dependo.372- | Intergenic | 6:118315676-118462772 | 147097 |
| GSH48 | opisthocomus | | | |
| SYNTX- | dependo.xx-scolopacidae | Intergenic | 12:25248929-25650604 | 401676 |
| GSH49 | | | | |
| SYNTX- | | Intronic | 10:132751700- | 27907 |
| GSH50 | | | 132779606 | |
| SYNTX- | dependo.45-rodent | Intergenic | 6:132812569-133242340 | 429772 |
| GSH51 | | | | |
| SYNTX- | Amdo 567 Procavia | Intergenic | 8:128433359-128439048 | 5690 |
| GSH52 | | | | |
| SYNTX- | proto.176-NanGal | Intronic | 7:125054826-125058864 | 4038 |
| GSH53 | | | | |
| SYNTX- | proto.2-MusSpr | Intronic | 12:41260344-41265604 | 5260 |
| GSH54 | | | | |
| SYNTX- | proto.181-PhaCin | Intronic | 10:4650078-4658002 | 7924 |
| GSH55 | | | | |
| SYNTX- | proto.218-VomUrs | Intronic | 8:119589710-119595739 | 6029 |
| GSH56 | | | | |

* The coordinates in Table 3 are from human genome assembly GRCh38/hg38.
* Included above are cDNA, ssDNA, and RNA nucleic acid molecules (e.g., thymidines replaced with uridines), nucleic acid molecules encoding orthologs or variants of the encoded proteins, as well as nucleic acid sequences comprising a nucleic acid sequence having at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed above, or a portion thereof. Such nucleic acid molecules can have a function of the full-length nucleic acid as described further herein.
* See Table 5 in Example 3 for exemplary characterizations of the representative GSH loci.

### Pulsatile Gene Expression and Tunable Gene Expression

In certain aspects, the vectors (e.g., nucleic acid vectors, viral vectors), cells, pharmaceutical compositions, and/or methods of the present disclosure utilize a pulsatile and/or tunable gene expression. As used herein, tunable gene expression allows regulation of the transgene expression at will, e.g., using a small molecule or an oligonucleotide (e.g., tetracycline or antisense oligonucleotides (ASO or AON), respectively) to turn on or turn off the expression of the transgene. While tunable gene expression is often achieved using an inducible promoter or a repressible promoter, the tunable regulation is intended to include the regulation of gene expression beyond transcription.

Accordingly, tunable gene expression is intended to encompass temporal regulation at transcriptional, post-transcriptional, translational, and/or post-translational levels. Tunable expression is compatible with spatial control of the gene expression. For example, spatial control of a transgene may be facilitated by placing a transgene under a tissue-specific promoter, which is then combined with an expression-modulating agent (e.g., tetracycline or ASO) that mediates temporal control.

Pulsatile gene expression refers to turning on and off the production of the transgene at regular intervals. Any tunable gene expression system may be utilized for pulsatile gene expression. In addition, it is contemplated herein that modulation of any gene expression described herein may be used in combination with pulsatile gene expression.

Pulsatile gene expression is important for the success of gene therapy. Obtaining physiological and long-term protein expression levels remains a major challenge in gene therapy applications. High-level expression of a transgene can induce ER stress and unfolded protein response months after treatment, leading to a pro-inflammatory state and cell death, jeopardizing the therapy's benefit. The pulsatile transgene expression strategy (PTES) can spare the target cell from overexpression stress, and allow long-term expression of the transgene without gradual reduction in expression over time. In addition, the pulsatile and/or tunable expression may improve, e.g., the efficiency of the production and/or stability of the protein encoded by the transgene.

In some embodiments, PTES described herein is a tunable expression system where the default state is off until a reagent turns-on or disinhibits expression, allowing calibration of dose to meet patients' specific needs, providing greater safety and long-term benefits. The timing of the pulses can be determined from the initial serum levels (t0) and the half-life (t1/2) of protein of interest (see Example 11).

### Exemplary Tunable Expression System

### Tetracycline-Controlled Operator System

A bacterial regulatory element, the Tn10-specified tetracycline-resistance operon of E. coli, can be used to regulate gene expression. For example, there are three exemplary configurations of this system: (1) The repression-based configuration, in which a Tet operator (TetO) is inserted between the constitutive promoter and gene of interest and where the binding of the tet repressor (TetR) to the operator suppresses downstream gene expression. In this system, the addition of tetracycline results in the disruption of the association between TetR and TetO, thereby triggering TetO-dependent gene expression. (2) Tet-off configuration, where tandem TetO sequences are positioned upstream of the minimal constitutive promoter followed by cDNA of gene of interest. Here, a chimeric protein consisting of TetR and VP16 (tTA), a eukaryotic transactivator derived from herpes simplex virus type 1, is converted into a transcriptional activator, and the expression plasmid is transfected together with the operator plasmid. Thus, culturing cells with tetracycline switches off the exogenous gene expression, while removing tetracycline switches it on. (3) Tet-on configuration, where the exogenous gene is expressed when tetracycline is added to the growth medium. Even though tetracycline is nontoxic to mammalian cells at the low concentration required to regulate TetO-dependent gene expression, its continuous presence may not be desired. Thus, a mutant tTA with four amino acid substitutions, termed rtTA, was developed by random mutagenesis of tTA. Unlike tTA, rtTA binds to TetO sequences in the presence of tetracycline, thereby activating the silent minimal promoter.

### Cumate-Controlled Operator System

The cumate-controlled operator originates from the p-cmt and p-cym operons in Pseudomonas putida. The corresponding repressor contains an N-terminal DNA-binding domain recognizing the imperfect repeat between the promoter and the beginning of the first gene in the p-cymene degradative pathway. Similarly to a tetracycline-controlled operator system, the cumate operator (CuO) and its repressor (CymR) can be engineered into three configurations: (1) The repressor configuration, which is realized by placing CuO downstream of a constitutive promoter, where the binding of CymR to CuO efficiently suppresses downstream gene expression. The addition of cumate releases CymR, thereby triggering downstream gene expression. (2) Activator configuration, where chimeric molecular (cTA) is formed via the fusion of CymR and VP16. In this configuration, a minimal promoter was placed downstream of the multimerized operator binding sites (6xCuO). (3) Reverse activator configuration, for which after the random mutagenesis and screening, cTA mutant (rcTA) that binds to CuO upon addition of cumate was generated. In this configuration, the addition of cumate triggered downstream gene expression.

### Protein-Protein Interaction-Based Chimeric System

1. Induction of Target Gene by Control of the Interaction Between FKBP12 and mTOR

Rapamycin and its analog FK506 bind to a cytosolic protein FKBP12. This complex further binds to mTOR, forming a tripartite complex. Therefore, fusing FKBP12 and mTOR with a DNA-binding domain of ZFHD1 and the activation domain of NF-κB p65 protein, respectively, bridges both domains to drive expression of the gene of interest in a rapamycin-dependent fashion. Due to the immunosuppressive and the cell cycle inhibitory effect of FK506 and rapamycin, a new synthetic compound, FKCsA, which is a heterodimer of FK506 and cyclosporin A (an immunosuppressant complexed with protein cyclophilin), was developed and was shown to exhibit neither toxicity nor immunosuppressive effects. To trigger gene expression, the addition of FKCsA to cells hinges FKBP12 fused with the Gal4 DNA-binding domain (Gal4DBD) and cyclophilin fused with VP16, thereby activating expression of the gene of interest downstream of upstream activation sequence (UAS, Gal4DBD binding site).

### 2. Induction of Target Gene by Control of the Interaction Between PYL1 and ABI1

Abscisic acid (ABA)-regulated interaction between two plant proteins is used to regulate gene expression in a temporal and quantitive manner in mammalian cells. The two proteins are PYL1 (abscisic acid receptor) and ABI1 (protein phosphatase 2C56), which are important players of the ABA signaling pathway required for stress responses and developmental decisions in plants. According to the crystal structure of PYL1-ABA-ABI1 complex, interacting complementary surfaces of PYL1 (amino acids 33 to 209) and ABI1 (amino acids 126 to 423) were chosen for chimeric protein construction. Similarly, Gal4DBD was fused with ABIL and VP16 with PYL1. Thus after transfecting this ABA-activator cassette and UAS-driven reporter into mammalian cells, ABA significantly induced the reporter's production. Compared to the rapamycin system, the ABA system has two compelling advantages: first, ABA is present in many foods containing plant extracts and oils—its lack of toxicity is supported by an extensive evaluation by the Environmental Protection Agency (EPA), secondly, since the ABA signaling pathway does not exist in mammalian cells, there should be no competing endogenous binding proteins as in the rapamycin systems. To further avoid any catalysis of possible unexpected substrates by ABI1, a mutation critical for its phosphatase activity was introduced into the chimeric protein.

### 3. Induction of Target Gene by Light Sensitive Protein-Protein Interactions

Two light-switchable transgene systems were developed by taking advantage of light-induced protein-protein interactions. The first one got inspiration from the molecular basis of the circadian rhythm of fungi. Vivid (VVD), a photoreceptor and light-oxygen-voltage (LOV) domain-containing protein from Neurospora crassa, forms a rapidly exchanging dimer upon blue-light activation. Thus, the chimeric protein consisting of VVD and Gal4 residues 1-65 dimerizes and becomes a transcriptional activator under blue light-illumination, while the active dimer disassociates in the absence of blue light. This means that the expression of the reporter downstream of UAS can be switched on and off in a spatiotemporal manner utilizing blue light. Moreover, mutagenesis optimization of VVD further reduced the background expression to a minimal level, making the system even more feasible. Another light-switchable transgene system (photoactivatable (PA)-Tet-OFF/ON) exploits the Arabidopsis thaliana-derived blue light-responsive heterodimer formation, consisting of the cryptochrome 2 (Cry2) photoreceptor and cryptochrome-interacting basic helix-loop-helix 1 (CIB1). Photolyase homology region (PHR) at Cry2's N-terminal part is the chromophore-binding domain that binds to Flavin adenine dinucleotide (FAD) by a noncovalent bond. CIB1 interacts with Cry2 in blue light-dependent manner. Thus, to make an inducible expression system, PHR was fused with the transcription activation domain of p65, and CIB1 was fused with the DNA binding, dimerization and Tetracycline-binding domains of TetR (residues 1-206). Accordingly, the reporter gene can be switched on with blue light illumination, while switching off can be achieved in two ways, either by the absence of the blue light or tetracycline addition. Meanwhile, a tetracycline insensitive mutation, H100Y, was established to make it purely dependent on illumination. Applying the same chimeric structure, but replacing TetR with rtTA, the reporter gene can be switched on with either blue light illumination or tetracycline, and switched off either by absence of the blue light or removal of tetracycline. Generally, two advantages of light-switchable transgene systems overwhelm all other systems. One is their rapid on and off cycle. Due to the nature of circadian rhythm, the two above-mentioned protein-protein interactions are dynamic, leading to a fast response and turnover. Even short pulses of light for 1-2 min are sufficient to induce luciferase expression, which has been shown to peak 1.1 h later and decline to the background level 3 h later. The other advantage is its precise spatial induction. Illumination within restricted areas or cell populations can be realized with advanced illumination sources, by which the reporter expression can be selectively induced in certain cells or subcellular regions of interest. These unique features will not only greatly facilitate the future cell-cell behavior studies, but also provide vast potential for clinical gene therapy.

### 4 Tamoxifen Controlled System

The tamoxifen inducible system, one of the best-characterized “reversible switch” models, has a number of beneficial features (e.g., reviewed by Whitfield et al. (2015) Cold Spring Harb Protoc. 2015 (3): 227-234). In this system, the hormone-binding domain of the mammalian estrogen receptor is used as a heterologous regulatory domain. Upon ligand binding, the receptor is released from its inhibitory complex and the fusion protein becomes functional. For example, a ligand-binding domain (LBD) of the estrogen receptor (ER) can be fused with a transgene, the product of which is a chimeric protein that can be activated by anti-estrogen tamoxifen or its derivative 4-OH tamoxifen (4-OH-TAM).

This system has been used in combination with a recombinase to generate a regulatable recombinase that modifies the genome. For example, either single or two plasmid systems can be used to achieve inducible gene expression. The first successful case was done in mouse embryonic cells. Two plasmids were transfected together. One was Cre-ER constitutive expressing plasmid, the other contained gene trap sequence flanked by LoxP, followed by β-galactosidase (LacZ) open reading frame. As a consequence, expression of LacZ could only be restored when Cre-loxP-mediated recombination was triggered and the gene trap sequence was excised. By these means, the reporter gene could be induced not only in undifferentiated embryonic stem cells and embryoid bodies, but also in all tissues of a 10-day-old chimeric fetus or specific differentiated adult tissues. In another example, to induce enhanced green fluorescent protein (EGFP) expression in baby hamster kidney (BHK) cells and to simplify the plasmid construction, Cre-ER cDNA flanked by LoxP sites were inserted between phosphoglycerate kinase (PGK) promoter and EGFP encoding sequence. In this system, Cre-ER functions as a gene trap to block the transcription of EGFP without 4-OH-TAM. Ignition of recombinase activity by 4-OH-TAM melts off the Cre-ER cassette and restores EGFP expression driven by PGK promoter. To exclude the effect exerted by endogenous steroids, three distinct ERs are mostly exploited: (1) mouse ER™ with a G525R mutation, (2) human ERT with G521R mutation and (3) human ERT2 containing three mutations G400V/M543/L544A.

### 5. Riboswitch-Regulatable Expression System

A riboswitch-regulatable expression system takes advantage of bacteria-derived RNA aptamers linked with hammerhead ribozymes (aptazymes). Aptamer acts as a molecular sensor and transducer for the whole apparatus, while ribozyme responds to the signal with conformation change and mRNA cleavage. For example, Gram-positive bacteria's aptazyme can directly sense excessive glucosamine-6-phosphate (GlcN6P) and cleave mRNA of the glms gene, whose protein product is an enzyme that converts fructose-6-phosphate (Fru6P) and glutamine to GlcN6P. These aptazymes, responding to tetracycline, theophylline, guanine, etc. were engineered to both knock down and overexpress the gene of interest (as reviewed by e.g., Yokobayashi et al. (2019) Curr Opin Chem Biol 52:72-78).

### 6 ASO (Antisense Oligonucleotides) Regulated Expression System

ASO can bind to DNA or RNA. ASO has demonstrated effective gene regulation acting at the RNA level to either activate the RISC complex and degrade the mRNA, or interfering with recognition of cis-acting elements. ASO are routinely formulated in lipid nanoparticles that efficiently transfect cells. The ASO are used for “knock-down” applications, either gain-of-function (i.e., dominant negative), transcripts, or homozygous recessive diseases. In diseases caused by dominant negative mutations where the ASO is not specific to the transcript from the mutant allele, e.g., Huntington's disease and other poly-glutamine expansion diseases, restoration of normal cell function may be accomplished using gene replacement using a vector—delivered transgene with alternative synonymouse codons that reduce sequence complementarity to exogenous ASO. Thus, the ASO depletes the transcripts from the endogenous alleles but the vector-driven transcripts are unaffected.

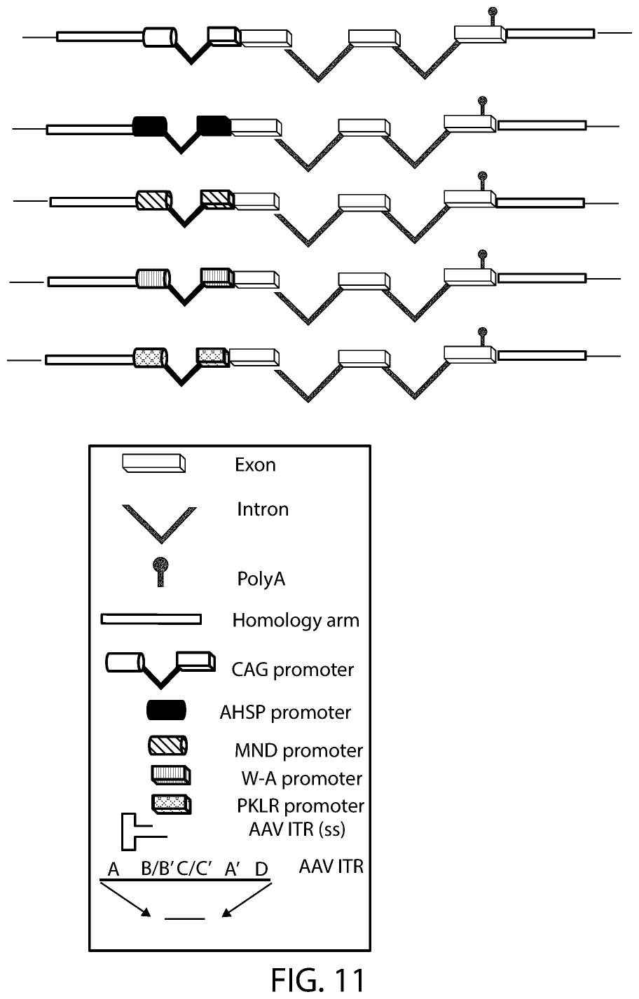

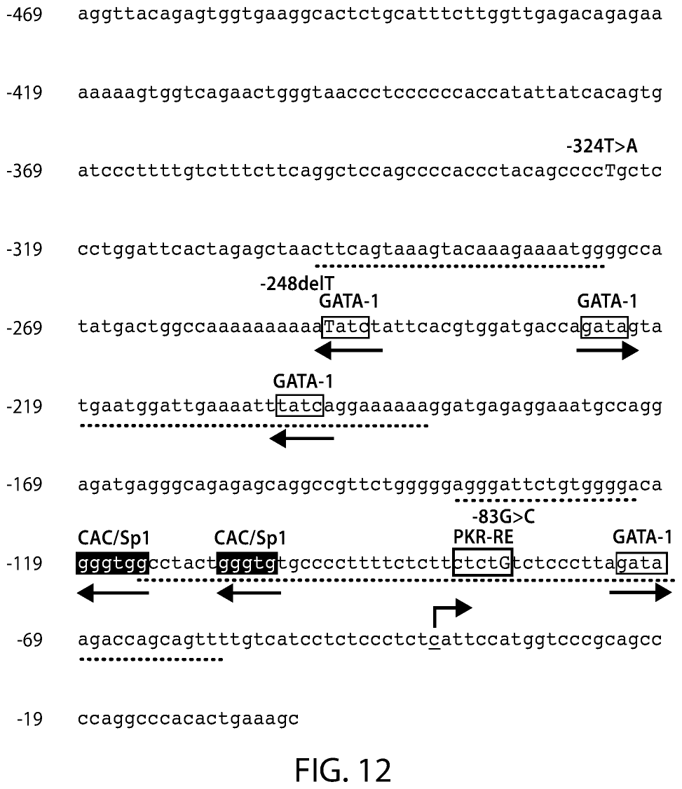

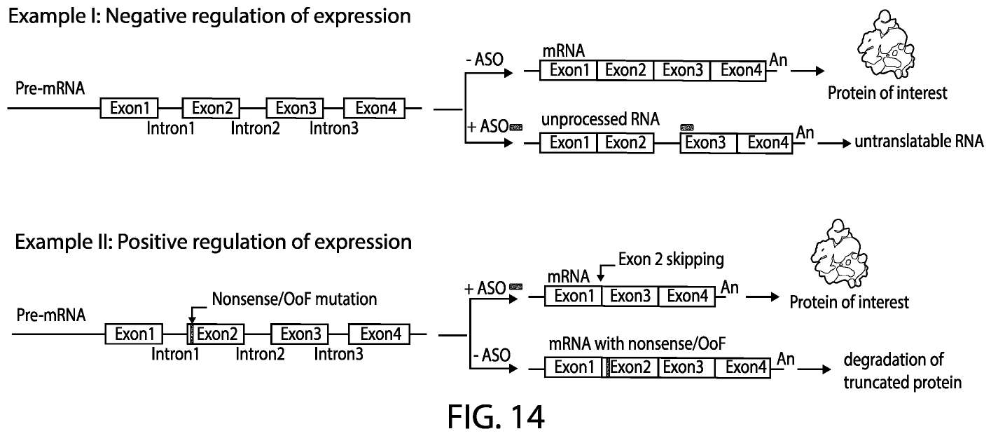

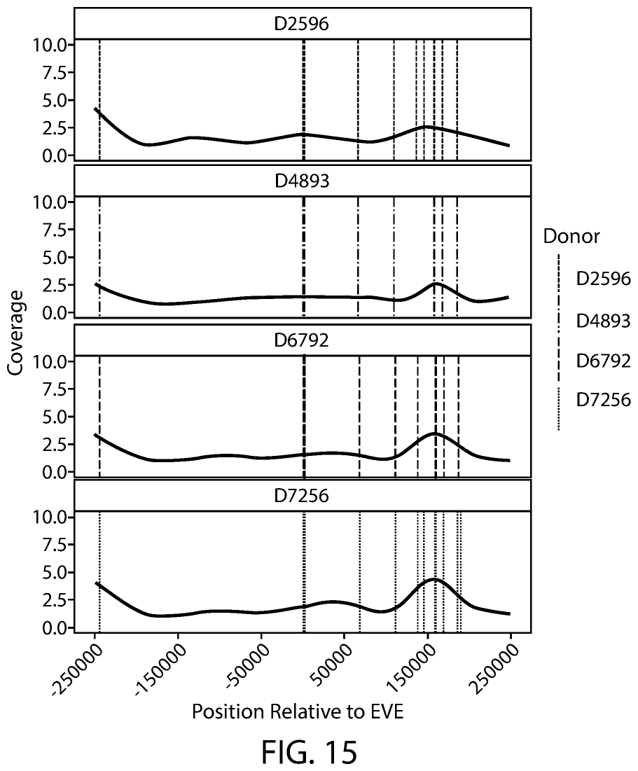

As illustrated in FIG. 14, ASO can modulate splicing to either negatively or positively regulate gene expression (see also Havens and Hastings (2016) Nucleic Acids Research 44:6549-6563). Example I of FIG. 11 shows that an ASO (an antisense oligonucleotides ASO or AON) can negatively regulate gene expression post-transcriptionally. Without ASO, a primary transcript is spliced into a translatable mRNA. The addition of an ASO (red line) complementary to the splice acceptor at the 3′ end of the intron/5′ end of Exon 2 interferes with splicing. Thus, in the presence of ASO, the intron remains in the transcript. This unprocessed RNA comprising the intron is either untranslatable or produces a non-functional protein upon translation.

Example II of FIG. 11 also illustrates that an ASO can positively affect gene expression post-transcriptionally. A primary transcript (left) contains 4 exons: exon 1, exon 3, and exon 4 encode the therapeutic protein, and exon 2 contains either a nonsense mutation(s) or an out-of-frame-mutation (OOF). Such exon 2 can be engineered into any transgene. Without the ASO, the transcript is processed into a mature mRNA comprising 4 exons, i.e., exon 2 with a nonsense mutation(s) or an OOF mutation remains. Thus, the resulting mRNA translates into a truncated or non-functional protein. By contrast, the addition of ASO interferes with splicing, and the mature mRNA consists of exon 1, exon 3, and exon 4, i.e., exon 2 with a nonsense mutation(s) or an OOF mutation is spliced out. Thus, at the default state (no ASO), the therapeutic protein is not produced. Only upon the addition of ASO, the therapeutic protein is produced, thereby resulting in positive regulation.

These approaches allow for knock-down of constitutively active transgene expression, i.e., default on. In some embodiments, the default on state is preferred. In other embodiments, a default off condition is preferred.

### Exemplary Pulsatile Gene Expression for Hemophilia A

In certain aspects, vectors (e.g., nucleic acid vectors, viral vectors), cells, pharmaceutical compositions, and methods provided herein use the pulsatile gene expression for gene therapy for a subject afflicted with hemophilia A. In some embodiments, an ASO regulated expression system is used to transduce a gene encoding human coagulation Factor VIII (FVIII) to hepatocytes in a subject afflicted with hemophilia A. In some embodiments, a pulsatile gene expression (the transgene encoding FVIII is turned on and off at certain intervals) is used to regulate the amount of FVIII produced (see Example 11). The delivery and regulation of the transgene encoding FVIII or an active fragment thereof (e.g., with its B-domain deletion), the compositions and methods described herein address a long-felt medical need for which there is still no solution.

In 2020, the FDA did not approve the Biomarin biologics license application (BLA) for Valoctocogene Roxaparvovec (or BMN270) as a treatment for hemophilia A (HemA). A recombinant adeno-associated virus type 5 (rAAV5) delivered a derivative of the gene for human coagulation factor VIII (FVIII) to the liver of HemA patients. At higher doses, FVIII was expressed and secreted into the circulation of patients at levels equal to or greater than physiological levels effectively “curing” the treated patients. However, long-term expression levels decreased 0.5 to 0.33 each year during the three-year follow-up. Although the FVIII expression remained at levels that are clinically beneficial, the FDA expressed concern that if expression continued to decline at the same rate, the patients would revert to their hemophiliac phenotype. There are no definitive explanations for the decremental expression pattern: previous clinical studies for hemophilia B established that loss of FIX expression was primarily attributed to acute inflammation elicited by processed AAV capsid antigens. However, prophylactic steroid treatment attenuated or eliminated the capsid immune response and is now routine for liver directed rAAV treatments. Several possible explanations that account for the loss of FVIII expression are contemplated herein. FVIII has been a difficult recombinant protein to produce in either microbial or eukaryotic expression systems. The development of the “B-domain” deleted version of FVIII reduced the size of the open-reading frame and improved the expression level. However, the FVIII expression levels were still substantially lower than other proteins. To overcome these low levels, Biomarin increased the vector dose in the clinical studies. Patients were treated with 6E+13 vector particles (referred to as vector genomes, or vg) per kg. Based on large animal models, a small minority of hepatocytes take-up (transduced) with rAAV5-FVIII and as a result of the large number of vg per cell, then express relatively large quantities of FVIII. The metabolic demand for FVIII expression likely disrupts the normal requirements for hepatocyte protein expression. The hepatocyte cellular compartments normally involved in protein folding and secretion may become congested with the FVIII. Endothelial cells that produce FVIII production are likely specialized for this activity and produce FVIII from the allele on the single X chromosome under the transcriptional control of the highly regulated native FVIII promoter.

Accordingly, in order to prevent gradual reduction in expression of the transgene encoding FVIII, the transgene is turned on and off at regular intervals to achieve a long-term efficacy. The timing of the pulses is determined based on the serum level and half-life of the FVIII protein (see Example 11 for details). For FVIII for hemophilia A prevention or treatment, the ideal state is off until transiently activated. ASO can be used to elicit either a negative or a positive effect by interfering with cis-acting elements in the primary transcript, thereby providing flexibility in regulation of the pulsatile gene expression.

### Viral Vectors

In certain aspects, provided herein are viral vectors comprising the nucleic acid vectors described herein (e.g., those comprising at least a portion of a GSH locus of the present disclosure, those nucleic acid vectors for integration into a GSH locus of the present disclosure, etc.). In some embodiments, the viral vector is selected from rAd, AAV, rHSV, retroviral vector, poxvirus vector, lentivirus, vaccinia virus vector, HSV Type 1 (HSV-1)-AAV hybrid vector, baculovirus expression vector system (BEVS), and variants thereof.

Specifically, a viral vector refers to a virus or viral chromosomal material into which a fragment of foreign DNA can be inserted for transfer into a cell. Any virus that includes a DNA stage in its life cycle may be used as a viral vector in the subject methods and compositions. For example, the virus may be a single strand DNA (ssDNA) virus or a double strand DNA (dsDNA) virus. Also suitable are RNA viruses that have a DNA stage in their lifecycle, for example, retroviruses, e.g. MMLV, lentivirus, which are reverse-transcribed into DNA. The virus can be an integrating virus or a non-integrating virus.

Viral vectors encompassed for use in the methods and compositions as disclosed herein are discussed in review article Hendrie, Paul C., and David W. Russell. “Gene targeting with viral vectors.” Molecular Therapy 12.1 (2005): 9-17 and Perez-Pinera, “Advances in targeted genome editing.” Current opinion in chemical biology 16.3 (2012): 268-277.

Adeno-associated virus (“AAV”) vectors are encompassed for use as nucleic acid vector compositions as disclosed herein, and are useful for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al, Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). At least six viral vector approaches are currently available for gene transfer in clinical trials, which utilize approaches that involve complementation of defective vectors by genes inserted into helper cell lines to generate the transducing agent.

In preferred embodiments, a viral vector is an adeno-associated virus. By adeno-associated virus, or “AAV” it is meant the virus itself or derivatives thereof. The term covers all subtypes and both naturally occurring and recombinant forms, except where required otherwise, for example, AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), AAV type 12 (AAV-12), AAV type 13 (AAV-13), avian AAV, bovine AAV, canine AAV, equine AAV, primate AAV, non-primate AAV, ovine AAV, a hybrid AAV (i.e., an AAV comprising a capsid protein of one AAV subtype and genomic material of another subtype), an AAV comprising a mutant AAV capsid protein or a chimeric AAV capsid (i.e. a capsid protein with regions or domains or individual amino acids that are derived from two or more different serotypes of AAV, e.g. AAV-DJ, AAV-LK3, AAV-LK19). “Primate AAV” refers to AAV that infect primates, “non-primate AAV” refers to AAV that infect non-primate mammals, “bovine AAV” refers to AAV that infect bovine mammals, etc.

A recombinant AAV vector or rAAV vector means an AAV virus or AAV viral chromosomal material comprising a polynucleotide sequence not of AAV origin (i.e., a polynucleotide heterologous to AAV), typically a nucleic acid sequence of interest to be integrated into the cell (e.g., a non-GSH nucleic acid). In general, the heterologous polynucleotide is flanked by at least one, and generally by two AAV inverted terminal repeat sequences (ITRs). In some instances, the recombinant viral vector also comprises viral genes important for the packaging of the recombinant viral vector material. By “packaging” it is meant a series of intracellular events that result in the assembly and encapsidation of a viral particle, e.g. an AAV viral particle. Examples of nucleic acid sequences important for AAV packaging (i.e., “packaging genes”) include the AAV “rep” and “cap” genes, which encode for replication and encapsidation proteins of adeno-associated virus, respectively. The term rAAV vector encompasses both rAAV vector particles and rAAV vector plasmids.

A viral particle refers to a single unit of virus comprising a capsid encapsidating a virus-based polynucleotide, e.g, the viral genome (as in a wild type virus), or, e.g., the subject targeting vector (as in a recombinant virus). An AAV viral particle refers to a viral particle composed of at least one AAV capsid protein (typically by all of the capsid proteins of a wild-type AAV) and an encapsidated polynucleotide AAV vector. If the particle comprises a heterologous polynucleotide (i.e. a polynucleotide other than a wild-type AAV genome, such as a transgene to be delivered to a mammalian cell), it is typically referred to as an rAAV vector particle or simply an rAAV vector. Thus, production of rAAV particle necessarily includes production of rAAV vector, as such a vector is contained within an rAAV particle.

In some embodiments, recombinant adeno-associated virus (“rAAV”) vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). All AAV serotypes, including AAV1, AAV2, AAV3, AAV4, AAV5, AAV6, AAV7, AAV8, AAV9, AAV10, AAV11, AAV12, AAV13, and AAVrh.10 and any novel AAV serotype can also be used in accordance with the present invention.

Replication-deficient recombinant adenoviral vectors (Ad) are also encompassed for use herein, can be produced at high titer and readily infect a number of different cell types. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Retroviral vectors are encompassed for use as nucleic acid vector compositions as disclosed herein. pLASN and MFG-S are examples of retroviral vectors that have been used in clinical trials (Dunbar et al, Blood 85:3048-305 (1995); Kohn et al., Nat. Med. 1:1017-102 (1995); Malech et al, PNAS 94:22 12133-12138 (1997)).

Vectors suitable in the methods and compositions as disclosed herein include lentivirus vectors, such as those disclosed in Picanco-Castro. “Advances in lentiviral vectors: a patent review.” Recent patents on DNA & gene sequences 6.2 (2012): 82-90. The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats (LTRs) with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et al, J. Virol. 66:1635-1640 (1992); Sommerfelt et al, Virol. 176:58-59′ (1990); Wilson et al, J. Virol. 63:2374-2378 (1989); Miller et al, J. Virol. 65:2220-2224 (1991); PCT/US94/05700). Other retroviral vectors for use herein include foamy viruses, as disclosed in Sweeney, Nathan Paul, et al. “Delivery of large transgene cassettes by foamy virus vector.” Scientific reports 7 (2017) 8085.

Lentiviral transfer vectors can be produced generally by methods well known in the art. See, e.g., U.S. Pat. Nos. 5,994,136; 6,165,782; and 6,428,953, US application 2014/0315294 and described in Merten et al “Production of lentiviral vectors.” Molecular Therapy-Methods & Clinical Development 3 (2016): 16017 and Merten, et al. “Large-scale manufacture and characterization of a lentiviral vector produced for clinical ex vivo gene therapy application.” Human gene therapy 22.3 (2010): 343-356, each of which are incorporated herein in their entirety by reference. In some embodiments, the lentivirus is an integrase deficient lentiviral vector (IDLV), IDLVs may be produced as described, for example using lentivirus vectors that include one or more mutations in the native lentivirus integrase gene, for instance as disclosed in Leavitt et al. (1996) J. Virol. 70 (2): 721-728; Philippe et al. (2006) Proc. Nat II Acad. ScL USA 103 (47): 17684-17689; and W O 06/010834. Lentiviruses for use in the methods and compositions as disclosed herein are disclosed in U.S. Pat. Nos. 6,207,455, 5,994,136, 7,250,299, 6,235,522, 6,312,682, 6,485,965, 5,817,491; 5,591,624.

Vectors suitable in the methods and compositions as disclosed herein include non-integrating lentivirus vectors (IDLV). See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222; U.S. Patent Publication No 2009/054985. In certain embodiments, the IDLV is an HIV lentiviral vector comprising a mutation at position 64 of the integrase protein (D64V), as described in Leavitt et al. (1996) J. Virol. 70 (2): 721-728. Additional IDLV vectors suitable for use herein are described in U.S. patent application Ser. No. 12/288,847, incorporated by reference herein.

Vectors suitable in the methods and compositions as disclosed herein include recombinant HCMV and RHCMV vectors, as disclosed in US 2013/0136,768.

Nucleic acid vectors useful herein for introduction of a nucleic acid of interest into a hematopoietic stem cell, e.g., CD34+ cells, include adenovirus Type 35. Nucleic acid vectors useful herein for introduction of a nucleic acid of interest into immune cells (e.g., T-cells) include non-integrating lentivirus vectors. See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222.

Vectors suitable in the methods and compositions as disclosed herein include baclulovirus expression vector systems (BEVS), which are discussed in Felberbaum, “The baculovirus expression vector system: a commercial manufacturing platform for viral vaccines and gene therapy vectors.” Biotechnology journal 10.5 (2015): 702-714.

Vectors suitable in the methods and compositions as disclosed herein include the HSV Type 1 (HSV-1)-AAV hybrid vectors, for example, as disclosed in Heister, Thomas, et al. “Herpes simplex virus type 1/adeno-associated virus hybrid vectors mediate site-specific integration at the adeno-associated virus preintegration site, AAVS1, on human chromosome 19.” Journal of virology 76.14 (2002): 7163-7173, and 5,965,441. Other hybrid vectors can be used, e.g., disclosed in U.S. Pat. No. 6,218,186.

Cells Comprising One or More Nucleic Acid Vectors and/or Viral Vectors

In certain aspects, provided herein are cells comprising at least one nucleic acid vector of the present disclosure or at least one viral vector of the present disclosure.

In some embodiments, the cell is selected from a cell line or a primary cell. In some embodiments, the cell is a mammalian cell, an insect cell, a bacterial cell, a yeast cell, or a plant cell, optionally wherein the mammalian cell is a human cell or a rodent cell. In some embodiments, the cell is an insect cell; and the insect cell is derived from a species of lepidoptera. In some embodiments, the species of lepidoptera is Spodoptera frugiperda. Spodoptera littoralis, Spodoptera exigua, or Trichoplusia ni. In some embodiments, the insect cell is Sf9.

In some embodiments, the cell is selected from a hematopoietic cell, hematopoietic progenitor cell, hematopoietic stem cell, erythroid lineage cell, megakaryocyte, erythroid progenitor cell (EPC), CD34+ cell, CD44+ cell, red blood cell, CD36+ cell, mesenchymal stem cell, nerve cell, intestinal cell, intestinal stem cell, gut epithelial cell, endothelial cell, enteroendocrine cell, lung cell, lung progenitor cell, enterocyte, liver cell (e.g., hepatocyte, hepatic stellate cells, Kupffer cells (KCs), liver sinusoidal endothelial cells (LSECs), liver progenitor cell), stem cell, progenitor cell, induced pluripotent stem cell (iPSC), skin fibroblast, macrophage, brain microvascular endothelial cell (BMVECs), neural stem cell, muscle satellite cell, epithelial cell, airway epithelial cell, muscle progenitor cell, erythroid progenitor cell, lymphoid progenitor cell, B lymphoblast cell, B cell, T cell, basophilic Endemic Burkitt Lymphoma (EBL), polychromatic erythroblast, epidermal stem cell, epithelial stem cell, embryonic stem cell, P63-positive keratinocyte-derived stem cell, keratinocyte, pancreatic β-cell, K cell, L cell, HEK293 cell, HEK293T cell, MDCK cell, Vero cell, CHO, BHK1, NS0, Sp2/0, HeLa, A549, and orthochromatic erythroblast.

Cells with at Least One Non-GSH Nucleic Acid Integrated at One or More GSH Loci

Viral vectors include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 5 1(1): 3 1-44 (1995); Haddada et al., in Current Topics in Microbiology and Immunology Doerfler and Bohm (eds.) (1995); and Yu et al., Gene Therapy 1:13-26 (1994).

Thus, in certain aspects, provided herein are cells comprising at least one non-GSH nucleic acid integrated into a GSH in the genome of a cell, wherein the GSH is selected from Table 3. In some embodiments, the GSH nucleic acid comprises an untranslated sequence or an intron. In some embodiments, the GSH is selected from SYNTX-GSH1, SYNTX-GSH2, SYNTX-GSH3, and SYNTX-GSH4. In some embodiments, the at least one non-GSH nucleic acid is integrated into one or more GSH loci described herein.

It is contemplated herein that cells may have integrated at least one of any one of the nucleic acid vectors described herein. In some embodiments, the any one of the nucleic acid vectors is delivered to the cell by any one of the viral vectors described herein.

In certain embodiments, the cell comprises the at least one non-GSH nucleic acid integrated into a GSH in a forward orientation. In some embodiments, the at least one non-GSH nucleic acid is integrated into a GSH in a reverse orientation.

In certain embodiments, the cell comprises at least one non-GSH nucleic acid integrated into a GSH, wherein the at least one non-GSH nucleic acid (a) is operably linked to a promoter, or (b) is not operably linked to a promoter.

In some embodiments, the at least one non-GSH nucleic acid is operably linked to a promoter, and the promoter is selected from: (a) a promoter heterologous to the nucleic acid to which it is operably linked; (b) a promoter that facilitates the tissue-specific expression of the nucleic acid; (c) a promoter that facilitates the constitutive expression of the nucleic acid; (d) an inducible promoter; (e) an immediate early promoter of an animal DNA virus; (f) an immediate early promoter of an insect virus; and (g) an insect cell promoter.

In some embodiments, the inducible promoter operably linked to at least one non-GSH nucleic acid is modulated by an agent selected from a small molecule, a metabolite, an oligonucleotide, a riboswitch, a peptide, a peptidomimetic, a hormone, a hormone analog, and light. In some embodiments, the agent is selected from tetracycline, cumate, tamoxifen, estrogen, and an antisense oligonucleotide (ASO), rapamycin, FKCsA, blue light, abscisic acid (ABA), and riboswitch.

In some embodiments, the promoter that facilitates tissue-specific expression of the at least one non-GSH nucleic acid is a promoter that facilitates tissue-specific expression in a hematopoietic stem cell, a hematopoietic CD34+ cell, and epidermal stem cell, an epithelial stem cell, neural stem cell, a lung progenitor cell, a muscle satellite cell, an intestinal K cell, a neuronal cell, an airway epithelial cell, or a liver progenitor cell.

In some embodiments, the promoter that is operably linked to at least one non-GSH nucleic acid is selected from the CMV promoter, β-globin promoter, CAG promoter, AHSP promoter, MND promoter, Wiskott-Aldrich promoter, PKLR promoter, polyhedron (polh) promoter, and immediately early 1 gene (IE-1) promoter.

In certain embodiments, a cell comprises the at least one non-GSH nucleic acid integrated into a GSH, wherein the at least one non-GSH nucleic acid comprises a sequence that encodes a coding RNA. In some embodiments, the sequence encoding a coding RNA is codon-optimized for expression in a target cell. In some embodiments, the at least one non-GSH nucleic acid encoding a coding RNA further comprises a sequence encoding a signal peptide.

In some embodiments, a cell comprises the at least one non-GSH nucleic acid integrated into a GSH, wherein the at least one non-GSH nucleic acid encodes a coding RNA comprises a sequence encoding: (a) a protein or a fragment thereof, preferably a human protein or a fragment thereof; (b) a therapeutic protein or a fragment thereof, an antigen-binding protein, or a peptide; (c) a suicide gene, optionally Herpes Simplex Virus-1 Thymidine Kinase (HSV-TK); (d) a viral protein or a fragment thereof; (e) a nuclease, optionally a Transcription Activator-Like Effector Nuclease (TALEN), a zinc-finger nuclease (ZFN), a meganuclease, a megaTAL, or a CRISPR endonuclease, (e.g., a Cas9 endonuclease or a variant thereof); (f) a marker, e.g., luciferase or GFP; and/or (g) a drug resistance protein, e.g., antibiotic resistance gene, e.g., neomycin resistance.

The viral protein or a fragment thereof may comprise a structural protein (e.g., VP1, VP2, VP3) or a non-structural protein (e.g., Rep protein). In some embodiments, the viral protein or a fragment thereof comprises: (a) a parvovirus protein or a fragment thereof, optionally VP1, VP2, VP3, NS1, or Rep; (b) a retrovirus protein or a fragment thereof, optionally an envelope protein, gag, pol, or VSV-G; (c) an adenovirus protein or a fragment thereof, optionally E1A, E1B, E2A, E2B, E3, E4, or a structural protein (e.g., A, B, C); and/or (d) a herpes simplex virus protein or a fragment thereof, optionally ICP27, ICP4, or pac.

In some embodiments, a cell comprises at least one non-GSH nucleic acid that encodes a viral protein that is a surface protein of a virus. In some embodiments, the at least one non-GSH nucleic acid encoding a viral protein encodes a surface protein, or a fragment thereof, of a virus. In some embodiments, (a) the surface protein or a fragment thereof is an immunogenic surface protein that elicits immune response in a host, (b) the surface protein or a fragment thereof further comprises a signal peptide, (c) the gene encoding the surface protein or a fragment thereof is operably linked to an inducible promoter, and/or (d) the nucleic acid encoding the surface protein or fragment thereof further comprises a suicide gene. Cells comprising such nucleic acd are useful not only for producing recombinant viral proteins in vitro for use as a vaccine, but useful also for implanting into a subject for expression of a viral protein in vivo for in vivo immunization. The in vivo production of viral proteins may be under an inducible promoter, such that the amount of immunogen produced in vivo, as well as the duration of production, can be fine-tuned using a signal or agent that modulates the inducible promoter (see e.g., the section on Pulsatile Expression System described herein).

In some embodiments, such cells for producing vaccines in vitro or for in vivo immunization express the viral surface protein, wherein the surface protein is of a coronavirus (e.g., MERS, SARS), influenza virus, respiratory syncytial virus, hepatitis A, hepatitis B, hepatitis C, hepatitis D, hepatitis E, human papillomavirus, dengue virus serotype 1, dengue virus serotype 2, dengue virus serotype 3, dengue virus serotype 4, zika, virus, West Nile virus, yellow fever virus, Chikungunya virus, Mayaro virus, Ebola virus, Marburg virus, or Nipa virus. In some embodiments, the surface protein is the spike protein of SARS-COV-2.

In some embodiments, a cell comprises at least one non-GSH nucleic acid integrated into a GSH, wherein the at least one non-GSH nucleic acid encodes a polypeptide or a fragment thereof. In preferred embodiments, such polypeptide or a fragment thereof is a therapeutic protein or a fragment thereof. In some embodiments, the at least one non-GSH nucleic acid comprising a sequence encoding a protein, or a fragment thereof, is selected from a hemoglobin gene (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), GBA1, preproinsulin, insulin, GIP, GLP-1, CEP290, ATPB1, ATPB11, ABCB4, CPS1, ATP7B, KRT5, KRT14, PLEC1, Col7A1, ITGB4, ITGA6, LAMA3, LAMB3, LAMC2, KIND1, INS, F8 or a fragment thereof (e.g., fragment encoding B-domain deleted polypeptide (e.g., VIII SQ, p-VIII)), IRGM, NOD2, ATG2B, ATG9, ATG5, ATG7, ATG16L1, BECN1, EI24/PIG8, TECPR2, WDR45/WIP14, CHMP2B, CHMP4B, Dynein, EPG5, HspB8, LAMP2, LC3b UVRAG, VCP/p97, ZFYVE26, PARK2/Parkin, PARK6/PINK1, SQSTM1/p62, SMURF, AMPK, ULK1, RPE65, CHM, RPGR, PDE6B, CNGA3, GUCY2D, RS1, ABCA4, MYO7A, HFE, hepcidin, a gene encoding a soluble form (e.g., of the TNFα receptor, IL-6 receptor, IL-12 receptor, or IL-1β receptor), and cystic fibrosis transmembrane conductance regulator (CFTR).

In some embodiments, the at least one non-GSH nucleic acid comprises a sequence encoding a suicide protein.

In some embodiments, a cell comprises at least one non-GSH nucleic acid integrated into a GSH, wherein the at least one non-GSH nucleic acid encodes an antigen-binding protein. In some embodiments, the antigen-binding protein is an antibody or an antigen-binding fragment thereof, optionally wherein the antibody or an antigen-binding fragment thereof is selected from an antibody, Fv, F(ab)2, Fab′, dsFv, scFv, sc (Fv)2, half antibody-scFv, tandem scFv, Fab/scFv-Fc, tandem Fab′, single-chain diabody, tandem diabody (TandAb), Fab/scFv-Fc, scFv-Fc, heterodimeric IgG (CrossMab), DART, and diabody.

In some embodiments, the antigen-binding protein specifically binds TNFα, CD20, a cytokine (e.g., IL-1, IL-6, BLyS, APRIL, IFN-gamma, etc.), Her2, RANKL, IL-6R, GM-CSF, CCR5, or a pathogen (e.g., bacterial toxin, viral capsid protein, etc.).

In some embodiments, the antigen-binding protein is selected from adalimumab, etanercept, infliximab, certolizumab, golimumab, anakinra, rituximab, abatacept, tocilizumab, natalizumab, canakinumab, atacicept, belimumab, ocrelizumab, ofatumumab, fontolizumab, trastuzumab, denosumab, sarilumab, lenzilumab, gimsilumab, siltuximab, leronlimab, and an antigen-binding fragment thereof.

Further contemplated herein is a cell that comprises at least one non-GSH nucleic acid integrated into a GSH, wherein the at least one non-GSH nucleic acid comprises a sequence encoding a non-coding RNA. In some embodiments, the non-coding RNA comprises lncRNA, piRNA, miRNA, shRNA, siRNA, antisense RNA, snoRNA, snRNA, scaRNA, and/or guide RNA. In some embodiments, the non-coding RNA targets a gene selected from DMT-1, ferroportin, TNFα receptor, IL-6 receptor, IL-12 receptor, IL-1B receptor, a gene encoding a mutated protein (e.g., a mutated HFE, CFTR).

In some embodiments, a cell comprises at least one non-GSH nucleic acid integrated into a GSH, wherein the at least one non-GSH nucleic acid increases or restores the expression of an endogenous gene of a target cell. In some embodiments, a cell comprises at least one non-GSH nucleic acid integrated into a GSH, wherein the at least one non-GSH nucleic acid decreases or eliminates the expression of an endogenous gene of a target cell.

In some embodiments, a cell comprises at least one non-GSH nucleic acid integrated into a GSH, wherein the at least one non-GSH nucleic acid further comprises: (a) a transcription regulatory element (e.g., an enhancer, a transcription termination sequence, an untranslated region (5′ or 3′ UTR), a proximal promoter element, a locus control region (e.g., a β-globin LCR or a DNase hypersensitive site (HS) of β-globin LCR), a polyadenylation signal sequence), and/or (b) a translation regulatory element (e.g., Kozak sequence, woodchuck hepatitis virus post-transcriptional regulatory element).

In some embodiments, the cell is selected from a cell line or a primary cell. In some embodiments, the cell is a mammalian cell, an insect cell, a bacterial cell, a yeast cell, or a plant cell, optionally wherein the mammalian cell is a human cell or a rodent cell. In some embodiments, the cell is an insect cell; and the insect cell is derived from a species of lepidoptera. In some embodiments, the species of lepidoptera is Spodoptera frugiperda, Spodoptera littoralis, Spodoptera exigua, or Trichoplusia ni. In some embodiments, the insect cell is Sf9.

In some embodiments, the cell is selected from a hematopoietic cell, hematopoietic progenitor cell, hematopoietic stem cell, erythroid lineage cell, megakaryocyte, erythroid progenitor cell (EPC), CD34+ cell, CD44+ cell, red blood cell, CD36+ cell, mesenchymal stem cell, nerve cell, intestinal cell, intestinal stem cell, gut epithelial cell, endothelial cell, enteroendocrine cell, lung cell, lung progenitor cell, enterocyte, liver cell (e.g., hepatocyte, hepatic stellate cells, Kupffer cells (KCs), liver sinusoidal endothelial cells (LSECs), liver progenitor cell), stem cell, progenitor cell, induced pluripotent stem cell (iPSC), skin fibroblast, macrophage, brain microvascular endothelial cell (BMVECs), neural stem cell, muscle satellite cell, epithelial cell, airway epithelial cell, muscle progenitor cell, erythroid progenitor cell, lymphoid progenitor cell, B lymphoblast cell, B cell, T cell, basophilic Endemic Burkitt Lymphoma (EBL), polychromatic erythroblast, epidermal stem cell, epithelial stem cell, embryonic stem cell, P63-positive keratinocyte-derived stem cell, keratinocyte, pancreatic B-cell, K cell, L cell, HEK293 cell, HEK293T cell, MDCK cell, Vero cell, CHO, BHK1, NS0, Sp2/0, HeLa, A549, and orthochromatic erythroblast.

Additional descriptions of the cells that comprise the nucleic acid vector or viral vector of the present disclosure; or cells that comprise at least one non-GSH nucleic acid integrated into a GSH, are provided below.

### Cells

Provided herein are cells comprising a nucleic acid, nucleic acid vector, or viral vector of the present disclosure. A further object of the present invention relates to a cell which has been transfected, infected, transduced, or transformed by a nucleic acid, a nucleic acid vector, and/or viral vector according to the invention. The term “transformation” means the introduction of a “foreign” (i.e. extrinsic or extracellular) gene, DNA or RNA sequence to a cell, so that the cell will express the introduced gene or sequence to produce a desired substance, typically a protein or enzyme coded by the introduced gene or sequence. A cell that receives and expresses introduced DNA or RNA has been “transformed.”

The nucleic acids or the nucleic acid vectors of the present invention may be used to produce a recombinant polypeptide of the invention in a suitable expression system. The term “expression system” means a cell and compatible vector under suitable conditions, e.g. for the expression of a protein coded for by foreign DNA carried by the vector and introduced to the cell.

Common expression systems include E. coli cells and plasmid vectors, insect cells and Baculovirus vectors, and mammalian cells and vectors. Other examples of cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al: 1980), rat YB2/3HL.P2.G11.16Ag.20 cell (ATCC CRL 1662, hereinafter referred to as “YB2/0 cell”), and the like. The YB2/0 cell is preferred, since ADCC activity of chimeric or humanized antibodies is enhanced when expressed in this cell.

The present invention also relates to a method of producing a recombinant cell expressing an antibody or a polypeptide of the invention according to the invention, said method comprising the steps consisting of (i) introducing in vitro or ex vivo a recombinant nucleic acid, a nucleic acid vector or a viral vector as described herein into a competent cell, (ii) culturing in vitro or ex vivo the recombinant cell obtained and (iii), optionally, selecting the cells which express and/or secrete antigen-binding protein (e.g., antibody) or polypeptide (e.g., insulin). Such recombinant cells can be used for the production of various polypeptides described herein.

As used herein, the cell includes any type of cell that can contain the presently disclosed vector and is capable of producing an expression product encoded by the nucleic acid (e.g., mRNA, protein). The cell in some aspects is an adherent cell or a suspended cell, i.e., a cell that grows in suspension. The cell in various aspects is a cultured cell or a primary cell, i.e., isolated directly from an organism, e.g., a human. The cell can be of any cell type, can originate from any type of tissue, and can be of any developmental stage.

In certain aspects, the antigen-binding protein is a glycosylated protein and the cell is a glycosylation-competent cell. In various aspects, the glycosylation-competent cell is an eukaryotic cell, including, but not limited to, a yeast cell, filamentous fungi cell, protozoa cell, algae cell, insect cell, or mammalian cell. Such cells are described in the art. See, e.g., Frenzel, et al., Front Immunol 4:217 (2013). In various aspects, the eukaryotic cells are mammalian cells. In various aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NS0, GS-NS0, Sp2/0), cells engineered to be deficient in dihydrofolatereductase (DHFR) activity (e.g., DUKX-X11, DG44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinoma human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells (Gaillet et al. 2007; Khan, Adv Pharm Bull 3 (2): 257-263 (2013)).

In some embodiments, for purposes of amplifying or replicating the vector, the cell is in some aspects is a prokaryotic cell, e.g., a bacterial cell.

Also provided by the present disclosure is a population of cells comprising at least one cell described herein. The population of cells in some aspects is a heterogeneous population comprising the cell comprising vectors described, in addition to at least one other cell, which does not comprise any of the vectors. Alternatively, in some aspects, the population of cells is a substantially homogeneous population, in which the population comprises mainly cells (e.g., consisting essentially of) comprising the vector. The population in some aspects is a clonal population of cells, in which all cells of the population are clones of a single cell comprising a vector, such that all cells of the population comprise the vector. In various embodiments of the present disclosure, the population of cells is a clonal population comprising cells comprising a vector as described herein.

In certain aspects the cell is a human cell that is autologous or allogeneic to the subject. In some embodiments, a nucleic acid of the present invention is transduced via a viral vector or transformed in other suitable methods (e.g., electroporation, etc.). Such cells are transferred (e.g., grafted, implanted, etc.) to the subject for a prolonged treatment of the disease or condition, e.g., cancer.

### Transgenic Organism

In certain aspects, provided herein is a transgenic organism comprising at least one non-GSH nucleic acid integrated into a GSH in the genome of a cell, wherein the GSH is selected from Table 3. In some embodiments, the GSH is selected from SYNTX-GSH1, SYNTX-GSH2, SYNTX-GSH3, and SYNTX-GSH4.

In some embodiments, the transgenic organism comprises any one of nucleic acid vectors, viral vectors, and/or cells of the present disclosure. In some embodiments, the transgenic organism comprises the cell of the present disclosure.

The transgenic organism may be derived from any organism that includes unicellular and multicellular organisms. Such organisms encompasses animals, plants, fungi, bacteria, protists, fish, etc. In some embodiments, the transgenic organism is a mammal or plant. In some embodiments, the transgenic organism is a fungus (e.g., yeast), bacteria, or protest. In some embodiments, the transgenic organism is a fish. In some embodiments, the transgenic organism is a rodent (e.g., mouse, rat). In some embodiments, the transgenic organism is a rodent or a plant, optionally wherein the rodent is a mouse. In some embodiments, the transgenic organism is a mammal or a plant, optionally wherein the mammal is a rodent (e.g., mouse, rat), a goat, a sheep, a chicken, a llama, or a rabbit.

Genetic modification of the germ line of an organism to create a transgenic organism can be accomplished by introducing any one of the nucleic acid vectors and viral vectors of the present disclosure using methods described herein as well as those well known in the art.

### Pharmaceutical Compositions

In certain aspects, provided herein are pharmaceutical compositions comprising any one of the nucleic acid vectors of the present disclosure, any one of the viral vectors of the present disclosure, and/or any one of the cells of the present disclosure. Any combination of the nucleic acid vectors, viral vectors, and cells are contemplated herein, and such combination may provide a potent therapeutic pharmaceutical composition.

The pharmaceutical composition may further comprise a carrier and/or a diluent. As used herein the pharmaceutically acceptable carrier is intended to include any and all solvents, dispersion media, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. For determining compatibility, various relevant factors, such as osmolarity, viscosity, and/or baricity can be considered. Supplementary active compounds can also be incorporated into the compositions.

A pharmaceutical composition of the present invention is formulated to be compatible with its intended route of administration. Examples of routes of administration include parenteral, e.g., intravenous, intradermal, subcutaneous, oral, intranasal (e.g., inhalation), transdermal, transmucosal, intravascular, intracerebral, parenteral, intraperitoneal, epidural, intraspinal, intrasternal, intra-articular, intra-synovial, intratumoral, intrathecal, intra-arterial, intracardiac, intramuscular, intrapulmonary, and rectal administration. In certain embodiments, a direct injection into the bone marrow is contemplated. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For example, Ringer's solution and lactated Ringer's solution are USP approved for formulating IV therapeutics, and those solutions are used in some embodiments. In certain embodiments, the excipient and vector compatibility to retain biological activity is established according to suitable methods. For intravenous administration or injection to the bone marrow, suitable carriers include physiological saline, bacteriostatic water, Cremophor EL™ (BASF, Parsippany, NJ) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Inhibition of the action of microorganisms can be achieved by various antibacterial and antifungal agents, for example, parabens, chlorobutanol, phenol, ascorbic acid, thimerosal, and the like, to the extent that they do not affect the integrity/activity of the viral compositions described herein. In many cases, it is preferable to include isotonic agents, for example, sugars, polyalcohols such as manitol, sorbitol, sodium chloride in the composition.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above.

For administration by inhalation, the viral vectors or nucleic acid vectors described herein are delivered in the form of an aerosol spray from pressured container or dispenser which contains a suitable propellant, e.g., a gas such as carbon dioxide, or a nebulizer.

Systemic administration can also be by transmucosal means. For transmucosal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

### Delivery of Nucleic Acid Vectors

Various techniques and methods are known in the art for delivering nucleic acids to cells, and are encompassed for use in the delivery of the nucleic acid vectors described herein, including non-viral vectors comprising a portion of the GSH or nucleic acid vectors comprising 5′- and 3′ GSH-specific homology arms. For example, nucleic acids can be formulated into lipid nanoparticles (LNPs), lipidoids, liposomes, lipid nanoparticles, lipoplexes, or core-shell nanoparticles. Typically, LNPs are composed of nucleic acid molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol). Exemplary lipid nanoparticles and methods for preparing the same are described, for example, in WO2015/074085, WO2016081029, WO2015/199952, WO2017/117528, WO2017/075531, WO2017/004143, WO2012/040184, WO2012/061259, WO2011/149733, WO2013/158579, WO2014/130607, WO2011/022460, WO2013/148541, WO2013/116126, WO2011/153120, WO2012/044638, WO2012/054365, WO2008/042973, WO2010/129709, WO2010/144740, WO2012/099755, WO2013/049328, WO2013/086322, WO2013/086354, WO2013/086373, WO2014/008334, WO2011/075656, WO2011/071860, WO2009/132131, WO2010/088537, WO2010/054401, WO2010/054384, WO2010/054406, WO2010/054405, WO2010/048536, WO2009/082607, WO2012/016184, WO2014/152211,
WO2017/049074, WO1996/040964, WO1999/018933, WO2009/086558, WO2010/129687, WO2010/147992, WO2010/042877, WO2009/108235, WO2014/081887, WO2005/120461, WO2011/000106, WO2011/000107, WO2015/011633, WO2005/120152, WO2011/141705, WO2016/197133, WO2015/011633, WO2013/126803, WO2012/000104, WO2011/141705, WO2006/007712, WO2011/038160, WO2005/121348, WO2005/120152, WO2011/066651, WO2009/127060, WO2011/141704, WO2006/074546, WO2005/121348, WO2006/069782, WO2009/027337, WO2012/030901, WO2012/031043, WO2012/031046, WO2013/006825, WO2013/033563, WO2013/040429, WO2014/043544, WO2016/130963, WO2017/181026, and WO2013/089151, contents of all of which is incorporated herein by reference in their entireties. In some embodiments, the lipid nanoparticle, in addition to the nucleic acid, comprises lipids in the following molar ratio: 50% cationic lipid, 10% non-ionic lipid (e.g., phospholipid, such as distearoylphosphatidylcholine (DSPC)), 38.5% cholesterol and 1.5% PEG-lipid (e.g., 2-[2-(w-methoxy (polyethyleneglycol2000) ethoxy]-N, N-ditetradecylacetamide (PEG2000-DMA)).

Another method for delivering nucleic acids to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332, WO2006/112872, WO2004/090108, WO2004/091515, WO2017/177326, contents of all of which is incorporated herein by reference in their entirety.

Nucleic acids can also be delivered to a cell by electroporation. Generally, electroporation uses pulsed electric current to increase the permeability of cells, thereby allowing the nucleic acid to move across the plasma membrane. Electroporation techniques are well known in the art and are used to deliver nucleic acids in vivo and clinically. See, for example, Andre et al., Curr Gene Ther. 2010 10:267-280; Chiarella et al, Curr Gene Ther. 2010 10:281-286; Hojman, Curr Gene Ther. 2010 10:128-138; contents of all of which are herein incorporated by reference in their entirety. Electroporation devices are sold by many companies worldwide including, but not limited to BTX® Instruments (Holliston, MA) (e.g., the AgilePulse In Vivo System) and Inovio (Blue Bell, PA) (e.g., Inovio SP-5P intramuscular delivery device or the CELLECTRA® 3000 intradermal delivery device). Electroporation can be used after, before and/or during administration of the nucleic acid vector. Additional exemplary methods and apparatus for delivering nucleic acids utilizing electroporation are described, for example, in U.S. Pat. Nos. 5,273,525, 6,520,950, 6,654,636 and 6,972,013, contents of all of which are incorporated herein by reference in their entirety.

Nucleic acids can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), FIPOFECTAMINE™ (Thermo Fisher Scientific), FIPOFECTIN™ (Thermo Fisher Scientific), DMRIE-C, CEFFFECTIN™ (Thermo Fisher Scientific), OFIGOFECTAMINE™ (Thermo Fisher Scientific), FIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SIFENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.). Nucleic acids, can also be delivered to a cell via microfluidics methods known to those of skill in the art.

Methods of non-viral delivery of nucleic acids in vivo or ex vivo include electroporation, lipofection (see, U.S. Pat. Nos. 5,049,386; 4,946,787 and commercially available reagents such as Transfectam™ and Lipofectin™), microinjection, biolistics, virosomes, liposomes (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787), immunoliposomes, polycation or lipidmucleic acid conjugates, naked DNA, artificial virions, viral vector systems (e.g., retroviral, lentivirus, adenoviral, adeno-associated, vaccinia and herpes simplex virus vectors as described in WO2007/014275) and agent-enhanced uptake of DNA. Sonoporation using, e.g., the Sonitron 2000 system (Rich-Mar) can also be used for delivery of nucleic acids.

Vectors (e.g., retroviruses, adenoviruses, liposomes, etc.) comprising nucleic acids as described herein can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of a nucleic acid vector composition as disclosed herein into hematopoietic stem cells are disclosed, for example, in U.S. Pat. No. 5,928,638.

The nucleic acid vector compositions as disclosed herein can be used for ex vivo cell transfection for diagnostics, research, or for gene therapy (e.g., via re-infusion of the transfected cells into the host organism). In some embodiments, cells are isolated from the subject organism, transfected with a nucleic acid vector a composition as disclosed herein, and re-infused back into the subject organism (e.g., patient or subject). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In some embodiments, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panb cells), GR-1 (granulocytes), and lad (differentiated antigen presenting cells) (see Inaba et al., J. Exp. Med. 176:1693-1702 (1992)). In some embodiments, the cell to be used is an oocyte. In other embodiments, cells derived from model organisms may be used. These can include cells derived from xenopus, insect cells (e.g., drosophilia) and nematode cells.

### Kits

In certain aspects, provided here are kits comprising any one of any one of the nucleic acid vectors of the present disclosure, any one of the viral vectors of the present disclosure, any one of the cells of the present disclosure, and/or any one of the pharmaceutical compositions of the present disclosure.

In some embodiments, kits for insertion of a gene or nucleic acid sequence into a target GSH identified according to the methods as disclosed herein, as well as primer sets to determine integration of the gene or nucleic acid sequence.

In some embodiment, the kit comprises: (a) a vector composition as described herein, and primer pairs to determine integration by homologous recombination of nucleic acid located between the restriction site located between the 3′ GSH-specific homology arm and the 5′ GSH-specific homology arm of the vector. In some embodiments, the kit comprises primer pairs that span the site of integration, where the primer pair comprises at least a GSH 5′ primer and at least one GSH 3′ primer, wherein the GSH is identified according to the methods as disclosed herein, wherein the at least one GSH 5′ primer binds to a region of the GSH upstream of the site of integration, and the at least one GSH 3′ primer is at least binds to a region of the GSH downstream of the site of integration. Such primer pairs can function to act as a negative control and do produce a short PCR product when no integration has occurred, and produce no, or a long PCR product incorporating the inserted nucleic acid when nucleic acid insertion has occurred.

In some embodiments, the kit can comprise (a) a GSH-specific single guide and an RNA guided nucleic acid sequence comprised in one or more GSH vectors; and (b) GSH knock-in vector comprising GSH vector wherein one or more of the sequences of (a) or (b) are comprised on a vector as described herein. In some embodiments, the GSH vector is a GSH-CRISPR-Cas vector or other GSH-gene editing vector as comprising a gene editing gene as described herein. In some embodiments, the GSH CRISPR-Cas vector comprises a GSH-sgRNA nucleic acid sequence and Cas9 nucleic acid sequence.

In other embodiments, the kit can further comprise a GSH knockin donor vector comprising a GSH 5′ homology arm and a GSH 3′ homology arm, wherein the GSH 5′ homology arm and the GSH 3′ homology arm are at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% complementary to a sequence in the genomic safe harbor (GSH) identified according to the methods as disclosed herein, and where the GSH 5′ and 3′ homology arms allow (i.e., guide) insertion, by homologous recombination, of the nucleic acid sequence located between the GSH 5′ homology arm and a GSH 3′ homology arm into a loci located within the genomic safe harbor. As an exemplary example, in some embodiments, the GSH Cas9 knockin donor vector is a SYNTX-GSH1 Cas9 knockin donor vector comprising a SYNTX-GSH1 5′ homology arm and a SYNTX-GSH1 3′ homology arm, wherein the SYNTX-GSH1 5′ homology arm and the SYNTX-GSH1 3′ homology arm are at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% complementary to the SYNTX-GSH1 genomic safe harbor loci, and wherein the SYNTX-GSH1 5′ and 3′ homology arms guide insertion, by homologous recombination, of the nucleic acid located between the GSH 5′ homology arm and a GSH 3′ homology arm into a loci within the SYNTX-GSH1 genomic safe harbor.

In some embodiments, the kit comprises a GSH vector which is GSH Cas9 knock in donor vector.

In some embodiments, the kit further comprises at least one GSH 5′ primer and at least one GSH 3′ primer, wherein the at least one GSH 5′ primer is at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% complementary to a region of the GSH upstream of the site of integration, and the at least one GSH 3′ primer is at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% complementary to a region of the GSH downstream of the site of integration.

In some embodiments, the kit can comprise two primer pairs, each primer pair functioning as a positive control. For example, in some embodiments, the kit comprises (a) at least two GSH 5′ primers comprising a forward GSH 5′ primer that binds to a region of the GSH upstream of the site of integration, and a reverse GSH 5′ primer that binds to a sequence in the nucleic acid inserted at the site of integration in the GSH sequence, and (b) at least two GSH 3′ primers comprising a forward GSH 3′ primer that binds to a sequence located at the 3′ end of the nucleic acid inserted at the site of integration in the GSH sequence, and a reverse GSH 3′ primer binds to a region of the GSH downstream of the site of integration. In such an embodiment, the primer pairs can function to act as a positive and produce a PCR product only when integration has occurred, and no PCT product is produced when integration has not occurred.

In some embodiments, the kit can comprise at least two GSH 5′ primers comprising: a forward GSH 5′ primer that is at least 80% complementary to a region of the GSH upstream of the site of integration, and a reverse GSH 5′ primer that is at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% complementary to a sequence in the nucleic acid inserted at the site of integration in the GSH sequence.

In some embodiments, the kit can further comprise at least two GSH 3′ primers comprising: a forward GSH 3′ primer that is at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% complementary to a sequence located at the 3′ end of the nucleic acid inserted at the site of integration in the GSH sequence, and a reverse GSH 3′ primer that is at least, about, or no more than 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% complementary to a region of the GSH down-stream of the site of integration.

In some embodiments, the kit comprises any one of the nucleic acid vectors described herein.

In some embodiments, the kit comprises any one of the viral vectors described herein.

In some embodiments, the kit comprises any one of the any one of the cells described herein.

In some embodiments, the kit comprises any one of the any one of the pharmaceutical compositions of the present disclosure.

In some embodiments, the kit comprises any combination of the nucleic acid vectors, viral vectors, cells, and pharmaceutical compositions.

The nucleic acid, viral vector, cell, and/or pharmaceutical composition can be packaged in a suitable container. A kit can include additional components to facilitate the particular application for which the kit is designed. In addition, a kit encompassed by the present disclosure can also include instructional materials disclosing or describing the use of the kit.

### Use of GSH in Manufacturing Biologics

Provided herein are use of the GSH loci identified herein for preparing biologics. Notably, the GSH loci identified herein are particularly useful in allowing large-scale manufacturing of biologics by providing cells with stable integration of genes expressing biologics.

Protein based therapeutics, including antibodies, peptides and recombinant proteins, represent the majority of new products in development by the pharmaceutical industry (Ho & Chien 2014, PMID: 24186148). Such products are produced in a variety of platforms, including non-mammalian (bacteria, yeast, plants and insect cells), and mammalian systems (rodent and human derived cells). Mammalian expression systems are usually preferred platform for manufacturing biopharmaceuticals, as these cells or cell lines are able to produce large and complex proteins with post-translational modifications similar to those found in humans. Among the variety of mammalian cell lines used for biologics manufacturing, human-derived cell lines are attractive as substrates for therapeutic glycoproteins production, as their glycosylation machinery eliminates risk of immunogenicity, which is found in byproducts derived from different cells, such as rodent derived cell lines (e.g., CHO, BHK1, NS0, Sp2/0). These non-human cell lines possess different post-translational modification pathways that can generate immunogenic glycans such as galactose-α1,3-galactose (α-galactose) and N-glycolylneuraminic acid (NGNA) (Butler and Spearman 2014, PMID: 25005678). Since there is a prevalence of circulating antibodies against both of these N-glycans in the human population, such non-human cell lines need to be screened for clones with acceptable glycosylation profile (Dumont, J. et al PMID: 26383226).

Chinese Hamster Ovary (CHO) cells, are aneuploid cells commonly used in the production of therapeutic proteins. CHO cell chromosomes carry structural abnormality and undergo changes in structure and number during cell proliferation. During proliferation, they continuously undergo genomic changes such as mutations, deletions, duplications, and other structural alterations due to errors in DNA replication and repair, and mistakes in chromosome segregation. As a result, these cells, along with other commonly used cell lines such as HEK293, MDCK, and Vero cells, have a wide distribution of chromosome number. Accordingly, these cell lines are associated with heterogeneity in the form of genomic and epigenomic variation or changes to cell phenotype or productivity.

Such heterogeneity that can affect the production of biologics is exacerbated by random integration of a transgene expressing a biologic. The current process for human cell line generation is based on random integration of the gene of interest into the genome, resulting in recombinant clones with high genomic and phenotypic variability, referred to as clonal variation. This variability affects the product's predictive value, it constrains process streamlining, and the achievement of cost-effective therapeutic glycoprotein production.

In addition, expression of a randomly integrated transgene is unpredictable and tends to be unstable overtime due to epigenetic effects. Further, random integration often yields multiple integrants per cell, and this can result in the disruption or activation of host cell genes. The biopharmaceutical industry devotes considerable resources to improving the yields and quality of recombinant proteins, particularly monoclonal antibodies. This process often begins with the selection of a high-yielding cell clone from the heterogenous population of stable cells. Clonal variation can be partly explained by the plasticity of the host cell genome and epigenetic imprinting. This is reflected in recurrent chromosomal rearrangement, high mutation rate and genome instability (Vcelar et al. 2018 PMID: 29328552) as well as suppressing expression of non-essential genes that negatively affect transgene expression. Genomic variation also occurs due to random integration of the vector, which can be inserted in multiple copies in different genomic loci, known as “position effect” and highlight the importance of the surrounding genomic environment (Wilson, C. et al 1990 PMID: 2275824). Furthermore, epigenetic regulation can also influence the expression of the transgene and be influenced by environmental conditions such as oxygen and nutrient levels or by accumulation of toxic byproducts during the production process. Clonal heterogeneity requires time-consuming and labor-intensive screening to find cell lines with the desired performance. The clonal selection process may involve single-cell cloning using high-throughput screening: however, this is an inherently a random process.

By contrast, a GSH locus can be reliably used for predictable expression. First, it eliminates the genomic heterogeneity induced by random integration of the transgene. Such is mediated by high fidelity homologous recombination and/or nuclease-initiated recombination (e.g., CRISPR). Second, the transgene is inserted in a genomic location that allows not only stable integration but also stable expression. There is no concern for the transgene disrupting an important gene in cells that are chosen to produce a biologic. This stable expression is also predictable. Since GSH provides a known transcriptional environment, there is no “position effect” or silencing of the transgene by e.g., the repressive (e.g., heterochromatic) environment nearby. Thus, the transgene insertion at a GSH locus does not affect cell cycle homeostasis and allows high bio-product yield.

Accordingly, provided herein are methods of manufacturing a biologic, the method comprising: (a) culturing (i) the cell comprising any one of the nucleic acid vectors described herein, (ii) the cell comprising any one of the viral vectors described herein, or (iii) any one of the cells described herein; and recovering the expressed biologic; or (b) recovering the expressed biologic from any one of the transgenic organisms contemplated herein.

In some embodiments, the biologic is an antigen-binding protein. In some embodiments, the biologic is an antibody or an antigen-binding fragment thereof, optionally wherein the antibody or an antigen-binding fragment thereof is selected from an antibody, Fv, F(ab)2, Fab′, dsFv, scFv, sc (Fv)2, half antibody-scFv, tandem scFv, Fab/scFv-Fc, tandem Fab′, single-chain diabody, tandem diabody (TandAb), Fab/scFv-Fc, scFv-Fc, heterodimeric IgG (CrossMab), DART, and diabody.

In some embodiments, the biologic specifically binds TNFα, CD20, a cytokine (e.g., IL-1, IL-6, BLyS, APRIL, IFN-gamma, etc.), Her2, RANKL, IL-6R, GM-CSF, or CCR5.

In some embodiments, the biologic is selected from adalimumab, etanercept, infliximab, certolizumab, golimumab, anakinra, rituximab, abatacept, tocilizumab, natalizumab, canakinumab, atacicept, belimumab, ocrelizumab, ofatumumab, fontolizumab, trastuzumab, denosumab, sarilumab, lenzilumab, gimsilumab, siltuximab, leronlimab, and an antigen-binding fragment thereof.

In some embodiments, the biologic is a therapeutic protein, optionally wherein the therapeutic protein is an insulin.

### Antigen-Binding Proteins

The antigen-binding proteins of the present disclosure can take any one of many forms of antigen-binding proteins known in the art. In various embodiments, the antigen-binding proteins of the present disclosure take the form of an antibody, or antigen-binding antibody fragment, an engineered antibody protein product (e.g., those comprising a fragment of antibody), a ligand-binding or receptor-binding protein or a fragment thereof, or a fusion protein.

As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4^{th} ed. Elsevier Science Ltd./Garland Publishing, (1999)).

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342:877-883), within a framework region (designated framework regions 1-4, FR1, FR2, FR3, and FR4, by Kabat et al., 1991; see also Chothia and Lesk, 1987, supra).

CDR refers to a complementarity determining region (CDR) of which three make up the binding character of a light chain variable region (CDR-L1, CDR-L2 and CDR-L3) and three make up the binding character of a heavy chain variable region (CDR-H1, CDR-H2 and CDR-H3). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, contact or any other boundary definitions. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat, Chothia, and/or MacCallum et al., (Kabat et al., in “Sequences of Proteins of Immunological Interest,” 5th Edition, U.S. Department of Health and Human Services, 1992; Chothia et al. (1987) J. Mol. Biol. 196, 901; and MacCallum et al., J. Mol. Biol. (1996) 262, 732, each of which is incorporated by reference in its entirety).

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in various embodiments, the antibody is an antibody of isotype IgA, IgD, IgE, IgG, or IgM, including any one of IgG1, IgG2, IgG3 or IgG4. In various aspects, the antibody comprises a constant region comprising one or more amino acid modifications, relative to the naturally-occurring counterpart, in order to improve half-life/stability or to render the antibody more suitable for expression/manufacturability. In various instances, the antibody comprises a constant region wherein the C-terminal Lys residue that is present in the naturally-occurring counterpart is removed or clipped.

The antibody can be a monoclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In certain aspects, the antigen-binding protein is an antibody, such as a human antibody. In certain aspects, the antigen-binding protein is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence. Information, including sequence information for human antibody heavy and light chain constant regions is publicly available through the Uniprot database as well as other databases well-known to those in the field of antibody engineering and production. For example, the IgG2 constant region is available from the Uniprot database as Uniprot number P01859, incorporated herein by reference.

An antibody can be cleaved into fragments by enzymes, such as, e.g., papain and pepsin. Papain cleaves an antibody to produce two Fab′ fragments and a single Fc fragment. Pepsin cleaves an antibody to produce a F(ab)₂ fragment and a pFc′ fragment. In various aspects of the present disclosure, the antigen-binding protein of the present disclosure is an antigen-binding fragment of an antibody (a.k.a., antigen-binding antibody fragment, antigen-binding fragment, antigen-binding portion). In various instances, the antigen-binding antibody fragment is a Fab′ fragment or a F(ab)₂ fragment.

The architecture of antibodies has been exploited to create a growing range of alternative antibody formats that spans a molecular-weight range of at least about 12-150 kDa and has a valency (n) range from monomeric (n=1), to dimeric (n=2), to trimeric (n=3), to tetrameric (n=4), and potentially higher; such alternative antibody formats are referred to herein as “antibody protein products.” Antibody protein products include those based on the full antibody structure and those that mimic antibody fragments which retain full antigen-binding capacity, e.g., scFvs, Fabs and VHH/VH (discussed below). The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab′ fragment. Both scFv and Fab′ fragments can be easily produced in host cells, e.g., prokaryotic host cells. Other antibody protein products include disulfide-bond stabilized scFv (ds-scFv), single chain Fab′ (scFab′), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4 (5): 586-591 (2012).

Other antibody protein products include a single chain antibody (SCA); a diabody; a triabody; a tetrabody, and the like.

In various aspects, the antigen-binding protein of the present disclosure comprises, consists essentially of, or consists of any one of these antibody protein products.

In various aspects, the antigen-binding protein of the present disclosure comprises, consists essentially of, or consists of any one of an scFv, Fab′, F(ab)2, VHH/VH, Fv fragment, ds-scFv, scFab′, half antibody-scFv, heterodimeric Fab/scFv-Fc, heterodimeric scFv-Fc, heterodimeric IgG (CrossMab), tandem scFv, tandem biparatopic scFv, Fab/scFv-Fc, tandem Fab′, single-chain diabody, dimeric antibody, multimeric antibody (e.g., a diabody, triabody, tetrabody), miniAb, peptibody VHH/VH of camelid heavy chain antibody, sdAb, diabody (single-chain diabody, homodimeric diabody, heterodimeric diabody, tandem diabody (TandAb), diabody that self-dimerizes), a triabody, a tetrabody. An ordinarily skilled artisan would understand that any bispecific antigen-binding protein formats can be used to generate biparatopic antigen-binding protein formats. In some embodiments, the antigen-binding protein is a dual-affinity re-targeting antibody (DART). In some embodiments, the antigen-binding protein is a bispecific T-cell engager (BiTE).

### Exemplary Biologics

Exemplary antigen-binding proteins include, for example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like.

Exemplary biologics may comprise any one of the therapeutic proteins or a fragment thereof as described herein or those known in the art. For example, a biologic may comprise a recombinant polypeptide or a fragment thereof selected from a hemoglobin gene (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), GBA1, preproinsulin, insulin, GIP, GLP-1, CEP290, ATPB1, ATPB11, ABCB4, CPS1, ATP7B, KRT5, KRT14, PLEC1, Col7A1, ITGB4, ITGA6, LAMA3, LAMB3, LAMC2, KIND1, INS, F8 or a fragment thereof (e.g., fragment encoding B-domain deleted polypeptide (e.g., VIII SQ, p-VIII)), IRGM, NOD2, ATG2B, ATG9, ATG5, ATG7, ATG16L1, BECN1, EI24/PIG8, TECPR2, WDR45/WIP14, CHMP2B, CHMP4B, Dynein, EPG5, HspB8, LAMP2, LC3b UVRAG, VCP/p97, ZFYVE26, PARK2/Parkin, PARK6/PINK1, SQSTM1/p62, SMURF, AMPK, ULK1, RPE65, CHM, RPGR, PDE6B, CNGA3, GUCY2D, RS1, ABCA4, MYO7A, HFE, hepcidin, a gene encoding a soluble form (e.g., of the TNFα receptor, IL-6 receptor, IL-12 receptor, or IL-1β receptor), and cystic fibrosis transmembrane conductance regulator (CFTR).

A complete list of FDA-approved biologics is available at World Wide Web at fda.gov/vaccines-blood-biologics/development-approval-process-cber/biological-approvals-year; and in the Purple Book (World Wide Web at purplebooksearch.fda.gov/). As used herein, the biologics encompass biosimilars.

### Manufacturing Methods

Also provided herein are methods of producing a biologic. In some embodiments, the method comprises culturing a host cell comprising a nucleic acid comprising a nucleotide sequence encoding a biologic in a cell culture medium and harvesting the secreted biologic from the cell culture medium. The host cell can be any of the host cells described herein. In various aspects, the host cell is selected from the group consisting of: CHO cells, NS0 cells, COS cells, VERO cells, and BHK cells. In various aspects, the step of culturing a host cell comprises culturing the host cell in a growth medium to support the growth and expansion of the host cell. In various aspects, the growth medium increases cell density, culture viability and productivity in a timely manner. In various aspects, the growth medium comprises amino acids, vitamins, inorganic salts, glucose, and serum as a source of growth factors, hormones, and attachment factors. In various aspects, the growth medium is a fully chemically defined media consisting of amino acids, vitamins, trace elements, inorganic salts, lipids and insulin or insulin-like growth factors. In addition to nutrients, the growth medium also helps maintain pH and osmolality. Several growth media are commercially available and are described in the art. See, e.g., Arora, “Cell Culture Media: A Review” Mater Methods 3:175 (2013).

In various aspects, the method comprises culturing the host cell in a feed medium. In various aspects, the method comprises culturing in a feed medium in a fed-batch mode. Methods of recombinant protein production are known in the art. See, e.g., Li et al., “Cell culture processes for monoclonal antibody production” MAbs 2 (5): 466-477 (2010).

The method making a biologic can comprise one or more steps for purifying the protein from a cell culture or the supernatant thereof and preferably recovering the purified protein. In various aspects, the method comprises one or more chromatography steps, e.g., affinity chromatography (e.g., protein A affinity chromatography, nickel resin for Histidine (His) tags), ion exchange chromatography, hydrophobic interaction chromatography. In various aspects, the method comprises purifying the protein using a Protein A affinity chromatography resin.

In various embodiments, the method further comprises steps for formulating the purified protein, etc., thereby obtaining a formulation comprising the purified protein. Such steps are described in Formulation and Process Development Strategies for Manufacturing, eds. Jameel and Hershenson, John Wiley & Sons, Inc. (Hoboken, NJ), 2010.

In various aspects, the biologic is a fusion protein. For example, a biologic can be an antigen-binding protein linked to a polypeptide (e.g., an Fc domain). Thus, the present disclosure further provides methods of producing a fusion protein. In various embodiments, the method comprises culturing a host cell comprising a nucleic acid comprising a nucleotide sequence encoding the fusion protein as described herein in a cell culture medium and harvesting the fusion protein from the cell culture medium.

### Use of GSH in Manufacturing Viral Vectors

Recombinant viral vectors (e.g., AAV vectors, retrovirus vectors, lentiviral vectors, etc.) are important tools in therapy and research. For example, recombinant AAV vectors are a clinically validated tool for in vivo gene transfer. Although the applications of AAV vectors offer great potential for many genetic diseases, current vector production methods still have room for improvement to meet the demands for not only human trials, but also for preclinical studies of basic biology, toxicology, and efficacy, in particular studies involving certain genetic diseases that require large quantities of high-quality vectors. For example, gene therapy for muscular dystrophies requires whole-body gene transfer in muscle, which is the largest organ in the body. Other genetic diseases that affect a large population such as sickle cell anemia or cystic fibrosis will require large preparation of recombinant vectors.

One of the most used methods for AAV production is the human embryonic kidney derived cells (HEK293) platform. The most widely used protocol of vector production is based on the helper-virus-free transient transfection method with all cis and trans components (vector plasmid and packaging plasmids, along with helper genes isolated from adenovirus) in host cells such as HEK293 cells. While the transient-transfection method is simple in vector plasmid construction and generates high-titer AAV vectors that are free of adenovirus, it has limited scalability and is not cost effective to supply clinical studies.

A second strategy is the recombinant herpes simplex virus (rHSV)-based AAV production system, which utilizes rHSV vectors to bring the AAV vector and the Rep and Cap genes into the cells.

The third method is based on the AAV producer cell lines derived from HeLa or A549, which stably harbored AAV Rep/cap genes and the gene of interest. The AAV vector cassette was either stably integrated in the host genome (Clark et al., 1995, PMID: 8590738) or introduced by an adenovirus that contained the cassette. Stable cell lines in continuous culture suffer from genetic instability as the number of passages increases. Randomly integrated viral genes can increase cell instability, reducing the ability of a stable cell propagation untimely affecting vector productivity. The selection of high-producing and stable cell clones is expensive and can take months. Furthermore, cell propagation may alter the recombinant protein homeostasis, post-translational modifications and secretion.

The use of GSH (e.g., integration of a gene encoding e.g., a viral capsid and/or recombination protein (e.g., gag, pol, rep, etc.) at the GSH loci) to generate AAV vectors producing stable cell lines ensures the quality of production cells over the intended passages to reach high vector productivity. Also, the use of GSH minimize perturbance of cell proteostasis during propagation, increasing product reproducibility across different production batches. A similar rationale can be applied in the manufacturing of other viral vectors such as Adeno virus-derived vectors, retrovirus and lentivirus-derived vectors, herpes virus-derived vectors and alphavirus-derived vectors such as Semliki forest virus (SFV) vectors where one or more components necessary for vector production are inserted in defined GSH loci. The expression of those components can be modulated (e.g., using an inducible promoter or early vs. late promoters) in order to mitigate an unwanted early expression to reach a certain number of host cells before the amplification of vector components and subsequent transgene packaging begin. The process of vectors manufacturing in mammalian cell lines can significantly benefit from the use of GSH by increasing cell stability, productivity, reproducibility, and product safety, directly impacting patients benefits while reducing costs associated with manufacturing and quality controls. Thus, in contrast to the randomly generated producer cell lines, the directed recombination to a GSH for rAAV production would accelerate the process by months or even years.

Thus, in certain aspects, provided herein are methods of manufacturing a viral vector. For example, a nucleic acid sequence necessary for viral assembly, e.g., those encoding one or more viral structural proteins (gag, VP1, VP2, VP3, etc.) and/or one or more replication proteins operably linked to at least one expression control sequence for expression in a host cell can be integrated into GSH loci in a host cell. Such cells can be provided with a nucleic acid comprising at least one function virus origin of replication, optionally further comprising a non-GSH nucleic acid for integration at the GSH site, and produce a viral vector.

Accordingly, in some embodiments, the method comprises: (1) providing a host cell comprising (i) a nucleic acid sequence comprising at least one functional virus origin of replication (e.g., at least one ITR nucleotide sequence), optionally further comprising a nucleic acid operably linked to a promoter for expression in a target cell, (ii) a nucleic acid sequence comprising at least one gene encoding one or more viral structural proteins (e.g., capsid proteins, e.g., gag, VP1, VP2, VP3, a variant thereof), operably linked to at least one expression control sequence for expression in a host cell, and (iii) a nucleic acid sequence comprising at least one gene encoding one or more viral replication proteins (e.g., Rep, pol) operably linked to at least one expression control sequence for expression in a host cell, optionally wherein the at least one replication protein comprises (a) a Rep52 or a Rep40 coding sequence or a fragment thereof that encodes a functional replication protein, operably linked to at least one expression control sequence for expression in a host cell, and/or (b) a Rep78 or a Rep68 coding sequence operably linked to at least one expression control sequence for expression in a host cell: wherein at least one of (i), (ii), and (iii) is stably integrated into at least one GSH selected from Table 3 in the host cell genome, and the at least one vector, if/when present, comprises the remainder of the (i), (ii), and (iii) that is not stably integrated in the host cell genome; and (2) maintaining the host cell under conditions such that a recombinant viral vector is produced.

In some embodiments, (ii) or (iii) is integrated into a GSH. In some embodiments, (ii) and (iii) are integrated into a GSH.

In some embodiments, the at least one functional virus origin of replication (e.g., at least one ITR nucleotide sequence) comprises: (a) a dependoparvovirus ITR, and/or (b) an AAV ITR, optionally an AAV2 ITR.

In certain embodiments, the ITR is a terminal palindrome with Rep binding elements and trs that is structurally similar to the wild-type ITR. The ITR may be selected from any one of AAV1-AAV13 and AAVrh.10. In certain embodiments, the ITR has the AAV2 RBE and trs. In some embodiments, the ITR is a chimera of different AAVs. In some embodiments, the ITR and the Rep protein are from AAV5. In some embodiments, the ITR is synthetic and is comprised of RBE motifs and trs GGTTGG, AGTTGG, AGTTGA, . . . RRTTRR. The typical T-shaped structure of the terminal palindrome consisting of the B/B′ and C/C′ stems may also be synthetically modified with substitutions and insertions that maintain the overall secondary structure based on folding prediction (available at URL (http) of unafold.rna.albany.edu/?q=mfold/DNA-Folding-Form). The stability of the ITR secondary structure is designated by the Gibbs free energy, delta G, with lower values, i.e., more negative, indicating greater stability. The full-length, 145 nt ITR has a computed ΔG=−69.91 kcal/mol. The B and C stems: GCCCGGGCAAAGCCCGGGCGTCGGGCGACCTTTGGTCGCCCG have ΔG=−22.44 kcal/mol. Substitutions and insertions that result in a structure with ΔG=−15 kcal/mol to −30 kcal/mol are functionally equivalent and not distinct from the wild-type Dependoparvovirus ITRs.

In some embodiments, the at least one expression control sequence for expression in the host cell comprises: (a) a promoter, and/or (b) a Kozak-like expression control sequence.

In some embodiments, the promoter comprises: (a) an immediate early promoter of an animal DNA virus, (b) an immediate early promoter of an insect virus, (c) an insect cell promoter, or (d) an inducible promoter. In some embodiments, the animal DNA virus is cytomegalovirus (CMV), a Dependoparvovirus, or AAV. In some embodiments, the insect virus promoter is from a lepidopteran virus or a baculovirus, optionally wherein the baculovirus is Autographa californica multicapsid nucleopolyhedrovirus (AcMNPV). In some embodiments, the promoter is a polyhedrin (polh) or immediately early 1 gene (IE-1) promoter.

In some embodiments, the promoter is an inducible promoter. In some embodiments, the inducible promoter is modulated by an agent selected from a small molecule, a metabolite, an oligonucleotide, a riboswitch, a peptide, a peptidomimetic, a hormone, a hormone analog, and light. In some embodiments, the agent is selected from tetracycline, cumate, tamoxifen, estrogen, and an antisense oligonucleotide (ASO), rapamycin, FKCsA, blue light, abscisic acid (ABA), and riboswitch.

In some embodiments, the method comprises (a) the viral replication protein that is an AAV replication protein, optionally Rep52 and/or Rep78; and or (b) the viral structural protein that is an AAV capsid protein. In some embodiments, the AAV replication protein or the AAV capsid protein is of AAV2.

In some embodiments, the host cell is a mammalian cell or an insect cell.

In some embodiments, the host cell is a mammalian cell; and the mammalian cell is a human cell or a rodent cell. In some embodiments, the mammalian cell is selected from HEK293, HEK293T, HeLa, and A549.

In some embodiments, the host cell is an insect cell; and the insect cell is derived from a species of lepidoptera. In some embodiments, the species of lepidoptera is Spodoptera frugiperda, Spodoptera littoralis, Spodoptera exigua, or Trichoplusia ni. In some embodiments, the insect cell is Sf9.

In some embodiments, the viral vector is selected from adeno virus-derived vectors (e.g., AAV), retrovirus, lentivirus-derived vectors (e.g., lentivirus), herpes virus-derived vectors, and alphavirus-derived vectors (e.g., Semliki forest virus (SFV) vector).

It is contemplated herein that such method of manufacturing viral vectors is for use in manufacturing any or all viral vectors described herein as well as those known in the art.

### Use of GSH in Preparing Vaccines Against Infection

In certain aspects, provided herein are methods and compositions for immunizing a subject against infections (e.g., bacterial infections, fungal infections, viral infections).

In some embodiments, the compositions (e.g., nucleic acid vectors, viral vectors, and cells comprising a non-GSH nucleic acid integrated into a GSH locus) and methods provided herein facilitate production of recombinant proteins, e.g., immunogenic surface proteins of virus, bacteria, or fungus, that can be used as a vaccine, e.g., by administering to a subject in one or more doses to induce immune response and/or produce antibodies against the immunogenic proteins.

In some embodiments, the compositions and methods provided herein produce antigen-binding proteins against one or more surface proteins of virus, bacteria, or fungus; or toxins produced by bacteria or fungus (e.g., Tetanus toxin, Diphtheria toxin, Botulinum toxin, Pseudomonas exotoxin A), the introduction of which can protect a subject from infection. In some embodiments, such antigen-binding protein are produced in vitro and administered to a subject. In other embodiments, cells comprising such antigen-binding protein (e.g., the gene encoding said protein can be integrated into a GSH locus described herein) can be administered to a subject. In some embodiments, such gene is under a tissue-specific promoter or an inducible promoter.

In some embodiments, a cell can be engineered to integrate at a GSH locus of the present disclosure, a nucleic acid that encodes a surface protein of a virus, bacteria, or fungus. In preferred embodiments, the surface protein is of a virus. Such a cell or a pharmaceutical composition comprising such a cell may be administered to a subject as a source of immunogenic viral protein for in vivo immunization. In some embodiments, the cell is autologous to the subject. In other embodiments, the cell is allogeneic to the subject. Such cells may further comprise a suicide gene (e.g., integrated at GSH) such that after its use in in vivo immunization, such cells can be eliminated by turning on the suicide gene.

In some embodiments, (a) the surface protein or a fragment thereof is an immunogenic surface protein that elicits immune response in a host, (b) the surface protein or a fragment thereof further comprises a signal peptide, (c) the nucleic acid encoding the surface protein or a fragment thereof is operably linked to an inducible promoter, and/or (d) the nucleic acid encoding the surface protein or a fragment thereof further comprises a suicide gene. In preferred embodiments, the in vivo production of viral proteins may be under an inducible promoter, such that the amount of immunogen produced in vivo, as well as the duration of production, can be fine-tuned using a signal or agent that modulates the inducible promoter (see e.g., the section on Pulsatile Expression System described herein).

In some embodiments, such cells for producing vaccines in vitro or for in vivo immunization express the viral surface protein, wherein the surface protein is of a coronavirus (e.g., MERS, SARS), influenza virus, respiratory syncytial virus, hepatitis A, hepatitis B, hepatitis C, hepatitis D, hepatitis E, human papillomavirus, dengue virus serotype 1, dengue virus serotype 2, dengue virus serotype 3, dengue virus serotype 4, zika, virus, West Nile virus, yellow fever virus, Chikungunya virus, Mayaro virus, Ebola virus, Marburg virus, or Nipa virus. In some embodiments, the surface protein is the spike protein of SARS-COV-2.

### Use of GSH in Preventing or Treating Diseases (e.g., Gene Therapy)

In certain aspects, provided herein are methods of preventing or treating diseases, comprising administering to a subject in need thereof an effective amount of any one of the nucleic acid vector, the viral vector, the cell, and/or the pharmaceutical composition of the present disclosure. It is contemplated herein that the compositions and methods provided herein are suitable for preventing or treating any disease of the present disclosure (e.g., see Exemplary Diseases).

In some embodiments, the disease is selected from an infection, endothelial dysfunction, cystic fibrosis, cardiovascular disease, renal disease, cancer, hemoglobinopathy, anemia, hemophilia (e.g., hemophilia A), myeloproliferative disorder, coagulopathy, sickle cell disease, alpha-thalassemia, beta-thalassemia, Fanconi anemia, familial intrahepatic cholestasis, skin genetic disorder (e.g., epidermolysis bullosa), ocular genetic disease (e.g., inherited retinal dystrophies, e.g., Leber congenital amaurosis (LCA), retinitis pigmentosa (RP), choroideremia, achromatopsia, retinoschisis, Stargardt disease, Usher syndrome type 1B), Fabry, Gaucher, Nieman-Pick A, Nieman-Pick B, GM1 Gangliosidosis, Mucopolysaccharidosis (MPS) I (Hurler, Scheie, Hurler/Scheie), MPS II (Hunter), MPS VI (Maroteaux-Lamy), hematologic cancer, hemochromatosis, hereditary hemochromatosis, juvenile hemochromatosis, cirrhosis, hepatocellular carcinoma, pancreatitis, diabetes mellitus, cardiomyopathy, arthritis, hypogonadism, heart disease, heart attack, hypothyroidism, glucose intolerance, arthropathy, liver fibrosis, Wilson's disease, ulcerative colitis, Crohn's disease, Tay-Sachs disease, neurodegenerative disorder, Spinal muscular atrophy type 1, Huntington's disease, Canavan's disease, rheumatoid arthritis, inflammatory bowel disease, psoriatic arthritis, juvenile chronic arthritis, psoriasis, and ankylosing spondylitis, and autoimmune disease, neurodegenerative disease (e.g., Alzheimer's disease, Parkinson's disease, Huntington's disease, ataxias), inflammatory disease, inflammatory bowel disease, Crohn's disease, rheumatoid arthritis, lupus, multiple sclerosis, chronic obstructive pulmonary disease/COPD, pulmonary fibrosis, Sjogren's disease, hyperglycemic disorders, type I diabetes, type II diabetes, insulin resistance, hyperinsulinemia, insulin-resistant diabetes (e.g. Mendenhall's Syndrome, Werner Syndrome, leprechaunism, and lipoatrophic diabetes), dyslipidemia, hyperlipidemia, elevated low-density lipoprotein (LDL), depressed high density lipoprotein (HDL), elevated triglycerides, metabolic syndrome, liver disease, renal disease, cardiovascular disease, ischemia, stroke, complications during reperfusion, muscle degeneration, atrophy, symptoms of aging (e.g., muscle atrophy, frailty, metabolic disorders, low grade inflammation, atherosclerosis, stroke, age-associated dementia and sporadic form of Alzheimer's disease, pre-cancerous states, and psychiatric conditions including depression), spinal cord injury, arteriosclerosis, infectious diseases (e.g., bacterial, fungal, viral), AIDS, tuberculosis, defects in embryogenesis, infertility, lysosomal storage diseases, activator deficiency/GM2 gangliosidosis, alpha-mannosidosis, aspartylglucoaminuria, cholesteryl ester storage disease, chronic hexosaminidase A deficiency, cystinosis, Danon disease, Farber disease, fucosidosis, galactosialidosis, Gaucher Disease (Types I, II and III), GM1 Gangliosidosis, (infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Neuronal ceroid lipofuscinoses, CLN6 disease, Jansky-Bielschowsky disease, Pompe disease, pycnodysostosis, Sandhoff disease, Schindler disease, and Wolman disease.

In some embodiments, the infection is a bacterial infection, fungal infection, or a viral infection.

In some embodiments, the infection is the viral infection; and the viral infection is by a coronavirus (e.g., MERS, SARS), influenza virus, respiratory syncytial virus, hepatitis A, hepatitis B, hepatitis C, hepatitis D, hepatitis E, human papillomavirus, dengue virus serotype 1, dengue virus serotype 2, dengue virus serotype 3, dengue virus serotype 4, zika, virus, West Nile virus, yellow fever virus, Chikungunya virus, Mayaro virus, Ebola virus, Marburg virus, or Nipa virus. In some embodiments, the viral infection is by SARS-CoV-2.

In some embodiments, the nucleic acid vector, the cell, and/or the pharmaceutical composition is administered to the subject via intravascular, intracerebral, parenteral, intraperitoneal, intravenous, epidural, intraspinal, intrasternal, intra-articular, intra-synovial, intrathecal, intratumoral, intra-arterial, intracardiac, intramuscular, intranasal, intrapulmonary, skin graft, or oral administration.

In some embodiments, the cell is autologous or allogeneic to the subject.

In certain aspects, further provided herein are methods of modulating the level and/or activity of a protein in a cell, the method comprising introducing any one of the nucleic acid vector, the viral vector, and/or the pharmaceutical composition of the present disclosure.

In some embodiments, the level and/or activity of the protein is increased. In other embodiments, the level and/or activity is decreased or eliminated.

There are advantages of using the transduced cells in vitro or ex vivo for a therapy. First, the successful integration of the transgene in the GSH loci of the target cell genome can be verified before administering them to the patient. Second, the transduced cells can be administered to a subject in need thereof without the recombinant virions. This eliminate any concern for triggering immune response or inducing neutralizing antibodies that inactivate recombinant virions. Accordingly, the transduced cells can be safely redosed or the dose can be titrated without any adverse effect.

In some embodiments, the method comprises administering to a subject in need thereof, a viral vector a nucleic encoding (a) CFTR or a fragment thereof, (b) at least one non-coding RNA (e.g., piRNA, miRNA, shRNA, siRNA, gRNA, antisense RNA) that targets an endogenous mutant form of CFTR, (c) a CRISPR/Cas system that targets an endogenous mutant form of CFTR; and/or (d) any combination of any one of the nucleic acids listed in (a) to (c). As described herein, such viral vector comprises the said nucleic acids flanked by the GSH sequences such that they integrate into the GSH of the present disclosure. In some embodiments, such viral vectors or the nucleic acid vector comprising the said nucleic acids, are transduced into the cells in vitro, and the transduced cells are administered to a subject. In preferred embodiments, the cells are autologous to the subject. In some embodiments, the at least one nucleic acid vector, viral vector, or pharmaceutical composition is delivered to the lung via an intranasal or intrapulmonary administration. In some embodiments, the at least one nucleic acid vector, viral vector, or pharmaceutical composition (a) increases the expression of CFTR or fragment thereof; and/or (b) decreases the expression of an endogenous mutant form of CFTR in the cell. In some embodiments, the nucleic acid vector, viral vector, or pharmaceutical composition prevents or treats cystic fibrosis.

An ordinarily skilled artisan would appreciate that a subject with any mutant form of an endogenous protein many benefit from introducing a nucleic acid vector or viral vector comprising a nucleic acid encoding (a) wild-type protein or a functional equivalent thereof (e.g., fragment), (b) at least one non-coding RNA that targets an endogenous nucleic acid encoding the mutant protein, (c) a CRISPR/Cas system that targets an endogenous nucleic acid encoding the mutant protein, and/or (d) any combination of any of the nucleic acids listed in (a) to (c). Accordingly, such method can be applied to a subject afflicted with any disease that would benefit from replacing the mutant protein with a wild-type protein or a functional equivalent thereof.

In some embodiments, the methods of preventing or treating a disease further include re-administering at least one nucleic acid vector, viral vector, pharmaceutical composition, or cells. In some embodiments, the re-administering the at least one additional amount is performed after an attenuation in the treatment subsequent to administering the initial effective amount of the nucleic acid vector, viral vector, pharmaceutical composition, or cells. In some embodiments, the at least one additional amount is the same as the initial effective amount. In some embodiments, the at least one additional amount is more than the initial effective amount. In some embodiments, the at least one additional amount is less than the initial effective amount. In certain embodiments, the at least one additional amount is increased or decreased based on the expression of an endogenous gene and/or the nucleic acid of the nucleic acid vector, viral vector, pharmaceutical composition, or cells. The endogenous gene includes a biomarker gene whose expression is, e.g., indicative of or relevant to diagnosis and/or prognosis of the disease.

In certain aspects, the methods of preventing or treating a disease further comprise administering to the subject or contacting the cells with an agent that modulates the expression of the nucleic acid. In some embodiments, the agent is selected from a small molecule, a metabolite, an oligonucleotide, a riboswitch, a peptide, a peptidomimetic, a hormone, a hormone analog, and light. In some embodiments, the agent is selected from tetracycline, cumate, tamoxifen, estrogen, and an antisense oligonucleotide (ASO). In some embodiments, the methods further comprise re-administering the agent one or more times at intervals. In some embodiments, the re-administration of the agent results in pulsatile expression of the nucleic acid. In some embodiments, the time between the intervals and/or the amount of the agent is increased or decreased based on the serum concentration and/or half-life of the protein expressed from the nucleic acid.

### Exemplary Diseases

### Use of GSH in Gene Therapy for Skin Genetic Disorders-Epidermolysis Bullosa (EB)

In certain aspects, the methods and compositions described herein can be used to prevent and/or treat different skin disorders such as EB.

Human epidermis is mainly composed of keratinocytes organized in distinct stratified cellular layers. The adhesion of basal keratinocytes to the epidermal basement membrane is mediated by the hemidesmosomes (HDs), which are multiprotein complexes linking the epithelial intermediate filament network to the dermal anchoring fibrils. Hemidesmosomes are formed by the clustering of several cytoplasmic and transmembrane proteins. The cytoplasmic HD plaque components, which include HD1/plectin and the bullous pemphigoid antigen 1 (BP230), act as linkers for elements of the cytoskeleton at the cytoplasmic surface of plasma membrane. The transmembrane constituents of HDs, which include the a64 integrin and the bullous pemphigoid antigen 2 (BP180), serve as cell receptors connecting the cell interior to extracellular matrix proteins. Hemidesmosome-mediated adhesion relies on the binding of the α6β4 integrin to laminin-5, a major basal lamina component formed by distinct polypeptides, α3, β3, and γ2, encoded by 3 different genes known as LAMA3, LAMB3, and LAMC2, respectively. Laminin-5 interacts physically with α6β4 integrin on the basal surface of epidermal keratinocytes to promote HD formation as well as with the amino-terminal NC-1 domain of type VII collagen in dermal anchoring fibrils to enhance basement membrane zone integrity. The relevance of these proteins in maintaining the integrity of the skin has been proven by the identification of somatic mutations present in patients with epidermolysis bullosa (EB).

At least 16 genetic mutations in various genes (e.g., KRT5, KRT14, PLEC1, Col7A1, ITGB4, ITGA6, LAMA3, LAMB3, LAMC2, and KIND1) have been associated with different types of EB. Since keratinocytes are responsible for the synthesis of proteins involved in maintaining the dermal-epidermal junction, a gene therapeutic intervention to prevent or treat this disease requires the genetic modification of these cells.

Since keratinocytes are responsible for the synthesis of proteins involved in maintaining the dermal-epidermal junction, a gene therapeutic intervention to treat this disease will require the genetic modification of these cells. Modification of keratinocytes for skin disorders such as EB therefore requires the stable integration of the transgene into the genome (e.g., GSH loci of the present disclosure) of an epidermal stem cell, that is, the holoclone-forming cell. P63-positive keratinocytes derived stem cells holoclones have the maximum proliferative capacity and are considered epithelial stem cells. The use of GSH loci allows stable and persistent transgene expression throughout differentiation of keratinocytes, without affecting the differentiation process and allowing a maximum proliferative capacity to regenerate skin allografts. This method can considerably benefits EB patients.

Accordingly, in certain aspects, provided herein are methods of preventing or treating epidermolysis bullosa, wherein the at least one nucleic acid vector, viral vector, pharmaceutical composition, and/or cells comprising a nucleic acid encoding KRT5, KRT14, PLEC1, Col7A1, ITGB4, ITGA6, LAMA3, LAMB3, LAMC2, and/or KIND1 is administered to a subject. In some embodiments, the cell is an epidermal stem cell. In some embodiments, the epidermal stem cell is a holoclone-forming cell. In some embodiments, the holoclone-forming cells are P63-positive keratinocytes-derived stem cells. In some embodiments, the cell is a keratinocyte. In some embodiments, the nucleic acid encoding KRT5, KRT14, PLEC1, Col7A1, ITGB4, ITGA6, LAMA3, LAMB3, LAMC2, and/or KIND1 is under a tissue-specific promoter, optionally a tissue-specific promoter for an epidermal stem cell, a holoclone-forming cell, a P63-positive keratinocytes-derived stem cell, and/or a keratinocyte. In some such embodiments, the modified epidermal stem cells, P63-positive keratinocyte-derived stem cells, or keratinocytes are applied to the skin surface as a skin graft.

### Use of GSH to Express Pre-Pro-Insulin in Intestinal Endocrine K and L Cells—Type I Diabetes

In certain aspects, the methods and compositions described herein can be used to prevent and/or treat diseases with abnormal level of insulin, such as type I diabetes.

Enteroendocrine cells in the small intestine, especially in the duodenum and jejunum, appear as attractive targets for an insulin gene transfer strategy to treat patients with type 1 diabetes mellitus. K cells and L cells are innately specialized to respond to nutrients in the lumen, especially glucose, secreting GIP and GLP-1 into the blood, potentiating the glucose-induced insulin response. In normal individuals, the kinetics and plasma concentrations attained for GIP, GLP-1 and insulin following a meal are remarkably similar (Orskov et al., 1996, Fujita et al., 2004) and so are those of GIP and GLP-1 in patients with type 1 diabetes mellitus (Vilsboll et al., 2003). Furthermore, K cells and L cells synthesize the PC1/3 and PC2 peptidases that allow proinsulin processing into mature insulin. Finally, K cells and L cells are not destroyed by the immune system of patients with type 1 diabetes mellitus (Vilsboll et al., 2003).

Gastrointestinal enteroendocrine K cells and L cells release the glucose-dependent insulinotropic peptide (GIP) and glucagon-like peptide 1 (GLP-1), respectively. Due to their common developmental origin, pancreatic β-cells, K cells and L cells show marked similarities, which include: (i) the expression of the PC1/3 and PC2 peptidases needed for the conversion of proinsulin to insulin, (ii) the presence of GLUT-2 glucose transporter, (iii) a glucosedependent mechanism for hormone secretion, with granules that can store and readily secrete their respective hormones (Spooner et al., 1970, Baggio & Drucker 2007). Nonetheless, gastrointestinal enteroendocrine cells are not susceptible to the autoimmune-mediated destruction of pancreatic β-cells observed in patients with type 1 diabetes mellitus (Vilsboll et al., 2003). Interestingly, in healthy individuals, plasma GIP and GLP-1 levels kinetically match the changes in plasma insulin levels following meals (Fujita et al., 2004). Thus, engineering the gastrointestinal enteroendocrine cells of patients with type 1 diabetes mellitus to express the preproinsulin gene (e.g., by introducing an insulin gene, INS, encoding a preproinsulin protein or transcript variants thereof, e.g., NP_000198.1, NP_001172026.1, NP_001172027.1, and/or NP_001278826.1 would achieve normalization of postprandrial blood glucose.

### Use of GSH in Gene Therapy Applications for Gaucher Disease

In certain aspects, the methods and compositions described herein can be used to prevent and/or treat Guacher disease.

Gaucher disease (GD, OMIM #230800, ORPHA355) is the most common sphingolipidosis. GD is a rare, autosomal, recessive genetic disease caused by mutations in the GBA1 gene, located on chromosome 1 (1q21). This leads to a markedly decreased activity of the lysosomal enzyme, glucocerebrosidase (GCase, also called glucosylceramidase or acid β-glucosidase), which hydrolyzes glucosylceramide (GlcCer) into ceramide and glucose. More than 300 GBA mutations have been described in the GBA1 gene (PMID: 18338393). The disease phenotype is variable, but three clinical forms have been identified: type 1 is the most common and typically causes no neurological damage, whereas types 2 and 3 are characterized by neurological impairment. However, these distinctions are not absolute, and it is increasingly recognized that neuropathic GD represents a phenotypic continuum, ranging from extra pyramidal syndrome in type 1, at the mild end, to hydrops fetalis at the severe end of type 2.

Mutations in the GBA1 gene lead to a marked decrease in GCase activity. The consequences of this deficiency are generally attributed to the accumulation of the GCase substrate, GlcCer, in macrophages, inducing their transformation into Gaucher cells. Gaucher cells mainly infiltrate bone marrow, the spleen, and liver, but they also infiltrate other organs like the brain and are considered the main factors in the disease's symptoms. The monocyte/macrophage lineage is preferentially altered because of their role in eliminating erythroid and leukocytes, which contain large amounts of glycosphingolipids, a source of GlcCer. The pathophysiological mechanisms of neurological involvement remain poorly explained: GlcCer turnover in neurons is low and its accumulation is only significant when residual GCase activity is drastically decreased, i.e., only with some types of GBA1 mutations. It is likely that Gaucher cells that infiltrate the brain, can set a pro-inflammatory state leading to neurological complications. Numerous cytokines, chemokines and other molecules—including IL-1β, IL-6, IL-8, TNFα (Tumor Necrosis Factor), M-CSF (Macrophage-Colony Stimulating Factor), MIP-1β, IL-18, IL-10, TGFβ, CCL-18, chitotriosidase, CD14s, and CD163s—are present in increased amounts in Gaucher patients' plasma and could be implicated in hematological and tissue complications.

A gene replacement therapy offers a therapeutic alternative to repair human GBA expression and function by e.g., ex vivo correction of the GBA1 gene in autologous CD34+ stem cells. After insertion of a corrected GBA1 gene in a genomic safe harbor locus (GSH) of the present disclosure, positive CD34+ cells clones can be isolated and amplified without altering cells homeostasis. Engineered cells (e.g., transduced with the viral vectors or cells comprising nucleic acid vectors of the present disclosure) can be infused back into the patient where they can engraft back in the bone marrow and offer a stable clonally derived cell lineage with corrected GBA expression able to process glucosylceramide to ceramide, thus decreasing the accumulation of toxic by products in the lysosome of corrected cells. The use of GSH loci to insert the GBA gene in CD34+ stem cells allow a safe differentiation to multiple cell lineages including monocytes and macrophages, the main drivers of severe GD pathology, while having a physiological protein expression level that can minimize GD neurological complications.

### Use of GSH in Gene Therapy for Ocular Genetic Diseases:

In certain aspects, the methods and compositions described herein can be used to prevent and/or treat ocular diseases such as Inherited Retinal Dystrophies (IRDs).

Inherited retinal dystrophies (IRDs) comprise a group of rare disorders associated with genetic defects that cause progressive retinal degeneration. Patients have severe, bilateral and irreversible vision loss beginning in early to mid-life. There are more than 200 gene defects associated with the most common IRD. The ability to convert a differentiated somatic cell from a patient into a pluripotent stem cell provides new tools to treat multiple IRDs. Cells derived from these induced pluripotent stem cells (iPSCs) are now being used to screen and test the therapeutic and toxic effects of potential pharmacologic agents and gene therapies. More importantly, iPSCs can also be used to provide an easily accessible source of tissue for autologous cellular therapy. To date, the greatest potential benefit of iPSC technology is in the treatment of retinal diseases.

The retina is a complex neurovascular tissue within the eye. It contains a network of neurons nourished by the retinal and choroidal circulations. Specialized neuronal cells, called rod and cone photoreceptors, capture light that enters into the eye. Through phototransduction within the photoreceptors and downstream neural processing by the bipolar, amacrine, horizontal and ganglion cells within the retina, light signals are transmitted to the primary and secondary visual cortex of the brain to enable visual sensation (Chen et al., 2019 PMCID: PMC4470196). The functions of these specialized neuronal cells are supported by the Muller glial cells and the retinal pigment epithelium (RPE).

An alternative method to obtain patient-specific retinal cells (e.g., autologous to the subject) is to use patient-derived adult stem cells for differentiation into retinal lineages. Skin fibroblasts are routinely isolated from patients and can be transformed to pluripotent stem cells (iPSC) by transient expression of the Yamanaka factors. The combination of cellular and gene therapies to transplant corrected autologous cells has the potential to address multiple genetic retinopathies. Autologous iPSC can be transduced with gene therapy vectors to insert functional genes in specific genomic safe harbor loci.

The use of GSHs is critical to allow a safe and predictable iPSC differentiation to the desired final cell type (e.g. RPE, photoreceptors), without an undesired effect such as incomplete differentiation, clonal expansion of the targeted cells, or affecting transgene expression. Ultimately, the use of characterized GSH provide an important tool for the generation of long-term and patient-specific therapeutic treatment for inherited retinal dystrophies.

Accordingly, a nucleic acid encoding a protein deficient in patients afflicted with IRDs is integrated into a GSH locus of the present disclosure. In some embodiments, the nucleic acid encodes RPE65. A gene therapy for RPE65 has been FDA-approved for Leber congenital amaurosis (LCA) or retinitis pigmentosa (RP), which can present with severe vision loss that starts in early childhood. In some embodiments, the nucleic acid encodes CHM that treats choroideremia, which is an X-linked progressive degeneration of the retina. In some embodiments, the nucleic acid encodes RPGR that treats an X-linked RP. In some embodiments, the nucleic acid encodes PDE6B that treats RP. In some embodiments, the nucleic acid encodes CNGA3, which treats achromatopsia. In some embodiments, the nucleic acid encodes GUCY2D that treats LCA. In some embodiments, the nucleic acid encodes RS1, which treats X-linked retinoschisis, a disease characterized by early onset splitting of the retinal layers. In some embodiments, the nucleic acid encodes ABCA4 that treats Stargardt disease, the most common retinal dystrophy. In some embodiments, the nucleic acid encodes MYO7A that treats Usher syndrome type 1B. Patients afflicted with this disease have congenital hearing loss, early vision loss from RP, and vestibular dysfunction.

### Use of GSH in Gene Therapy for Hemochromatosis

In certain aspects, the methods and compositions described herein can be used to prevent and/or treat hemochromatosis.

Hereditary hemochromatosis (HH) is an autosomal recessive genetic disorder and the most prevalent genetic disease in Caucasians (Centers for Disease Control and Preventions: world wide web at cdc.gov). An estimated one million people in the United States have hereditary hemochromatosis, surpassing the prevalence of cystic fibrosis and muscular dystrophy combined (Bacon, Powell et al. 1999). HH is characterized by dysregulation in iron absorption. In HH patients, iron absorption is defective and the body absorbs iron in excess. High levels of intracellular iron deposition induce the formation of genotoxic oxygen radicals and lipoperoxidation, which establishes a pro-inflammatory response that result in chronic damage to a number of organs. The clinical features of the disease arise as result of decades of continuous accumulation of iron in parenchymal cells of the liver, heart and pancreas. In the most advanced form, HH is manifested as cirrhosis, hepatocellular cancer, diabetes mellitus, hypogonadism, cardiomyopathy, arthritis, and skin pigmentation. Enterocytes in the intestinal villi mediate the apical uptake of iron from the intestinal lumen: iron is then exported from the cells into the circulation. The apical divalent metal transporter-1 (DMT1) transports iron from the lumen into the cells, while ferroportin, a basolateral membrane bound transporter, export iron from the enterocytes into the circulation (Ezquer, Nunez et al. 2006). HH patients show an increased transepithelial iron uptake, which leads to body iron accumulation and the subsequent chronic complications (cirrhosis, hepatocellular carcinoma, pancreatitis, cardiomyopathy, arthritis and diabetes).

The most common cause of hereditary hemochromatosis is a mutation of the human homeostatic iron regulator (HFE) gene, identified on chromosome 6. Mutations in HFE are responsible for almost 90% of HH cases. The HFE gene encodes a major histocompatibility complex MHC class I-like molecule. HFE binds to β2-microglobulin, which determines its localization to the plasma membrane (Waheed, Parkkila et al. 1997). The main mutation described for HFE in association with HH is a single nucleotide change in exon 4 that results in a tyrosine for cysteine amino acid substitution at position 282 (C282Y) of the unprocessed HFE protein (Feder, Gnirke et al. 1996). This mutation affects its proper post-translational processing in the Golgi apparatus, disrupting its interaction with β2-microglobulin, and its subsequent localization in the cellular membrane. (Feder, Tsuchihashi et al. 1997, Waheed, Parkkila et al. 1997). A second mutation in the HFE gene, in which an aspartic acid moiety replaces histidine at position 63 (H63D) of the HFE protein, has also been reported (Gochee, Powell et al. 2002). The mutated and unfolded HFE protein is then accumulated in the ER-Golgi network, inducing the activation of the unfolded protein response (UPR), thus, exacerbating the pro-inflammatory program and subsequent outcome of the disease (de Almeida and de Sousa 2008, Liu, Lee et al. 2011). HFE coordinates the activity of both the iron import and iron export machinery in intestinal cells and is part of a multi-protein complex involved in transcriptional regulation of the hepcidin gene in the liver. Loss of HFE function is also associated with a drastic reduction in hepcidin expression, a negative regulator of iron uptake. Lack of HFE or hepcidin consequently results in an elevated incorporation of dietary iron and accumulation in different organs.

Another more severe form of the disease is Juvenile hemochromatosis (JH). This type of hemochromatosis is inherited and described as type II hemochromatosis. Type II hemochromatosis is categorized as type IIa or type IIb depending on the affected genes. In types IIa and IIb, the early iron overload onset occurs before 30 years of age. The consequences are severe heart disease or heart attack, hypothyroidism, little to no menstruation or hypogonadism. Hemochromatosis type IIa, results from an autosomal recessive mutation in the hepcidin gene, in chromosome 19.

Juvenile hemochromatosis is characterized by onset of severe iron overload occurring typically in the first to third decade of life. Males and females are equally affected. Prominent clinical features include hypogonadotropic hypogonadism, cardiomyopathy, glucose intolerance and diabetes, arthropathy, and liver fibrosis or cirrhosis. Hepatocellular cancer has been reported occasionally, while cardiac involvement is the main cause of morbidity and mortality.

Interestingly, the only accepted treatment for this disease is medieval, and involves periodic bleeding (phlebotomy) to reduce the iron load that is borne primarily through noncovalent coordination with heme molecules in red blood cells. At present, initially one or two units of blood (500-1000 mL) each containing 200-250 mg of iron are removed weekly until serum ferritin levels are reduced below 50 ng/ml and transferrin saturation drops to a value below 30% (requiring 2 to 3 years). Less aggressive bleeding, but life-long maintenance therapy, is then mandatory to keep the transferrin saturation value below 50% and the serum ferritin levels below 100 ng/ml (Wojcik, Speechley et al. 2002).

A therapy for hemochromatosis of different etiologies is the inhibition of DMT1 protein synthesis by the use of a siRNA in the enterocyte, which markedly inhibit apical iron uptake by intestinal epithelial cells (Ezquer, Nunez et al. 2006). The divalent metal transporter DMT-1 recently has been shown to also transport copper ions (Arredondo et al., 2003), thus inhibition of DMT-1 gene expression is of value in reducing liver injury in Wilson's disease, a condition in which copper export from cells is diminished. Decreasing the uncontrolled iron uptake in the enterocytes of HH patients will restrict the iron accumulation in several affected organs.

Another approach to control the iron load is through inhibition of ferroportin gene expression in enterocytes, to reduce the basolateral iron export. In this case, absorbed iron would only accumulate inside the enterocyte. Additionally, the accumulation of iron should lead to a reduction in the expression of the apical DMT-1 transporter gene by the IRE/IRP mechanism, producing a dual inhibitory effect. Further, any accumulated iron would be lost into the intestinal lumen by the normal slough of enterocytes.

The methods and compositions of the present disclosure, e.g., a nucleic acid vector, viral vector, pharmaceutical composition, and/or cell, wherein the wild-type HFE is integrated in the GSH locus described herein in enterocytes, can restore the HFE activity and also positively modulate the expression of DMT-1 and ferroportin, thereby having a broad therapeutic effect. A combinatorial strategy using one or more compositions described herein that co-express and/or co-administer wild-type HFE and an siRNA to silence DMT-1 can also enhance the clinical benefit.

The peptide hepcidin is a key regulator of iron metabolism. It is synthesized predominantly in the liver and secreted as a 20-25 amino acid peptide. Mutations of the hepcidin gene are responsible for juvenile hemochromatosis (Roetto, Papanikolaou et al. 2003). HFE modulates the expression of hepcidin in the liver. Hepcidin negatively regulates iron release from reticuloendothelial macrophages and from the enterocytes that mediate intestinal absorption of iron (Nemeth, Tuttle et al. 2004, Nemeth, Roetto et al. 2005, Rivera, Liu et al. 2005). Stable integration of a nucleic acid that express hepcidin to a GSH locus of the present disclosure in the liver can reduce the uptake of iron by the body and reduce the toxicity associated with iron overload, thereby preventing all form of hemochromatosis.

In certain aspects, provided herein are methods of preventing or treating a disease using at least one composition (e.g., a nucleic acid vector, viral vector, pharmaceutical composition, and/or cells) comprising a nucleic acid encoding (a) hepcidin or a fragment thereof, and/or homeostatic iron regulator (HFE) or a fragment thereof; (b) at least one non-coding RNA (e.g., piRNA, miRNA, shRNA, siRNA, gRNA, antisense RNA) that targets DMT-1, ferroportin, and/or an endogenous mutant form of HFE; (c) a CRISPR/Cas system that targets DMT-1, ferroportin, and/or an endogenous mutant form of HFE; and/or (d) any combination of any one of the nucleic acids listed in (a) to (c).

In some embodiments, the fragment is a biologically active fragment.

In some embodiments, the subject is administered with the at least composition (e.g., a nucleic acid vector, viral vector, pharmaceutical composition, and/or cells (e.g., hepatocyte, enterocyte)) comprising a nucleic acid encoding:
a) hepcidin or a fragment thereof (e.g., in hepatocyte);
b) HFE or a fragment thereof (e.g., in hepatocyte or enterocyte);
c) at least one non-coding RNA (e.g., piRNA, miRNA, shRNA, gRNA, siRNA, antisense RNA) that targets an endogenous mutant form of HFE (e.g., in hepatocyte or enterocyte);
d) at least one non-coding RNA (e.g., piRNA, miRNA, shRNA, siRNA, gRNA, antisense RNA) that targets DMT-1 (e.g., in enterocyte);
e) at least one non-coding RNA (e.g., piRNA, miRNA, shRNA, siRNA, gRNA, antisense RNA) that targets ferroportin (e.g., in enterocyte); or
f) a combination of two or more of any one of a) to e).d

In some embodiments, the method comprises a combination of two or more of any one of b) to e).

In some embodiments, the recombinant virion or pharmaceutical composition a) increases the expression of HFE or a fragment thereof, and/or hepcidin or a fragment thereof in the cell; and/or b) decreases the expression of DMT-1, ferroportin, and/or an endogenous mutant form of HFE in the cell. In some embodiments, the at least one composition (e.g., a nucleic acid vector, viral vector, pharmaceutical composition, and/or cells) prevents or treats hemochromatosis, hereditary hemochromatosis, juvenile hemochromatosis, and/or Wilson's disease.

### Inflammatory Bowel Disease (IBD)

Inflammatory Bowel Diseases (IBD) include a series of disorders that involve chronic inflammation of the human digestive tract. The most common forms of IBDs are ulcerative colitis and Crohn's disease. These are complex, multifactorial disorders characterized by chronic relapsing intestinal inflammation. Although etiology remains largely unknown, recent research has suggested that genetic factors, environment, microbiota, and autoimmune responses are contributory factors in the pathogenesis (Hendrickson, Gokhale et al. 2002). An estimated 3 million people in the U.S. have been diagnosed with IBD (world wide web at cdc.gov/ibd/data-statistics.htm), with 70,000 new cases of Crohn's disease or ulcerative colitis diagnosed each year. There is currently no cure for these painful disorders and the treatments represent an estimated annual financial healthcare burden of 6.3 billion dollar (Limanskiy, Vyas et al. 2019). The multifactorial components associated with IBD converge in the activation of a pro-inflammatory program, fundamentally mediated by genes activated by the NFkB pathway. The main pro-inflammatory cytokines induced during IBD that mediate the IBD pathobiology are TNFα, IL-1β, IL-12 and IL-6.

In some embodiments, at least one composition (e.g., a nucleic acid vector, viral vector, pharmaceutical composition, and/or cells) is used to express a soluble form of the TNFα receptor, soluble form of the IL-6 receptor, soluble form of IL-12 receptor, and/or the soluble form of IL-1β receptor. These soluble forms of said receptors can be secreted to the small intestine lamina propia where they specifically neutralize the ligands (e.g., pro-inflammatory cytokines).

A soluble form of the membrane-bound receptors can be expressed by delivering a gene encoding a soluble secreted form of the receptor. For example, a 17-kDa soluble moiety of TNFα is known to be released from cells after proteolytic cleavage of the 26-kDa type II transmembrane isoform by TNFα-converting enzyme (TACE; ADAM-17) (Kriegler et al. (1988) Cell 53:45-53). Thus, a recombinant virion of the present disclosure comprising a gene encoding the 17-kDa moiety (or any desired portion of the extracellular domain, e.g., the portion that interacts with the ligand to be antagonized/neutralized) fused to a signal peptide (e.g., IL-2 signal peptide: see e.g., Ardestani et al. (2013) Cancer Res. 73:3938-3950) can be delivered in vivo to a subject in need thereof (e.g., a subject afflicted with IBD or other inflammatory disorders) to express the soluble form of TNFα in said subject. Alternatively, either autologous or allogeneic cells can be transduced in vitro or ex vivo with such a virion comprising a gene encoding a secreted soluble form of a membrane protein, and said cells can be transferred to a subject in need thereof to treat the subject. Similar strategies can be used for any membrane bound protein.

In certain aspects, provided herein is at least one composition (e.g., a nucleic acid vector, viral vector, pharmaceutical composition, and/or cells) comprising a nucleic acid encoding (a) a soluble form of the TNFα receptor, a soluble form of the IL-6 receptor, a soluble form of the IL-12 receptor, and/or a soluble form of the IL-1β receptor; (b) at least one non-coding RNA (e.g., piRNA, miRNA, shRNA, siRNA, gRNA, antisense RNA) that targets the TNFα receptor, IL-6 receptor, IL-12 receptor, and/or IL-1β receptor; (c) a CRISPR/Cas system that targets the TNFα receptor, IL-6 receptor, IL-12 receptor, and/or IL-1β receptor; and/or (d) any combination of any one of the nucleic acids listed in (a) to (c).

In some embodiments, the at least one composition (e.g., a nucleic acid vector, viral vector, pharmaceutical composition, and/or cells) a) increases the expression of a soluble form of the TNFα receptor, a soluble form of the IL-6 receptor, a soluble form of the IL-12 receptor, or a soluble form of the IL-1β receptor in the cell; and/or b) decreases the expression of the TNFα receptor, IL-6 receptor, IL-12 receptor, or IL-1β receptor in the cell.

In some embodiments, the at least one composition (e.g., a nucleic acid vector, viral vector, pharmaceutical composition, and/or cells) prevents or treats rheumatoid arthritis, inflammatory bowel disease, psoriatic arthritis, juvenile chronic arthritis, psoriasis, and/or ankylosing spondylitis.

Accordingly, the at least one composition (e.g., a nucleic acid vector, viral vector, pharmaceutical composition, and/or cells) of the present disclosure comprising the said therapeutic genes and/or agents modulate chronic inflammation in a subject and provide therapeutic benefit by decreasing the activation of T cells, NK cells, and other effector immune cells, and allow subsequent repair of the damaged epithelial barrier. The therapeutic benefit can be further enhanced by the combination strategies provided herein.

### Autophagy-Related Diseases

The methods and at least one composition (e.g., a nucleic acid vector, viral vector, pharmaceutical composition, and/or cells) of the present disclosure that utilize the GSH loci described herein can be used to modulate the critical components of the autophagy-lysosome pathway. Autophagy plays crucial roles in differentiation and development, cellular and tissue homeostasis, protein and organelle quality control, metabolism, immunity, and protection against aging and diverse diseases. The macro-autophagy form of autophagy (hereinafter referred to as autophagy) is an evolutionarily conserved lysosomal degradation pathway that controls cellular bioenergetics (by recycling cytoplasmic components) and cytoplasmic quality (by eliminating protein aggregates, damaged organelles, lipid droplets, and intracellular pathogens) (Levine, Packer et al. 2015). In addition, independently of lysosomal degradation, the autophagic machinery can be deployed in the process of phagocytosis, apoptotic corpse clearance, secretion, exocytosis, antigen presentation, and regulation of inflammatory signaling. As a result of the broad range of cellular functions, the autophagy pathway plays a key role in protection against aging and certain cancers, infections, neurodegenerative disorders, metabolic diseases, inflammatory diseases, and muscle diseases (Levine, Packer et al. 2015).

Numerous diseases are associated with the accumulation of undesired, potentially cytotoxic cellular debris, such as misfolded-protein aggregates, nucleic acids and/or pieces of damaged organelles such as mitochondria. Autophagy also degrades lipids, allowing catabolic utilization of the fatty acids, and exerts a profound impact on fatty acid metabolic diseases such as gangliodosis, e.g., GM1, Tay-Sachs disease. Several rare autosomal disorders such as lysosomal storage disorders, are associated with the failure to degrade accumulated “cellular garbage” which generally results in the initiation of a low level but chronic inflammatory program with multiple devastating consequences such as tissue damage and cancer.

The accumulated cytoplasmic materials, known as damage associated molecular patterns (DAMPs), are considered to be ligands of a myriad of pattern recognition receptors (PRRs) that include TLRs 1-10, cGAS, IFI16, RIG-I, MDA5, NLRP family of the inflammasome proteins. Upon sensing of foreign and self-molecules, PRRs induce multiple signaling cascades with an autocrine and paracrine ability to execute fundamental cellular processes such as activation of the NFkB signaling pathway, IFN-I pathway, IFN-II pathway, IFN-III pathway, and autophagy pathways that include the AMPK, Beclin-I, PI3K pathways. Different events have been proposed to initiate the autophagy program, such as nutrient starvation conditions or exercise. AMPK activators, such as the blood glucose regulatory drug Metformin, are known to activate autophagy and increased the life span of experimental animals. The first molecular events in the activation of autophagy are the formation of an intracellular, cytosolic, double membrane structure (the autophagosome) by different cascade events that trigger congregation of proteins, such as the Atg family of proteins. The autophagosome encloses DAMPs and/or PAMPs present in the cells, the phenomenon known as the membrane nucleation stage. The next step in the autophagy pathway is the elongation and closure of the autophagosome. Finally, this matured and completely formed antophagosomes fuse with lysosomes, which contain broadly acting nucleases and proteases in a low pH environment, forming the autolysosome where the cargo is degraded into soluble and non-toxic, constituent components, thus decreasing the cytoplasmic abundance of DAMPs.

The induction of autophagy in specific tissues including liver, central nervous system (CNS) or gut, can greatly benefit patients suffering a myriad of different chronic disorders. Thus, provided herein is at least one composition (e.g., a nucleic acid vector, viral vector, pharmaceutical composition, and/or cells) comprising a nucleic acid encoding a protein or a fragment thereof selected from IRGM, NOD2, ATG2B, ATG9, ATG5, ATG7, ATG16L1, BECN1, EI24/PIG8, TECPR2, WDR45/WIP14, CHMP2B, CHMP4B, Dynein, EPG5, HspB8, LAMP2, LC3b UVRAG, VCP/p97, ZFYVE26, PARK2/Parkin, PARK6/PINK1, SQSTM1/p62, SMURF, AMPK, and ULK1. In some embodiments, the at least one composition (e.g., a nucleic acid vector, viral vector, pharmaceutical composition, and/or cells) increases the expression of said protein or a fragment thereof in the cells. In some embodiments, the at least one composition (e.g., a nucleic acid vector, viral vector, pharmaceutical composition, and/or cells) modulates autophagy. In some embodiments, the at least one composition (e.g., a nucleic acid vector, viral vector, pharmaceutical composition, and/or cells) prevents or treats an autophagy-related disease.

In some embodiments, the autophagy-related disease is selected from selected from cancer, neurodegenerative disease (e.g., Alzheimer's disease, Parkinson's disease, Huntington's disease, ataxias), inflammatory disease, inflammatory bowel disease, Crohn's disease, rheumatoid arthritis, lupus, multiple sclerosis, chronic obstructive pulmony disease/COPD, pulmonary fibrosis, cystic fibrosis, Sjogren's disease, hyperglycemic disorders, type I diabetes, type II diabetes, insulin resistance, hyperinsulinemia, insulin-resistant diabetes (e.g. Mendenhall's Syndrome, Werner Syndrome, leprechaunism, and lipoatrophic diabetes), dyslipidemia, hyperlipidemia, elevated low-density lipoprotein (LDL), depressed high density lipoprotein (HDL), elevated triglycerides, metabolic syndrome, liver disease, renal disease, cardiovascular disease, ischemia, stroke, complications during reperfusion, muscle degeneration, atrophy, symptoms of aging (e.g., muscle atrophy, frailty, metabolic disorders, low grade inflammation, atherosclerosis, stroke, age-associated dementia and sporadic form of Alzheimer's disease, pre-cancerous states, and psychiatric conditions including depression), spinal cord injury, arteriosclerosis, infectious diseases (e.g., bacterial, fungal, viral), AIDS, tuberculosis, defects in embryogenesis, infertility, lysosomal storage diseases, activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM1 Gangliosidosis, (infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs, and Wolman disease.

As used herein, the term “autophagy-related diseases” refers to diseases that result from disruption in autophagy or cellular self-digestion. Autophagic dysfunction is associated with cancer, neurodegeneration, microbial infection and aging, among numerous other disease states and/or conditions. Although autophagy plays a principal role as a protective process for the cell, it also plays a role in cell death. Disease states and/or conditions which are mediated through autophagy (which refers to the fact that the disease state or condition may manifest itself as a function of the increase or decrease in autophagy in the patient or subject to be treated and treatment or prevention requires administration of an inhibitor or agonist of autophagy in the patient or subject) include, for example, cancer, including metastasis of cancer, lysosomal storage diseases (discussed hereinbelow), neurodegeneration (including, for example, Alzheimer's disease, Parkinson's disease, Huntington's disease: other ataxias), immune response (T cell maturation, B cell and T cell homeostasis, counters damaging inflammation) and chronic inflammatory diseases (may promote excessive cytokines when autophagy is defective), including, for example, inflammatory bowel disease, including Crohn's disease, rheumatoid arthritis, lupus, multiple sclerosis, chronic obstructive pulmony disease/COPD, pulmonary fibrosis, cystic fibrosis, Sjogren's disease: hyperglycemic disorders, type I diabetes, type II diabetes, affecting lipid metabolism islet function and/or structure, excessive autophagy may lead to pancreatic b-cell death and related hyperglycemic disorders, including severe insulin resistance, hyperinsulinemia, insulin-resistant diabetes (e.g. Mendenhall's Syndrome, Werner Syndrome, leprechaunism, and lipoatrophic diabetes) and dyslipidemia (e.g. hyperlipidemia as expressed by obese subjects, elevated low-density lipoprotein (LDL), depressed high density lipoprotein (HDL), and elevated triglycerides) and metabolic syndrome, liver disease (excessive autophagic removal of cellular entities-endoplasmic reticulum), renal disease (apoptosis in plaques, glomerular disease), cardiovascular disease (especially including ischemia, stroke, pressure overload and complications during reperfusion), muscle degeneration and atrophy, symptoms of aging (including amelioration or the delay in onset or severity or frequency of aging-related symptoms and chronic conditions including muscle atrophy, frailty, metabolic disorders, low grade inflammation, atherosclerosis and associated conditions such as cardiac and neurological both central and peripheral manifestations including stroke, age-associated dementia and sporadic form of Alzheimer's disease, pre-cancerous states, and psychiatric conditions including depression), stroke and spinal cord injury, arteriosclerosis, infectious diseases (microbial infections, removes microbes, provides a protective inflammatory response to microbial products, limits adaptation of autophagy of host by microbe for enhancement of microbial growth, regulation of innate immunity) including bacterial, fungal, cellular and viral (including secondary disease states or conditions associated with infectious diseases), including AIDS and tuberculosis, among others, development (including erythrocyte differentiation), embryogenesis/fertility/infertility (embryo implantation and neonate survival after termination of transplacental supply of nutrients, removal of dead cells during programmed cell death) and aging (increased autophagy leads to the removal of damaged organelles or aggregated macromolecules to increase health and prolong life, but increased levels of autophagy in children/young adults may lead to muscle and organ wasting resulting in aging/progeria).

The term “lysosomal storage disorder” refers to a disease state or condition that results from a defect in lysosomal storage. These disease states or conditions generally occur when the lysosome malfunctions. Lysosomal storage disorders are caused by lysosomal dysfunction usually as a consequence of deficiency of an enzyme required for the metabolism of lipids, glycoproteins or mucopolysaccharides. The incidence of lysosomal storage disorder (collectively) occurs at an incidence of about 1:5,000-1:10,000. The lysosome is commonly referred to as the cell's recycling center because it processes unwanted material into substances that the cell can utilize. Lysosomes break down this unwanted matter via high specialized enzymes. Lysosomal disorders generally are triggered when a particular enzyme exists in too small an amount or is missing altogether. When this happens, substances accumulate in the cell. In other words, when the lysosome doesn't function normally, excess products destined for breakdown and recycling are stored in the cell. Lysosomal storage disorders are genetic diseases, but these may be treated using autophagy modulators (autostatins) as described herein. All of these diseases share a common biochemical characteristic, i.e., that all lysosomal disorders originate from an abnormal accumulation of substances inside the lysosome. Lysosomal storage diseases mostly affect children who often die as a consequence at an early stage of life, many within a few months or years of birth. Many other children die of this disease following years of suffering from various symptoms of their particular disorder.

Examples of lysosomal storage diseases include, for example, activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM1 Gangliosidosis, including infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs, and Wolman disease, among others.

### Infection

In some embodiments, the methods and compositions described herein relate to the treatment or prevention of bacterial infection, bacterial septic shock, fungal infection, and/or viral infection.

In some embodiments, the methods and compositions described herein relate to the treatment or prevention of a viral infection such as a respiratory viral infection, such as a coronavirus infection (e.g., a MERS (Middle East Respiratory Syndrome) infection, a severe acute respiratory syndrome (SARS) infection, such as a SARS-COV-2 infection), an influenza infection, and/or a respiratory syncytial virus infection. In some embodiments, the methods and solid dosage forms described herein provided herein are for the treatment of a coronavirus infection (e.g., a MERS infection, a severe acute respiratory syndrome (SARS) infection, such as a SARS-COV-2 infection). In some embodiments, provided herein are methods and compositions for treating COVID-19.

In some embodiments, the infection is the viral infection; and the viral infection is by a coronavirus (e.g., MERS, SARS), influenza virus, respiratory syncytial virus, hepatitis A, hepatitis B, hepatitis C, hepatitis D, hepatitis E, human papillomavirus, dengue virus serotype 1, dengue virus serotype 2, dengue virus serotype 3, dengue virus serotype 4, zika, virus, West Nile virus, yellow fever virus, Chikungunya virus, Mayaro virus, Ebola virus, Marburg virus, or Nipa virus. In some embodiments, the viral infection is by SARS-CoV-2.

### Inflammatory Disorders

The methods and/or at least one composition (e.g., a nucleic acid vector, viral vector, pharmaceutical composition, and/or cells) described herein can be used, for example, for preventing or treating (reducing, partially or completely, the adverse effects of) an autoimmune disease, such as chronic inflammatory bowel disease, systemic lupus erythematosus, psoriasis, muckle-wells syndrome, rheumatoid arthritis, multiple sclerosis, or Hashimoto's disease; an allergic disease, such as a food allergy, pollenosis, or asthma; an infectious disease, e.g., infection with Clostridium difficile; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease, such as chronic obstructive pulmonary disease); a pharmaceutical composition for suppressing rejection in organ transplantation or other situations in which tissue rejection might occur; a pharmaceutical composition for improving immune functions; or a pharmaceutical composition for suppressing the proliferation or function of immune cells.

In some embodiments, the methods and compositions provided herein are useful for the treatment or prevention of inflammation. In certain embodiments, the inflammation of any tissue and organs of the body, including musculoskeletal inflammation, vascular inflammation, neural inflammation, digestive system inflammation, ocular inflammation, inflammation of the reproductive system, and other inflammation, as discussed below.

Immune disorders of the musculoskeletal system include, but are not limited, to those conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular immune disorders refers to a immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis

Examples of nervous system immune disorders which may be treated with the methods and compositions described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

Examples of digestive system immune disorders which may be treated with the methods and pharmaceutical compositions described herein include, but are not limited to, cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

Examples of reproductive system immune disorders which may be treated with the methods and pharmaceutical compositions described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

The methods and at least one composition (e.g., a nucleic acid vector, viral vector, pharmaceutical composition, and/or cells) described herein may be used to prevent or treat autoimmune conditions having an inflammatory component. Such conditions include, but are not limited to, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, type 2 diabetes, giant cell arteritis, goodpasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

The methods and at least one composition (e.g., a nucleic acid vector, viral vector, pharmaceutical composition, and/or cells) described herein may be used to prevent or treat T-cell mediated hypersensitivity diseases having an inflammatory component. Such conditions include, but are not limited to, contact hypersensitivity, contact dermatitis (including that due to poison ivy), uticaria, skin allergies, respiratory allergies (hay fever, allergic rhinitis, house dustmite allergy) and gluten-sensitive enteropathy (Celiac disease).

Other immune disorders which may be treated with the methods and pharmaceutical compositions include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenia purpura in adults, secondary thrombocytopenia in adults, acquired (autoimmune) haemolytic anemia, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosus, psoriasis, chronic obstructive pulmonary disease, and inflammation accompanying infectious conditions (e.g., sepsis).

### Neurodegenerative & Neuroinflammatory Disorders

The methods and/or at least one composition (e.g., a nucleic acid vector, viral vector, pharmaceutical composition, and/or cells) described herein may be used to prevent or treat neurodegenerative and neurological diseases. In certain embodiments, the neurodegenerative and/or neurological disease is Parkinson's disease, Alzheimer's disease, prion disease, Huntington's disease, motor neuron diseases (MND), spinocerebellar ataxia, spinal muscular atrophy, dystonia, idiopathicintracranial hypertension, epilepsy, nervous system disease, central nervous system disease, movement disorders, multiple sclerosis, encephalopathy, peripheral neuropathy, post-operative cognitive dysfunction, frontotemporal dementia, stroke, transient ischemic attack, vascular dementia, Creutzfeldt-Jakob disease, multiple sclerosis, prion disease, Pick's disease, corticobasal degeneration, Parkinson's disease, Lewy body dementia, progressive supranuclear palsy, dementia pugilistica (chronic traumatic encephalopathy), frontotemporal dementia, parkinsonism linked to chromosome 17, Lytico-Bodig disease, Tangle-predominant dementia, ganglioglioma, gangliocytoma, meningioangiomatosis, subacute sclerosing panencephalitis, lead encephalopathy, tuberous sclerosis, Hallervorden-Spatz disease, lipofuscinosis, argyrophilic grain disease, and frontotemporal lobar degeneration.

The methods and/or at least one composition (e.g., a nucleic acid vector, viral vector, pharmaceutical composition, and/or cells) described herein may be used to prevent or treat neuroinflammation and/or neuroinflammatory diseases, e.g., using a recombinant virion of the present disclosure to deliver a nucleic acid comprising a gene encoding one or more cytokines that alleviate inflammation. Neuroinflammatory diseases include, but not limited to, an autoimmune disease, an inflammatory disease, a neurogenerative disease, a neuromuscular disease, or a psychiatric disease. In some embodiments, the methods and compositions provided herein are useful for treatment or prevention of the inflammation of central nervous system, including brain inflammation, peripheral nerves inflammation, neural inflammation, spinal cord inflammation, ocular inflammation, and/or other inflammation.

Examples of disorders associated with neuroinflammation or neuroinflammatory disorders which may be treated with the methods and compositions described herein include, but are not limited to, encephalitis (inflammation of the brain), encephalomyelitis (inflammation of the brain and spinal cord), meningitis (inflammation of the membranes that surround the brain and spinal cord), Guillain-Barre syndrome, neuromyotonia, narcolepsy, multiple sclerosis, myelitis, schizophrenia, acute disseminated encephalomyelitis (ADEM), accute optic neuritis (AON), transverse myelitis, neuromyelitis optica (NMO), Alzheimer's disease, Parkinson's disease, amyotrophic lateral sclerosis, frontotemporal lobar dementia, optic neuritis, neuromyelitis optica spectrum disorder (NMOSD), auto-immune encephalitis, anti-NMDA receptor encephalitis, Rasmussen's encephalitis, acute necrotizing encephalopathy of childhood (ANEC), opsoclonus-myoclonus ataxia syndrome, traumatic brain injury, Huntington's disease, depression, anxiety, migraine, myasthenia gravis, acute ischemic stroke, epilepsy, synucleinopathies, frontotemporal dementia, progressive nonfluent aphasia, semantic dementia, Nodding syndrome, cerebral ischemia, neuropathic pain, autism spectrum disorder, fibromyalgia syndrome, progressive supranuclear palsy, corticobasal degeneration, systemic lupus erythematosus, prion disease, motor neurone diseases (MND), spinocerebellar ataxia, spinal muscular atrophy, dystonia, idiopathicintracranial hypertension, nervous system disease, central nervous system disease, movement disorders, encephalopathy, peripheral neuropathy, or post-operative cognitive dysfunction.

### Cancer

As described herein, the methods and/or at least one composition (e.g., a nucleic acid vector, viral vector, pharmaceutical composition, and/or cells) provided herein may comprise integration of a nucleic acid encoding e.g., a tumor suppressor at a GSH locus of the present disclosure. Similarly, the methods and/or at least one composition (e.g., a nucleic acid vector, viral vector, pharmaceutical composition, and/or cells) provided herein may comprise integration of a nucleic acid encoding a non-coding RNA (e.g., piRNA, miRNA, shRNA, siRNA, gRNA, antisense RNA) that downregulates e.g., an oncogene.

Cancer, tumor, or hyperproliferative disorder refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, (e.g., multiple myeloma, Diffuse large B-cell lymphoma (DLBCL), Follicular lymphoma, Chronic lymphocytic leukemia (CLL), small lymphocytic lymphoma (SLL), Mantle cell lymphoma (MCL), Marginal zone lymphomas, Burkitt lymphoma, Waldenström's macroglobulinemia, Hairy cell leukemia, Primary central nervous system (CNS) lymphoma, Primary intraocular lymphoma, the heavy chain diseases, such as, for example, alpha chain disease, gamma chain disease, and mu chain disease, benign monoclonal gammopathy, and immunocytic amyloidosis), T cell cancer (e.g., T-lymphoblastic lymphoma/leukemia, non-Hodgkin lymphomas, Peripheral T-cell lymphomas, Cutaneous T-cell lymphomas (e.g., mycosis fungoides, Sezary syndrome), Adult T-cell leukemia/lymphoma, Angioimmunoblastic T-cell lymphoma, Extranodal natural killer/T-cell lymphoma, Enteropathy-associated intestinal T-cell lymphoma (EATL), Anaplastic large cell lymphoma (ALCL), Hodgkin lymphoma), melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma (SCLC), bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma; leukemias, e.g., acute lymphocytic leukemia and acute myelocytic leukemia (myeloblastic, promyelocytic, myelomonocytic, monocytic and erythroleukemia): chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

### Familial Intrahepatic Cholestasis

The methods and/or compositions described herein may be used to prevent or treat familial intrahepatic cholestasis (PFIC), a genetic disease associated with mutations in the ATPB1, ATPB11 and ABCB4 genes which results in PFIC type 1, 2 and 3, respectively. This rare autosomal recessive disease drives the disruption of the bile secretory pathway, characterized by ductular proliferation in the liver and progressive intrahepatic cholestasis with elevated gamma-glutamyltranspeptidase (GGT) activity. ABCB4 mutations are the most prevalent forms of the disease. The ABCB4 gene is located on chromosome 7q21.1 and encodes for the lipid floppase MDR3 protein, involved in causing PFIC3. MDR3 is primarily expressed at the canalicular membrane of the liver and acts as a phospholipid translocator, i.e., phosphatidylcholine (PC). MDR3 protects the hepatocytemembrane from detergent activity of bile salts. The PFIC3 defect is characterized by reduced secretion of phosphatidylcholine (PC) into bile, thus impairing the bile secretory transport system (Davit-Spraul, et al., PMID: 20422496). Reduced PC secretion causes toxicity in the liver which results in the activation of a pro-inflammatory program with a concomitant destruction of hepatocytes that further progresses to intrahepatic liver cirrhosis. Other less prevalent forms of the disease are caused by mutations in ATPB1 and ATPB11 genes which result in similar outcomes. Accordingly, a gene therapy for ATPB1, ATPB11, and/or ABCB4 is useful in preventing and/or treating familial intrahepatic cholestasis.

### Wilson Disease

The methods and/or compositions described herein may be used to prevent or treat Wilson Disease (WD). WD is a monogenic, autosomal recessively inherited condition, associated with mutations in the ATP7B gene, which encode a copper-transporting P-type ATPase. More than 600 pathogenic variants in ATP7B have been identified, with single-nucleotide missense and nonsense mutations being the most common, followed by insertions/deletions, and, rarely, splice site mutations. ATP7B is most highly expressed in the liver, but is also found in the kidney, placenta, mammary glands, brain, and lung. ATPB7 disruption leads to increased intracellular copper levels. Human dietary intake of copper is about 1.5-2.5 mg/day, which is absorbed in the stomach and duodenum, bound to circulating albumin, and transported to the liver for regulation and excretion. The antioxidant protein 1 (ATOX1) delivers copper to ATPB7 by copper-dependent protein-protein interaction. Within hepatocytes, ATP7B performs two important functions in either the trans-Golgi network (TGN) or in cytoplasmic vesicles. In the TGN, ATP7B activates ceruloplasmin by packaging six copper molecules into apoceruloplasmin, which is then secreted into the plasma. In the cytoplasm, ATP7B sequesters excess copper into vesicles and excretes it via exocytosis across the apical canalicular membrane into bile (Bull et al., 1993; Tanzi et al., 1993; Yamaguchi et al., 1999; Cater et al., 2007). Due to the binary role of the ATP7B transporter in both the synthesis and excretion of copper, defects in its function lead to copper accumulation triggering oxidative stress and free radical formation as well as mitochondrial dysfunction arising independently of oxidative stress. The combined effects results in the induction of a pro-inflammatory state and subsequent cell death in hepatic and brain tissue as well as other organs.

### Lysosomal Storage Disorders

The methods and/or compositions described herein may be used to prevent or treat lysosomal storage diseases (LSD). These are inherited metabolic diseases that are characterized by an abnormal build-up of various toxic materials in the body's cells as a result of enzyme deficiencies. The methods and compositions described herein may be used to prevent or treat carbamoyl phosphate synthetase 1 deficiency (CPS1D), a rare autosomal recessive disorder, characterized by a destructive metabolic disease dominated by severe hyperammonemia that affect multiple organs, including in some cases changes in brain white matter. CPS1 plays a paramount role in liver ureagenesis since it catalyzes the first and rate-limiting step of the urea cycle, the major pathway for nitrogen disposal in humans. CPS1 deficiency leads to urea cycle disorder and accumulation of ammonia. Therefore, marked hyperammonemia and decreased downstream production of the urea cycle can be observed in patients with CPS1 deficiency. The superabundant ammonia can enter the central nervous system and exerts its toxic effects on the brain. Accumulation of ammonia induces toxicity and lead to cell death.

### Hematologic Diseases

In certain aspects, in addition to the hematologic diseases described below, the methods and/or compositions described herein can be used for treatment or prevention of a disease such as endothelial dysfunction, cystic fibrosis, cardiovascular disease, peripheral vascular disease, stroke, heart disease (e.g., including congenital heart disease), diabetes, insulin resistance, chronic kidney failure, atherosclerosis, tumor growth (e.g., including those of endothelial cells), metastasis, hypertension (e.g., pulmonary arterial hypertension, other forms of pulmonary hypertension), atherosclerosis, restenosis, Hepatitis C, liver cirrhosis, hyperlipidemia, hypercholesterolemia, metabolic syndrome, renal disease, inflammation, and venous thrombosis.

In certain aspects, a hematologic disease includes any one of the following: hemoglobinopathy (e.g., sickle cell disease, thalassemia, methemoglobinemia), anemia (iron-deficiency anemia, megaloblastic anemia, hemolytic anemias, myelodysplastic syndrome, myelofibrosis, neutropenia, agranulocytosis, Glanzmann's thrombasthenia, thrombocytopenia, Wiskott-Aldrich syndrome, myeloproliferative disorders (e.g., polycythemia vera, erythrocytosis, leukocytosis, thrombocytosis), coagulopathies, a hematologic cancer, hemochromatosis, asplenia, hypersplenism (e.g., Gaucher's disease), hemophagocytic lymphohistiocytosis, tempi syndrome, and AIDS.

In some embodiments, the exemplary hemolytic anemia includes: Hereditary spherocytosis, Hereditary elliptocytosis, Congenital dyserythropoietic anemia, Glucose-6-phosphate dehydrogenase deficiency (G6PD), pyruvate kinase deficiency, autoimmune hemolytic anemia (e.g., idiopathic anemia, Systemic lupus erythematosus (SLE), Evans syndrome, Cold agglutinin disease, Paroxysmal cold hemoglobinuria, Infectious mononucleosis), alloimmune hemolytic anemia (e.g., hemolytic disease of the newborn, such as Rh disease, ABO hemolytic disease of the newborn, anti-Kell hemolytic disease of the new born, Rhesus c hemolytic disease of the newborn, Rhesus E hemolytic disease of the newborn), Paroxysmal nocturnal hemoglobinuria, Microangiopathic hemolytic anemia, Fanconi anemia, Diamond-Blackfan anemia, and Acquired pure red cell aplasia.

In some embodiments, the exemplary coagulopathy includes: thrombocytosis, disseminated intravascular coagulation, hemophilia (e.g., hemophilia A, hemophilia B, hemophilia C), von Willebrand disease, and antiphospholipid syndrome.

In some embodiments, the exemplary hematologic cancer includes: Hodgkin's disease, Non-Hodgkin's lymphoma, Burkitt's lymphoma, Anaplastic large cell lymphoma, Splenic marginal zone lymphoma, T-cell lymphoma (e.g., Hepatosplenic T-cell lymphoma, Angioimmunoblastic T-cell lymphoma, Cutaneous T-cell lymphoma), Multiple myeloma, Waldenström macroglobulinemia, Plasmacytoma, Acute lymphocytic leukemia (ALL), Chronic lymphocytic leukemia (CLL), Acute myelogenous leukemia (AML), Acute megakaryoblastic leukemia, Chronic Idiopathic Myelofibrosis, Chronic myelogenous leukemia (CML), T-cell prolymphocytic leukemia, B-cell prolymphocytic leukemia, Chronic neutrophilic leukemia, Hairy cell leukemia, T-cell large granular lymphocyte leukemia, AIDS-related lymphoma, Sezary syndrome, Waldenström Macroglobulinemia, Chronic Myeloproliferative Neoplasms, Langerhans Cell Histiocytosis, Myelodysplastic Syndromes, and Aggressive NK-cell leukemia.

As used herein, the hemoglobinopathy includes any disorder involving the presence of an abnormal hemoglobin molecule in the blood. Examples of hemoglobinopathies included, but are not limited to, hemoglobin C disease, hemoglobin sickle cell disease (SCD), sickle cell anemia, and thalassemias. Also included are hemoglobinopathies in which a combination of abnormal hemoglobins are present in the blood (e.g., sickle cell/Hb-C disease).

As used herein, thalassemia refers to a hereditary disorder characterized by defective production of hemoglobin. Examples of thalassemias include α- and β-thalassemia. β-thalassemias are caused by a mutation in the beta globin chain, and can occur in a major or minor form. In the major form of β-thalassemia, children are normal at birth, but develop anemia during the first year of life. The mild form of β-thalassemia produces small red blood cells and the thalassemias are caused by deletion of a gene or genes from the globin chain, α-thalassemia typically results from deletions involving the HBA1 and HBA2 genes. Both of these genes encode α-globin, which is a component (subunit) of hemoglobin. There are two copies of the HBA1 gene and two copies of the HBA2 gene in each cellular genome. As a result, there are four alleles that produce α-globin. The different types of a thalassemia result from the loss of some or all of these alleles. Hb Bart syndrome, the most severe form of a thalassemia, results from the loss of all four α-globin alleles. HbH disease is caused by a loss of three of the four α-globin alleles. In these two conditions, a shortage of α-globin prevents cells from making normal hemoglobin. Instead, cells produce abnormal forms of hemoglobin called hemoglobin Bart (Hb Bart) or hemoglobin H (HbH). These abnormal hemoglobin molecules cannot effectively carry oxygen to the body's tissues. The substitution of Hb Bart or HbH for normal hemoglobin causes anemia and the other serious health problems associated with a thalassemia.

As used herein, the sickle cell disease refers to a group of autosomal recessive genetic blood disorders, which results from mutations in a globin gene and which is characterized by red blood cells that under hypoxic conditions, convert from the typical biconcave form into an abnormal, rigid, sickle shape that cannot course through capillaries, thereby exacerbating the hypoxia. They are defined by the presence of βs-gene coding for a β-globin chain variant in which glutamic acid is substituted by valine at amino acid position 6 of the peptide, and second β-gene that has a mutation mat allows for the crystallization of HbS leading to a clinical phenotype. Sickle cell anemia refers to a specific form of sickle cell disease in patients who are homozygous for the mutation that causes HbS. Other common forms of sickle cell disease include HbS/β-thalassemia, HbS/HbC and HbS/HbD.

In certain embodiments, methods and compositions are provided herein to treat, prevent, or ameliorate a hemoglobinopathy that is selected from the group consisting of: hemoglobin C disease, hemoglobin sickle cell disease (SCD), sickle cell anemia, hereditary anemia, thalassemia, β-thalassemia, thalassemia major, thalassemia intermedia, α-thalassemia, and hemoglobin H disease. In some embodiments, the hemoglobinopathy is β-thalassemia. In some embodiments, the hemoglobinopathy is sickle cell anemia. In various embodiments, the viral vectors described herein are administered in vivo by direct injection to a cell, tissue, or organ of a subject in need of gene therapy. In various other embodiments, cells are transduced in vitro or ex vivo with the recombinant virions described herein. The cells are then administered to a subject in need of gene therapy, e.g., within a pharmaceutical formulation disclosed herein.

As described above, provided herein are methods and compositions of preventing or treating a hemoglobinopathy in a subject. In various embodiments, the method comprises administering an effective amount of a cell transduced with the viral vectors described herein or a population of the said cells (e.g., HSCs, CD34+ or CD36 cells, erythroid lineage cells, embryonic stem cells, or iPSCs) to the subject. For treatment or prevention, the amount administered can be an amount effective in producing the desired clinical benefit. An effective amount can be provided in one or a series of administrations. An effective amount can be provided in a bolus or by continuous perfusion. An effective amount can be administered to a subject in one or more doses. In terms of treatment or prevention, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the ordinary skill of one in the art. Several factors are typically taken into account when determining an appropriate dosage to achieve an effective amount. These factors include age, sex and weight of the subject, the condition being treated, the severity of the condition.

### Hemophilia A

Hemophilia A is an inherited bleeding disorder in which the blood does not clot normally. People with hemophilia A bleed more than normal after an injury, surgery, or dental procedure. This disorder can be severe, moderate, or mild. In severe cases, heavy bleeding occurs after minor injury or even when there is no injury (spontaneous bleeding). Bleeding into the joints, muscles, brain, or organs can cause pain and other serious complications. In milder forms, there is no spontaneous bleeding, and the disorder might only be diagnosed after a surgery or serious injury. Hemophilia A is caused by having low levels of a protein called factor VIII. Factor VIII is needed to form blood clots. The disorder is inherited in an X-linked recessive manner and is caused by changes (mutations) in the F8 gene. The diagnosis of hemophilia A is made through clinical symptoms and specific laboratory tests to measure the amount of clotting factors in the blood. The main prevention or treatment is replacement therapy, during which clotting factor VIII is dripped or injected slowly into a vein. Hemophilia A mainly affects males. With prevention or treatment, most people with this disorder do well. Some people with severe hemophilia A may have a shortened lifespan due to the presence of other health conditions and rare complications of the disorder.

Patients afflicted with hemophilia A stands to benefit from gene therapy that introduces the F8 transgene encoding a full length factor VIII (FVIII) or a B-domain-deleted FVIII (e.g., FVIII-SQ, p-VIII, p-VIII-LMW; Sandberg et al. (2001) Thromb Haemost 85:93-100), which retains activity necessary to provide therapeutic benefits in human (Rangarajan et al. (2017) N Engl J Med 377:2519-30). The recombinant virions, pharmaceutical compositions, and methods of the present disclosure provide improved viral vectors and prevention/treatment methods for patients afflicted with hemophilia A, in part due to the ability of the recombinant virions to package larger genes compared with AAV, low immunogenicity, and pulsatile gene regulation (see Example 9 and section “Pulsatile Gene Expression or Inducible Gene Expression”).

In some embodiments, the disease treated includes one selected from those presented in Table 4.

TABLE 4

| | | | | |
|---|---|---|---|---|
| | Defective | | Major Organ Systems | CNS |
| Disease | Protein | Materials Stored | Affected | Pathology |

Sphingolipidoses

| | | | | |
|---|---|---|---|---|
| Fabry | α-Galactosidase A | Globotriasylceramide | Kidney, heart | + |
| | | and blood Group B sub- | | |
| | | stances | | |
| Gaucher | β-Glucosidase | Glucosylceramide | Spleen, liver, bone mar- | +(types 2 and 3) |
| | | glucosylsphingosine | row | |
| Nieman-Pick A | Sphingomyelinase | Sphingomyelin | Spleen, liver, bone mar- | +(type A) |
| and B | | | row, lung (type B) | |
| GMI Gangliosidosis | β-Galactosidase | GM I ganglioside | Skeleton, heart | + |

Mucopolysaccharidoses

| | | | | |
|---|---|---|---|---|
| MPS I (Hurler, Scheie, | α-Iduronidase | Dermatan sulphate and | Organomegaly, skeleton, | + |
| Hurler/Scheie) | | heparan sulphate | eye | |
| MPS II (Hunter) | Iduronate-2-sulphatase | Dermatan sulphate and | Organomegaly, heart | + |
| | | heparan sulphate | | |
| MPS VI (Maroteaux- | acetylgalactosamine-4- | Dermatan sulphate | Spleen, liver, heart, | − |
| Lamy) | sulphatase (arylsulpha- | | macrocephaly, | |
| | tase B) | | | |

In some embodiments, following administration of one or more of the presently disclosed cells, peripheral blood of the subject is collected and hemoglobin level is measured. A therapeutically relevant level of hemoglobin is produced following administration of the viral vectors or the cells transduced with the viral vectors. Therapeutically relevant level of hemoglobin is a level of hemoglobin that is sufficient (1) to improve anemia, (2) to improve or restore the ability of the subject to produce red blood cells containing normal hemoglobin, (3) to improve or correct ineffective erythropoiesis in the subject, (4) to improve or correct extra-medullary hematopoiesis (e.g., splenic and hepatic extra-medullary hematopoiesis), and/or(S) to reduce iron accumulation, e.g., in peripheral tissues and organs. Therapeutically relevant level of hemoglobin can be at least about 7 g/dL Hb, at least about 7.5 g/dL Hb, at least about 8 g/dL Hb, at least about 8.5 g/dL Hb, at least about 9 g/dL Hb, at least about 9.5 g/dL Hb, at least about 10 g/dL Hb, at least about 10.5 g/dL Hb, at least about 11 g/dL Hb, at least about 11.5 g/dL Hb, at least about 12 g/dL Hb, at least about 12.5 g/dL Hb, at least about 13 g/dL Hb, at least about 13.5 g/dL Hb, at least about 14 g/dL Hb, at least about 14.5 g/dL Hb, or at least about 15 g/dL Hb. Additionally or alternatively, therapeutically relevant level of hemoglobin can be from about 7 g/dL Hb to about 7.5 g/dL Hb, from about 7.5 g/dL Hb to about 8 g/dL Hb, from about 8 g/dL Hb to about 8.5 g/dL Hb, from about 8.5 g/dL Hb to about 9 g/dL Hb, from about 9 g/dL Hb to about 9.5 g/dL Hb, from about 9.5 g/dL Hb to about 10 g/dL Hb, from about 10 g/dL Hb to about 10.5 g/dL Hb, from about 10.5 g/dL Hb to about 11 g/dL Hb, from about 11 g/dL Hb to about 11.5 g/dL Hb, from about 11.5 g/dL Hb to about 12 g/dL Hb, from about 12 g/dL Hb to about 12.5 g/dL Hb, from about 12.5 g/dL Hb to about 13 g/dL Hb, from about 13 g/dL Hb to about 13.5 g/dL Hb, from about 13.5 g/dL Hb to about 14 g/dL Hb, from about 14 g/dL Hb to about 14.5 g/dL Hb, from about 14.5 g/dL Hb to about 15 g/dL Hb, from about 7 g/dL Hb to about 8 g/dL Hb, from about 8 g/dL Hb to about 9 g/dL Hb, from about 9 g/dL Hb to about 10 g/dL Hb, from about 10 g/dL Hb to about 11 g/dL Hb, from about 11 g/dL Hb to about 12 g/dL Hb, from about 12 g/dL Hb to about 13 g/dL Hb, from about 13 g/dL Hb to about 14 g/dL Hb, from about 14 g/dL Hb to about 15 g/dL Hb, from about 7 g/dL Hb to about 9 g/dL Hb, from about 9 g/dL Hb to about 11 g/dL Hb, from about 11 g/dL Hb to about 13 g/dL Hb, or from about 13 g/dL Hb to about 15 g/dL Hb. In certain embodiments, the therapeutically relevant level of hemoglobin is maintained in the subject for at least 3 days, for at least 1 week, for at least 2 weeks, for at least 1 month, for at least 2 months, for at least 4 months, for at least about 6 months, for at least about 12 months (or 1 year), for at least about 24 months (or 2 years). In certain embodiments, the therapeutically relevant level of hemoglobin is maintained in the subject for up to about 6 months, for up to about 12 months (or 1 year), for up to about 24 months (or 2 years). In certain embodiments, the therapeutically relevant level of hemoglobin is maintained in the subject for about 3 days, for about 1 week, for about 2 weeks, for about 1 month, for about 2 months, for about 4 months, for about 6 months, for about 12 months (or 1 year), for about 24 months (or 2 years). In certain embodiments, the therapeutically relevant level of hemoglobin is maintained in the subject for from about 6 months to about 12 months (e.g., from about 6 months to about 8 months, from about 8 months to about 10 months, from about 10 months to about 12 months), from about 12 months to about 18 months (e.g., from about 12 months to about 14 months, from about 14 months to about 16 months, or from about 16 months to about 18 months), or from about 18 months to about 24 months (e.g., from about 18 months to about 20 months, from about 20 months to about 22 months, or from about 22 months to about 24 months).

In certain embodiments, the cell is autologous to the subject being administered with the cell. In some embodiments, the cell is from the bone marrow or mobilized cells in the peripheral circulation, autologous to the subject being administered with the cell. In other embodiments, the cell is allogeneic to the subject being administered with the cell. In some embodiments, the cell is from the bone marrow autologous to the subject being administered with the cell.

The present disclosure also provides a method of increasing the proportion of red blood cells or erythrocytes compared to white blood cells or leukocytes in a subject. In various embodiments, the method comprises administering an effective amount of the at least one composition (a nucleic acid vector, viral vector, pharmaceutical composition, and/or cell (e.g., HSCs, CD34+ or CD36 cells, erythroid lineage cells, embryonic stem cells, or iPSCs)) described herein to the subject, wherein the proportion of red blood cell progeny cells of the hematopoietic stem cells are increased compared to white blood cell progeny cells of the hematopoietic stem cells in the subject.

The quantity of cells to be administered will vary for the subject and/or the disease being prevented or treated. In some embodiments, from about 1×10⁴ to about 1×10⁵ cells/kg, from about 1×10⁵ to about 1×10⁶ cells/kg, from about 1×10⁶ to about 1×10⁷ cells/kg, from about 1×10⁷ to about 1×10⁸ cells/kg, from about 1×10⁸ to about 1×10⁹ cells/kg, or from about 1×10⁹ to about 1×10¹⁰ cells/kg of the presently disclosed cells are administered to a subject. Depending on the needs, the subject may need multiple doses of the cells. The precise determination of what would be considered an effective dose may be based on factors individual to each subject, including their size, age, sex, weight, and condition of the particular subject. Dosages can be readily ascertained by those skilled in the art from this disclosure and the knowledge in the art.

Without being bound to any particular theory, an important advantage provided by the compositions and methods described herein is an efficient way of treating a subject afflicted with any disease (e.g., a hemoglobinopathy, cystic fibrosis, hemochromatosis) or preventing any disease in a subject, e.g., those at risk of developing such disease by utilizing the GSH loci of the present disclosure. The at risk subjects can be identified by certain genetic mutations they carry, and/or environmental or physical factors (e.g., sex, age of the subject). The highly efficient and safe gene therapy is achieved by using the compositions and methods described herein. For example, the targeted integration of the nucleic acid (e.g., therapeutic nucleic acid) to a GSH reduces the chances of deleterious mutation, transformation, or oncogene activation of cellular genes in cells.

### Exemplary Embodiments

1. A method of identifying a genomic safe harbor (GSH) locus, comprising:
   (a) inducing a random insertion of at least one marker gene into a genome in a cell;
   (b) determining the stability and/or level of the marker gene expression; and
   (c) identifying a genomic locus, wherein the inserted marker gene shows the stable and/or high level of the expression, as a GSH.
2. The method of 1, further comprising:
   (a) identifying a genomic locus, wherein the inserted marker gene does not affect cell viability; and/or
   (b) identifying a genomic locus, wherein the inserted marker does not affect the cell's ability to differentiate (e.g., pluripotency, multipotency).
3. The method of 1 or 2, wherein the cell is selected from a cell line, a primary cell, a stem cell, or a progenitor cell, optionally wherein the cell is a stem cell or a progenitor cell.
4. The method of any one of 1-3, wherein the cell is selected from an embryonic stem cell, a tissue-specific stem cell, a mesenchymal stem cell, an induced pluripotent stem cell (iPSC), a hematopoietic stem cell, a hematopoietic CD34+ cell, and epidermal stem cell, an epithelial stem cell, neural stem cell, a lung progenitor cell, and a liver progenitor cell.
5. The method of any one of 1-4, wherein the cell is a mammalian cell, optionally wherein the mammalian cell is a mouse cell, a dog cell, a pig cell, a non-human primate (NHP) cell, or a human cell.
6. The method of any one of 1-5, wherein the random insertion is induced by:
   (a) transfecting the cell with a nucleic acid molecule comprising the marker gene, optionally wherein the nucleic acid is a plasmid; or
   (b) transducing the cell with an integrating virus comprising the marker gene.
7. The method of any one of 1-6, wherein the random insertion is induced by transducing the cell with an integrating virus comprising the marker gene; and the integrating virus is a retrovirus, optionally wherein the retrovirus is a gamma retrovirus.
8. The method of any one of 1-7, wherein the at least one marker gene comprises a screenable marker and/or a selectable marker, optionally wherein
   (a) the screenable marker gene encodes a green fluorescent protein (GFP), beta-galactosidase, luciferase, and/or beta-glucuronidase; and/or
   (b) the selectable marker gene is an antibiotic resistance gene, optionally wherein the antibiotic resistance gene encodes blasticidin S-deaminase or amino 3′-glycosyl phosphotransferase (neomycin resistance gene).
9. The method of any one of 1-8, wherein the marker gene is not operably linked to a promoter.
10. The method of any one of 1-8, wherein the marker gene is operably linked to a promoter, optionally wherein the promoter is a tissue-specific promoter.
11. The method of any one of 1-10, wherein the GSH is intronic, exonic, or intergenic.
12. A method of identifying a GSH locus, the method comprising:
   (a) determining the presence and location of an endogenous virus element (EVE) in the genome of a metazoan species;
   (b) determining intergenic or intronic boundaries proximal to the EVE; and
   (c) identifying an intergenic or intronic locus comprising the EVE as a GSH locus.
13. The method of 12, wherein
   (a) the presence and location of an EVE are determined by searching in silico for sequences homologous to a virus element; and/or
   (b) the intergenic or intronic boundaries proximal to the EVE are determined by aligning the sequences flanking the EVE and its orthologous sequences of one or more species whose intergenic or intronic boundaries are known.
14. A method of identifying a GSH locus in an orthologous organism, the method comprising:
   (a) identifying a GSH locus in Species A according to the method of any one of 1-13;
   (b) determining the location of (i) at least one cis-acting element proximal to the GSH locus in Species A and (ii) the corresponding cis-acting element(s) in Species B; and
   (c) identifying a locus in Species B as a GSH locus, wherein the distance between the locus and the at least one cis-acting element in Species B is substantially proportional to the distance between the GSH locus and the corresponding cis-acting element(s) in Species A.
15. The method of 14, wherein the at least one cis-acting element is selected from a splicing donor site, a splicing acceptor site, a polypyrimidine tract, a polyadenylation signal, an enhancer, a promoter, a terminator, a splicing regulatory element, an intronic splicing enhancer, and an intronic splicing silencer.
16. The method of 14 or 15, wherein the at least one cis-acting element comprises two or more cis-acting elements.
17. The method of any one of 14-16, wherein the at least one cis-acting element comprises two cis-acting elements; and the first cis-acting element is located upstream (i.e., 5′ to) of the GSH locus, and the second cis-acting element is located downstream (i.e., 3′ to) of the GSH locus.
18. The method of 17, wherein the distance between the at least one cis-acting element and the GSH locus relative to the distance between two cis-acting elements in Species B is substantially proportional to the distance between the corresponding cis-acting element and the GSH locus relative to the distance between two cis-acting elements in Species A.
19. The method of any one of 14-18, wherein the distance between the at least one cis-acting element to the GSH locus in Species B is at least 20% but no more than 500% of the distance between the at least one cis-acting element to the GSH locus in Species A.
20. The method of any one of 14-19, wherein the distance between the at least one cis-acting element to the GSH locus in Species B is at least 80% but no more than 250% of the distance between the at least one cis-acting element to the GSH locus in Species A.
21. The method of any one of 12-20, wherein the GSH locus is in a mammalian genome, optionally wherein the mammalian genome is a mouse genome, a dog genome, a pig genome, a NHP genome, or a human genome.
22. The method of any one of 12-21, wherein the EVE or the virus element
   (a) comprises a provirus or a fragment of a viral genome;
   (b) comprises a viral nucleic acid, viral DNA, or a DNA copy of viral RNA; and/or
   (c) encodes a structural or a non-structural viral protein, or a fragment thereof.
23. The method of any one of 12-22, wherein the EVE comprises viral nucleic acid from a retrovirus, a non-retrovirus, parvovirus, or circovirus.
24. The method of 23, wherein
   (a) the parvovirus is selected from B19, minute virus of mice (mvm), RA-1, AAV, bufavirus, hokovirus, bocavirus, and any one of the parvoviruses listed in Tables 1A-1D, optionally wherein the parvovirus is AAV; and/or
   (b) the circovirus is porcine circovirus (PCV) (e.g., PCV-1, PCV-2).
25. The method of any one of 14-24, wherein the metazoan species is selected from Cetacea, Chiropetera, Lagomorpha, and Macropodiadae.
26. The method of any one of 1-11, further comprising the method of any one of 12-25.
27. The method of any one of 1-26, further comprising performing at least one in vitro, ex vivo, and/or in vivo assay.
28. The method of 27, wherein the at least one in vitro, ex vivo, and/or in vivo assay is selected from:
   (a) de novo targeted insertion of a marker gene into the locus in a cell (e.g., human cell) and determine (i) the cell viability, (ii) the insertion efficiency and/or (iii) marker gene expression;
   (b) targeted insertion of a marker gene into the locus in a progenitor cell or stem cell and differentiate in vitro and determine (i) marker gene expression in all developmental lineages, and/or (ii) whether the insertion of the marker gene affects differentiation of the said progenitor cell or stem cell;
   (c) targeted insertion of a marker gene into the locus in a progenitor cell or stem cell and engraft the cell into immune-depleted mice and assess marker gene expression in all developmental lineages in vivo;
   (d) targeted insertion of a marker gene into the locus in a cell and determine the global cellular transcriptional profile (e.g., using RNAseq or microarray); and
   (e) generate a transgenic knock-in mouse wherein the genomic DNA of the mouse has a marker gene inserted in the locus, optionally wherein the marker gene is operatively linked to a tissue specific or inducible promoter.
29. The method of 28, wherein the progenitor cell or the stem cell is selected from an embryonic stem cell, a tissue-specific stem cell, a mesenchymal stem cell, an induced pluripotent stem cell (iPSC), a hematopoietic stem cell, a hematopoietic CD34+ cell, and epidermal stem cell, an epithelial stem cell, neural stem cell, a lung progenitor cell, muscle satellite cell, intestinal K cell, and a liver progenitor cell.
30. A nucleic acid vector, comprising at least a portion of the GSH nucleic acid identified in the method of any one of 1-29.
31. The nucleic acid vector of 30, wherein the GSH nucleic acid comprises an untranslated sequence or an intron.
32. The nucleic acid vector of 30 or 31, wherein the GSH comprises a sequence that is at least 65% identical to the sequence of any one of GSH or a fragment thereof listed in Table 3.
33. The nucleic acid vector of any one of 30-32, wherein the GSH comprises a sequence that is at least 65% identical to the sequence of the genomic DNA or a fragment thereof of SYNTX-GSH1, SYNTX-GSH2, SYNTX-GSH3, or SYNTX-GSH4.
34. The nucleic acid vector of any one of 30-33, further comprising at least one non-GSH nucleic acid, e.g., a nucleic acid having sequences that are heterologous to GSH, e.g., nucleic acid sequences not natively present in the GSH locus, e.g., a transgene.
35. The nucleic acid vector of 34, wherein the at least one non-GSH nucleic acid is flanked by a GSH 5′ homology arm and/or a GSH 3′ homology arm, wherein the homology arm comprises a nucleic acid sequence that is at least about 65% identical to the target GSH nucleic acid.
36. The nucleic acid vector of 35, wherein the GSH homology arm is between 10-5000 base pairs in length, optionally wherein the GSH homology arm is between 100-1500 base pairs in length.
37. The nucleic acid vector of 35, wherein the GSH homology arm is at least 30 base pairs in length.
38. The nucleic acid vector of any one of 35-37, wherein the GSH homology arm is sufficient in length to mediate homology-dependent integration into the GSH locus in the genome of a cell.
39. The nucleic acid vector of any one of 35-38, wherein the at least one non-GSH nucleic acid is in an orientation for integration in the GSH in a forward orientation.
40. The nucleic acid vector of any one of 35-38, wherein the at least one non-GSH nucleic acid is in an orientation for integration in the GSH in a reverse orientation.
41. The nucleic acid vector of any one of 34-40, wherein the at least one non-GSH nucleic acid (a) is operably linked to a promoter, or (b) is not operably linked to a promoter.
42. The nucleic acid vector of 41, wherein the at least one non-GSH nucleic acid is operably linked to a promoter, and the promoter is selected from:
   (a) a promoter heterologous to the nucleic acid to which it is operably linked;
   (b) a promoter that facilitates the tissue-specific expression of the nucleic acid;
   (c) a promoter that facilitates the constitutive expression of the nucleic acid;
   (d) an inducible promoter;
   (e) an immediate early promoter of an animal DNA virus;
   (f) an immediate early promoter of an insect virus; and
   (g) an insect cell promoter.
43. The nucleic acid vector of 42, wherein the inducible promoter is modulated by an agent selected from a small molecule, a metabolite, an oligonucleotide, a riboswitch, a peptide, a peptidomimetic, a hormone, a hormone analog, and light.
44, The nucleic acid vector of 43, wherein the agent is selected from tetracycline, cumate, tamoxifen, estrogen, and an antisense oligonucleotide (ASO), rapamycin, FKCsA, blue light, abscisic acid (ABA), and riboswitch.
45. The nucleic acid vector of 42, wherein the promoter facilitates tissue-specific expression in a hematopoietic stem cell, a hematopoietic CD34+ cell, and epidermal stem cell, an epithelial stem cell, neural stem cell, a lung progenitor cell, a muscle satellite cell, an intestinal K cell, a neuronal cell, an airway epithelial cell, or a liver progenitor cell.
46. The nucleic acid vector of 41 or 42, wherein the promoter is selected from the CMV promoter, β-globin promoter, CAG promoter, AHSP promoter, MND promoter, Wiskott-Aldrich promoter, PKLR promoter, polyhedron (polh) promoter, and immediately early 1 gene (IE-1) promoter.
47. The nucleic acid vector of any one of 34-46, wherein the at least one non-GSH nucleic acid comprises a sequence that encodes a coding RNA.
48. The nucleic acid vector of 47, wherein the sequence encoding a coding RNA is codon-optimized for expression in a target cell.
49. The nucleic acid vector of 47 or 48, wherein the at least one non-GSH nucleic acid encoding a coding RNA further comprises a sequence encoding a signal peptide.
50. The nucleic acid vector of any one of 34-49, wherein the at least one non-GSH nucleic acid comprises a sequence encoding:
   (a) a protein or a fragment thereof, preferably a human protein or a fragment thereof;
   (b) a therapeutic protein or a fragment thereof, an antigen-binding protein, or a peptide;
   (c) a suicide gene, optionally Herpes Simplex Virus-1 Thymidine Kinase (HSV-TK);
   (d) a viral protein or a fragment thereof;
   (e) a nuclease, optionally a Transcription Activator-Like Effector Nuclease (TALEN), a zinc-finger nuclease (ZFN), a meganuclease, a megaTAL, or a CRISPR endonuclease, (e.g., a Cas9 endonuclease or a variant thereof);
   (f) a marker, e.g., luciferase or GFP; and/or
   (g) a drug resistance protein, e.g., antibiotic resistance gene, e.g., neomycin resistance.
51. The nucleic acid vector of 50, wherein the viral protein or a fragment thereof comprises a structural protein (e.g., VP1, VP2, VP3) or a non-structural protein (e.g., Rep protein).
52. The nucleic acid vector of 50 or 51, wherein the viral protein or a fragment thereof comprises:
   (a) a parvovirus protein or a fragment thereof, optionally VP1, VP2, VP3, NS1, or Rep;
   (b) a retrovirus protein or a fragment thereof, optionally an envelope protein, gag, pol, or VSV-G;
   (c) an adenovirus protein or a fragment thereof, optionally E1A, E1B, E2A, E2B, E3, E4, or a structural protein (e.g., A, B, C); and/or
   (d) a herpes simplex virus protein or a fragment thereof, optionally ICP27, ICP4, or pac.
53. The nucleic acid vector of any one of 50-52, wherein the at least one non-GSH nucleic acid encoding a viral protein encodes a surface protein, or a fragment thereof, of a virus.
54. The nucleic acid vector of 53, wherein (a) the surface protein or a fragment thereof is an immunogenic surface protein that elicits immune response in a host, (b) the surface protein or a fragment thereof further comprises a signal peptide, (c) the gene encoding the surface protein or fragment thereof is operably linked to an inducible promoter, and/or (d) the nucleic acid encoding the surface protein or a fragment thereof further comprises a suicide gene.
55. The nucleic acid vector of 53 or 54, wherein the surface protein is of a coronavirus (e.g., MERS, SARS), influenza virus, respiratory syncytial virus, hepatitis A, hepatitis B, hepatitis C, hepatitis D, hepatitis E, human papillomavirus, dengue virus serotype 1, dengue virus serotype 2, dengue virus serotype 3, dengue virus serotype 4, zika, virus, West Nile virus, yellow fever virus, Chikungunya virus, Mayaro virus, Ebola virus, Marburg virus, or Nipa virus.
56. The nucleic acid vector of any one of 53-55, wherein the surface protein is the spike protein of SARS-COV-2.
57. The nucleic acid vector of 50, wherein the at least one non-GSH nucleic acid comprising a sequence encoding a protein, or a fragment thereof, is selected from a hemoglobin gene (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), GBA1, preproinsulin, insulin, GIP, GLP-1, CEP290, ATPB1, ATPB11, ABCB4, CPS1, ATP7B, KRT5, KRT14, PLEC1, Col7A1, ITGB4, ITGA6, LAMA3, LAMB3, LAMC2, KIND1, INS, F8 or a fragment thereof (e.g., fragment encoding B-domain deleted polypeptide (e.g., VIII SQ, p-VIII)), IRGM, NOD2, ATG2B, ATG9, ATG5, ATG7, ATG16L1, BECN1, EI24/PIG8, TECPR2, WDR45/WIP14, CHMP2B, CHMP4B, Dynein, EPG5, HspB8, LAMP2, LC3b UVRAG, VCP/p97, ZFYVE26, PARK2/Parkin, PARK6/PINK1, SQSTM1/p62, SMURF, AMPK, ULK1, RPE65, CHM, RPGR, PDE6B, CNGA3, GUCY2D, RS1, ABCA4, MYO7A, HFE, hepcidin, a gene encoding a soluble form (e.g., of the TNFα receptor, IL-6 receptor, IL-12 receptor, or IL-1β receptor), and cystic fibrosis transmembrane conductance regulator (CFTR).
58. The nucleic acid vector of 50, wherein the antigen-binding protein is an antibody or an antigen-binding fragment thereof, optionally wherein the antibody or an antigen-binding fragment thereof is selected from an antibody, Fv, F(ab)2, Fab′, dsFv, scFv, sc (Fv)2, half antibody-scFv, tandem scFv, Fab/scFv-Fc, tandem Fab′, single-chain diabody, tandem diabody (TandAb), Fab/scFv-Fc, scFv-Fc, heterodimeric IgG (CrossMab), DART, and diabody.
59. The nucleic acid vector of 50 or 51, wherein the antigen-binding protein specifically binds TNFα, CD20, a cytokine (e.g., IL-1, IL-6, BLyS, APRIL, IFN-gamma, etc.), Her2, RANKL, IL-6R, GM-CSF, CCR5, or a pathogen (e.g., bacterial toxin, viral capsid protein, etc.).
60. The nucleic acid vector of any one of 50, 58, and 59, wherein the antigen-binding protein is selected from adalimumab, etanercept, infliximab, certolizumab, golimumab, anakinra, rituximab, abatacept, tocilizumab, natalizumab, canakinumab, atacicept, belimumab, ocrelizumab, ofatumumab, fontolizumab, trastuzumab, denosumab, sarilumab, lenzilumab, gimsilumab, siltuximab, leronlimab, and an antigen-binding fragment thereof.
61. The nucleic acid vector of any one of 34-46, wherein the at least one non-GSH nucleic acid comprises a sequence encoding a non-coding RNA, optionally wherein the non-coding RNA comprises antisense polynucleotides, lncRNA, piRNA, miRNA, shRNA, siRNA, antisense RNA, snoRNA, snRNA, scaRNA, and/or guide RNA.
62. The nucleic acid vector of 61, wherein the non-coding RNA targets a gene selected from DMT-1, ferroportin, TNFα receptor, IL-6 receptor, IL-12 receptor, IL-1β receptor, and a gene encoding a mutated protein (e.g., a mutated HFE, CFTR).
63. The nucleic acid vector of any one of 34-62, wherein the at least one non-GSH nucleic acid increases or restores the expression of an endogenous gene of a target cell.
64. The nucleic acid vector of any one of 34-62, wherein the at least one non-GSH nucleic acid decreases or eliminates the expression of an endogenous gene of a target cell.
65. The nucleic acid vector of any one of 30-64, further comprising:
   (a) a transcription regulatory element (e.g., an enhancer, a transcription termination sequence, an untranslated region (5′ or 3′ UTR), a proximal promoter element, a locus control region (e.g., a β-globin LCR or a DNase hypersensitive site (HS) of β-globin LCR), a polyadenylation signal sequence), and/or
   (b) a translation regulatory element (e.g., Kozak sequence, woodchuck hepatitis virus post-transcriptional regulatory element).
66. The nucleic acid vector of any of 30-65, wherein the nucleic acid vector is selected from a plasmid, minicircle, comsid, artificial chromosome (e.g., BAC), linear covalently closed (LCC) DNA vector (e.g., minicircles, minivectors and miniknots), a linear covalently closed (LCC) vector (e.g., MIDGE, MiLV, ministering, miniplasmids), a mini-intronic plasmid, a pDNA expression vector, or variants thereof.
67. A viral vector comprising at least a portion of the GSH nucleic acid identified in the method of any one of 1-29; at least a portion of the GSH in the nucleic acid vector of any one of 30-66; at least a portion of any one of the GSHs listed in Table 3; and/or the nucleic acid vector of any one of 30-66.
68. The viral vector of 67, wherein the viral vector is selected from rAd, AAV, rHSV, retroviral vector, poxvirus vector, lentivirus, vaccinia virus vector, HSV Type 1 (HSV-1)-AAV hybrid vector, baculovirus expression vector system (BEVS), and variants thereof.
69. A cell, comprising the nucleic acid vector of any one of 30-66, or the viral vector of 67 or 68.
70. The cell of 69, wherein the cell is selected from a cell line or a primary cell.
71. The cell of 69-70, wherein the cell is a mammalian cell, an insect cell, a bacterial cell, a yeast cell, or a plant cell, optionally wherein the mammalian cell is a human cell or a rodent cell.
72. The cell of any one of 69-71, wherein the cell is an insect cell; and the insect cell is derived from a species of lepidoptera.
73. The cell of 72, wherein the species of lepidoptera is Spodoptera frugiperda, Spodoptera littoralis, Spodoptera exigua, or Trichoplusia ni.
74. The cell of any one of 69-73, wherein the insect cell is Sf9.
75. The cell of any one of 69-74, wherein the cell is selected from a hematopoietic cell, hematopoietic progenitor cell, hematopoietic stem cell, erythroid lineage cell, megakaryocyte, erythroid progenitor cell (EPC), CD34+ cell, CD44+ cell, red blood cell, CD36+ cell, mesenchymal stem cell, nerve cell, intestinal cell, intestinal stem cell, gut epithelial cell, endothelial cell, enteroendocrine cell, lung cell, lung progenitor cell, enterocyte, liver cell (e.g., hepatocyte, hepatic stellate cells, Kupffer cells (KCs), liver sinusoidal endothelial cells (LSECs), liver progenitor cell), stem cell, progenitor cell, induced pluripotent stem cell (iPSC), skin fibroblast, macrophage, brain microvascular endothelial cell (BMVECs), neural stem cell, muscle satellite cell, epithelial cell, airway epithelial cell, muscle progenitor cell, erythroid progenitor cell, lymphoid progenitor cell, B lymphoblast cell, B cell, T cell, basophilic Endemic Burkitt Lymphoma (EBL), polychromatic erythroblast, epidermal stem cell, epithelial stem cell, embryonic stem cell, P63-positive keratinocyte-derived stem cell, keratinocyte, pancreatic β-cell, K cell, L cell, HEK293 cell, HEK293T cell, MDCK cell, Vero cell, CHO, BHK1, NS0, Sp2/0, HeLa, A549, and orthochromatic erythroblast.
76. A cell, comprising at least one non-GSH nucleic acid integrated into a GSH in the genome of a cell, wherein the GSH is selected from Table 3.
77. The cell of 76, wherein the GSH nucleic acid comprises an untranslated sequence or an intron.
78. The cell of 76 or 77, wherein the GSH is selected from SYNTX-GSH1, SYNTX-GSH2, SYNTX-GSH3, and SYNTX-GSH4.
79. The cell of any one of 76-78, wherein the at least one non-GSH nucleic acid is integrated into the GSH in a forward orientation.
80. The cell of any one of 76-78, wherein the at least one non-GSH nucleic acid is integrated into the GSH in a reverse orientation.
81. The cell of any one of 76-80, wherein the at least one non-GSH nucleic acid (a) is operably linked to a promoter, or (b) is not operably linked to a promoter.
82. The cell of 81, wherein the at least one non-GSH nucleic acid is operably linked to a promoter, and the promoter is selected from:
   (a) a promoter heterologous to the nucleic acid to which it is operably linked;
   (b) a promoter that facilitates the tissue-specific expression of the nucleic acid;
   (c) a promoter that facilitates the constitutive expression of the nucleic acid;
   (d) an inducible promoter;
   (e) an immediate early promoter of an animal DNA virus;
   (f) an immediate early promoter of an insect virus; and
   (g) an insect cell promoter.
83. The cell of 82, wherein the inducible promoter is modulated by an agent selected from a small molecule, a metabolite, an oligonucleotide, a riboswitch, a peptide, a peptidomimetic, a hormone, a hormone analog, and light.
84, The cell of 83, wherein the agent is selected from tetracycline, cumate, tamoxifen, estrogen, and an antisense oligonucleotide (ASO), rapamycin, FKCsA, blue light, abscisic acid (ABA), and riboswitch.
85. The cell of 82, wherein the promoter facilitates tissue-specific expression in a hematopoietic stem cell, a hematopoietic CD34+ cell, and epidermal stem cell, an epithelial stem cell, neural stem cell, a lung progenitor cell, a muscle satellite cell, an intestinal K cell, a neuronal cell, an airway epithelial cell, or a liver progenitor cell.
86. The cell of 81 or 82, wherein the promoter is selected from the CMV promoter, β-globin promoter, CAG promoter, AHSP promoter, MND promoter, Wiskott-Aldrich promoter, PKLR promoter, polyhedron (polh) promoter, and immediately early 1 gene (IE-1) promoter.
87. The cell of any one of 52-58, wherein the at least one non-GSH nucleic acid comprises a sequence that encodes a coding RNA.
88. The cell of 87, wherein the sequence encoding a coding RNA is codon-optimized for expression in a target cell.
89. The cell of 87 or 88, wherein the at least one non-GSH nucleic acid encoding a coding RNA further comprises a sequence encoding a signal peptide.
90. The cell of any one of 76-89, wherein the at least one non-GSH nucleic acid encodes a coding RNA comprises a sequence encoding:
   (a) a protein or a fragment thereof, preferably a human protein or a fragment thereof;
   (b) a therapeutic protein or a fragment thereof, an antigen-binding protein, or a peptide;
   (c) a suicide gene, optionally Herpes Simplex Virus-1 Thymidine Kinase (HSV-TK);
   (d) a viral protein or a fragment thereof;
   (e) a nuclease, optionally a Transcription Activator-Like Effector Nuclease (TALEN), a zinc-finger nuclease (ZFN), a meganuclease, a megaTAL, or a CRISPR endonuclease, (e.g., a Cas9 endonuclease or a variant thereof);
   (f) a marker, e.g., luciferase or GFP; and/or
   (g) a drug resistance protein, e.g., antibiotic resistance gene, e.g., neomycin resistance.
91. The cell of 90, wherein the viral protein or a fragment thereof comprises a structural protein (e.g., VP1, VP2, VP3) or a non-structural protein (e.g., Rep protein).
92. The cell of 90 or 91, wherein the viral protein or a fragment thereof comprises:
   (a) a parvovirus protein or a fragment thereof, optionally VP1, VP2, VP3, NS1, or Rep;
   (b) a retrovirus protein or a fragment thereof, optionally an envelope protein, gag, pol, or VSV-G;
   (c) an adenovirus protein or a fragment thereof, optionally E1A, E1B, E2A, E2B, E3, E4, or a structural protein (e.g., A, B, C); and/or
   (d) a herpes simplex virus protein or a fragment thereof, optionally ICP27, ICP4, or pac.
93. The cell of any one of 90-92, wherein the gene encoding a viral protein encodes a surface protein, or a fragment thereof, of a virus.
94. The cell of 93, wherein (a) the surface protein is an immunogenic surface protein or a fragment thereof that elicits immune response, (b) the surface protein or a fragment thereof further comprises a signal peptide, (c) the gene is operably linked to an inducible promoter, and/or (d) the nucleic acid encoding the surface protein or a fragment thereof further comprises a suicide gene.
95. The cell of 93 or 94, wherein the surface protein is of a coronavirus (e.g., MERS, SARS), influenza virus, respiratory syncytial virus, hepatitis A, hepatitis B, hepatitis C, hepatitis D, hepatitis E, human papillomavirus, dengue virus serotype 1, dengue virus serotype 2, dengue virus serotype 3, dengue virus serotype 4, zika, virus, West Nile virus, yellow fever virus, Chikungunya virus, Mayaro virus, Ebola virus, Marburg virus, or Nipa virus.
96. The cell of any one of 93-95, wherein the surface protein is the spike protein of SARS-COV-2.
97. The cell of 90, wherein the at least one non-GSH nucleic acid comprising a sequence encoding a protein, or a fragment thereof, is selected from a hemoglobin gene (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), GBA1, preproinsulin, insulin, GIP, GLP-1, CEP290, ATPB1, ATPB11, ABCB4, CPS1, ATP7B, KRT5, KRT14, PLEC1, Col7A1, ITGB4, ITGA6, LAMA3, LAMB3, LAMC2, KIND1, INS, F8 or a fragment thereof (e.g., fragment encoding B-domain deleted polypeptide (e.g., VIII SQ, p-VIII)), IRGM, NOD2, ATG2B, ATG9, ATG5, ATG7, ATG16L1, BECN1, EI24/PIG8, TECPR2, WDR45/WIP14, CHMP2B, CHMP4B, Dynein, EPG5, HspB8, LAMP2, LC3b UVRAG, VCP/p97, ZFYVE26, PARK2/Parkin, PARK6/PINK1, SQSTM1/p62, SMURF, AMPK, ULK1, RPE65, CHM, RPGR, PDE6B, CNGA3, GUCY2D, RS1, ABCA4, MYO7A, HFE, hepcidin, a gene encoding a soluble form (e.g., of the TNFα receptor, IL-6 receptor, IL-12 receptor, or IL-1β receptor), and cystic fibrosis transmembrane conductance regulator (CFTR).
98. The cell of 90, wherein the antigen-binding protein is an antibody or an antigen-binding fragment thereof, optionally wherein the antibody or an antigen-binding fragment thereof is selected from an antibody, Fv, F(ab)₂, Fab′, dsFv, scFv, sc (Fv)2, half antibody-scFv, tandem scFv, Fab/scFv-Fc, tandem Fab′, single-chain diabody, tandem diabody (TandAb), Fab/scFv-Fc, scFv-Fc, heterodimeric IgG (CrossMab), DART, and diabody.
99. The cell of 90 or 91, wherein the antigen-binding protein specifically binds TNFα, CD20, a cytokine (e.g., IL-1, IL-6, BLyS, APRIL, IFN-gamma, etc.), Her2, RANKL, IL-6R, GM-CSF, CCR5, or a pathogen (e.g., bacterial toxin, viral capsid protein, etc.).
100. The cell of any one of 90, 98, and 99, wherein the antigen-binding protein is selected from adalimumab, etanercept, infliximab, certolizumab, golimumab, anakinra, rituximab, abatacept, tocilizumab, natalizumab, canakinumab, atacicept, belimumab, ocrelizumab, ofatumumab, fontolizumab, trastuzumab, denosumab, sarilumab, lenzilumab, gimsilumab, siltuximab, leronlimab, and an antigen-binding fragment thereof.
101. The cell of any one of 76-86, wherein the at least one non-GSH nucleic acid comprises a sequence encoding a non-coding RNA, optionally wherein the non-coding RNA comprises lncRNA, piRNA, miRNA, shRNA, siRNA, antisense RNA, snoRNA, snRNA, scaRNA, and/or guide RNA.
102. The cell of 101, wherein the non-coding RNA targets a gene selected from DMT-1, ferroportin, TNFα receptor, IL-6 receptor, IL-12 receptor, IL-1β receptor, a gene encoding a mutated protein (e.g., a mutated HFE, CFTR).
103. The cell of any one of 76-102, wherein the at least one non-GSH nucleic acid increases or restores the expression of an endogenous gene of a target cell.
104. The cell of any one of 76-102, wherein the at least one non-GSH nucleic acid decreases or eliminates the expression of an endogenous gene of a target cell.
105. The cell of any one of 76-104, wherein the at least one non-GSH nucleic acid further comprises:
   (a) a transcription regulatory element (e.g., an enhancer, a transcription termination sequence, an untranslated region (5′ or 3′ UTR), a proximal promoter element, a locus control region (e.g., a β-globin LCR or a DNase hypersensitive site (HS) of β-globin LCR), a polyadenylation signal sequence), and/or
   (b) a translation regulatory element (e.g., Kozak sequence, woodchuck hepatitis virus post-transcriptional regulatory element).
106. The cell of any one of 76-105, wherein the cell is selected from a cell line or a primary cell.
107. The cell of any one of 76-106, wherein the cell is a mammalian cell, an insect cell, a bacterial cell, a yeast cell, or a plant cell, optionally wherein the mammalian cell is a human cell or a rodent cell.
108. The cell of any one of 76-107, wherein the cell is an insect cell; and the insect cell is derived from a species of lepidoptera.
109. The cell of 108, wherein the species of lepidoptera is Spodoptera frugiperda, Spodoptera littoralis, Spodoptera exigua, or Trichoplusia ni.
110. The cell of any one of 107-109, wherein the insect cell is Sf9.
111. The cell of any one of 76-110, wherein the cell is selected from a hematopoietic cell, hematopoietic progenitor cell, hematopoietic stem cell, erythroid lineage cell, megakaryocyte, erythroid progenitor cell (EPC), CD34+ cell, CD44+ cell, red blood cell, CD36+ cell, mesenchymal stem cell, nerve cell, intestinal cell, intestinal stem cell, gut epithelial cell, endothelial cell, enteroendocrine cell, lung cell, lung progenitor cell, enterocyte, liver cell (e.g., hepatocyte, hepatic stellate cells, Kupffer cells (KCs), liver sinusoidal endothelial cells (LSECs), liver progenitor cell), stem cell, progenitor cell, induced pluripotent stem cell (iPSC), skin fibroblast, macrophage, brain microvascular endothelial cell (BMVECs), neural stem cell, muscle satellite cell, epithelial cell, airway epithelial cell, muscle progenitor cell, erythroid progenitor cell, lymphoid progenitor cell, B lymphoblast cell, B cell, T cell, basophilic Endemic Burkitt Lymphoma (EBL), polychromatic erythroblast, epidermal stem cell, epithelial stem cell, embryonic stem cell, P63-positive keratinocyte-derived stem cell, keratinocyte, pancreatic β-cell, K cell, L cell, HEK293 cell, HEK293T cell, MDCK cell, Vero cell, CHO, BHK1, NS0, Sp2/0, HeLa, A549, and orthochromatic erythroblast.
112. A pharmaceutical composition, comprising the nucleic acid vector of any one of 30-66, the viral vector of 67 or 68, and/or the cell of any one of 69-111.
113. A transgenic organism comprising at least one non-GSH nucleic acid integrated into a GSH in the genome of a cell, wherein the GSH is selected from Table 3.
114. The transgenic organism of 113, wherein the GSH is selected from SYNTX-GSH1, SYNTX-GSH2, SYNTX-GSH3, and SYNTX-GSH4.
115. A transgenic organism, comprising the cell of any one of 69-114.
116. The transgenic organism of 115, wherein the organism is a mammal or a plant, optionally wherein the mammal is a rodent (e.g., mouse, rat), a goat, a sheep, a chicken, a llama, or a rabbit.
117. A method of inserting at least one non-GSH nucleic acid into a GSH locus of a cell, the method comprising introducing the nucleic acid vector of any one of 30-66, the viral vector of 67 or 68, or a pharmaceutical composition of 112 into the cell, whereby homologous recombination of the GSH 5′ homology arm and the GSH 3′ homology arm flanking the non-GSH nucleic acid with the GSH locus in the genome integrates the non-GSH nucleic acid into the GSH locus.
118. The method of 117, wherein the non-GSH nucleic acid is integrated into the GSH in a forward orientation.
119. The method of 117, wherein the non-GSH nucleic acid is integrated into the GSH in a reverse orientation.
120. A method of preventing or treating a disease, comprising administering to a subject in need thereof an effective amount of the nucleic acid vector of any one of 30-66, the viral vector of 67 or 68, the cell of any one of 69-111, and/or the pharmaceutical composition of 112.
121. The method of 120, wherein the disease is selected from an infection, endothelial dysfunction, cystic fibrosis, cardiovascular disease, renal disease, cancer, hemoglobinopathy, anemia, hemophilia (e.g., hemophilia A), myeloproliferative disorder, coagulopathy, sickle cell disease, alpha-thalassemia, beta-thalassemia, Fanconi anemia, familial intrahepatic cholestasis, skin genetic disorder (e.g., epidermolysis bullosa), ocular genetic disease (e.g., inherited retinal dystrophies, e.g., Leber congenital amaurosis (LCA), retinitis pigmentosa (RP), choroideremia, achromatopsia, retinoschisis, Stargardt disease, Usher syndrome type 1B), Fabry, Gaucher, Nieman-Pick A, Nieman-Pick B, GM1 Gangliosidosis, Mucopolysaccharidosis (MPS) I (Hurler, Scheie, Hurler/Scheie), MPS II (Hunter), MPS VI (Maroteaux-Lamy), hematologic cancer, hemochromatosis, hereditary hemochromatosis, juvenile hemochromatosis, cirrhosis, hepatocellular carcinoma, pancreatitis, diabetes mellitus, cardiomyopathy, arthritis, hypogonadism, heart disease, heart attack, hypothyroidism, glucose intolerance, arthropathy, liver fibrosis, Wilson's disease, ulcerative colitis, Crohn's disease, Tay-Sachs disease, neurodegenerative disorder, Spinal muscular atrophy type 1, Huntington's disease, Canavan's disease, rheumatoid arthritis, inflammatory bowel disease, psoriatic arthritis, juvenile chronic arthritis, psoriasis, and ankylosing spondylitis, and autoimmune disease, neurodegenerative disease (e.g., Alzheimer's disease, Parkinson's disease, Huntington's disease, ataxias), inflammatory disease, inflammatory bowel disease, Crohn's disease, rheumatoid arthritis, lupus, multiple sclerosis, chronic obstructive pulmonary disease/COPD, pulmonary fibrosis, Sjogren's disease, hyperglycemic disorders, type I diabetes, type II diabetes, insulin resistance, hyperinsulinemia, insulin-resistant diabetes (e.g. Mendenhall's Syndrome, Werner Syndrome, leprechaunism, and lipoatrophic diabetes), dyslipidemia, hyperlipidemia, elevated low-density lipoprotein (LDL), depressed high density lipoprotein (HDL), elevated triglycerides, metabolic syndrome, liver disease, renal disease, cardiovascular disease, ischemia, stroke, complications during reperfusion, muscle degeneration, atrophy, symptoms of aging (e.g., muscle atrophy, frailty, metabolic disorders, low grade inflammation, atherosclerosis, stroke, age-associated dementia and sporadic form of Alzheimer's disease, pre-cancerous states, and psychiatric conditions including depression), spinal cord injury, arteriosclerosis, infectious diseases (e.g., bacterial, fungal, viral), AIDS, tuberculosis, defects in embryogenesis, infertility, lysosomal storage diseases, activator deficiency/GM2 gangliosidosis, alpha-mannosidosis, aspartylglucoaminuria, cholesteryl ester storage disease, chronic hexosaminidase A deficiency, cystinosis, Danon disease, Farber disease, fucosidosis, galactosialidosis, Gaucher Disease (Types I, II and III), GM1 Gangliosidosis, (infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Neuronal ceroid lipofuscinoses, CLN6 disease, Jansky-Bielschowsky disease, Pompe disease, pycnodysostosis, Sandhoff disease, Schindler disease, and Wolman disease.
122. The method of 121, wherein the infection is a bacterial infection, fungal infection, or a viral infection.
123. The method of 121 or 122, wherein the infection is the viral infection; and the viral infection is by a coronavirus (e.g., MERS, SARS), influenza virus, respiratory syncytial virus, hepatitis A, hepatitis B, hepatitis C, hepatitis D, hepatitis E, human papillomavirus, dengue virus serotype 1, dengue virus serotype 2, dengue virus serotype 3, dengue virus serotype 4, zika, virus, West Nile virus, yellow fever virus, Chikungunya virus, Mayaro virus, Ebola virus, Marburg virus, or Nipa virus.
124. The method of 122 or 123, wherein the viral infection is by SARS-COV-2.
125. The method of any one of 120-124, wherein the nucleic acid vector, the cell, and/or the pharmaceutical composition is administered to the subject via intravascular, intracerebral, parenteral, intraperitoneal, intravenous, epidural, intraspinal, intrasternal, intra-articular, intra-synovial, intrathecal, intratumoral, intra-arterial, intracardiac, intramuscular, intranasal, intrapulmonary, skin graft, or oral administration.
126. The method of any one of 120-125, wherein the cell is autologous or allogeneic to the subject.
127. A method of modulating the level and/or activity of a protein in a cell, the method comprising introducing the nucleic acid vector of any one of 30-66, the viral vector of 67 or 68, and/or the pharmaceutical composition of 112 to the cell.
128. The method of 127, wherein the level and/or activity is increased.
129. The method of 128, wherein the level and/or activity is decreased or eliminated.
130. A method of manufacturing a biologic, the method comprising:
   (a) culturing (i) the cell comprising the nucleic acid vector of any one of 30-66, (ii) the cell comprising the viral vector of 67 or 68, or (iii) the cell of any one of 69-111; and recovering the expressed biologic; or
   (b) recovering the expressed biologic from the transgenic organism of 115 or 116.
131. The method of 130, wherein the biologic is an antigen-binding protein.
132. The method of 130 or 131, wherein the biologic is an antibody or an antigen-binding fragment thereof, optionally wherein the antibody or an antigen-binding fragment thereof is selected from an antibody, Fv, F(ab)2, Fab′, dsFv, scFv, sc (Fv)2, half antibody-scFv, tandem scFv, Fab/scFv-Fc, tandem Fab′, single-chain diabody, tandem diabody (TandAb), Fab/scFv-Fc, scFv-Fc, heterodimeric IgG (CrossMab), DART, and diabody. 133. The method of any one of 130-132, wherein the biologic specifically binds TNFα, CD20, a cytokine (e.g., IL-1, IL-6, BLyS, APRIL, IFN-gamma, etc.), Her2, RANKL, IL-6R, GM-CSF, or CCR5.
134. The method of any one of 130-133, wherein the biologic is selected from adalimumab, etanercept, infliximab, certolizumab, golimumab, anakinra, rituximab, abatacept, tocilizumab, natalizumab, canakinumab, atacicept, belimumab, ocrelizumab, ofatumumab, fontolizumab, trastuzumab, denosumab, sarilumab, lenzilumab, gimsilumab, siltuximab, leronlimab, and an antigen-binding fragment thereof.
135. The method of any one of 130-134, wherein the biologic is a therapeutic protein, optionally wherein the therapeutic protein is an insulin.
136. A method of manufacturing a viral vector (e.g., gene therapy or vaccine), the method comprising:
   (1) providing a host cell comprising
      (i) a nucleic acid sequence comprising at least one functional virus origin of replication (e.g., at least one ITR nucleotide sequence),
         optionally further comprising a nucleic acid operably linked to a promoter for expression in a target cell,
      (ii) a nucleic acid sequence comprising at least one gene encoding one or more viral structural proteins (e.g., capsid proteins, e.g., gag, VP1,VP2, VP3, a variant thereof), operably linked to at least one expression control sequence for expression in a host cell, and
      (iii) a nucleic acid sequence comprising at least one gene encoding one or more replication proteins (e.g., Rep, pol) operably linked to at least one expression control sequence for expression in a host cell,
         optionally wherein the at least one replication protein comprises (a) a Rep52 or a Rep40 coding sequence or a fragment thereof that encodes a functional replication protein, operably linked to at least one expression control sequence for expression in a host cell, and/or (b) a Rep78 or a Rep68 coding sequence operably linked to at least one expression control sequence for expression in a host cell;
         wherein at least one of (i), (ii), and (iii) is stably integrated into at least one GSH selected from Table 3 in the host cell genome, and the at least one vector, if/when present, comprises the remainder of the (i), (ii), and (iii) that is not stably integrated in the host cell genome; and
   (2) maintaining the host cell under conditions such that a recombinant viral vector is produced.
137. The method of 136, wherein (ii) or (iii) is integrated into a GSH.
138. The method of 136, wherein (ii) and (iii) are integrated into a GSH.
139. The method of any one of 136-138, wherein the at least one functional virus origin of replication (e.g., at least one ITR nucleotide sequence) comprises:
   (a) a dependoparvovirus ITR, and/or
   (b) an AAV ITR, optionally an AAV2 ITR.
140. The method of any one of 136-139, wherein the at least one expression control sequence for expression in the host cell comprises:
   (a) a promoter, and/or
   (b) a Kozak-like expression control sequence.
141. The method of 140, wherein the promoter comprises:
   (a) an immediate early promoter of an animal DNA virus,
   (b) an immediate early promoter of an insect virus,
   (c) an insect cell promoter, or
   (d) an inducible promoter.
142. The method of 141, wherein the animal DNA virus is cytomegalovirus (CMV), a dependoparvovirus, or AAV.
143. The method of 141, wherein the insect virus is a lepidopteran virus or a baculovirus, optionally wherein the baculovirus is Autographa californica multicapsid nucleopolyhedrovirus (AcMNPV).
144. The method of 140 or 141, wherein the promoter is a polyhedrin (polh) or immediately early 1 gene (IE-1) promoter.
145. The method of 140 or 141, wherein the promoter is an inducible promoter.
146. The method of 145, wherein the inducible promoter is modulated by an agent selected from a small molecule, a metabolite, an oligonucleotide, a riboswitch, a peptide, a peptidomimetic, a hormone, a hormone analog, and light.
147. The method of 146, wherein the agent is selected from tetracycline, cumate, tamoxifen, estrogen, and an antisense oligonucleotide (ASO), rapamycin, FKCsA, blue light, abscisic acid (ABA), and riboswitch.
148. The method of any one of 136-147, wherein:
   (a) the viral replication protein is an AAV replication protein, optionally Rep52 and/or Rep78 proteins; and/or
   (b) the viral structural protein is an AAV capsid protein.
149. The method of 148, wherein the AAV is AAV2.
150. The method of any one of 136-149, wherein the method manufactures the viral vector of 67 or 68.
151. The method of any one of 136-150, wherein the host cell is a mammalian cell or an insect cell.
152. The method of 151, wherein the host cell is a mammalian cell; and the mammalian cell is a human cell or a rodent cell.
153. The method of 151 or 152, wherein the mammalian cell is selected from HEK293, HEK293T, HeLa, and A549.
154. The method of 151, wherein the host cell is an insect cell; and the insect cell is derived from a species of lepidoptera.
155. The method of 154, wherein the species of lepidoptera is Spodoptera frugiperda, Spodoptera littoralis, Spodoptera exigua, or Trichoplusia ni.
156. The method of any one of 151, 154, and 155, wherein the insect cell is Sf9.
157. The method of any one of 136-156, wherein the viral vector is selected from adeno virus-derived vectors (e.g., AAV), retrovirus, lentivirus-derived vectors (e.g., lentivirus), herpes virus-derived vectors, and alphavirus-derived vectors (e.g., Semliki forest virus (SFV) vector).
158. A kit, comprising the nucleic acid vector of any one of 30-66, the viral vector of 67 or 68, the cell of any one of 69-111, and/or the pharmaceutical composition of 112.

### EXAMPLES

### Example 1: Identifying GSH Loci by Determining the Presence and Location of EVEs

### Genome Screening

Chromosome assemblies and whole genome shotgun assemblies of 44 species (Table S1 of Katzourakis and Gifford (2010) PLOS Genetics 6 (11): e1001191) were screened in silico using tBLASTn and a library of representative peptide sequences derived from mammalian virus groups with genomes <100 Kb in total length (selected from the 2009 International Committee on Taxonomy of Viruses (ICTV) master species list). Host genome sequences spanning high-identity (i.e., e-values, 0.0001) matches to viral peptides were extracted, and a putative viral ORF was inferred using Blast and manual editing. Putative EVE peptides were then used to screen the Genbank non-redundant (nr) database in a reciprocal tBLASTn search. Matches to retroviruses, viral cloning vectors, and non-specific matches to host loci were filtered and discarded. The remaining sequences were considered viral if they unambiguously matched viral proteins in the Genbank and PFAM databases. Genetic structures for these elements were determined by comparison of the putative EVE peptide sequence to the nucleotide sequence of a viral type species representing the most closely related viral genus recognized by ICTV. Boundaries between viral and genomic regions were identified by analysis of sequences flanking matches to viral peptides, the genomes of the host species, and closely related host species. Sequences that flanked viral insertions were considered genomic if they: (i) were present as empty insertion sites in a related host species; (ii) disclosed highly significant similarity (i.e. e-values <1×10⁻⁹) to host proteins; or (iii) non-viral and highly repetitive (>50 copies per host genome). Insertions were considered endogenous when >100 bp of genomic flanking sequence could be identified either side of a viral match. Insertions for which >100 bp of unambiguous (i.e. >80% nucleotide identity) flanking sequence was identified in host sister taxa were considered orthologous insertions. PERL scripts were used to automate BLAST searches and sequence extraction.

### Phylogenetic Analysis

Putative EVE sequences inferred using Blast were aligned with closely related viruses using MUSCLE and MAAFT, and manually edited (Edgar (2004) Nucleic Acids Res 32:1792-1797). Maximum likelihood (ML) phylogenies were estimated using amino acid sequence alignments with RAXML (Stamatakis (2006) Bioinformatics 22:2688-2690), implementing in each case the best fitting substitution model as determined by ProtTest (Abascal et al. (2005) Bioinformatics 21:2104-2105). Support for the ML trees was evaluated with 1000 nonparametric bootstrap replicates. The best fitting models for the datasets were: Parvoviridae: Dependovirus NS1 gene (JTT+C, 332 amino acids across 17 taxa), Parvoviridae: parvovirus NS1 gene, (JTT+C, 293 amino acids across 13 taxa), Circoviridae: Rep gene (Blosum62+C+F, 235 amino acids across 14 taxa), Hepadnaviridae: polymerase gene (JTT+C+F, 661 amino acids across 9 taxa), Orthomyxoviridae: GP gene (WAG+C+F, 482 amino acids across 5 taxa), Reoviridae: VP5 gene (Dayhoff+C+F, 171 amino acids across 4 taxa), Bunyaviridae: phlebovirus NP gene (LG+C, 247 amino acids across 12 taxa), Bunyaviridae: nairovirus NP gene (LG+C, 446 amino acids across 5 taxa), Flaviviridae: mostly NS3 gene (LG+C+F, 1846 amino acids across 8 taxa), Filoviridae: NP gene (JTT+C, 369 amino acids across 29 taxa), Filoviridae: L gene (LG+C+F, 517 amino acids across 9 taxa), Bornaviridae: NP gene (JTT+C, 147 amino acids across 73 taxa), Bornaviridae: L gene (JTT+C+F, 1243 amino acids across 12 taxa), Rhabdoviridae: NP gene (LG+C, 220 amino acids across 34 taxa), Rhabdoviridae: L gene (LG+C+F, 383 amino acids across 26 taxa).

### Example 2: Methods of Identifying GSH Loci in an Orthologous Organism

### Position Relative to Cis-Acting Elements (Introns of Similar Size)

Lacking sequence homology between a host (in which an EVE is identified using any one of the methods described herein) and a non-host species, the location of the EVE insertion in a non-host species is imprecisely determined. An approximation can be made using relative position of the EVE insertion. For example, a host and a non-host each has a 1200 nucleotide (nt)-intron based on orthologous host and closely-related non-host genome sequence. In the host species, the EVE is inserted into the intron at a position that is 800 nt from the splice donor site and 400 from the splice acceptor site. Lacking sequence identity, e.g., ≤60% identity, it is designated herein that there is a GSH in the non-host intron, for example, that is 800 nt from the splice donor site and 400 nt from the splice acceptor site. Other cis acting elements and motifs may be used for determining the position of a GSH locus.

Proportional Distance from Cis-Acting Elements (Introns of Different Size)

When a host species intron lacks sequence identity and is different in length than a non-host intron, the proportional distance of the EVE insertion site and a genetic landmark, such as cis-acting elements (e.g., a splicing donor site or a splicing acceptor site), is used. For example, a host species has an intron that is 1200 nt-long but now the orthologous non-host intron is 2400 nt-long, the proportional distance is used. In the host species, the EVE inserted at 800 from the splicing donor site is located at 2/3rds intron size (800/1200). The proportional distance 2/3rds, in the non-host intron is 1600 nt from the splicing donor site. Thus, the GSH locus in the non-host species is 1600 nt from the splicing donor site and 800 nt from the splicing acceptor site.

### Example 3: Characterization of Novel GSH Loci

### Assessing the Impact of Different GSH on the Marker Gene Expression and Cell Differentiation

Human primary CD34+ HSC were used to evaluate the impact of transgenesis into different putative GSH. Homology arms and guide RNAs for CRISPR/Cas9 mediated gene insertion were designed and synthesized using online guide RNA prediction software (ChopChop, Broad, IDT). A reporter gene was inserted into the putative GSH locus and transformed cells were either seeded in methylcellulose supplemented with cytokines (CFU assay) or maintained in liquid medium supplemented with cytokines to promote differentiation into erythroid progenitors (erythroid differentiation).

### CFU Assay

Evaluation of stem cell differentiation was performed by colony forming units (CFU) assay where the color and morphology of stem cells was monitored by visualization under the microscope. Identification of committed erythroid progenitors such as CFU-GEMM, BFU-E, or CFU-E was performed by identification of characteristic features such as cell morphology and cell color of the cell colonies (FIG. 4A-FIG. 4C). In parallel, expression of GFP was monitored under UV light.

### Erythroid Differentiation

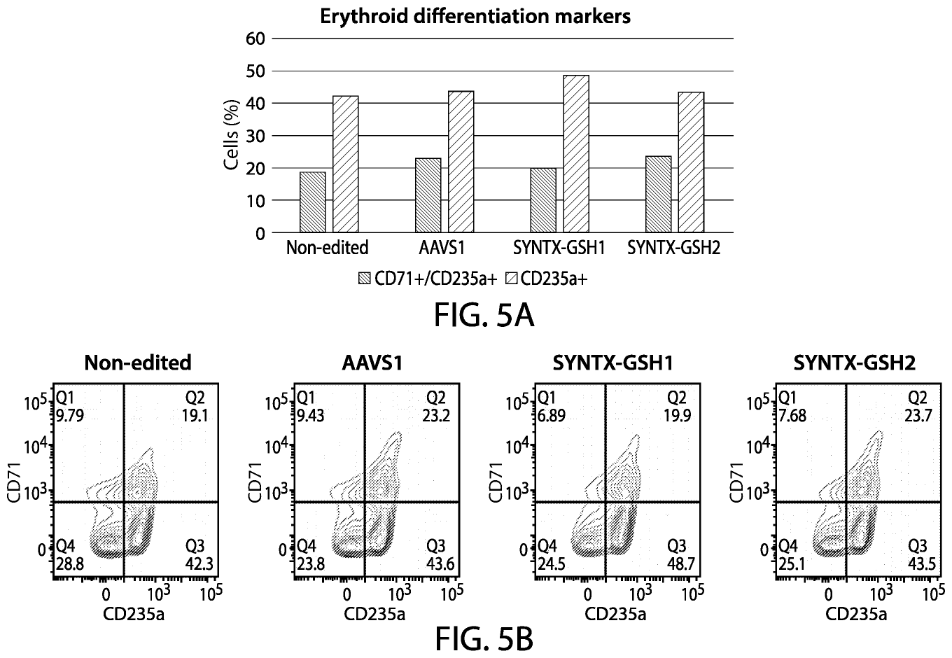

Quantification of two different bona-fide cell markers for erythropoiesis (CD71 and CD235) was performed by flow cytometry (FIG. 5A and FIG. 5B), indicating successful commitment of progenitor cells.

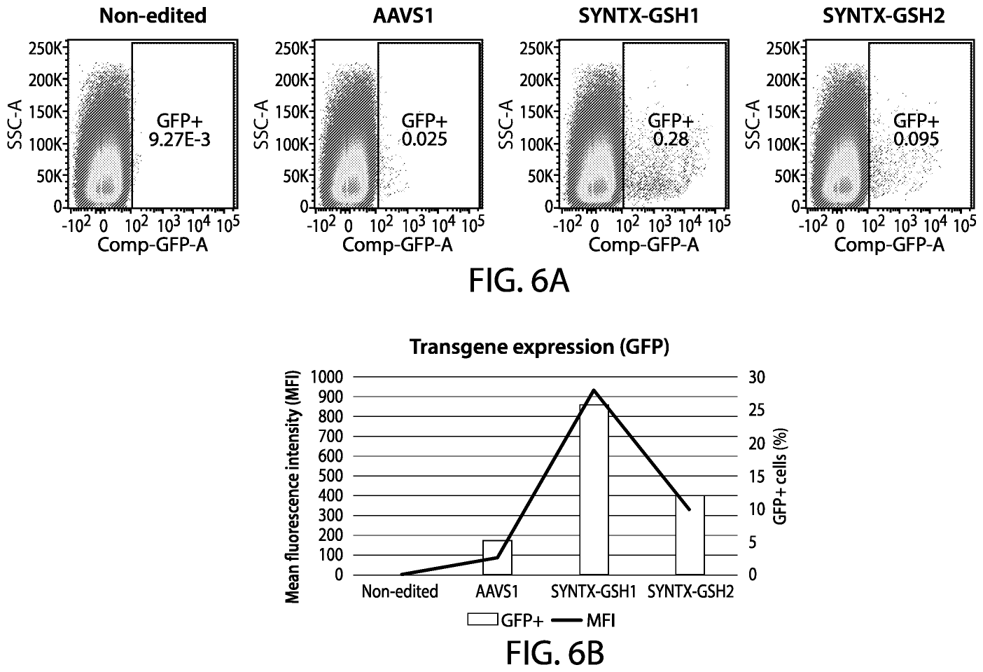

Result: no significant difference was observed among the evaluated conditions, WT (non-edited), AAVS1 edited, SYNTX-GSH1 edited and SYNTX-GSH2 edited. The results shown in FIG. 4A-FIG. 4C and FIG. 5A-FIG. 5B demonstrate that the novel putative safe harbor loci, SYNTX-GSH1 and SYNTX-GSH2, did not perturb the ability of primary human HSC to differentiate into erythrocytes. Stability of the GFP-expressing cells was monitored over 14 days after transgene addition by flow cytometry (FIG. 6A-FIG. 6B)

Results: Over the indicated period of time, cells edited into the SYNTX-GSH1 locus showed the higher percentage of GFP positive cells, followed by cells edited into SYNTX-GSH2 locus (FIG. 6A-FIG. 6B). These results demonstrate that gene editing into the novel GSH allowed a more stable and safe transgenesis than editing into the AAVS1 control locus. The identified loci (SYNTX-GSHs) can then be used as GSH for permanent transgenesis of stem cells and used for different ex vivo gene therapies.

TABLE 5
Exemplary characterizations of the representative GSH loci.

| NAME | CELL LINE EXPERIMENTS | PRIMARY CELL EXPERIMENTS |
|---|---|---|
| SYNTX- | No-template experiments to | Edited human CD34+ cells to stably |
| GSH1 | determine best gRNA. Edited | express GFP from this site and |
| | HEK293 cells to express GFP | demonstrated no impairment in ability to |
| | from this site showing stable | differentiate into erythroid lineage cells. |
| | expression > 30 days. | |
| SYNTX- | No-template experiments to | Edited human CD34+ cells to stably |
| GSH2 | determine best gRNA. Edited | express GFP from this site and |
| | HEK293 cells to express GFP | demonstrated no impairment in ability to |
| | from this site showing stable | differentiate into erythroid lineage cells. |
| | expression > 30 days. | |
| SYNTX- | No-template experiments to | |
| GSH3 | determine best gRNA. Edited | |
| | HEK293 cells to express GFP | |
| | from this site showing stable | |
| | expression > 30 days. | |
| SYNTX- | No template experiments to | |
| GSH4 | determine best gRNA. Edited | |
| | HEK293 cells to express GFP | |
| | from this site showing stable | |
| | expression > 30 days. | |
| SYNTX- | No-template experiments to | |
| GSH5 | determine best gRNA. | |
| SYNTX- | No-template experiments to | Edited human CD34+ cells to stably |
| GSH6 | determine best gRNA. Edited | express GFP from this site and |
| | 293T cells to express GFP from | demonstrated no impairment in ability to |
| | this site showing stable | differentiate to myeloid and erythroid |
| | expression > 20 days. | lineage cells. |
| SYNTX- | No-template experiments to | |
| GSH17 | determine best gRNA. Edited | |
| | 293T cells to express GFP from | |
| | this site showing stable | |
| | expression > 20 days. | |
| SYNTX- | No template experiments to | |
| GSH19 | determine best gRNA. | |
| SYNTX- | No-template experiments to | |
| GSH20 | determine best gRNA. | |
| SYNTX- | No-template experiments to | |
| GSH21 | determine best gRNA. Edited | |
| | 293T cells to express GFP from | |
| | this site showing stable | |
| | expression > 20 days. | |
| SYNTX- | No-template experiments to | Edited human CD34+ cells to stably |
| GSH22 | determine best gRNA. | express GFP from this site and |
| | | demonstrated no impairment in ability to |
| | | differentiate to myeloid and erythroid |
| | | lineage cells. |
| SYNTX- | No-template experiments to | |
| GSH23 | determine best gRNA. | |
| SYNTX- | No-template experiments to | |
| GSH31 | determine best gRNA. | |
| SYNTX- | No-template experiments to | |
| GSH32 | determine best gRNA. Edited | |
| | 293T cells to express GFP from | |
| | this site showing stable | |
| | expression > 20 days. | |
| SYNTX- | No template experiments to | Edited human CD34+ cells to stably |
| GSH38 | determine best gRNA. | express GFP from this site and |
| | | demonstrated no impairment in ability to |
| | | differentiate to myeloid and erythroid |
| | | lineage cells. |
| SYNTX- | No-template experiments to | |
| GSH42 | determine best gRNA. | |
| SYNTX- | No template experiments to | Edited human CD34+ cells to stably |
| GSH52 | determine best gRNA. | express GFP from this site and |
| | | demonstrated no impairment in ability to |
| | | differentiate to myeloid and erythroid |
| | | lineage cells. |
| SYNTX- | No-template experiments to | Edited human CD34+ cells to stably |
| GSH53 | determine best gRNA. | express GFP from this site and |
| | | demonstrated no impairment in ability to |
| | | differentiate to myeloid and erythroid |
| | | lineage cells. |
| SYNTX- | No-template experiments to | |
| GSH54 | determine best gRNA. | |
| SYNTX- | No-template experiments to | Edited human CD34+ cells to stably |
| GSH55 | determine best gRNA. | express GFP from this site and |
| | | demonstrated no impairment in ability to |
| | | differentiate to myeloid and erythroid |
| | | lineage cells. |
| SYNTX- | No-template experiments to | |
| GSH56 | determine best gRNA. | |

* The phrase “No-template experiments to determine best gRNA” indicates that different gRNAs for a genomice safe harbor have been tested to 1) confirm that the GSH site can be edited via CRISPR/Cas9; and 2) determine which gRNA gives the highest rate of double-stranded breaks as higher rates can improve homology-dependent repair (HDR) editing rates.

### Example 4: Assessing the Impact of Gene Addition into GSH on Global Cellular Transcriptome

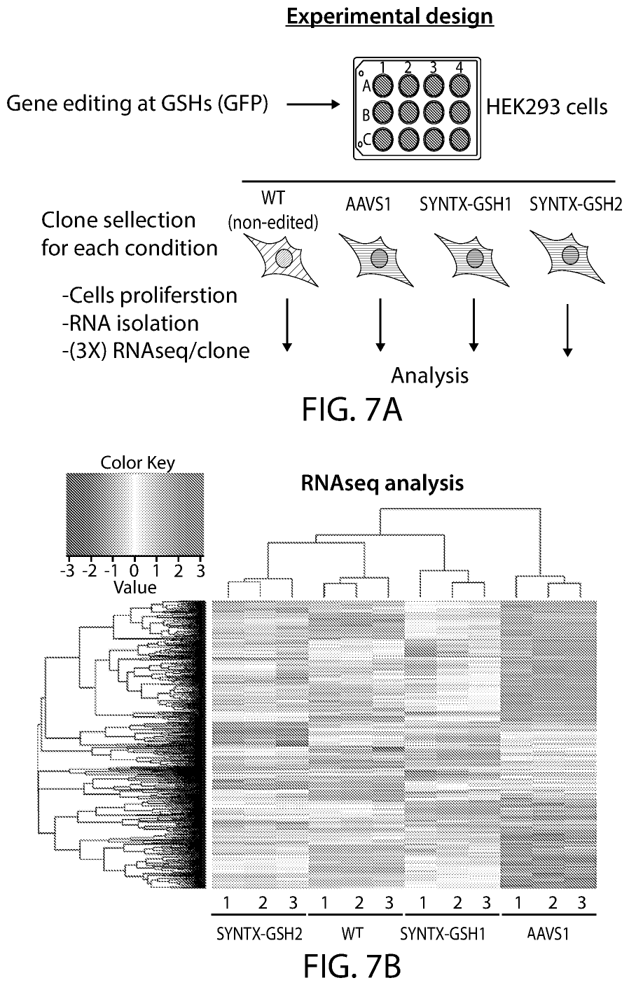

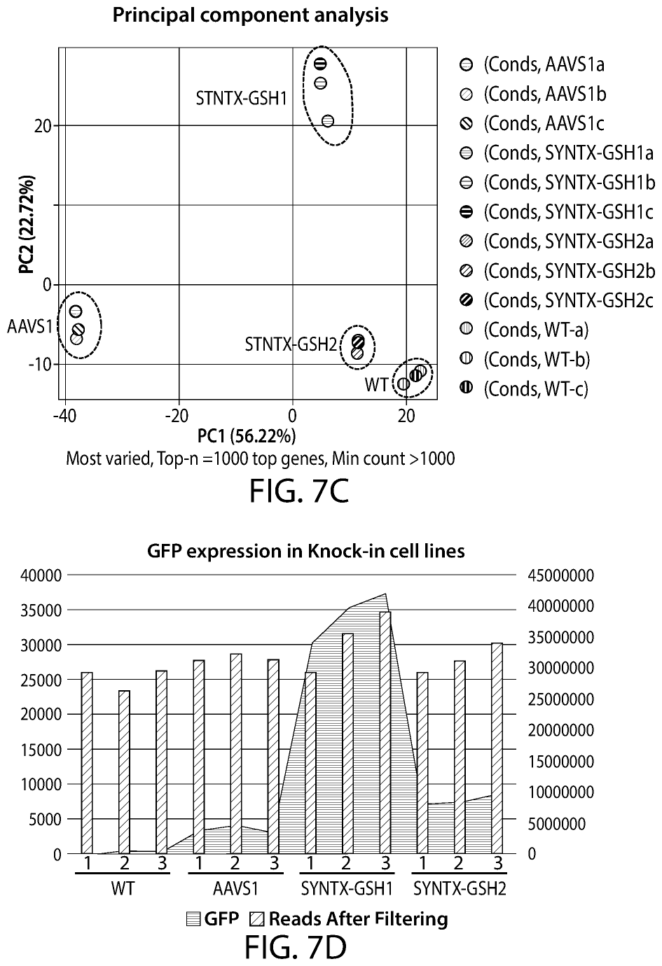

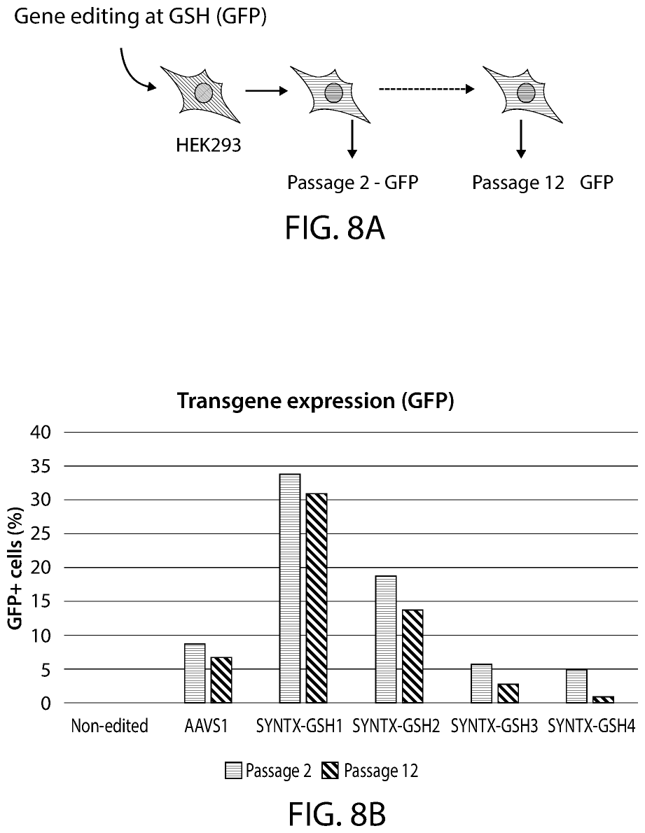

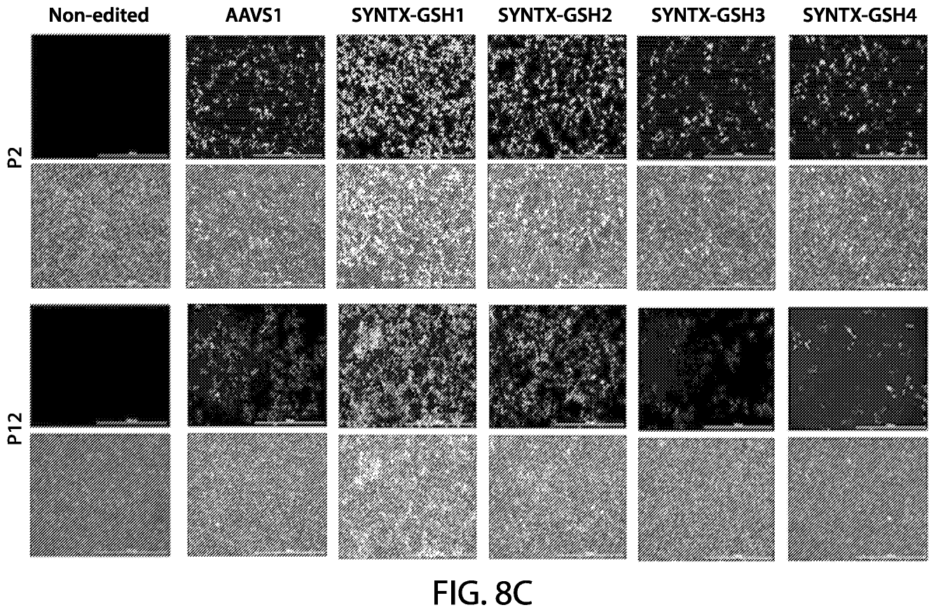

Human derived HEK293 cells were used to evaluate global gene expression after insertion of a reporter gene (GFP) into different GSH loci. HEK293 cells were edited by CRISPR/Cas9 gene insertion as described before in the indicated loci (AAVS1, SYNTX-GSH1 and SYNTX-GSH2). Non-edited cells, indicated as WT, were used as a control for basal gene expression. Briefly, positive GFP cells were cloned and amplified until reaching the necessary number of cells for processing. Total RNA was extracted and used to create mRNA libraries following standard procedures. RNAseq was performed in triplicate for each condition. Expression levels were assessed and compared among the different cell clones (FIG. 7B-FIG. 7D).

Result: The transcriptional landscape observed for each condition, clearly showed that gene insertion into the AAVS1 locus is the most distant from base condition (WT, non-edited), i.e., most disruptive, followed by cells with insertion into SYNTX-GSH1. Insertion into SYNTX-GSH2 shows minimal disturbance of the cell transcriptome, with a similar expression patter to WT non-edited cells, demonstrating that the proposed loci (SYNTX-GSH1 and SYNTX-GSH2) are behave as safe sites for transgene integration in human cells. This data is supported by principal component analysis (FIG. 7C) which quantifies the difference of the top 1000 most variable genes among the evaluated conditions (WT, AAVS1, SYNTX-GSH1 and SYNTX-GSH2) indicating that SYNTX-GSH1 and SYNTX-GSH2 are safer integration loci than the AAVS1 locus.

Finally, evaluation of transgene expression (GFP), corroborate previous results, demonstrating that SYNTX-GSH1 and SYNTX-GSH2 promote higher transgene expression than benchmark AAVS1 locus.

### Example 5: HEK293 Cells Edited at Genomic Safe Harbor Loci-Stability of GFP Expression

Assessing the GSH performance by stability of GFP expression over cell passages Human derived HEK293 cells were used to evaluate the impact of gene editing into different selected GSH on stability of transgene expression (GFP) over several cell passages. Homology arms and guide RNAs for CRISPR/Cas9-mediated gene insertion were designed and synthesized using an online guide RNA prediction software (ChopChop and Broad). A reporter gene (GFP) was inserted into different putative GSH loci. Non-edited cells were used as base control (WT), and gene addition was performed into AAVS1 locus (control), SYNTX-GSH1, SYNTX-GSH2, SYNTX-GSH3, and SYNTX-GSH4. Cells in all conditions were maintained for over 12 passages, representing a 30 days culture period and GFP was monitored by using a UV-light microscope.

Results: Gene addition into SYNTX-GSH1 demonstrated the highest GFP expression in early passages and during the evaluated period of time (P12), followed by cells edited into SYNTX-GSH2 locus. These two loci showed higher and more stable GFP expression than AAVS1 control. The other evaluated intergenic loci showed lower GFP expression levels and stability. These data, confirm the permissiveness and safety of the evaluated GSH (e.g., SYNTX-GSH1 and SYNTX-GSH2) for gene addition without producing a drastic perturbation of cell homeostasis and favoring stable and high level of transgene expression.

See also Table 5 for exemplary characterizations of the representative GSH loci.

### Example 6: Purification of CD34+ Cells

CD34+ cells for use in the disclosed methods can be purified according to suitable methods, such as those described in the following articles: Hayakama et al., Busulfan produces efficient human cell engraftment in NOD/LtSz-scid IL2Ry null mice, Stem Cells 27 (1): 175-182 (2009); Ochi et al., Multicolor Staining of Globin Subtypes Reveals Impaired Globin Switching During Erythropoiesis in Human Pluripotent Stem Cells, Stem Cells Translational Medicine 3:792-800 (2014); and McIntosh et al., Nonirradiated NOD, B6.SCID 112ry^{-l-}Kit^{W4l/W4l} (NBSGW) Mice Support Multilineage Engraftment of Human Hematopoietic Cells, Stem Cell Reports 4:171-180 (2015).

### Example 7: In Vitro or Ex Vivo Transduction of Erythroid Progenitor Cells Using the Viral Vectors

The recombinant viral vector (AAV) is used to transduce erythroid progenitor cells. Transgene expression in genotypically corrected cells facilitates rescue of the phenotype of the differentiated cells and lead to clinical improvement.

Hemaglobinopathies caused by gain of function mutations are inherited as autosomal recessive traits. Heterozygous individuals tend to be either asymptomatic or mildly affected, whereas individuals with mutations in both alleles are severely affected. Thus, correcting or replacing a single allele is clinically beneficial.

Since both beta-thalassemia and sickle cell disease (SCD) are caused by different mutations in the genes that express hemoglobin beta (HbB), a gene replacement strategy benefits patients with either disease. There are clinical studies for SCD using lentivirus vector (LV) that deliver the HbB expression cassette. The b-globin open reading frame (ORF) is regulated by the globin allele locus control region (LCR) and b-globin promoter. In order to fit into the LV, the minimal LCR has been mapped to three DNAse hypersensitive sites (HS) that inhibit DNA methylation and the formation of heterochromatin. Randomly integrating LV may integrate into heterochromatin resulting in shut-off of b-globin expression in the erythrocyte progenitor cells (e.g., erythroblasts), and thus, no phenotypic correction.

The LCR elements, HS, maintain the open, euchromatin structure of LV DNA.

Inserting the HbB cassette into a genomic safe harbor (GSH) locus. In contrast to transposable elements which constitute approximately 45% of the mammalian genome, heritable integrated parvovirus genomes (or endogenous virus elements, EVEs) occur in very few loci across hundreds of species. The EVEs are genomic markers of sites that tolerate insertion of foreign DNA without affecting embryogenesis, development, maturation, etc. on the short time-line and evolution/speciation on a geologic time-line. Presumably due to the disruptive effects of foreign DNA insertion, there are very few EVE loci that have accumulated in many diverse species over 100 million years. Despite the many species among the highly diverse phylogenetic taxa that harbor EVEs, there appear to be a limited number of genomic loci affected facilitating an empirical analysis of EVEs as GSHs in model systems, e.g., mouse. The conservation of the EVE loci among mammalian species allows us to determine the homologous sites in the human and mouse genomes. However, it is likely that not all GSHs will support long-term, stable expression all tissue types. Using in silico analysis, including RNAseq and ATAC-seq databases, GSH loci can be mapped to subgenomic regions that are actively expressed in the target tissue. Thus, for beta-globinopathies, erythroblasts are particularly interesting.

Utilizing GSH loci that are actively chromatin regions actively expressed chromatin in erythroblasts, circumvents the necessity of using the LCR elements to ensure euchromatinization where the LV integrated.

The process of homology directed repair (HDR) with a targeting nuclease improves the efficiency and specificity of recombination. “Homology arms” flanking the therapeutic gene, directs the vector DNA to the targeted locus. Recombination either by cellular DNA repair pathway enzymes, or an artificial process, e.g., CRISPR/Cas9 nuclease, integrates the transgene into the GSH.

In addition to b-globin promoter, other promoters have been used for long-term, high-level expression in numerous cell types and also in transgenic mouse strains.

For example, hemoglobin is a heterotetramer composed of 2× HbA and 2× HbB chains. In the absence of HbB, the HbA chain self-associates and form cytotoxic aggregates. The alpha-hemoglobin stabilizing protein (AHSP) is co-expressed in pro-erythrocytes to prevent aggregation of a-globin subunits. The AHSP promoter is highly active in erythrocyte precursors and is well characterized.

As another example, the CAG promoter enhancer is a synthetic promoter engineered from the cytomegalovirus enhancer fused to the chicken beta-globin promoter and exon 1 and intron 1 and splice acceptor of exon 2.

As another example, the MND promoter is active hematopoietic cells

As another example, the Wiskott-Aldrich promoter is active in hematopoietic cells.

As another example, the PKLR promoter is active in hematopoietic cells

Peripheral blood stem cells (PBSCs) are isolated by leukophresis.

Cryopreserved peripheral blood cells in Hemofreeze bags are recovered by rapid thawing in a 37° C. water bath. These thawed cells are suspended in 4% HSA at 4° C. and washed twice by centrifugation at 450 g for 5 min at 4° C. The platelets are removed twice by overlaying on 10% HSA and centrifugation at 450 g for 15 min at 4° C. The erythrocytes are removed by overlaying on Ficoll-Hypaque (FH: 1.077 g/cm³; Pharmacia Fine Chemicals, Piscataway, NJ, USA) and centrifugation at 400 g for 25 min at 4° C. The interface mononuclear cells (Pl-, FH cells) are collected, washed twice in washing solution and resuspended in 4% HSA at 4° C. (MN cells). A nylon-fiber syringe (NF-S) is used to remove adherent cells. Five grams of NF is packed into a 50 mL disposable syringe. The mono nuclear cells were transferred to an additional 50 ml syringe and gently infused into the NF-S, then were incubated at 4° C. for 5 min. The MN cells are then collected into a 50 mL syringe through a plunger of the NF-S, and the cells are pooled in 50 mL of a conical tube. These pooled cells are centrifuged at 400 g for 5 min at 4° C., and resuspended in 4% HSA at 4° C. (NF cells). The cell suspension is then immediately processed for CD34+ selection on the Isolex Magnetic Cell Separation System (Isolex 50; Baxter Healthcare, Immunotherapy Division, Newbury, UK) following the manufacturer's instructions. Briefly, cells are incubated with 9C5 murine immunoglobulin G1 (IgG1) anti-human CD34 antibody (10 mg/l×108 NF cells) for 15 min at 4° C. with slow endover-end rotation. After sensitization, the cells are washed with 4% HSA at 4° C. to remove any excess/unbound antibody. The Dynabeads (Oslo, Norway) are then added to the washed, sensitized cells at a final bead/cell ratio of 1:10. After mixing at 4° C. for 30 min, the cell-bound microspheres and free microspheres become attached to the wall via the magnet (Dynal MPC-1, Dynal, Fort Lee, NJ, USA) and any free cells that do not bind to the microspheres are removed. This washing procedure is repeated twice with 4% HSA at 4° C. The linkage between Dynabeads and CD34+ cells is cleaved by a PR34+ Stem Cell Releasing Agent for 30 min at 4° C. The free Dynabeads are removed from the CD34+ cells via the magnet. D-PBS containing 1% ACD-A and 1% HSA at 25° C. is used for collection of cells. The resulted cell product is controlled by Flow cytometry.

See Table 5 for exemplary characterizations of the representative GSH loci.

### Example 8: Expression of the Nucleic Acid Vectors In Vivo

In vivo protein expression from vectors described above is determined in mice. As described above, the HbB gene cassette is engineered to comprise a 5′ and 3′ GSH-specific homology arm (e.g., SYNTX-GSH1GSH locus or any one of those listed in Table 3). In some experiments, the 5′- and 3′ GSH-specific homology arms are large (up to 2 Kb each). In some experiments, the vector further comprises a sequence encoding a CRISPR/Cas9 nuclease and a gRNA that creates DNA cleavage to initiate a homologous recombination between the homology arm with the GSH locus. In some experiments, the nucleic acid vector is delivered in lipid nanoparticles (LNPs). In other experiments, the nucleic acid vector is packaged into a viral vector according to the method described herein and/or the method known in the art.

In some experiments, a negative control is established, e.g., with a control vector having scrambled homology arm sequences or no homology arms to check the efficiency of recombination may be more appropriate. The nucleic acid vector comprising the HbB gene cassette further comprises a promoter, WPRE element, and pA.

A nuclease expressing unit can be delivered in trans, e.g., in a separate nucleic acid vector or a viral vector, such Cas9 mRNA, zinc-finger nucleases (ZFN), transcription activator-like effector nucleases (TALEN), mutated “nickase” endonuclease, class II CRISPR/Cas system (CPF1). In experiments, LNPs can be used as a delivery option. The transport into the nuclei can be increased by using a nuclear localization signal (NLS) fused into the 5′ or 3′ enzyme peptide sequence, according to methods commonly known to persons of ordinary skill in the art. In other embodiments, the NLS can be inserted internally such that the NLS is exposed on the surface of the nuclease and does not interfere with its function as a nuclease.

Where appropriate for the nuclease, to induce double-stranded break (DSB) at the desired site one or more single guided RNA are delivered in trans as well: Either as an sgRNA expressing vector or chemically synthesized synthetic sgRNA. (sgRNA=single guide-RNA target sequence) as described herein. sgRNA can be selected using freely available software/algorithm, e.g., such as attools.genome-engineering.org, can be used to select suitable single guide-RNA sequences.

The 5′ GSH-specific homology arm can be approximately 350 bp long, and can be in range between 10 to 5000 bp, as described herein. In some experiments, the 3′ GSH-specific homology arm can be the same length or longer or shorter than the 5′ GSH-specific homology arm, and can be approximately 2000 bp long, or in the range of between 50 to 2000 bp, as described herein. Details study regarding length of homology arms and recombination frequency is e.g., reported by Jian-Ping Zhang et al., Genome Biology, 2017.

The nucleic acid vector in nanoparticles or the viral vectors (e.g., AAV vectors) are administered to the mouse by tail vein injection. This delivery modality gives access to all organs in the body.

### Example 9: Construction of a Viral Vector

### A Nucleic Acid for the Viral Vector

A vector genome design consists of inverted terminal repeats (ITRs), e.g., the ITR conformers of the AAV terminal palindrome and an expression or transcription cassette. The generic expression cassettes consist of regulatory elements, typically characterized as enhancer and promoter elements. The region transcribed by the RNA polymerase complex consists of cis acting regulatory elements e.g., TATA-box, and 5′ untranslated exonic sequences, intronic sequences, translated exonic sequences, 3′ untranslated region, polyadenylation signal sequence. Post-transcriptional elements include a Kozak motif for translational initiation and the woodchuck hepatitis virus post-transcriptional regulatory element. The specific vector is chemically synthesized using a commercial service provider and ligated into a plasmid for propagation in Escherichia coli. The plasmid minimally contains multiple cloning sites, at least one antibiotic resistance gene, a plasmid origin of replication, and sequences to facilitate recombination into a baculovirus genome. Two commonly used approaches are: (1) A bacterial system in which the E. coli harbors a baculovirus genome (bacmid) that uses transposase mediated recombination to transfer the plasmid genes into the bacmid. E. coli with the recombinant bacmid is detectable by growth on agar plates prepared with selective media. The “positive” colonies are expanded in suspension culture medium and the bacmid harvested after about 3 days post-inoculation. Sf9 cells are then transfected with the bacmid which in the permissive insect cell, produce infectious, recombinant baculovirus particles. (2) Alternatively, the vector DNA is inserted into a shuttle plasmid that has several hundred basepairs of baculovirus DNA flanking the insert. Co-transfection of Sf9 cells with the shuttle plasmid and linearized baculovirus subgenomic DNA restores the deleted baculovirus elements producing infectious, recombinant baculovirus. The ≤6 kb vector DNA resides in the baculovirus genome (ca. 135 kb) and is propagated as baculovirus unless the Sf9 cell expresses the AAV non-structural or Rep proteins. The Rep protein then acts on the ITR allowing resolution of the vector and baculovirus genomes where the vector genome then replicates autonomously of the baculovirus genome (FIG. 1B).

### Nucleic Acid Composed of DNA

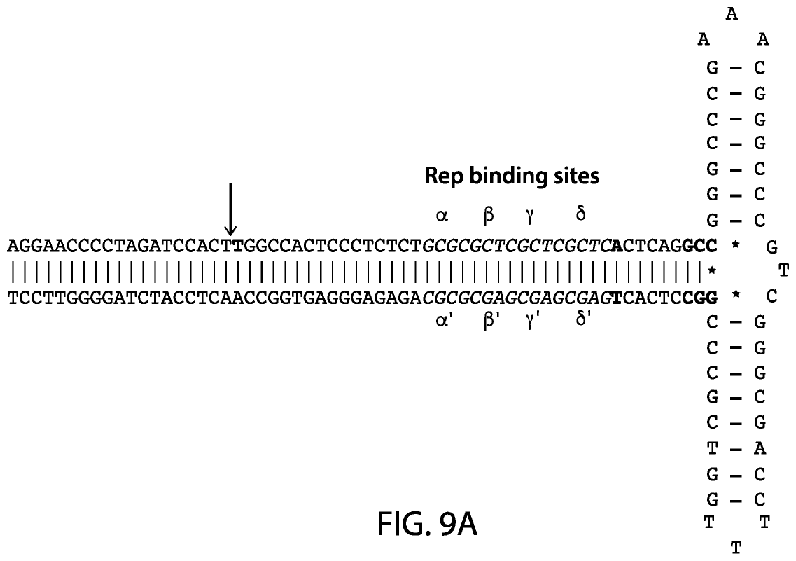

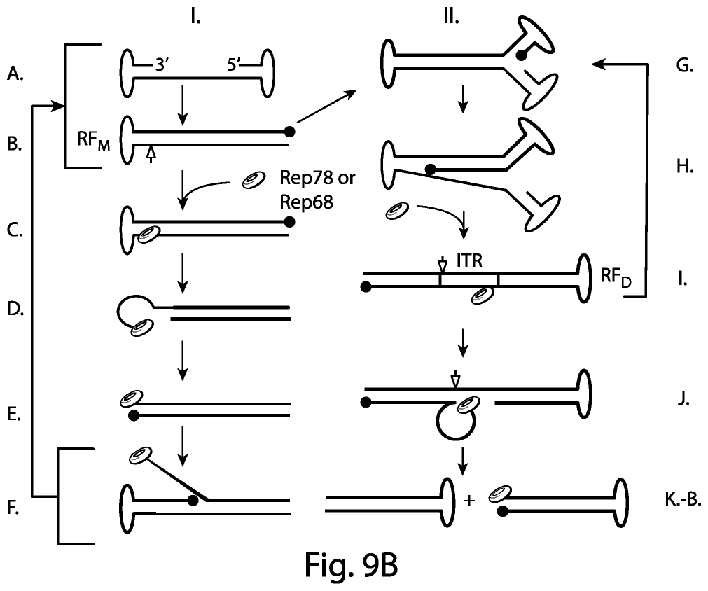

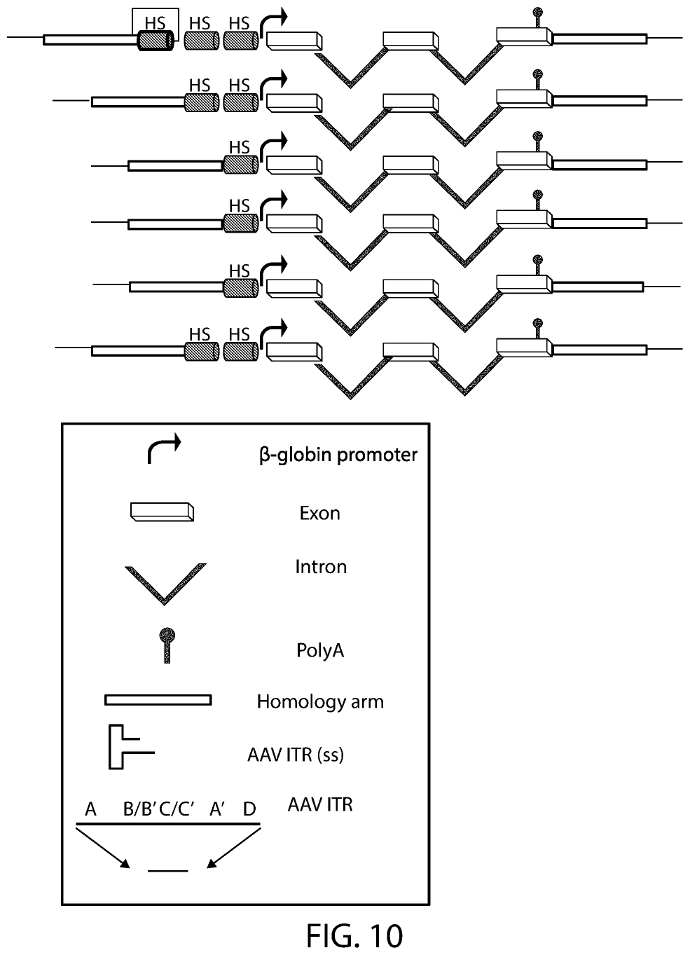

DNA can be either single-stranded or self-complimentary (i.e., intramolecular duplex). As illustrated in FIG. 9B, Rep-mediated replication of the vector DNA proceeds through several intermediates. These replicative intermediates are processed into single-stranded virion genomes, however, the fecundity of products may overwhelm processing into single-stranded virion genomes. In this case, the replicative intermediate consisting of an intramolecular duplex molecule, represented as the RFm (FIG. 9B), is packaged into the AAV capsid. Packaging of the self-complementary vector genomes occurs despite the presence of functional ITRs.

DNA can have a Rep protein-dependent origin of replication (ori). The ori can consist of Rep binding elements (RBEs), and within a terminal palindrome. The terminal palindrome, referred to as the inverted terminal repeats (ITRs), can consist of an overall palindromic sequence with two internal palindromes. The ITR can have cis-acting motifs required for replication and encapsidation in capsids.

RBE represents Rep binding elements canonical GCTC; RBE′ represents non-canonical RBE, unpaired TTT at the tip of the ITR cross-arm; and trs represents terminal resolution site 5′AGTTGG, GGTTGG, etc. The catalytic tyrosine of Rep (Y156) cleaves the trs and forms a covalent link with the scissile, 5′ thymidine. Mutation of the trs leads to inefficient or loss of cleavage resulting in self-complimentary DNA. Alternatively, self-complementary virion genomes result from encapsidation of the incomplete processing of the RFm.

### DNA Replication of the Viral Vector

Replication utilizing AAV ITR is referred to as “rolling hairpin” replication. As single-stranded virion DNA, the ITRs form an energetically stable, T-shaped structure (FIG. 9A) that serves as a primer for DNA extension by the host-cell DNA polymerase complex (FIG. 9B). DNA synthesis is leading strand, processive process resulting in a duplex intermediate where the complementary strands are covalently linked through the ITR (FIG. 9B). The p5 Rep protein binds are structurally related to rolling-circle replication (RCR) proteins, bind to the ITR forming a multi-subunit complex. The helicase activity of the Rep proteins unwinds the ITR creating a single-stranded bubble with the terminal resolution site (5′-GGT|TGA-3′). The phosphodiester bond between the thymidines is attacked by the hydroxyl group of the Rep protein catalytic tyrosine (AAV2=Y156) forming a tyrosine-thymidine diester with the 5′-thymidine. A cellular DNA polymerase complex extends the newly created 3-OH at the terminal resolution site restoring the terminal sequence to the template strand (FIG. 9B). Resolution of the nucleoprotein complex occurs through an unknown process.

### Encapsidation

Encapsidation or packaging of DNA into an icosahedral virus capsid is an active process requiring a source of energy to overcome the repulsive force created by backpressure of compressing DNA into a confined volume. The ATPase activities of the NS/Rep proteins translate the stored chemical energy of the trinucleotide by hydrolyzing the gamma phosphate. The backpressure generated determines the length of DNA that can be accommodated in the capsid, i.e., the motive force of the ATPase/helicase can “push” up to 12 pN, for example, which may be reached once 4,800 nucleotides are packaged. AAV p19 Rep proteins are monomeric, non-processive helicases that are necessary for efficient encapsidation. Although there are scant data that support physical interactions between Rep and capsid, the overcoming the backpressure requires that stable interactions form between the packaging helicase(s) and the capsid. The nature of these interactions are unknown and nuclear factors may stabilize or mediate the interactions between the non-structural proteins and capsids.

### Example 10: Producing the Viral Vectors Using Insect Cells

Sf9 cells, in which at least one nucleic acid encoding a viral replication protein (Rep) and/or a viral capsid protein (VP1, VP2, VP3, etc.) is integrated into a GSH locus (e.g., SYNTX-GSH1 locus), are prepared. The Sf9 cells are grown in serum-free insect cell culture medium (HyClone SFX-Insect Cell Culture Medium) and transferred from an erlenmyer shake flask (Corning) to a Wave single-use bioreactor (GE Healthcare). Cell density and viability are determined daily using a Cellometer Autor 2000 (Nexelcom). Volume is adjusted to maintain a cell density of 2 to 5 million cells per mL. At the final volume (10 L) and density of 2.5 million cells per mL, the baculovirus infected insect cells (BIICs) are added (cryopreserved, 100× concentrated cell “plugs”) 1:10,000 (v:v). The highly diluted BIICs release Rep-VP-Bac, NS-Bac, and vg-Bac that are at very low multiplicity of infection (MOI) and virtually no cells are co-infected during the primary infection. However, subsequent infection cycles release large numbers of each of the requisite baculovirus achieving a very high MOI ensuring that each cell is infected with numerous virus particles. The cells are maintained in culture for four days or until viability drops to ≤30%.

### Example 11: Purification of the Viral Vectors

The viral vectors or viral particles are partitioned in both the cellular and extracellular fractions. To recover the maximum number of particles, the entire biomass including cell culture medium is processed. To release the intracellular viral vectors, Triton-X 100 (x %) is added to the bioreactor with continued agitation for 1 hr. The temperature is increased from 27° C. to 37° C., then Benzonase (EMD Merck) or Turbonuclease (Accelagen, Inc.) is added (2u per mL) to the bioreactor with continued agitation. The biomass is clarified using a staged depth filter, then filter sterilized (0.2 μm) and collected in a sterile bioprocessing bag. The viral vectors are recovered using sequential column chromatography using immune-affinity chromatography medium and Q-Sepharose anion exchange. Chromatograms displaying and recording UV absorption, pH, and conductivity are used to determine completion of the washing and elution steps. Relative efficiency of each step is determined by western blot analysis and quantitatively by ddPCR or qPCR analysis aliquots of the input material (“Load”), the flow-through, the wash, and the elution.

Immune-affinity chromatography uses a “nanobody,” the VhH region of a single-domain immunoglobulin produced in llamas and other camelid species. To produce the nanobody, an antibody provider immunizes llamas with the viral vectors, i.e., assembled capsids with no virion genome. The viral vectors are prepared in Sf9 cells infected with the VP-Bac and purified using cesium chloride isopycnic gradients, followed by size exclusion chromatography (Superdex 200). Following a prime (1×)/boost (2×) immunization protocol the antibody service provider bleeds the llama and isolates peripheral blood mononuclear cells or mRNA extracted from nucleated blood cells. Reverse transcription using primers specific for the conserved VhH CDR flanking regions (FR1 and FR 4) produces cDNA that is cloned into plasmids used to generate the T7Select 10-3b phage display library (EMD-Millipore). Following several rounds of panning to enrich for phage that interact with the capsid of the viral vector, phage clones are isolated from plaques. E. coli infected with the recombinant phage are mixed into agarose and applied as an overlay onto LB-agar plates. The E. coli grow to confluency establishing a “lawn” where lysed bacteria and appear as plaques on the plate. To identify phage that bind to viral vector, nitrocellulose filters placed on surface of the agar plates to transfer proteins from the plaques to the filter. The filters are incubated with the viral vector capsids modified with a covalently linked horseradish peroxidase (HRP) (EZLink Plus Activated Peroxidase Kit, ThermoFisher) and washed with phosphate buffered saline. HRP activity can be detected with either a chromogenic (Novex HRP Chromogenic Substrate, ThermoFisher) or chemiluminescent substrate (Pierce ECL Western Blotting Substrate, ThermoFisher). The sequences of the cDNA in the phage are determined and ligated into a bacterial expression plasmid and expressed with a 6xHis tag for purification. The chelating column-purified nanobody is covalently linked to chromatography medium, NHS-activated Sepharose 4 Fast Flow (GE Healthcare).

The viral vectors are recovered from the clarified Sf9 cell lysate by binding, washing, and eluting from the nanobody-Sepharose column. The efficiency of binding is determined by western blotting the column load and flow through. The wash step is considered complete when the UV280 nm absorbance returns to baseline (i.e., pre-load) values. An acidic pH shift releases the viral particles that are eluted from the nanobody-Sepharose medium. The eluate is collected in 50 nM Tris-Cl, pH 7.2 to neutralize the elution medium.

The concentration of the viral vector particles is determined using the viral vector-specific ELISA and qPCR which can be used to estimate the percentage of filled particles, i.e., vector genome-containing.

### Example 12: Pulsatile Gene Expression

A viral vector comprising a nucleic acid encoding Factor VIII (FVIII), F8 or a fragment encoding a B-domain deleted polypetide, flanked by 5′ and 3′ homology arms with homology to a SYNTX-GSH1 locus, is used to transduce hepatocytes as a therapy for hemophilia A. The homology arms allow homologous recombination-mediated insertion of the nucleic acid encoding FVIII, F8, or a fragment encoding a B-domain-deleted polypeptide stably into the SYNTX-GSH1 locus. FVIII is an essential blood-clotting protein, also known as anti-hemophilic factor (AHF). In humans, factor VIII is encoded by the F8 gene. Defects in this gene result in hemophilia A, a recessive X-linked coagulation disorder. Factor VIII is produced in liver sinusoidal cells and endothelial cells outside the liver throughout the body.

Attempts have been made previously to increase the expression of F8 gene to treat hemophilia A. For example, Valoctocogene Roxaparvovec (also known as BMN270 or), an adenovirus-associated virus (AAV5) vector-mediated gene transfer of human Factor VIII was tested in patients with severe haemophilia A (ClinicalTrials.gov Identifiers: NCT02576795; NCT03370913; NCT03392974; NCT03520712). However, FDA rejected its approval in 2020, requesting long-term safety and efficacy data. The long-term data may be needed to ease the concerns over the increased dosage that may subsequently result in gradual gene expression of the transgene.

FVIII has been a difficult recombinant protein to produce in either microbial or eukaryotic expression systems. The development of the “B-domain” deleted improved expression levels and reduced the size of the open-reading frame, however, FVIII expression levels were substantially lower than other proteins. To overcome these low levels, the clinical dose of Valoctocogene Roxaparvovec viral vector was increased. Patients were treated with 6E+13 vector particles (referred to as vector genomes, or vg) per kg. Based on large animal models, a small minority of hepatocytes were transduced with rAAV5-FVIII. As a result of the large number of vg per cell, the transduced cell expresses relatively large quantities of FVIII. The metabolic demand for FVIII expression likely disrupts the normal requirements for hepatocyte protein expression. The hepatocyte cellular compartments normally involved in protein folding and secretion may become congested with the FVIII. Endothelial cells that produce FVIII production are likely specialized for this activity and produce FVIII from the allele on the single X chromosome under the transcriptional control of the highly regulated native FVIII promoter.

Accordingly, it is hypothesized herein that the perturbations of the hepatocyte homeostasis create cellular stress that induces an inflammatory state. The metabolic and protein folding/export burdens are exacerbated by the use of constitutive, highly active promoters used in the rAAV-FVIII vectors. The inflammation and cytokine production may lead to cell turnover or cell death.

To circumvent this problem and to address the long-felt need for a therapy for hemophilia A, a viral vector is engineered to comprise (a) the gene F8, or (b) the gene F8 with B-domain deletion, and as described above, flanked by 5′ and 3′ homology arm with homology to the SYNTX-GSH1 locus. In contrast to the constitutive and highly active promoter used in the clinical trial for Valoctocogene Roxaparvovec, the viral vector is prepared with an inducible expression system.

An inducible expression system keeps the F8 gene at the default transcriptionally off state until a reagent turns-on or disinhibits expression (see e.g., FIG. 14). Pulsatile expression spares the hepatocytes from over-expression stress. The timing of the pulses (i.e., the timing of turning on the gene expression) can be determined from the initial serum levels (t0) and the half-life (t1/2) of FVIII. The t1/2 is estimated to be 9 to 14 days, thus a 14-day (2 wks) t1/2 is used, and mild hemophilia is defined as FVIII levels ≥5% normal.
Transgene expression=150%
68 days to decline to 5%
   Here, the expression is induced monthly that results in therapeutic levels of FVIII.

A wide range of ASO chemistries (antisense oligo nucleotides ASO or AON) have been developed that increase the t1/2 in the cell. Here, an ASO chemistry with relatively short t1/2 is used to achieve a pulse of FVIII expression which diminishes as the ASO is cleared from the cell. The optimal t1/2 is determined empirically based on among others, the transduced cell number, promoter activity, and kinetics of transcript maturation.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the present invention described herein. Such equivalents are intended to be encompassed by the following claims.

What is claimed is:

1. A method of identifying a genomic safe harbor (GSH) locus, comprising:
(a) inducing a random insertion of at least one marker gene into a genome in a cell;
(b) determining the stability and/or level of the marker gene expression; and
(c) identifying a genomic locus, wherein the inserted marker gene shows the stable and/or high level of the expression, as a GSH.

2. The method of claim 1, further comprising:
(a) identifying a genomic locus, wherein the inserted marker gene does not affect cell viability; and/or
(b) identifying a genomic locus, wherein the inserted marker does not affect the cell's ability to differentiate (e.g., pluripotency, multipotency).

3. The method of claim 1 or 2, wherein the cell is selected from a cell line, a primary cell, a stem cell, or a progenitor cell, optionally wherein the cell is a stem cell or a progenitor cell.

4. The method of any one of claims 1-3, wherein the cell is selected from an embryonic stem cell, a tissue-specific stem cell, a mesenchymal stem cell, an induced pluripotent stem cell (iPSC), a hematopoietic stem cell, a hematopoietic CD34+ cell, and epidermal stem cell, an epithelial stem cell, neural stem cell, a lung progenitor cell, and a liver progenitor cell.

5. The method of any one of claims 1-4, wherein the cell is a mammalian cell, optionally wherein the mammalian cell is a mouse cell, a dog cell, a pig cell, a non-human primate (NHP) cell, or a human cell.

6. The method of any one of claims 1-5, wherein the random insertion is induced by:
(a) transfecting the cell with a nucleic acid molecule comprising the marker gene, optionally wherein the nucleic acid is a plasmid; or
(b) transducing the cell with an integrating virus comprising the marker gene.

7. The method of any one of claims 1-6, wherein the random insertion is induced by transducing the cell with an integrating virus comprising the marker gene; and the integrating virus is a retrovirus, optionally wherein the retrovirus is a gamma retrovirus.

8. The method of any one of claims 1-7, wherein the at least one marker gene comprises a screenable marker and/or a selectable marker, optionally wherein
(a) the screenable marker gene encodes a green fluorescent protein (GFP), beta-galactosidase, luciferase, and/or beta-glucuronidase; and/or
(b) the selectable marker gene is an antibiotic resistance gene, optionally wherein the antibiotic resistance gene encodes blasticidin S-deaminase or amino 3′-glycosyl phosphotransferase (neomycin resistance gene).

9. The method of any one of claims 1-8, wherein the marker gene is not operably linked to a promoter.

10. The method of any one of claims 1-8, wherein the marker gene is operably linked to a promoter, optionally wherein the promoter is a tissue-specific promoter.

11. The method of any one of claims 1-10, wherein the GSH is intronic, exonic, or intergenic.

12. A method of identifying a GSH locus, the method comprising:
(a) determining the presence and location of an endogenous virus element (EVE) in the genome of a metazoan species;
(b) determining intergenic or intronic boundaries proximal to the EVE; and
(c) identifying an intergenic or intronic locus comprising the EVE as a GSH locus.

13. The method of claim 12, wherein
(a) the presence and location of an EVE are determined by searching in silico for sequences homologous to a virus element; and/or
(b) the intergenic or intronic boundaries proximal to the EVE are determined by aligning the sequences flanking the EVE and its orthologous sequences of one or more species whose intergenic or intronic boundaries are known.

14. A method of identifying a GSH locus in an orthologous organism, the method comprising:
(a) identifying a GSH locus in Species A according to the method of any one of claims 1-13;
(b) determining the location of (i) at least one cis-acting element proximal to the GSH locus in Species A and (ii) the corresponding cis-acting element(s) in Species B; and
(c) identifying a locus in Species B as a GSH locus, wherein the distance between the locus and the at least one cis-acting element in Species B is substantially proportional to the distance between the GSH locus and the corresponding cis-acting element(s) in Species A.

15. The method of claim 14, wherein the at least one cis-acting element is selected from a splicing donor site, a splicing acceptor site, a polypyrimidine tract, a polyadenylation signal, an enhancer, a promoter, a terminator, a splicing regulatory element, an intronic splicing enhancer, and an intronic splicing silencer.

16. The method of claim 14 or 15, wherein the at least one cis-acting element comprises two or more cis-acting elements.

17. The method of any one of claims 14-16, wherein the at least one cis-acting element comprises two cis-acting elements; and the first cis-acting element is located upstream (i.e., 5′ to) of the GSH locus, and the second cis-acting element is located downstream (i.e., 3′ to) of the GSH locus.

18. The method of claim 17, wherein the distance between the at least one cis-acting element and the GSH locus relative to the distance between two cis-acting elements in Species B is substantially proportional to the distance between the corresponding cis-acting element and the GSH locus relative to the distance between two cis-acting elements in Species A.

19. The method of any one of claims 14-18, wherein the distance between the at least one cis-acting element to the GSH locus in Species B is at least 20% but no more than 500% of the distance between the at least one cis-acting element to the GSH locus in Species A.

20. The method of any one of claims 14-19, wherein the distance between the at least one cis-acting element to the GSH locus in Species B is at least 80% but no more than 250% of the distance between the at least one cis-acting element to the GSH locus in Species A.

21. The method of any one of claims 12-20, wherein the GSH locus is in a mammalian genome, optionally wherein the mammalian genome is a mouse genome, a dog genome, a pig genome, a NHP genome, or a human genome.

22. The method of any one of claims 12-21, wherein the EVE or the virus element
(a) comprises a provirus or a fragment of a viral genome;
(b) comprises a viral nucleic acid, viral DNA, or a DNA copy of viral RNA; and/or
(c) encodes a structural or a non-structural viral protein, or a fragment thereof.

23. The method of any one of claims 12-22, wherein the EVE comprises viral nucleic acid from a retrovirus, a non-retrovirus, parvovirus, or circovirus.

24. The method of claim 23, wherein
(a) the parvovirus is selected from B19, minute virus of mice (mvm), RA-1, AAV, bufavirus, hokovirus, bocavirus, and any one of the parvoviruses listed in Tables 1A-1D, optionally wherein the parvovirus is AAV; and/or
(b) the circovirus is porcine circovirus (PCV) (e.g., PCV-1, PCV-2).

25. The method of any one of claims 14-24, wherein the metazoan species is selected from Cetacea, Chiropetera, Lagomorpha, and Macropodiadae.

26. The method of any one of claims 1-11, further comprising the method of any one of claims 12-25.

27. The method of any one of claims 1-26, further comprising performing at least one in vitro, ex vivo, and/or in vivo assay.

28. The method of claim 27, wherein the at least one in vitro, ex vivo, and/or in vivo assay is selected from:
(a) de novo targeted insertion of a marker gene into the locus in a cell (e.g., human cell) and determine (i) the cell viability, (ii) the insertion efficiency and/or (iii) marker gene expression;
(b) targeted insertion of a marker gene into the locus in a progenitor cell or stem cell and differentiate in vitro and determine (i) marker gene expression in all developmental lineages, and/or (ii) whether the insertion of the marker gene affects differentiation of the said progenitor cell or stem cell;
(c) targeted insertion of a marker gene into the locus in a progenitor cell or stem cell and engraft the cell into immune-depleted mice and assess marker gene expression in all developmental lineages in vivo;
(d) targeted insertion of a marker gene into the locus in a cell and determine the global cellular transcriptional profile (e.g., using RNAseq or microarray); and
(e) generate a transgenic knock-in mouse wherein the genomic DNA of the mouse has a marker gene inserted in the locus, optionally wherein the marker gene is operatively linked to a tissue specific or inducible promoter.

29. The method of claim 28, wherein the progenitor cell or the stem cell is selected from an embryonic stem cell, a tissue-specific stem cell, a mesenchymal stem cell, an induced pluripotent stem cell (iPSC), a hematopoietic stem cell, a hematopoietic CD34+ cell, and epidermal stem cell, an epithelial stem cell, neural stem cell, a lung progenitor cell, muscle satellite cell, intestinal K cell, and a liver progenitor cell.

30. A nucleic acid vector, comprising at least a portion of the GSH nucleic acid identified in the method of any one of claims 1-29.

31. The nucleic acid vector of claim 30, wherein the GSH nucleic acid comprises an untranslated sequence or an intron.

32. The nucleic acid vector of claim 30 or 31, wherein the GSH comprises a sequence that is at least 65% identical to the sequence of any one of GSH or a fragment thereof listed in Table 3.

33. The nucleic acid vector of any one of claims 30-32, wherein the GSH comprises a sequence that is at least 65% identical to the sequence of the genomic DNA or a fragment thereof of SYNTX-GSH1, SYNTX-GSH2, SYNTX-GSH3, or SYNTX-GSH4.

34. The nucleic acid vector of any one of claims 30-33, further comprising at least one non-GSH nucleic acid, e.g., a nucleic acid having sequences that are heterologous to GSH, e.g., nucleic acid sequences not natively present in the GSH locus, e.g., a transgene.

35. The nucleic acid vector of claim 34, wherein the at least one non-GSH nucleic acid is flanked by a GSH 5′ homology arm and/or a GSH 3′ homology arm, wherein the homology arm comprises a nucleic acid sequence that is at least about 65% identical to the target GSH nucleic acid.

36. The nucleic acid vector of claim 35, wherein the GSH homology arm is between 10-5000 base pairs in length, optionally wherein the GSH homology arm is between 100-1500 base pairs in length.

37. The nucleic acid vector of claim 35, wherein the GSH homology arm is at least 30 base pairs in length.

38. The nucleic acid vector of any one of claims 35-37, wherein the GSH homology arm is sufficient in length to mediate homology-dependent integration into the GSH locus in the genome of a cell.

39. The nucleic acid vector of any one of claims 35-38, wherein the at least one non-GSH nucleic acid is in an orientation for integration in the GSH in a forward orientation.

40. The nucleic acid vector of any one of claims 35-38, wherein the at least one non-GSH nucleic acid is in an orientation for integration in the GSH in a reverse orientation.

41. The nucleic acid vector of any one of claims 34-40, wherein the at least one non-GSH nucleic acid (a) is operably linked to a promoter, or (b) is not operably linked to a promoter.

42. The nucleic acid vector of claim 41, wherein the at least one non-GSH nucleic acid is operably linked to a promoter, and the promoter is selected from:
(a) a promoter heterologous to the nucleic acid to which it is operably linked;
(b) a promoter that facilitates the tissue-specific expression of the nucleic acid;
(c) a promoter that facilitates the constitutive expression of the nucleic acid;
(d) an inducible promoter;
(e) an immediate early promoter of an animal DNA virus;
(f) an immediate early promoter of an insect virus; and
(g) an insect cell promoter.

43. The nucleic acid vector of claim 42, wherein the inducible promoter is modulated by an agent selected from a small molecule, a metabolite, an oligonucleotide, a riboswitch, a peptide, a peptidomimetic, a hormone, a hormone analog, and light.

44. The nucleic acid vector of claim 43, wherein the agent is selected from tetracycline, cumate, tamoxifen, estrogen, and an antisense oligonucleotide (ASO), rapamycin, FKCsA, blue light, abscisic acid (ABA), and riboswitch.

45. The nucleic acid vector of claim 42, wherein the promoter facilitates tissue-specific expression in a hematopoietic stem cell, a hematopoietic CD34+ cell, and epidermal stem cell, an epithelial stem cell, neural stem cell, a lung progenitor cell, a muscle satellite cell, an intestinal K cell, a neuronal cell, an airway epithelial cell, or a liver progenitor cell.

46. The nucleic acid vector of claim 41 or 42, wherein the promoter is selected from the CMV promoter, β-globin promoter, CAG promoter, AHSP promoter, MND promoter, Wiskott-Aldrich promoter, PKLR promoter, polyhedron (polh) promoter, and immediately early 1 gene (IE-1) promoter.

47. The nucleic acid vector of any one of claims 34-46, wherein the at least one non-GSH nucleic acid comprises a sequence that encodes a coding RNA.

48. The nucleic acid vector of claim 47, wherein the sequence encoding a coding RNA is codon-optimized for expression in a target cell.

49. The nucleic acid vector of claim 47 or 48, wherein the at least one non-GSH nucleic acid encoding a coding RNA further comprises a sequence encoding a signal peptide.

50. The nucleic acid vector of any one of claims 34-49, wherein the at least one non-GSH nucleic acid comprises a sequence encoding:
(a) a protein or a fragment thereof, preferably a human protein or a fragment thereof;
(b) a therapeutic protein or a fragment thereof, an antigen-binding protein, or a peptide;
(c) a suicide gene, optionally Herpes Simplex Virus-1 Thymidine Kinase (HSV-TK);
(d) a viral protein or a fragment thereof;
(e) a nuclease, optionally a Transcription Activator-Like Effector Nuclease (TALEN), a zinc-finger nuclease (ZFN), a meganuclease, a megaTAL, or a CRISPR endonuclease, (e.g., a Cas9 endonuclease or a variant thereof);
(f) a marker, e.g., luciferase or GFP; and/or
(g) a drug resistance protein, e.g., antibiotic resistance gene, e.g., neomycin resistance.

51. The nucleic acid vector of claim 50, wherein the viral protein or a fragment thereof comprises a structural protein (e.g., VP1, VP2, VP3) or a non-structural protein (e.g., Rep protein).

52. The nucleic acid vector of claim 50 or 51, wherein the viral protein or a fragment thereof comprises:
(a) a parvovirus protein or a fragment thereof, optionally VP1, VP2, VP3, NS1, or Rep;
(b) a retrovirus protein or a fragment thereof, optionally an envelope protein, gag, pol, or VSV-G;
(c) an adenovirus protein or a fragment thereof, optionally E1A, E1B, E2A, E2B, E3, E4, or a structural protein (e.g., A, B, C); and/or
(d) a herpes simplex virus protein or a fragment thereof, optionally ICP27, ICP4, or pac.

53. The nucleic acid vector of any one of claims 50-52, wherein the at least one non-GSH nucleic acid encoding a viral protein encodes a surface protein, or a fragment thereof, of a virus.

54. The nucleic acid vector of claim 53, wherein (a) the surface protein or a fragment thereof is an immunogenic surface protein that elicits immune response in a host, (b) the surface protein or a fragment thereof further comprises a signal peptide, (c) the gene encoding the surface protein or fragment thereof is operably linked to an inducible promoter, and/or (d) the nucleic acid encoding the surface protein or a fragment thereof further comprises a suicide gene.

55. The nucleic acid vector of claim 53 or 54, wherein the surface protein is of a coronavirus (e.g., MERS, SARS), influenza virus, respiratory syncytial virus, hepatitis A, hepatitis B, hepatitis C, hepatitis D, hepatitis E, human papillomavirus, dengue virus serotype 1, dengue virus serotype 2, dengue virus serotype 3, dengue virus serotype 4, zika, virus, West Nile virus, yellow fever virus, Chikungunya virus, Mayaro virus, Ebola virus, Marburg virus, or Nipa virus.

56. The nucleic acid vector of any one of claims 53-55, wherein the surface protein is the spike protein of SARS-COV-2.

57. The nucleic acid vector of claim 50, wherein the at least one non-GSH nucleic acid comprising a sequence encoding a protein, or a fragment thereof, is selected from a hemoglobin gene (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), GBA1, preproinsulin, insulin, GIP, GLP-1, CEP290, ATPB1, ATPB11, ABCB4, CPS1, ATP7B, KRT5, KRT14, PLEC1, Col7A1, ITGB4, ITGA6, LAMA3, LAMB3, LAMC2, KIND1, INS, F8 or a fragment thereof (e.g., fragment encoding B-domain deleted polypeptide (e.g., VIII SQ, p-VIII)), IRGM, NOD2, ATG2B, ATG9, ATG5, ATG7, ATG16L1, BECN1, EI24/PIG8, TECPR2, WDR45/WIP14, CHMP2B, CHMP4B, Dynein, EPG5, HspB8, LAMP2, LC3b UVRAG, VCP/p97, ZFYVE26, PARK2/Parkin, PARK6/PINK1, SQSTM1/p62, SMURF, AMPK, ULK1, RPE65, CHM, RPGR, PDE6B, CNGA3, GUCY2D, RS1, ABCA4, MYO7A, HFE, hepcidin, a gene encoding a soluble form (e.g., of the TNFα receptor, IL-6 receptor, IL-12 receptor, or IL-1β receptor), and cystic fibrosis transmembrane conductance regulator (CFTR).

58. The nucleic acid vector of claim 50, wherein the antigen-binding protein is an antibody or an antigen-binding fragment thereof, optionally wherein the antibody or an antigen-binding fragment thereof is selected from an antibody, Fv, F(ab)2, Fab′, dsFv, scFv, sc (Fv)2, half antibody-scFv, tandem scFv, Fab/scFv-Fc, tandem Fab′, single-chain diabody, tandem diabody (TandAb), Fab/scFv-Fc, scFv-Fc, heterodimeric IgG (CrossMab), DART, and diabody.

59. The nucleic acid vector of claim 50 or 51, wherein the antigen-binding protein specifically binds TNFα, CD20, a cytokine (e.g., IL-1, IL-6, BLyS, APRIL, IFN-gamma, etc.), Her2, RANKL, IL-6R, GM-CSF, CCR5, or a pathogen (e.g., bacterial toxin, viral capsid protein, etc.).

60. The nucleic acid vector of any one of claims 50, 58, and 59, wherein the antigen-binding protein is selected from adalimumab, etanercept, infliximab, certolizumab, golimumab, anakinra, rituximab, abatacept, tocilizumab, natalizumab, canakinumab, atacicept, belimumab, ocrelizumab, ofatumumab, fontolizumab, trastuzumab, denosumab, sarilumab, lenzilumab, gimsilumab, siltuximab, leronlimab, and an antigen-binding fragment thereof.

61. The nucleic acid vector of any one of claims 34-46, wherein the at least one non-GSH nucleic acid comprises a sequence encoding a non-coding RNA, optionally wherein the non-coding RNA comprises antisense polynucleotides, lncRNA, piRNA, miRNA, shRNA, siRNA, antisense RNA, snoRNA, snRNA, scaRNA, and/or guide RNA.

62. The nucleic acid vector of claim 61, wherein the non-coding RNA targets a gene selected from DMT-1, ferroportin, TNFα receptor, IL-6 receptor, IL-12 receptor, IL-1β receptor, and a gene encoding a mutated protein (e.g., a mutated HFE, CFTR).

63. The nucleic acid vector of any one of claims 34-62, wherein the at least one non-GSH nucleic acid increases or restores the expression of an endogenous gene of a target cell.

64. The nucleic acid vector of any one of claims 34-62, wherein the at least one non-GSH nucleic acid decreases or eliminates the expression of an endogenous gene of a target cell.

65. The nucleic acid vector of any one of claims 30-64, further comprising:
(a) a transcription regulatory element (e.g., an enhancer, a transcription termination sequence, an untranslated region (5′ or 3′ UTR), a proximal promoter element, a locus control region (e.g., a β-globin LCR or a DNase hypersensitive site (HS) of β-globin LCR), a polyadenylation signal sequence), and/or
(b) a translation regulatory element (e.g., Kozak sequence, woodchuck hepatitis virus post-transcriptional regulatory element).

66. The nucleic acid vector of any of claims 30-65, wherein the nucleic acid vector is selected from a plasmid, minicircle, comsid, artificial chromosome (e.g., BAC), linear covalently closed (LCC) DNA vector (e.g., minicircles, minivectors and miniknots), a linear covalently closed (LCC) vector (e.g., MIDGE, MiLV, ministering, miniplasmids), a mini-intronic plasmid, a pDNA expression vector, or variants thereof.

67. A viral vector comprising at least a portion of the GSH nucleic acid identified in the method of any one of claims 1-29; at least a portion of the GSH in the nucleic acid vector of any one of claims 30-66; at least a portion of any one of the GSHs listed in Table 3; and/or the nucleic acid vector of any one of claims 30-66.

68. The viral vector of claim 67, wherein the viral vector is selected from rAd, AAV, rHSV, retroviral vector, poxvirus vector, lentivirus, vaccinia virus vector, HSV Type 1 (HSV-1)-AAV hybrid vector, baculovirus expression vector system (BEVS), and variants thereof.

69. A cell, comprising the nucleic acid vector of any one of claims 30-66, or the viral vector of claim 67 or 68.

70. The cell of claim 69, wherein the cell is selected from a cell line or a primary cell.

71. The cell of claim 69-70, wherein the cell is a mammalian cell, an insect cell, a bacterial cell, a yeast cell, or a plant cell, optionally wherein the mammalian cell is a human cell or a rodent cell.

72. The cell of any one of claims 69-71, wherein the cell is an insect cell; and the insect cell is derived from a species of lepidoptera.

73. The cell of claim 72, wherein the species of lepidoptera is Spodoptera frugiperda, Spodoptera littoralis, Spodoptera exigua, or Trichoplusia ni.

74. The cell of any one of claims 69-73, wherein the insect cell is Sf9.

75. The cell of any one of claims 69-74, wherein the cell is selected from a hematopoietic cell, hematopoietic progenitor cell, hematopoietic stem cell, erythroid lineage cell, megakaryocyte, erythroid progenitor cell (EPC), CD34+ cell, CD44+ cell, red blood cell, CD36+ cell, mesenchymal stem cell, nerve cell, intestinal cell, intestinal stem cell, gut epithelial cell, endothelial cell, enteroendocrine cell, lung cell, lung progenitor cell, enterocyte, liver cell (e.g., hepatocyte, hepatic stellate cells, Kupffer cells (KCs), liver sinusoidal endothelial cells (LSECs), liver progenitor cell), stem cell, progenitor cell, induced pluripotent stem cell (iPSC), skin fibroblast, macrophage, brain microvascular endothelial cell (BMVECs), neural stem cell, muscle satellite cell, epithelial cell, airway epithelial cell, muscle progenitor cell, erythroid progenitor cell, lymphoid progenitor cell, B lymphoblast cell, B cell, T cell, basophilic Endemic Burkitt Lymphoma (EBL), polychromatic erythroblast, epidermal stem cell, epithelial stem cell, embryonic stem cell, P63-positive keratinocyte-derived stem cell, keratinocyte, pancreatic β-cell, K cell, L cell, HEK293 cell, HEK293T cell, MDCK cell, Vero cell, CHO, BHK1, NS0, Sp2/0, HeLa, A549, and orthochromatic erythroblast.

76. A cell, comprising at least one non-GSH nucleic acid integrated into a GSH in the genome of a cell, wherein the GSH is selected from Table 3.

77. The cell of claim 76, wherein the GSH nucleic acid comprises an untranslated sequence or an intron.

78. The cell of claim 76 or 77, wherein the GSH is selected from SYNTX-GSH1, SYNTX-GSH2, SYNTX-GSH3, and SYNTX-GSH4.

79. The cell of any one of claims 76-78, wherein the at least one non-GSH nucleic acid is integrated into the GSH in a forward orientation.

80. The cell of any one of claims 76-78, wherein the at least one non-GSH nucleic acid is integrated into the GSH in a reverse orientation.

81. The cell of any one of claims 76-80, wherein the at least one non-GSH nucleic acid (a) is operably linked to a promoter, or (b) is not operably linked to a promoter.

82. The cell of claim 81, wherein the at least one non-GSH nucleic acid is operably linked to a promoter, and the promoter is selected from:
(a) a promoter heterologous to the nucleic acid to which it is operably linked;
(b) a promoter that facilitates the tissue-specific expression of the nucleic acid;
(c) a promoter that facilitates the constitutive expression of the nucleic acid;
(d) an inducible promoter;
(e) an immediate early promoter of an animal DNA virus;
(f) an immediate early promoter of an insect virus; and
(g) an insect cell promoter.

83. The cell of claim 82, wherein the inducible promoter is modulated by an agent selected from a small molecule, a metabolite, an oligonucleotide, a riboswitch, a peptide, a peptidomimetic, a hormone, a hormone analog, and light.

84. The cell of claim 83, wherein the agent is selected from tetracycline, cumate, tamoxifen, estrogen, and an antisense oligonucleotide (ASO), rapamycin, FKCsA, blue light, abscisic acid (ABA), and riboswitch.

85. The cell of claim 82, wherein the promoter facilitates tissue-specific expression in a hematopoietic stem cell, a hematopoietic CD34+ cell, and epidermal stem cell, an epithelial stem cell, neural stem cell, a lung progenitor cell, a muscle satellite cell, an intestinal K cell, a neuronal cell, an airway epithelial cell, or a liver progenitor cell.

86. The cell of claim 81 or 82, wherein the promoter is selected from the CMV promoter, β-globin promoter, CAG promoter, AHSP promoter, MND promoter, Wiskott-Aldrich promoter, PKLR promoter, polyhedron (polh) promoter, and immediately early 1 gene (IE-1) promoter.

87. The cell of any one of claims 52-58, wherein the at least one non-GSH nucleic acid comprises a sequence that encodes a coding RNA.

88. The cell of claim 87, wherein the sequence encoding a coding RNA is codon-optimized for expression in a target cell.

89. The cell of claim 87 or 88, wherein the at least one non-GSH nucleic acid encoding a coding RNA further comprises a sequence encoding a signal peptide.

90. The cell of any one of claims 76-89, wherein the at least one non-GSH nucleic acid encodes a coding RNA comprises a sequence encoding:
(a) a protein or a fragment thereof, preferably a human protein or a fragment thereof;
(b) a therapeutic protein or a fragment thereof, an antigen-binding protein, or a peptide;
(c) a suicide gene, optionally Herpes Simplex Virus-1 Thymidine Kinase (HSV-TK);
(d) a viral protein or a fragment thereof;
(e) a nuclease, optionally a Transcription Activator-Like Effector Nuclease (TALEN), a zinc-finger nuclease (ZFN), a meganuclease, a megaTAL, or a CRISPR endonuclease, (e.g., a Cas9 endonuclease or a variant thereof);
(f) a marker, e.g., luciferase or GFP; and/or
(g) a drug resistance protein, e.g., antibiotic resistance gene, e.g., neomycin resistance.

91. The cell of claim 90, wherein the viral protein or a fragment thereof comprises a structural protein (e.g., VP1, VP2, VP3) or a non-structural protein (e.g., Rep protein).

92. The cell of claim 90 or 91, wherein the viral protein or a fragment thereof comprises:
(a) a parvovirus protein or a fragment thereof, optionally VP1, VP2, VP3, NS1, or Rep;
(b) a retrovirus protein or a fragment thereof, optionally an envelope protein, gag, pol, or VSV-G;
(c) an adenovirus protein or a fragment thereof, optionally E1A, E1B, E2A, E2B, E3, E4, or a structural protein (e.g., A, B, C); and/or
(d) a herpes simplex virus protein or a fragment thereof, optionally ICP27, ICP4, or pac.

93. The cell of any one of claims 90-92, wherein the gene encoding a viral protein encodes a surface protein, or a fragment thereof, of a virus.

94. The cell of claim 93, wherein (a) the surface protein is an immunogenic surface protein or a fragment thereof that elicits immune response, (b) the surface protein or a fragment thereof further comprises a signal peptide, (c) the gene is operably linked to an inducible promoter, and/or (d) the nucleic acid encoding the surface protein or a fragment thereof further comprises a suicide gene.

95. The cell of claim 93 or 94, wherein the surface protein is of a coronavirus (e.g., MERS, SARS), influenza virus, respiratory syncytial virus, hepatitis A, hepatitis B, hepatitis C, hepatitis D, hepatitis E, human papillomavirus, dengue virus serotype 1, dengue virus serotype 2, dengue virus serotype 3, dengue virus serotype 4, zika, virus, West Nile virus, yellow fever virus, Chikungunya virus, Mayaro virus, Ebola virus, Marburg virus, or Nipa virus.

96. The cell of any one of claims 93-95, wherein the surface protein is the spike protein of SARS-COV-2.

97. The cell of claim 90, wherein the at least one non-GSH nucleic acid comprising a sequence encoding a protein, or a fragment thereof, is selected from a hemoglobin gene (HBA1, HBA2, HBB, HBG1, HBG2, HBD, HBE1, and/or HBZ), alpha-hemoglobin stabilizing protein (AHSP), coagulation factor VIII, coagulation factor IX, von Willebrand factor, dystrophin or truncated dystrophin, micro-dystrophin, utrophin or truncated utrophin, micro-utrophin, usherin (USH2A), GBA1, preproinsulin, insulin, GIP, GLP-1, CEP290, ATPB1, ATPB11, ABCB4, CPS1, ATP7B, KRT5, KRT14, PLEC1, Col7A1, ITGB4, ITGA6, LAMA3, LAMB3, LAMC2, KIND1, INS, F8 or a fragment thereof (e.g., fragment encoding B-domain deleted polypeptide (e.g., VIII SQ, p-VIII)), IRGM, NOD2, ATG2B, ATG9, ATG5, ATG7, ATG16L1, BECN1, EI24/PIG8, TECPR2, WDR45/WIP14, CHMP2B, CHMP4B, Dynein, EPG5, HspB8, LAMP2, LC3b UVRAG, VCP/p97, ZFYVE26, PARK2/Parkin, PARK6/PINK1, SQSTM1/p62, SMURF, AMPK, ULK1, RPE65, CHM, RPGR, PDE6B, CNGA3, GUCY2D, RS1, ABCA4, MYO7A, HFE, hepcidin, a gene encoding a soluble form (e.g., of the TNFα receptor, IL-6 receptor, IL-12 receptor, or IL-1β receptor), and cystic fibrosis transmembrane conductance regulator (CFTR).

98. The cell of claim 90, wherein the antigen-binding protein is an antibody or an antigen-binding fragment thereof, optionally wherein the antibody or an antigen-binding fragment thereof is selected from an antibody, Fv, F(ab)2, Fab′, dsFv, scFv, sc (Fv)2, half antibody-scFv, tandem scFv, Fab/scFv-Fc, tandem Fab′, single-chain diabody, tandem diabody (TandAb), Fab/scFv-Fc, scFv-Fc, heterodimeric IgG (CrossMab), DART, and diabody.

99. The cell of claim 90 or 91, wherein the antigen-binding protein specifically binds TNFα, CD20, a cytokine (e.g., IL-1, IL-6, BLyS, APRIL, IFN-gamma, etc.), Her2, RANKL, IL-6R, GM-CSF, CCR5, or a pathogen (e.g., bacterial toxin, viral capsid protein, etc.).

100. The cell of any one of claims 90, 98, and 99, wherein the antigen-binding protein is selected from adalimumab, etanercept, infliximab, certolizumab, golimumab, anakinra, rituximab, abatacept, tocilizumab, natalizumab, canakinumab, atacicept, belimumab, ocrelizumab, ofatumumab, fontolizumab, trastuzumab, denosumab, sarilumab, lenzilumab, gimsilumab, siltuximab, leronlimab, and an antigen-binding fragment thereof.

101. The cell of any one of claims 76-86, wherein the at least one non-GSH nucleic acid comprises a sequence encoding a non-coding RNA, optionally wherein the non-coding RNA comprises lncRNA, piRNA, miRNA, shRNA, siRNA, antisense RNA, snoRNA, snRNA, scaRNA, and/or guide RNA.

102. The cell of claim 101, wherein the non-coding RNA targets a gene selected from DMT-1, ferroportin, TNFα receptor, IL-6 receptor, IL-12 receptor, IL-1β receptor, a gene encoding a mutated protein (e.g., a mutated HFE, CFTR).

103. The cell of any one of claims 76-102, wherein the at least one non-GSH nucleic acid increases or restores the expression of an endogenous gene of a target cell.

104. The cell of any one of claims 76-102, wherein the at least one non-GSH nucleic acid decreases or eliminates the expression of an endogenous gene of a target cell.

105. The cell of any one of claims 76-104, wherein the at least one non-GSH nucleic acid further comprises:
(a) a transcription regulatory element (e.g., an enhancer, a transcription termination sequence, an untranslated region (5′ or 3′ UTR), a proximal promoter element, a locus control region (e.g., a β-globin LCR or a DNase hypersensitive site (HS) of β-globin LCR), a polyadenylation signal sequence), and/or
(b) a translation regulatory element (e.g., Kozak sequence, woodchuck hepatitis virus post-transcriptional regulatory element).

106. The cell of any one of claims 76-105, wherein the cell is selected from a cell line or a primary cell.

107. The cell of any one of claims 76-106, wherein the cell is a mammalian cell, an insect cell, a bacterial cell, a yeast cell, or a plant cell, optionally wherein the mammalian cell is a human cell or a rodent cell.

108. The cell of any one of claims 76-107, wherein the cell is an insect cell; and the insect cell is derived from a species of lepidoptera.

109. The cell of claim 108, wherein the species of lepidoptera is Spodoptera frugiperda, Spodoptera littoralis, Spodoptera exigua, or Trichoplusia ni.

110. The cell of any one of claims 107-109, wherein the insect cell is Sf9.

111. The cell of any one of claims 76-110, wherein the cell is selected from a hematopoietic cell, hematopoietic progenitor cell, hematopoietic stem cell, erythroid lineage cell, megakaryocyte, erythroid progenitor cell (EPC), CD34+ cell, CD44+ cell, red blood cell, CD36+ cell, mesenchymal stem cell, nerve cell, intestinal cell, intestinal stem cell, gut epithelial cell, endothelial cell, enteroendocrine cell, lung cell, lung progenitor cell, enterocyte, liver cell (e.g., hepatocyte, hepatic stellate cells, Kupffer cells (KCs), liver sinusoidal endothelial cells (LSECs), liver progenitor cell), stem cell, progenitor cell, induced pluripotent stem cell (iPSC), skin fibroblast, macrophage, brain microvascular endothelial cell (BMVECs), neural stem cell, muscle satellite cell, epithelial cell, airway epithelial cell, muscle progenitor cell, erythroid progenitor cell, lymphoid progenitor cell, B lymphoblast cell, B cell, T cell, basophilic Endemic Burkitt Lymphoma (EBL), polychromatic erythroblast, epidermal stem cell, epithelial stem cell, embryonic stem cell, P63-positive keratinocyte-derived stem cell, keratinocyte, pancreatic β-cell, K cell, L cell, HEK293 cell, HEK293T cell, MDCK cell, Vero cell, CHO, BHK1, NS0, Sp2/0, HeLa, A549, and orthochromatic erythroblast.

112. A pharmaceutical composition, comprising the nucleic acid vector of any one of claims 30-66, the viral vector of claim 67 or 68, and/or the cell of any one of claims 69-111.

113. A transgenic organism comprising at least one non-GSH nucleic acid integrated into a GSH in the genome of a cell, wherein the GSH is selected from Table 3.

114. The transgenic organism of claim 113, wherein the GSH is selected from SYNTX-GSH1, SYNTX-GSH2, SYNTX-GSH3, and SYNTX-GSH4.

115. A transgenic organism, comprising the cell of any one of claims 69-114.

116. The transgenic organism of claim 115, wherein the organism is a mammal or a plant, optionally wherein the mammal is a rodent (e.g., mouse, rat), a goat, a sheep, a chicken, a llama, or a rabbit.

117. A method of inserting at least one non-GSH nucleic acid into a GSH locus of a cell, the method comprising introducing the nucleic acid vector of any one of claims 30-66, the viral vector of claim 67 or 68, or a pharmaceutical composition of claim 112 into the cell, whereby homologous recombination of the GSH 5′ homology arm and the GSH 3′ homology arm flanking the non-GSH nucleic acid with the GSH locus in the genome integrates the non-GSH nucleic acid into the GSH locus.

118. The method of claim 117, wherein the non-GSH nucleic acid is integrated into the GSH in a forward orientation.

119. The method of claim 117, wherein the non-GSH nucleic acid is integrated into the GSH in a reverse orientation.

120. A method of preventing or treating a disease, comprising administering to a subject in need thereof an effective amount of the nucleic acid vector of any one of claims 30-66, the viral vector of claim 67 or 68, the cell of any one of claims 69-111, and/or the pharmaceutical composition of claim 112.

121. The method of claim 120, wherein the disease is selected from an infection, endothelial dysfunction, cystic fibrosis, cardiovascular disease, renal disease, cancer, hemoglobinopathy, anemia, hemophilia (e.g., hemophilia A), myeloproliferative disorder, coagulopathy, sickle cell disease, alpha-thalassemia, beta-thalassemia, Fanconi anemia, familial intrahepatic cholestasis, skin genetic disorder (e.g., epidermolysis bullosa), ocular genetic disease (e.g., inherited retinal dystrophies, e.g., Leber congenital amaurosis (LCA), retinitis pigmentosa (RP), choroideremia, achromatopsia, retinoschisis, Stargardt disease, Usher syndrome type 1B), Fabry, Gaucher, Nieman-Pick A, Nieman-Pick B, GM1 Gangliosidosis, Mucopolysaccharidosis (MPS) I (Hurler, Scheie, Hurler/Scheie), MPS II (Hunter), MPS VI (Maroteaux-Lamy), hematologic cancer, hemochromatosis, hereditary hemochromatosis, juvenile hemochromatosis, cirrhosis, hepatocellular carcinoma, pancreatitis, diabetes mellitus, cardiomyopathy, arthritis, hypogonadism, heart disease, heart attack, hypothyroidism, glucose intolerance, arthropathy, liver fibrosis, Wilson's disease, ulcerative colitis, Crohn's disease, Tay-Sachs disease, neurodegenerative disorder, Spinal muscular atrophy type 1, Huntington's disease, Canavan's disease, rheumatoid arthritis, inflammatory bowel disease, psoriatic arthritis, juvenile chronic arthritis, psoriasis, and ankylosing spondylitis, and autoimmune disease, neurodegenerative disease (e.g., Alzheimer's disease, Parkinson's disease, Huntington's disease, ataxias), inflammatory disease, inflammatory bowel disease, Crohn's disease, rheumatoid arthritis, lupus, multiple sclerosis, chronic obstructive pulmonary disease/COPD, pulmonary fibrosis, Sjogren's disease, hyperglycemic disorders, type I diabetes, type II diabetes, insulin resistance, hyperinsulinemia, insulin-resistant diabetes (e.g. Mendenhall's Syndrome, Werner Syndrome, leprechaunism, and lipoatrophic diabetes), dyslipidemia, hyperlipidemia, elevated low-density lipoprotein (LDL), depressed high density lipoprotein (HDL), elevated triglycerides, metabolic syndrome, liver disease, renal disease, cardiovascular disease, ischemia, stroke, complications during reperfusion, muscle degeneration, atrophy, symptoms of aging (e.g., muscle atrophy, frailty, metabolic disorders, low grade inflammation, atherosclerosis, stroke, age-associated dementia and sporadic form of Alzheimer's disease, pre-cancerous states, and psychiatric conditions including depression), spinal cord injury, arteriosclerosis, infectious diseases (e.g., bacterial, fungal, viral), AIDS, tuberculosis, defects in embryogenesis, infertility, lysosomal storage diseases, activator deficiency/GM2 gangliosidosis, alpha-mannosidosis, aspartylglucoaminuria, cholesteryl ester storage disease, chronic hexosaminidase A deficiency, cystinosis, Danon disease, Farber disease, fucosidosis, galactosialidosis, Gaucher Disease (Types I, II and III), GM1 Gangliosidosis, (infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Neuronal ceroid lipofuscinoses, CLN6 disease, Jansky-Bielschowsky disease, Pompe disease, pycnodysostosis, Sandhoff disease, Schindler disease, and Wolman disease.

122. The method of claim 121, wherein the infection is a bacterial infection, fungal infection, or a viral infection.

123. The method of claim 121 or 122, wherein the infection is the viral infection; and the viral infection is by a coronavirus (e.g., MERS, SARS), influenza virus, respiratory syncytial virus, hepatitis A, hepatitis B, hepatitis C, hepatitis D, hepatitis E, human papillomavirus, dengue virus serotype 1, dengue virus serotype 2, dengue virus serotype 3, dengue virus serotype 4, zika, virus, West Nile virus, yellow fever virus, Chikungunya virus, Mayaro virus, Ebola virus, Marburg virus, or Nipa virus.

124. The method of claim 122 or 123, wherein the viral infection is by SARS-COV-2.

125. The method of any one of claims 120-124, wherein the nucleic acid vector, the cell, and/or the pharmaceutical composition is administered to the subject via intravascular, intracerebral, parenteral, intraperitoneal, intravenous, epidural, intraspinal, intrasternal, intra-articular, intra-synovial, intrathecal, intratumoral, intra-arterial, intracardiac, intramuscular, intranasal, intrapulmonary, skin graft, or oral administration.

126. The method of any one of claims 120-125, wherein the cell is autologous or allogeneic to the subject.

127. A method of modulating the level and/or activity of a protein in a cell, the method comprising introducing the nucleic acid vector of any one of claims 30-66, the viral vector of claim 67 or 68, and/or the pharmaceutical composition of claim 112 to the cell.

128. The method of claim 127, wherein the level and/or activity is increased.

129. The method of claim 128, wherein the level and/or activity is decreased or eliminated.

130. A method of manufacturing a biologic, the method comprising:
(a) culturing (i) the cell comprising the nucleic acid vector of any one of claims 30-66, (ii) the cell comprising the viral vector of claim 67 or 68, or (iii) the cell of any one of claims 69-111; and recovering the expressed biologic; or
(b) recovering the expressed biologic from the transgenic organism of claim 115 or 116.

131. The method of claim 130, wherein the biologic is an antigen-binding protein.

132. The method of claim 130 or 131, wherein the biologic is an antibody or an antigen-binding fragment thereof, optionally wherein the antibody or an antigen-binding fragment thereof is selected from an antibody, Fv, F(ab)2, Fab′, dsFv, scFv, sc (Fv)2, half antibody-scFv, tandem scFv, Fab/scFv-Fc, tandem Fab′, single-chain diabody, tandem diabody (TandAb), Fab/scFv-Fc, scFv-Fc, heterodimeric IgG (CrossMab), DART, and diabody.

133. The method of any one of claims 130-132, wherein the biologic specifically binds TNFα, CD20, a cytokine (e.g., IL-1, IL-6, BLyS, APRIL, IFN-gamma, etc.), Her2, RANKL, IL-6R, GM-CSF, or CCR5.

134. The method of any one of claims 130-133, wherein the biologic is selected from adalimumab, etanercept, infliximab, certolizumab, golimumab, anakinra, rituximab, abatacept, tocilizumab, natalizumab, canakinumab, atacicept, belimumab, ocrelizumab, ofatumumab, fontolizumab, trastuzumab, denosumab, sarilumab, lenzilumab, gimsilumab, siltuximab, leronlimab, and an antigen-binding fragment thereof.

135. The method of any one of claims 130-134, wherein the biologic is a therapeutic protein, optionally wherein the therapeutic protein is an insulin.

136. A method of manufacturing a viral vector (e.g., gene therapy or vaccine), the method comprising:
(1) providing a host cell comprising
(i) a nucleic acid sequence comprising at least one functional virus origin of replication (e.g., at least one ITR nucleotide sequence),
optionally further comprising a nucleic acid operably linked to a promoter for expression in a target cell,
(ii) a nucleic acid sequence comprising at least one gene encoding one or more viral structural proteins (e.g., capsid proteins, e.g., gag, VP1,VP2, VP3, a variant thereof), operably linked to at least one expression control sequence for expression in a host cell, and
(iii) a nucleic acid sequence comprising at least one gene encoding one or more replication proteins (e.g., Rep, pol) operably linked to at least one expression control sequence for expression in a host cell,
optionally wherein the at least one replication protein comprises (a) a Rep52 or a Rep40 coding sequence or a fragment thereof that encodes a functional replication protein, operably linked to at least one expression control sequence for expression in a host cell, and/or (b) a Rep78 or a Rep68 coding sequence operably linked to at least one expression control sequence for expression in a host cell;
wherein at least one of (i), (ii), and (iii) is stably integrated into at least one GSH selected from Table 3 in the host cell genome, and the at least one vector, if/when present, comprises the remainder of the (i), (ii), and (iii) that is not stably integrated in the host cell genome; and
(2) maintaining the host cell under conditions such that a recombinant viral vector is produced.

137. The method of claim 136, wherein (ii) or (iii) is integrated into a GSH.

138. The method of claim 136, wherein (ii) and (iii) are integrated into a GSH.

139. The method of any one of claims 136-138, wherein the at least one functional virus origin of replication (e.g., at least one ITR nucleotide sequence) comprises:
(a) a dependoparvovirus ITR, and/or
(b) an AAV ITR, optionally an AAV2 ITR.

140. The method of any one of claims 136-139, wherein the at least one expression control sequence for expression in the host cell comprises:
(a) a promoter, and/or
(b) a Kozak-like expression control sequence.

141. The method of claim 140, wherein the promoter comprises:
(a) an immediate early promoter of an animal DNA virus,
(b) an immediate early promoter of an insect virus,
(c) an insect cell promoter, or
(d) an inducible promoter.

142. The method of claim 141, wherein the animal DNA virus is cytomegalovirus (CMV), a dependoparvovirus, or AAV.

143. The method of claim 141, wherein the insect virus is a lepidopteran virus or a baculovirus, optionally wherein the baculovirus is Autographa californica multicapsid nucleopolyhedrovirus (AcMNPV).

144. The method of claim 140 or 141, wherein the promoter is a polyhedrin (polh) or immediately early 1 gene (IE-1) promoter.

145. The method of claim 140 or 141, wherein the promoter is an inducible promoter.

146. The method of claim 145, wherein the inducible promoter is modulated by an agent selected from a small molecule, a metabolite, an oligonucleotide, a riboswitch, a peptide, a peptidomimetic, a hormone, a hormone analog, and light.

147. The method of claim 146, wherein the agent is selected from tetracycline, cumate, tamoxifen, estrogen, and an antisense oligonucleotide (ASO), rapamycin, FKCsA, blue light, abscisic acid (ABA), and riboswitch.

148. The method of any one of claims 136-147, wherein:
(a) the viral replication protein is an AAV replication protein, optionally Rep52 and/or Rep78 proteins; and/or
(b) the viral structural protein is an AAV capsid protein.

149. The method of claim 148, wherein the AAV is AAV2.

150. The method of any one of claims 136-149, wherein the method manufactures the viral vector of claim 67 or 68.

151. The method of any one of claims 136-150, wherein the host cell is a mammalian cell or an insect cell.

152. The method of claim 151, wherein the host cell is a mammalian cell; and the mammalian cell is a human cell or a rodent cell.

153. The method of claim 151 or 152, wherein the mammalian cell is selected from HEK293, HEK293T, HeLa, and A549.

154. The method of claim 151, wherein the host cell is an insect cell; and the insect cell is derived from a species of lepidoptera.

155. The method of claim 154, wherein the species of lepidoptera is Spodoptera frugiperda, Spodoptera littoralis, Spodoptera exigua, or Trichoplusia ni.

156. The method of any one of claims 151, 154, and 155, wherein the insect cell is Sf9.

157. The method of any one of claims 136-156, wherein the viral vector is selected from adeno virus-derived vectors (e.g., AAV), retrovirus, lentivirus-derived vectors (e.g., lentivirus), herpes virus-derived vectors, and alphavirus-derived vectors (e.g., Semliki forest virus (SFV) vector).

158. A kit, comprising the nucleic acid vector of any one of claims 30-66, the viral vector of claim 67 or 68, the cell of any one of claims 69-111, and/or the pharmaceutical composition of claim 112.
